SEQUENCE LISTING (172 sequences)

SEQ ID NO: 1 - DNA, 21 bp, Artificial
  siRNA
gccugaaccuucucuugagtt

SEQ ID NO: 2 - DNA, 21 bp, Artificial
  siRNA
ggacgguauggagcauguutt

SEQ ID NO: 3 - DNA, 21 bp, Artificial
  siRNA
cgaaaguuaacaaaacucctt

SEQ ID NO: 4 - DNA, 21 bp, Artificial
  siRNA
ccauucguuuauuagaaactt

SEQ ID NO: 5 - DNA, 21 bp, Artificial
  siRNA
cguaaaccuuccugaaagatt

SEQ ID NO: 6 - DNA, 21 bp, Artificial
  siRNA
ccuuuaauuaccuuccuagtt

SEQ ID NO: 7 - DNA, 21 bp, Artificial
  siRNA
gacucauugaacauaugcatt

SEQ ID NO: 8 - DNA, 21 bp, Artificial
  siRNA
gaauaugccuguuccugugtt

SEQ ID NO: 9 - DNA, 21 bp, Artificial
  siRNA
ggcccauauauugcauuuatt

SEQ ID NO: 10 - DNA, 21 bp, Artificial
  siRNA
ggugucauuugacaugguutt

SEQ ID NO: 11 - DNA, 21 bp, Artificial
  siRNA
ggaggcagauaaugcuggutt

SEQ ID NO: 12 - DNA, 21 bp, Artificial
  siRNA
ggcagacaagucaaccgugtt

SEQ ID NO: 13 - DNA, 21 bp, Artificial
  siRNA
ggcauggccacuacuuugutt

SEQ ID NO: 14 - DNA, 21 bp, Artificial
  siRNA
ggacagucaaggugauauatt

SEQ ID NO: 15 - DNA, 21 bp, Artificial
  siRNA
ggaaugcauguaugcugugtt

SEQ ID NO: 16 - DNA, 21 bp, Artificial
  siRNA
ccaauccuacuaauaaacctt

SEQ ID NO: 17 - DNA, 21 bp, Artificial
  siRNA
ggaauauauggcaacuuugtt

SEQ ID NO: 18 - DNA, 21 bp, Artificial
  siRNA
gggcacacuacacuauuaatt

SEQ ID NO: 19 - DNA, 21 bp, Artificial
  siRNA
cgaguuaccucguccgagutt

SEQ ID NO: 20 - DNA, 21 bp, Artificial
  siRNA
gcaugcuaugguguucuuctt

SEQ ID NO: 21 - DNA, 21 bp, Artificial
  siRNA
ggugcacaucuucucucugtt

SEQ ID NO: 22 - DNA, 21 bp, Artificial
  siRNA
ggccuacaucacauggauctt

SEQ ID NO: 23 - DNA, 21 bp, Artificial
  siRNA
gguaccuaucaguuuguautt

SEQ ID NO: 24 - DNA, 21 bp, Artificial
  siRNA
cgacgacauuuugugaauatt

SEQ ID NO: 25 - DNA, 21 bp, Artificial
  siRNA
ccgauuacuuaaguuuccgtt

SEQ ID NO: 26 - DNA, 21 bp, Artificial
  siRNA
gagagaaggcaugucuuugtt

SEQ ID NO: 27 - DNA, 21 bp, Artificial
  siRNA
gagaaggcaugucuuugggtt

SEQ ID NO: 28 - DNA, 21 bp, Artificial
  siRNA
gccagaauacaaguauguatt

SEQ ID NO: 29 - DNA, 21 bp, Artificial
  siRNA
ccauccugagagaugugautt

SEQ ID NO: 30 - DNA, 21 bp, Artificial
  siRNA
gcgaauuuugugugauuuctt

SEQ ID NO: 31 - DNA, 21 bp, Artificial
  siRNA
cccaagcacuuuaugcauatt

SEQ ID NO: 32 - DNA, 21 bp, Artificial
  siRNA
ggccaagcaauaucugucutt

SEQ ID NO: 33 - DNA, 21 bp, Artificial
  siRNA
gggucuucaggaaagucuctt

SEQ ID NO: 34 - DNA, 21 bp, Artificial
  siRNA
ccuaugugaaaggaacccatt

SEQ ID NO: 35 - DNA, 21 bp, Artificial
  siRNA
cgagaccuaugacuugcuctt

SEQ ID NO: 36 - DNA, 21 bp, Artificial
  siRNA
ggucugaaccaguggaugutt

SEQ ID NO: 37 - DNA, 21 bp, Artificial
  siRNA
ggaccagcaaaucacugcctt

SEQ ID NO: 38 - DNA, 21 bp, Artificial
  siRNA
ggagcagacuaggcauauctt

SEQ ID NO: 39 - DNA, 21 bp, Artificial
  siRNA
gcagacuaggcauaucuuutt

SEQ ID NO: 40 - DNA, 21 bp, Artificial
  siRNA
ggaggccauuuggucuucatt

SEQ ID NO: 41 - DNA, 21 bp, Artificial
  siRNA
ggcgauauggagguguacatt

SEQ ID NO: 42 - DNA, 21 bp, Artificial
  siRNA
cgguuucaugccgacuucatt

SEQ ID NO: 43 - DNA, 21 bp, Artificial
  siRNA
cgaagacagcaugauauautt

SEQ ID NO: 44 - DNA, 21 bp, Artificial
  siRNA
ggcagccagacugcauuuatt

SEQ ID NO: 45 - DNA, 21 bp, Artificial
  siRNA
ccaaccauaagaaaucagatt

SEQ ID NO: 46 - DNA, 21 bp, Artificial
  siRNA
gcucuaaggaagacuucaatt

SEQ ID NO: 47 - DNA, 21 bp, Artificial
  siRNA
ggaugcuauccagagggactt

SEQ ID NO: 48 - DNA, 21 bp, Artificial
  siRNA
ggaagacuucaagucccaatt

SEQ ID NO: 49 - DNA, 21 bp, Artificial
  siRNA
gguauauucauucaauguctt

SEQ ID NO: 50 - DNA, 21 bp, Artificial
  siRNA
ggacagugucgucguagaatt

SEQ ID NO: 51 - DNA, 21 bp, Artificial
  siRNA
ggcuggauggaugcauuuatt

SEQ ID NO: 52 - DNA, 21 bp, Artificial
  siRNA
ccugcuaagagaauuacaatt

SEQ ID NO: 53 - DNA, 21 bp, Artificial
  siRNA
ccauucuagauugcauuuatt

SEQ ID NO: 54 - DNA, 21 bp, Artificial
  siRNA
ggguucuucccauagcccatt

SEQ ID NO: 55 - DNA, 21 bp, Artificial
  siRNA
gcguggcaaaguccaagautt

SEQ ID NO: 56 - DNA, 21 bp, Artificial
  siRNA
ggcaugugguuuugaccuutt

SEQ ID NO: 57 - DNA, 21 bp, Artificial
  siRNA
ggugaucaagaagguaaagtt

SEQ ID NO: 58 - DNA, 21 bp, Artificial
  siRNA
cgugagccgaucauuaagctt

SEQ ID NO: 59 - DNA, 21 bp, Artificial
  siRNA
ggaaucauugcaugcuuaatt

SEQ ID NO: 60 - DNA, 21 bp, Artificial
  siRNA
gccaucaagaggagaaacgtt

SEQ ID NO: 61 - DNA, 21 bp, Artificial
  siRNA
gccuugauauaagguguautt

SEQ ID NO: 62 - DNA, 21 bp, Artificial
  siRNA
ccauuuggaguguuugaagtt

SEQ ID NO: 63 - DNA, 21 bp, Artificial
  siRNA
ggaaauaauaagggaguaatt

SEQ ID NO: 64 - DNA, 21 bp, Artificial
  siRNA
ggcuagccagagaauuacctt

SEQ ID NO: 65 - DNA, 21 bp, Artificial
  siRNA
guggcagugugaccaagaatt

SEQ ID NO: 66 - DNA, 21 bp, Artificial
  siRNA
gccaggacaccucaagucutt

SEQ ID NO: 67 - DNA, 21 bp, Artificial
  siRNA
ggcucugcuagaccaagaatt

SEQ ID NO: 68 - DNA, 21 bp, Artificial
  siRNA
gcagccuaaucgacauagatt

SEQ ID NO: 69 - DNA, 21 bp, Artificial
  siRNA
gcuccugaucaugguguautt

SEQ ID NO: 70 - DNA, 21 bp, Artificial
  siRNA
ccaacuuuuuaugucgguutt

SEQ ID NO: 71 - DNA, 21 bp, Artificial
  siRNA
ggagaaaaggagacuucaatt

SEQ ID NO: 72 - DNA, 21 bp, Artificial
  siRNA
ccucaucauguggagugcatt

SEQ ID NO: 73 - DNA, 21 bp, Artificial
  siRNA
cccuaagcaaugugcauactt

SEQ ID NO: 74 - DNA, 21 bp, Artificial
  siRNA
gcaaggcaaguucuaugactt

SEQ ID NO: 75 - DNA, 21 bp, Artificial
  siRNA
ggaagagaguuguauguactt

SEQ ID NO: 76 - DNA, 21 bp, Artificial
  siRNA
ggcuggagauuuucacauatt

SEQ ID NO: 77 - DNA, 21 bp, Artificial
  siRNA
ggaugugcugccuaaguautt

SEQ ID NO: 78 - DNA, 21 bp, Artificial
  siRNA
ggccuuugagguugugacutt

SEQ ID NO: 79 - DNA, 21 bp, Artificial
  siRNA
cguguaaauagugguaaautt

SEQ ID NO: 80 - DNA, 21 bp, Artificial
  siRNA
cgaacagugaucuacgucctt

SEQ ID NO: 81 - DNA, 21 bp, Artificial
  siRNA
ccgaucacaaagccuucuatt

SEQ ID NO: 82 - DNA, 21 bp, Artificial
  siRNA
gccuucuacguccuuuuuatt

SEQ ID NO: 83 - DNA, 21 bp, Artificial
  siRNA
gccaaaaaacuggguguactt

SEQ ID NO: 84 - DNA, 21 bp, Artificial
  siRNA
ccaacauugacuucuuacutt

SEQ ID NO: 85 - DNA, 21 bp, Artificial
  siRNA
ggugacacgcaugauccuctt

SEQ ID NO: 86 - DNA, 21 bp, Artificial
  siRNA
gcacuaccaaccuguucautt

SEQ ID NO: 87 - DNA, 21 bp, Artificial
  siRNA
gcauccugacgguugaugutt

SEQ ID NO: 88 - DNA, 21 bp, Artificial
  siRNA
ccuguguuucauccugugctt

SEQ ID NO: 89 - DNA, 21 bp, Artificial
  siRNA
gguuacaucgcauagugcctt

SEQ ID NO: 90 - DNA, 21 bp, Artificial
  siRNA
ggucuauucaccgccaucatt

SEQ ID NO: 91 - DNA, 21 bp, Artificial
  siRNA
ggagacaaagugcauauaatt

SEQ ID NO: 92 - DNA, 21 bp, Artificial
  siRNA
gcccaaaugcugcuguggutt

SEQ ID NO: 93 - DNA, 21 bp, Artificial
  siRNA
ccaggcauguguuggcuautt

SEQ ID NO: 94 - DNA, 21 bp, Artificial
  siRNA
ccuccugaucaauaaguautt

SEQ ID NO: 95 - DNA, 21 bp, Artificial
  siRNA
cgaacuccuuacuuaugaatt

SEQ ID NO: 96 - DNA, 21 bp, Artificial
  siRNA
ggaacgacaguggguagautt

SEQ ID NO: 97 - DNA, 21 bp, Artificial
  siRNA
ggcuggucucgugguaaaatt

SEQ ID NO: 98 - DNA, 21 bp, Artificial
  siRNA
gcgaguguuuacggguguutt

SEQ ID NO: 99 - DNA, 21 bp, Artificial
  siRNA
cccaugagcuccuaucaagtt

SEQ ID NO: 100 - DNA, 21 bp, Artificial
  siRNA
ggcacagguguaaauauagtt

SEQ ID NO: 101 - DNA, 21 bp, Artificial
  siRNA
cgugcucuucuguucucaatt

SEQ ID NO: 102 - DNA, 21 bp, Artificial
  siRNA
ggccaucaugaagauugcutt

SEQ ID NO: 103 - DNA, 21 bp, Artificial
  siRNA
gggacgaagaaaagguguutt

SEQ ID NO: 104 - DNA, 21 bp, Artificial
  siRNA
ggugaugaauuuaccaucatt

SEQ ID NO: 105 - DNA, 21 bp, Artificial
  siRNA
gcacuuucaaucgucaucatt

SEQ ID NO: 106 - DNA, 21 bp, Artificial
  siRNA
gcccaguagcagcuauagutt

SEQ ID NO: 107 - DNA, 21 bp, Artificial
  siRNA
ggagguggucuguaaaaggtt

SEQ ID NO: 108 - DNA, 21 bp, Artificial
  siRNA
ggcaucuagugacugucuatt

SEQ ID NO: 109 - DNA, 21 bp, Artificial
  siRNA
gggagacuugcuuacugugtt

SEQ ID NO: 110 - DNA, 21 bp, Artificial
  siRNA
gguucagaauucuuaaguctt

SEQ ID NO: 111 - DNA, 21 bp, Artificial
  siRNA
ggaugugcagaaagugcugtt

SEQ ID NO: 112 - DNA, 21 bp, Artificial
  siRNA
ccucucuacaccuaucuuatt

SEQ ID NO: 113 - DNA, 21 bp, Artificial
  siRNA
ccaaacuguacagacucuctt

SEQ ID NO: 114 - DNA, 21 bp, Artificial
  siRNA
ccagauauccucggcaacctt

SEQ ID NO: 115 - DNA, 21 bp, Artificial
  siRNA
gccaagcaggagauuaacatt

SEQ ID NO: 116 - DNA, 21 bp, Artificial
  siRNA
ggccauuggcuaauugcactt

SEQ ID NO: 117 - DNA, 21 bp, Artificial
  siRNA
ccgaugugaaaggcuguagtt

SEQ ID NO: 118 - DNA, 21 bp, Artificial
  siRNA
gcaagauugucauugacuutt

SEQ ID NO: 119 - DNA, 21 bp, Artificial
  siRNA
ggaguucuaugucugcauctt

SEQ ID NO: 120 - DNA, 21 bp, Artificial
  siRNA
gcacgaagucacaaucuuutt

SEQ ID NO: 121 - DNA, 21 bp, Artificial
  siRNA
gcaauucgagauuaccaggtt

SEQ ID NO: 122 - DNA, 21 bp, Artificial
  siRNA
cgacaacauuguugaguautt

SEQ ID NO: 123 - DNA, 21 bp, Artificial
  siRNA
gccaacgacuuagccuuactt

SEQ ID NO: 124 - DNA, 21 bp, Artificial
  siRNA
ccgaaagugugaccucugatt

SEQ ID NO: 125 - DNA, 22 bp, Artificial
  siRNA
ggcuaugccacuggacucattc

SEQ ID NO: 126 - DNA, 20 bp, Artificial
  siRNA
gaguuccugaugcauuautt

SEQ ID NO: 127 - DNA, 21 bp, Artificial
  siRNA
ggauuucauuucagguggatt

SEQ ID NO: 128 - DNA, 21 bp, Artificial
  siRNA
cccucacaauuugucucagtt

SEQ ID NO: 129 - DNA, 21 bp, Artificial
  siRNA
ggccauaguccuguucacctt

SEQ ID NO: 130 - DNA, 21 bp, Artificial
  siRNA
gagcuucuucaagcgcagctt

SEQ ID NO: 131 - DNA, 21 bp, Artificial
  siRNA
gcuguuuguguugaaugcgtt

SEQ ID NO: 132 - DNA, 21 bp, Artificial
  siRNA
gggauuuucgguuauuauatt

SEQ ID NO: 133 - DNA, 21 bp, Artificial
  siRNA
gggcucaguaaaauguggutt

SEQ ID NO: 134 - DNA, 21 bp, Artificial
  siRNA
gggaaagggauauguaauatt

SEQ ID NO: 135 - DNA, 21 bp, Artificial
  siRNA
gcaagauuugagcauauaatt

SEQ ID NO: 136 - DNA, 21 bp, Artificial
  siRNA
gguaaucuacagugagcugtt

SEQ ID NO: 137 - DNA, 21 bp, Artificial
  siRNA
ccaacauugcuggacauuutt

SEQ ID NO: 138 - DNA, 21 bp, Artificial
  siRNA
gcauugggauauuaaguagtt

SEQ ID NO: 139 - DNA, 21 bp, Artificial
  siRNA
guccugcucuccaucuuugtt

SEQ ID NO: 140 - DNA, 21 bp, Artificial
  siRNA
ccgcuaaacgagacagacatt

SEQ ID NO: 141 - DNA, 21 bp, Artificial
  siRNA
gggacagauccagauuaugtt

SEQ ID NO: 142 - DNA, 21 bp, Artificial
  siRNA
gguacgcuguuaaguauuatt

SEQ ID NO: 143 - DNA, 21 bp, Artificial
  siRNA
gcacaugagcaguaaaacatt

SEQ ID NO: 144 - DNA, 21 bp, Artificial
  siRNA
gcaagcggaauaucucgautt

SEQ ID NO: 145 - DNA, 21 bp, Artificial
  siRNA
ccuuuucuguauauagccatt

SEQ ID NO: 146 - DNA, 21 bp, Artificial
  siRNA
gcauaauaccaucacuacutt

SEQ ID NO: 147 - DNA, 21 bp, Artificial
  siRNA
gcuacuuuggccguaaggutt

SEQ ID NO: 148 - DNA, 21 bp, Artificial
  siRNA
gcucuucuuggaaccugagtt

SEQ ID NO: 149 - DNA, 21 bp, Artificial
  siRNA
gagaugacagcaaugaguctt

SEQ ID NO: 150 - DNA, 21 bp, Artificial
  siRNA
gcagcuaaagaucaucaagtt

SEQ ID NO: 151 - DNA, 21 bp, Artificial
  siRNA
gcuaaagaucaucaagaagtt

SEQ ID NO: 152 - DNA, 21 bp, Artificial
  siRNA
ggcaaucaaggguacauactt

SEQ ID NO: 153 - DNA, 21 bp, Artificial
  siRNA
ggcauuuuagaacacuuaatt

SEQ ID NO: 154 - DNA, 21 bp, Artificial
  siRNA
ggccauugauguuuugcuatt

SEQ ID NO: 155 - DNA, 21 bp, Artificial
  siRNA
cgaaguuaucagagaugcutt

SEQ ID NO: 156 - DNA, 21 bp, Artificial
  siRNA
ccuccagagaggguauacatt

SEQ ID NO: 157 - DNA, 21 bp, Artificial
  siRNA
gcagaucuucuccguuucatt

SEQ ID NO: 158 - DNA, 21 bp, Artificial
  siRNA
ccacuugacucucuguaaatt

SEQ ID NO: 159 - DNA, 21 bp, Artificial
  siRNA
ccgucuaugugucgguguutt

SEQ ID NO: 160 - DNA, 21 bp, Artificial
  siRNA
ccuaucacguggagcucaatt

SEQ ID NO: 161 - DNA, 21 bp, Artificial
  siRNA
gcauggcguguguuggguutt

SEQ ID NO: 162 - DNA, 21 bp, Artificial
  siRNA
ccauccuccucuucuuucutt

SEQ ID NO: 163 - DNA, 21 bp, Artificial
  siRNA
gcauggcauguguuggguutt

SEQ ID NO: 164 - DNA, 21 bp, Artificial
  siRNA
caggaaacccuucaaauagtt

SEQ ID NO: 165 - DNA, 21 bp, Artificial
  siRNA
ccauuaugagcccaagaautt

SEQ ID NO: 166 - DNA, 21 bp, Artificial
  siRNA
ggcugcaucgcucaaaucutt

SEQ ID NO: 167 - DNA, 21 bp, Artificial
  siRNA
gcugcauacaugugacauatt

SEQ ID NO: 168 - DNA, 21 bp, Artificial
  siRNA
gccuuaccauacuuugauctt

SEQ ID NO: 169 - DNA, 21 bp, Artificial
  siRNA
ccagggaaucucccuaacutt

SEQ ID NO: 170 - DNA, 21 bp, Artificial
  siRNA
cccagugguaugaggugaatt

SEQ ID NO: 171 - DNA, 21 bp, Artificial
  siRNA
guacguagaugagauugcctt

SEQ ID NO: 172 - DNA, 21 bp, Artificial
  siRNA
ccaaacagugcuguuuccctt

(19) United States
(12) Patent Application Publication
(10) Pub. No.: US 2009/0214477 A1
(43) Pub. Date: Aug. 27, 2009

(54) Human Marker Genes and Agents for Diagnosis, Treatment and Prophylaxis of Cardiovascular Disorders and Artherosclerosis

(75) Inventors: Ulrich Betz, Reinheim (DE); Donatella D'Urso, Düsseldorf (DE); Peter Kolkhof, Wuppertal (DE); Michael Seewald, Langenfeld (DE); Jochen Strayle, Wuppertal (DE); Anne Plötner, Frankfurt (DE); Michael Hannus, Dresden (DE)

Correspondence Address: HAHN & VOIGHT PLLC, 1012 14TH STREET, NW, SUITE 620, WASHINGTON, DC, 20005, US

(73) Assignee: Cenix BioScience GmbH, Dresden (DE)

(21) Appl. No.: 11/911,528

(22) PCT Filed: Apr. 8, 2006
(86) PCT No.: PCT/EP06/03219

§ 371 Date: Oct. 10, 2008

Related U.S. Application Data
(60) Provisional application No. 60/671,832, filed on Apr. 15, 2005.

(51) Int. Cl. A61K 48/00; C12Q 1/02; G01N 33/53; C12Q 1/66; A61K 31/7105; C12N 15/63; C12Q 1/68

(52) U.S. Cl. 424/93.2; 435/29; 435/7.1; 435/8; 514/44 A; 435/320.1; 435/6

(57) ABSTRACT

The invention relates to novel targets in the screening for compounds useful in the treatment and/or prophylaxis of a disease selected from the group comprising cardiovascular diseases, disorders of lipid metabolism or atherosclerosis. The invention relates to novel compounds for use as a medicament for diseases or conditions involving a disease selected from the group comprising cardiovascular diseases, disorders of lipid metabolism or atherosclerosis. The invention especially relates to antagonists and expression-inhibitory compounds that target G-protein coupled receptors (GPCRs), kinases and proteases, and to methods for identifying such compounds. The invention further relates to methods for identifying these antagonists and expression-inhibitory compounds, and methods for diagnosing a disease selected from the group comprising cardiovascular diseases, disorders of lipid metabolism or atherosclerosis or a susceptibility to such a condition.

### FIELD OF THE INVENTION

The invention relates to novel targets for the screening of compounds useful in the treatment and prophylaxis or prevention of cardiovascular diseases, preferably disorders of lipid metabolism and atherosclerosis. The invention also relates to novel compounds for use as a medicament for diseases or conditions involving Cardiovascular diseases, preferably disorders of lipid metabolism and atherosclerosis. The invention furthermore relates to antagonists and expression-inhibitory compounds that target G-protein coupled receptors (GPCRs), kinases and proteases of the invention, and to methods for identifying such compounds. The invention further relates to methods for identifying these antagonists and expression-inhibitory compounds, and methods for diagnosing Cardiovascular diseases, preferably disorders of lipid metabolism and atherosclerosis or a susceptibility to such a condition.

### BACKGROUND OF THE INVENTION

Atherosclerosis is by far the single most important pathological process in the development of coronary heart disease (CHD), which is the single most common cause of morbidity and mortality in both men and women in developed nations. Atherosclerosis is a complex disease with multiple risk factors. It has been reported that 80-90% of patients who develop significant CHD and >95% of patients who experience fatal CHD have major atherosclerotic risk factors.

With regard to present day treatment of dyslipidemia, numerous well-controlled outcome studies of lipid-altering drug mono-therapy in >50000 subjects have consistently demonstrated a relative risk reduction (compared to placebo) of only 20-40% after 3-6 years of therapy. Hypercholesterolemia, or raised blood cholesterol levels, is the most prevalent cardiovascular condition, with a total prevalent condition of 320 million patients in the 8 major pharmaceutical markets. Standard therapy for atherosclerosis include lipid-lowering drugs: HMG-CoA reductase inhibitors (statins), PPAR-alpha agonists (fibrates) and niacin. Statins are the most recently launched class of anti-hypercholesterolemics and now dominate the hypercholesterolemic market. The majority of patients observed in mono-therapy trials of lipid-altering drugs have not had their CHD prevented. This suggests that further absolute and relative CHD risk will only be achieved through extending the duration of lipid-altering therapy, achieving more aggressive lipid treatment goals or treating multiple lipid parameters. It may also be reasonable to conclude that the best way to further reduce CHD risk is to aggressively correct the abnormality or abnormalities which contribute most to the atherosclerotic process in the individual patient. This may occur through mono-therapy, or through a multifactorial approach with the use of compounds addressing multiple risk factors. The US National Cholesterol Education Program (NCEP) has issued new guidelines that could significantly enhance the number of patients prescribed hypolipidemics in the US. The NCEP continues to identify LDL cholesterol as the primary target of therapy. Acceptable levels of LDL cholesterol as well as HDL cholesterol and triglycerides are more stringent than those in earlier guidelines. Therefore, additional lipid lowering therapies are needed (e.g., currently, half of patients treated with statins do not reach the new target LDL level).

Taken together, the therapeutic strategies currently available for treating Atherosclerosis are not satisfactory. As a major drawback, their limited efficacy calls for additional strategies to identify new medicaments with improved efficacy against Atherosclerosis.

Current approaches to lowering low density lipoprotein (LDL) cholesterol and therefore preventing the progression of Atherosclerosis include Squalene Synthase Inhibitors, intestinal bile acid transport (IBAT) protein inhibitors and SREBP cleavage-activating protein (SCAP) activating ligands. Other current approaches that affect lipid metabolism are microsomal triglyceride transfer protein (MTP) inhibitors, acylcoenzyme A: cholesterol acyltransferase (ACAT) inhibitors and nicotinic acid receptor (HM 74) agonists. Molecular targets involved in high density lipoprotein (HDL) cholesterol metabolism include cholesteryl ester transfer protein (CETP) with effective inhibitors under development, ATP-binding cassette transporter (ABC) A1 as well as scavenger receptor class B Type 1 (SRB1). Nuclear receptors as PPARs, LXR and FXR are also targets of investigational agents.

Because of the small number of available targets and because of the limited success in screening methods using available targets, a great need is felt in the art for promising targets and novel screening methods for compounds highly active in the treatment or Atherosclerosis.

The underlying technical problem of the present invention, therefore, can be seen as being the provision of novel screening methods, compounds, and molecular targets for the identification of compounds useful in the treatment and/or prophylaxis or prevention of Cardiovascular diseases, preferably disorders of lipid metabolism and atherosclerosis.

This problem is solved by the subject matter of the independent and dependent claims of the present patent application.

### SUMMARY OF THE INVENTION

The invention relates to methods of screening compound libraries for compounds useful in the treatment and/or prophylaxis or prevention of Cardiovascular diseases, preferably disorders of lipid metabolism and atherosclerosis. The invention further relates to the molecular targets for use in said screening methods. Furthermore, the invention relates to kits and agents for use in screening methods of the invention, and to compounds found to bind to, or modulate, the molecular targets of the invention. In one aspect of the invention, it relates to methods of treatment of a subject in need, by administering agents that bind to, or modulate, targets of the invention. In another aspect of the invention, the invention relates to compounds that are identified using the methods according to the invention. The invention also relates to the use of any one of the target genes listed in Table 10, or of any one of the polypeptides encoded thereby, for the identification of compounds useful in the treatment and/or prophylaxis of Atherosclerosis. The invention furthermore relates to the use of a compound that decreases the activity and/or the expression of a polypeptide encoded by any one of the target genes listed in Table 10 in the manufacture of a medicament for the treatment and/or prophylaxis of Cardiovascular diseases, preferably disorders of lipid metabolism and atherosclerosis or a disease associated with Atherosclerosis. The invention furthermore relates to a method of reducing Cardiovascular diseases, preferably disorders of lipid metabolism and atherosclerosis in a subject, said method comprising the step of administering to a subject in need a pharmaceutical composition according to the invention.

### BRIEF DESCRIPTION OF THE TABLES

### Table 1-12

The target list comprises screening data and gene specific information for 1277 siRNAs targeting 528 different genes, selected as positives from the total number of screened genes (target genes).

The selected genes were found positive by at least one of the three siRNAs tested per gene. As selection criteria, positive siRNAs showed an LDL-DiI uptake value of more than 2 standard deviations above the overall screen average value, corresponding to at least 314% of the unspecific control mean LDL-DiI uptake value measured in each screening plate of the primary screen.

The target list consists of 12 tables:

Table 1 contains numerical first pass screening values for LDL-DiI uptake (column 3, “LDL-DiI mean %”) and cell density (column 4, “proliferation mean %”, values normalized to the unspecific control siRNA) as well as the gene symbol (column 6, “target symbol”) and a functional classification (column 5, “Tar get Class(es)”) of the target genes.

Table 2 contains complementary information on the target genes consisting of the gene symbol (“column3, “target symbol”), RefSeq number (column 4, “RefSeq accession”), Entrez Gene ID (column 5) and a functional description derived from NCBI (column 6, “Target description”).

Table 3 indicates the nucleotide sequence of the sense strand of positive siRNAs (column 3, “siRNA sequence (21-mer)”).

Table 4 indicates the average expression level of the target genes in 3 different cell types: HepG2 human hepatoma cell line (column 4), HuH human hepatoma cell line (column 6) and human primary hepatoma cells (column 8).

Table 5 contains numerical screening values from secondary screening for Transferrin uptake (column 5, “Transferrin Run1 Mean %”; column 7, “Transferrin Run2 Mean %”; values normalized to the unspecific control siRNA) and the corresponding standard deviation (column 6, “Transferrin Run1 SD %”, column 8, “Transferrin Run2 SD %”). Included is as well as the target number (column 1, “Target No”), gene symbol (column 2, “target symbol”), the Entrez Gene ID (column 3, “Gene ID”) and the corresponding siRNA identificator (column 4, “siRNA ID”) of the target genes.

Table 6 contains numerical screening values from third pass screening for LDL-DiI uptake (column 6, “LDL-DiI Run1 Mean %”; column 8, “LDL-DiI Run2 Mean %”, column 10, “LDL-DiI Run3 Mean %”; values normalized to the unspecific control siRNA) and the corresponding standard deviation (column 7, “LDL-DiI Run1 SD %”, column 9, “LDL-DiI Run2 SD %”, column 11, “LDL-DiI Run2 SD %”). Column 5 indicates the applied siRNA concentration for each siRNA Oligo (100 nM “100”, 30 nM “30”, and 10 nM “10”). Included is as well as the target number (column 1, “Target No”), gene symbol (column 2, “target symbol”), the Entrez Gene ID (column 3, “Gene ID”) and the corresponding siRNA identificator (column 4, “siRNA ID”) of the target genes.

Table 7 contains numerical screening values from third pass screening for cell density (column 6, “Proliferation Run1 Mean %”; column 8, “Proliferation Run2 Mean %”; column 10, “Proliferation Run3 Mean %”; values normalized to the unspecific control siRNA) and the corresponding standard deviation (column 7, “Proliferation Run1 SD %”, column 9, “Proliferation Run2 SD %”, column 11, “Proliferation Run3 SD %”). Column 5 indicates the applied siRNA concentration for each siRNA Oligo (100 nM “100”, 30 nM “30”, and 10 nM “10”). Included is as well as the target number (column 1, “Target No”), gene symbol (column 2, “target symbol”), the Entrez Gene ID (column 3, “Gene ID”) and the corresponding siRNA identificator (column 4, “siRNA ID”) of the target genes.

Table 8 contains numerical values from third pass screening for remaining target mRNA expressed (column 6, “% mRNA Mean”; values normalized to the unspecific control siRNA). Column 5 indicates the applied siRNA concentration for each siRNA Oligo (100 nM “100”, 30 nM “30”, and 10 nM “10”). Included is as well as the target number (column 1, “Target No”), gene symbol (column 2, “target symbol”), the Entrez Gene ID (column 3, “Gene ID”) and the corresponding siRNA identificator (column 4, “siRNA ID”) of the target genes.

Table 9 indicates the nucleotide sequence of the sense strand of those siRNAs (column 3, “siRNA sequence (21-mer)”) used for the generation of the data presented in table 5 to table 12 and indicates the corresponding SEQ ID NO of each siRNA sequence.

Table 10 contains complementary information on specifically interesting genes. consisting of the gene symbol (“column2, “target symbol”), the Entrez Gene ID (column 3, “Gene ID”), RefSeq number (column 4, “RefSeq accession”) and a functional description derived from NCBI (column 5, “Target description”).

Table 11 contains numerical screening values from secondary screening for LDL-DiI uptake (column 5, “LDL-DiI Run1 Mean %”; column 7, “LDL-DiI Run2 Mean %”, values normalized to the unspecific control siRNA) and the corresponding standard deviation (column 6, “LDL-DiI Run1 SD %”, column 8, “LDL-DiI Run2 SD %”). Included is as well as the target number (column 1, “Target No”), gene symbol (column 2, “target symbol”), the Entrez Gene ID (column 3, “Gene ID”) and the corresponding siRNA identificator (column 4, “siRNA ID”) of the target genes.

Table 12 contains numerical screening values from secondary screening for cell density (column 5, “Proliferation Run1 Mean %”; column 7, “Proliferation Run2 Mean %”; values normalized to the unspecific control siRNA) and the corresponding standard deviation (column 6, “Proliferation Run1 SD %”, column 8, “Proliferation Run2 SD %”). Included is as well as the target number (column 1, “Target No”), gene symbol (column 2, “target symbol”), the Entrez Gene ID (column 3, “Gene ID”) and the corresponding siRNA identificator (column 4, “siRNA ID”) of the target genes.

The first column (“Target No”) of all tables assigns serial numbers to all target genes. siRNAs directed against the same gene have the same serial gene number.

### DETAILED DESCRIPTION OF THE INVENTION

A human druggable genome siRNA library was screened in a cellular assay using Huh7 hepatoma cells. Read-out was expression of LDL-R as measured by binding of LDL-DiI. Targets whose downregulation resulted in an upregulation of LDL-R expression were scored as hits (see examples).

A “functional variant” of a first polynucleotide or polypeptide, within the meaning of the invention, shall be understood as being a second polynucleotide or polypeptide of preferably high sequence identity to said first polynucleotide or polypeptide, but being different in length and sequence, due to the addition and/or deletion and/or substitution of nucleotides or amino acid residues from said first polynucleotide or polypeptide, said second polynucleotide or polypeptide still having essentially the same characteristic biological activity as has the first polynucleotide or polypeptide. Such characteristic biological activity can be catalytic activity, binding properties, or other biological activities of the original molecule.

“Reference level”, within the meaning of the invention, shall be understood as being any reference level with which a measured level of, e.g., expression or activity can be compared to. Such reference levels can be obtained, e.g., from previous experiments or from literature.

“Wild-type level”, with respect to an expression level of a gene, shall be understood as being an expression level typically observed in wild-type organisms, i.e. in not recombinantly modified organisms of the same species.

“Binding affinity” of a molecule A to a protein P, within the meaning of the invention shall be understood as being the thermodynamic quantity that corresponds to the dissociation constant of the complex consisting of the molecule A and the protein P in a reaction A+P−−>AP under standard conditions. In this case the binding affinity is [A]*[B]/[AB], wherein square brackets symbolize the concentration of the respective species.

A “reporter gene” for a target protein, within the meaning of the invention, shall be understood as being a gene which is under control of a promotor which is influenced, directly or indirectly, by said target protein. Well known reporter genes are genes coding for fluorescent proteins under the control of a second messenger-dependent promotor.

“Nucleic acids”, within the meaning of the invention, shall be understood as being all known nucleic acids such as DNA, RNA, peptide nucleic acids, morpholinos, and nucleic acids with backbone structures other than phosphodiesters, such as phosphothiates or phosphoramidates.

The term “to comprise”, within the meaning of the invention, refers to nucleic acids in which the nucleic acids with the described sequences are functionally relevant, e.g. for diagnostic use or therapeutic use, such as vectors for therapeutic use or expression of corresponding proteins.

Preferably, any additional nucleic acids upstream or downstream of the sequence are not longer than 20 kb. The term “comprise” does not relate to large constructs accidentally including the sequence, such as genomic BAC or YAC clones.

“% identity” of a first sequence towards a second sequence, within the meaning of the invention, means the % identity which is calculated as follows: First the optimal global alignment between the two sequences is determined with the CLUSTALW algorithm [Thomson J D, Higgins D G, Gibson T J. 1994. ClustalW: improving the sensitivity of progressive multiple sequence alignment through sequence weighting, positions-specific gap penalties and weight matrix choice. Nucleic Acids Res., 22: 4673-4680], Version 1.8, applying the following command line syntax: ./clustalw-infile=./infile.txt -output=-outorder=aligned -pwmatrix=gonnet -pwdnamatrix=clustalw -pwgapopen=10.0 -pwgapext=0.1 -matrix=gonnet -gapopen=10.0 -gapext=0.05 -gapdist=8-hgapresidues=GPSNDQERK -maxdiv=40. Implementations of the CLUSTAL W algorithm are readily available at numerous sites on the internet, including, e.g., http://www.ebi.ac.uk. Thereafter, the number of matches in the alignment is determined by counting the number of identical nucleotides (or amino acid residues) in aligned positions. Finally, the total number of matches is divided by the number of nucleotides (or amino acid residues) of the longer of the two sequences, and multiplied by 100 to yield the % identity of the first sequence towards the second sequence.

“Arteriosclerosis”, within the meaning of the invention, is the thickening and hardening of the arteries due to the build-up of calcium deposits on the insides of the artery walls. Cardiovascular diseases, preferably disorders of lipid metabolism and atherosclerosis is a similar condition due to the build-up of fatty substances. Both conditions have similar effects on the circulation of the blood throughout the body. Heart disease, high blood pressure, stroke, and ischemia (starvation of the cells due to insufficient circulation) may be the result of arteriosclerosis and cardiovascular diseases, preferably disorders of lipid metabolism and atherosclerosis. Within the context of this invention, “Atherosclerosis” shall be understood as encompassing both, Atherosclerosis and Arteriosclerosis as defined above.

The “nucleic acid expression vector” may be an extra-chromosomal entity, the replication of which is independent of chromosomal replication, e.g. a plasmid. Alternatively, the vector may be one which, when introduced into a host cell, particularly into a mammalian host cell, is integrated into the host cell genome and replicated together with the chromosome(s) into which it has been integrated. Preferably, the “nucleic acid expression vector” may be an expression vector which is usually applied in gene therapeutic methods in humans, particularly a retroviral vector or an adenoviral vector.

The term “expression cassette” is defined herein to include all components which are necessary or advantageous for the expression of a specific target polypeptide. An “expression cassette” may include, but is not limited to, the nucleic acid sequence of interest itself (e.g. encoding or corresponding to the siRNA or polypeptide of interest) and “control sequences”. These “control sequences” may include, but are not limited to, a promoter that is operatively linked to the nucleic acid sequence of interest, a ribosome binding site, translation initiation and termination signals and, optionally, a repressor gene or various activator genes. Control sequences are referred to as “homologous”, if they are naturally linked to the nucleic acid sequence of interest and referred to as “heterologous” if this is not the case. The term “operably linked” indicates that the sequences are arranged so that they function in concert for their intended purpose, i.e. expression of the desired protein, or, in case of RNA, transcription of the desired RNA.

The term “antibody” as used herein includes both polyclonal and monoclonal antibodies, as well as fragments thereof, such as Fv, Fab and F(ab)₂ fragments that are capable of binding antigen or hapten. The present invention also contemplates “humanized” hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art.

The invention relates to
1. Method for identifying a compound as being useful in the treatment or prophylaxis of a disease, comprising the steps of
(a) providing a first cell expressing a target polypeptide selected from the group listed in Table 10, or a fragment, or a derivative thereof;
(b) exposing said first cell to a candidate compound;
(c) determining a first level of an activity or property, said activity or property being affected by an activity or property of said target polypeptide; and
(d) selecting or discarding said candidate compound, based on a comparison of said first level of said activity or property with a reference level of said activity or property;
characterised in that
said disease is a disease selected from the group comprising cardiovascular diseases, disorders of lipid metabolism or atherosclerosis.
   2. Use of a method of Count 1 for the screening for substances useful in the treatment or prophylaxis of A disease selected from the group comprising cardiovascular diseases, disorders of lipid metabolism or atherosclerosis.
   3. Method of Count 1 or use of Count 2, wherein said host cell expresses said target polypeptide above wild-type level.
   4. Method or use of any of Counts 1 to 3, wherein said target polypeptide expression is recombinant polypeptide expression.
   5. Method or use of any of Counts 1 to 4, wherein said compound is selected if said first level of said activity or property is lower than said reference level of said activity or property.
   6. Method or use of any of Counts 1 to 4, wherein said compound is selected if said first level of said activity or property is higher than said reference level of said activity or property.
   7. Method or use of any of Counts 1 to 6, wherein said reference level is a level obtained from a second cell expressing the target polypeptide at a lower level as compared to said first cell.
   8. Method or use of any of Counts 1 to 6, wherein said reference level is the level obtained with said first cell in the absence of the candidate compound.
   9. Method or use of any of Counts 1 to 8, wherein said method further comprises contacting the host cell with a known agonist or antagonist of the target polypeptide before determining the first level.
   10. Method or use of any of Counts 1 to 9, wherein said activity or property being affected by said activity or property of said target polypeptide is binding affinity of said compound to said target polypeptide.
   11. Use of a method, said method comprising the steps of
(a) culturing a population of cells expressing a target polypeptide listed in Table 10, or a functional fragment or derivative thereof;
(b) determining a first level of expression and/or activity of said target protein in said population of cells;
(c) exposing said population of cells to a compound, or a mixture of compounds;
(d) determining a second level of expression and/or activity of said target polypeptide in said population of cells during or after said exposure of said population of cells to the compound, or the mixture of compounds; and
(e) comparing said first and said second level;
   for the screening for substances useful in the treatment or prophylaxis of A disease selected from the group comprising cardiovascular diseases, disorders of lipid metabolism or atherosclerosis.
   12. Method or use of any of Counts 1 to 11, wherein said first level of an activity or property is determined with a reporter, said reporter being controlled by a promoter responsive to at least one second messenger.
   13. Method or use of Count 12, wherein said at least one second messenger is cyclic AMP, or Ca2+, or both.
   14. Method or use of Count 12 or 13, wherein said promoter is a cyclic AMP-responsive promoter, an NF-KB responsive promoter, a NF-AT responsive promoter, or a promoter responsive to transcription factors or to nuclear hormone receptors.
   15. Method or use of any of Counts 12 to 14, wherein the reporter is luciferase or beta-galactosidase.
   16. Method or use of any of Counts 1 to 15, wherein the compound is a low molecular weight compound.
   17. Method or use of any of Counts 1 to 15, wherein the compound is a polypeptide.
   18. Method or use of any of Counts 1 to 15, wherein the compound is a lipid.
   19. Method or use of any of Counts 1 to 15, wherein the compound is a natural compound.
   20. Method or use of any of Counts 1 to 15, wherein the compound is an antibody or a nanobody.
   21. Method for identifying a compound as being useful in the treatment or prophylaxis of a disease, comprising the steps of
(a) contacting said compound with a target polypeptide selected from the group listed in Table 10, or a fragment, or a derivative thereof;
(b) detect binding of said compound to said target polypeptide or detect a change in activity of said target polypeptide;
(c) selecting said compound If binding is detected in step (b) or if a change in activity is detected in step (b);
characterised in that
said disease is A disease selected from the group comprising cardiovascular diseases, disorders of lipid metabolism or atherosclerosis.
   22. Use of a method of count 21 for screening for compounds, useful in the treatment or prophylaxis of A disease selected from the group comprising cardiovascular diseases, disorders of lipid metabolism or atherosclerosis.
   23. Method or use of any of counts 21 to 22, wherein binding is detected in vitro.
   24. Method or use of any of counts 21 to 23, wherein said target polypeptide is a recombinant polypeptide.

25. Method or use of any of counts 21 to 24, wherein said compound is selected if the binding affinity is equal to or lower than 10 micromolar.

26. Method or use of any of counts 21 to 25, wherein said compound is a low molecular weight compound.
27. Method or use of any of counts 21 to 25, wherein said compound is a polypeptide, or a lipid, or a natural compound, or an antibody or a nanobody.
28. Use of a compound that inhibits an activity and/or the expression of any of the polypeptides listed in Table 10 in the manufacture of a medicament for the treatment or prophylxis of A disease selected from the group comprising cardiovascular diseases, disorders of lipid metabolism or atherosclerosis.
29. Use of Count 28, wherein said compound is identified according to any one of the methods or uses of Counts 1 to 27.
30. Use of an agent inhibiting the expression of a polypeptide selected from the group listed in Table 10 for the preparation of a medicament for the treatment or prophylaxis of A disease selected from the group comprising cardiovascular diseases, disorders of lipid metabolism or atherosclerosis.
31. Use of Count 30, wherein said agent is selected from the group consisting of an antisense RNA encoding said polypeptide;
a ribozyme that cleaves the polyribonucleotide encoding said polypeptide;
an antisense oligodeoxynucleotide (ODN) enconding said polypeptide;
a small interfering RNA (siRNA) that is sufficiently homologous to a portion of the polyribonucleotide such that said siRNA is capable of inhibiting the polyribonucleotide that would otherwise cause the production of said polypeptide;
a small interfering RNA (siRNA) having the sequence of any of SEQ ID NO:1 to 172;
a microRNA (miRNA) suitable for inhibition of a polypeptide selected from the group listed in Table 10; or
a short hairpin RNA (shRNA) suitable for silencing expression of a polypeptide selected from the group listed in table 10.
   32. Use of Count 31, wherein the nucleotide sequence of said agent is present in a vector.
   33. Use of Count 32, wherein the vector is an adenovirus, a retrovirus, an alphavirus, an adeno-associated virus (AAV), a lentivirus, a herpes simplex virus (HSV) or a sendai virus.
   34. Use of any of Counts 31 to 33, wherein said agent is siRNA, and said siRNA comprises a sense strand of 17 to 31 nucleotides which is identical to a region of the coding sequence, or its complementary sequence, of any of the polypeptides of Table 10.
   35. Use of Count 34, wherein the siRNA further comprises a cleavable loop region connecting the sense and the antisense strand.
   36. Vector comprising any of SEQ ID NO:1 to 172
   37. Use of a vector of Count 36 as a medicament.
   38. Use of a vector of Count 37 for the manufacture of a medicament useful in the treatment or prophylaxis of A disease selected from the group comprising cardiovascular diseases, disorders of lipid metabolism or atherosclerosis.
   39. Use according to Count 37 or 38, wherein the vector is an adenoviral, retroviral, adeno-associated viral, lentiviral or a sendaiviral vector.
   40. Method for diagnosing a pathological condition involving A disease selected from the group comprising cardiovascular diseases, disorders of lipid metabolism or atherosclerosis, or a susceptibility to said condition in a subject, comprising
(a) obtaining a sample of the subject's mRNA corresponding to a polypeptide selected from the group listed in Table 10, or a sample of the subject's genomic DNA corresponding to a polypeptide of Table 10;
(b) determining the nucleic acid sequence of said mRNA or said genomic DNA;
(c) obtaining the nucleic acid sequence encoding said polypeptide of Table 10 from a public database; and
(d) identifying any difference(s) between the nucleic acid sequences determined in step (b) and (c);
   wherein a pathological condition involving a disease selected from the group comprising cardiovascular diseases, disorders of lipid metabolism or a disease selected from the group comprising cardiovascular diseases, disorders of lipid metabolism or atherosclerosis, or a susceptibility to such a condition in a subject is diagnosed, if such difference(s) are identified in step (d).
   41. Method for diagnosing a pathological condition involving A disease selected from the group comprising cardiovascular diseases, disorders of lipid metabolism or atherosclerosis or a susceptibility to such a condition in a subject, comprising
(a) determining the amount of a polypeptide of Table 10 in a biological sample of said subject; and
(b) comparing the amount determined in (a) with a the amount of the polypeptide in a healthy subject;
   wherein an increase or a decrease of the amount of said polypeptide compared to the amount present in a healthy subject is indicative of the presence of the pathological condition.

One further embodiment of the invention is the use of the genes/proteins listed in Table 10 as therapeutical targets in the field of cardiovascular diseases, preferably lipid metabolism disorders or atherosclerosis.

Furthermore, those targets listed in Table 10 are preferred, which are highly expressed in HepG2 cells, Huh cells, primary hepatocytes, and whole liver cells. Those targets of Table 10, which show an average expression of above 1000 in HepG2 cells, Huh cells, primary hepatocytes, or whole liver cells, in Table 4, are preferred targets of the invention. Even more preferred are targets of Table 10, which show an average expression of above 1000 in at least two, or three or (most preferred) four cell types, in a list of cell types consisting of HepG2 cells, Huh cells, primary hepatocytes, and whole liver cells, in Table 4.

Furthermore, those targets listed in Table 10 are preferred, which show an increase in LDL-DiI uptake with more than one siRNA oligo in the primary and/or secondary screening (Table 1 and Table 11) and show no significant alteration in cellular proliferation (Table 12). Furthermore, those targets listed in Table 10 are preferred, which show increased LDL-DiI uptake (Table 11) without any similarly strong increase in Transferrin uptake (Table 5). Furthermore, those targets listed in Table 10 are preferred, which show a strongly increased LDL-DiI uptake (Table 11) with at least one siRNA oligo.

According to a further preferred embodiment, the nucleic acid molecules may also have the antisense-sequence of any of the sequences of the invention. According to a further embodiment, fragments or functional variants of the nucleic acid molecules as described above may be used. According to a further embodiment, the nucleic acid molecule comprises a nucleotide sequence which is capable of hybridizing with the nucleic acid sequences of the invention under conditions of medium/high stringency. In such hybrids, duplex formation and stability depend on substantial complementarity between the two strands of the hybrid and a certain degree of mismatch can be tolerated. Therefore, the nucleic acid molecules and probes of the present invention may include mutations (both single and multiple), deletions, insertions of the above identified sequences, and combinations thereof, as long as said sequence variants still have substantial sequence similarity to the original sequence which permits the formation of stable hybrids with the target nucleotide sequence of interest. Suitable experimental conditions for determining whether a given DNA or RNA sequence “hybridizes” to a specified polynucleotide or oligonucleotide probe involve pre-soaking of the filter containing the DNA or RNA to examine for hybridization in 5×SSC (sodium chloride/sodium citrate) buffer for 10 minutes, and pre-hybridization of the filter in a solution of 5×SSC, 5×Denhardt's solution, 0.5% SDS and 100 mg/ml of denaturated sonicated salmon sperm DNA (Maniatis et al., 1989), followed by hybridization in the same solution containing a concentration of 10 ng/ml of a random primed (Feinberg, A. P. and Vogelstein, B. (1983), Anal. Biochem. 132:6-13), ³²P-dCTP-labeled (specific activity >1×10⁹ cpm/μg) probe for 12 hours at approximately 45° C. The filter is then washed twice for 30 minutes in 2×SSC, 0.5% SDS at least 55° C. (low stringency), at least 60° C. (medium stringency), preferably at least 65° C. (medium/high stringency), more preferably at least 70° C. (high stringency) or most preferably at least 75° C. (very high stringency). Molecules to which the probe hybridizes under the chosen conditions are detected using an x-ray film or a “phosphor imager”. “Suitable conditions” for the production of the above double-stranded RNA-molecule are all in vivo or in vitro conditions that according to the state of art allow the expression of a first and a second RNA-strand with the above sequences and lengths that—when hybridized—form a double-stranded RNA-molecule. Particularly preferred “suitable conditions” for the production of the above double-stranded RNA-molecule are the “in vivo conditions” in a living human or animal cell or the “in vitro conditions” in cultured human or animal cells.

The isolated nucleic acid molecules of the invention, or their modulators/regulators may be used for treating or diagnosing Cardiovascular diseases, preferably disorders of lipid metabolism and atherosclerosis either in vitro or in vivo.

Treatment and/or prophylaxis of Artherosclerosis using said nucleic acid molecules can be achieved in different ways familiar to the person skilled in the art. For example, the isolated nucleic acid molecules may be inserted downstream of a strong promotor to overexpress the corresponding protein or polypeptide. Overexpression of the protein or polypeptide may lead to suppression of the endogenous protein's biological function. By introducing deletions or other mutations into the nucleic acids, or by using suitable fragments, it is possible to generate sequences encoding dominant-negative peptides or polypeptides. Such dominant-negative peptides or polypeptides can inhibit the function of the corresponding endogenous protein.

According to a further preferred embodiment, the invention relates to the use of the above identified nucleic acid molecules or functional variants thereof in form of RNA, particularly antisense RNA and double-stranded RNA, for the manufacture of a medicament for the treatment and/or prophylaxis of Artherosclerosis. Also ribozymes can be generated for the above identified sequences and used to degrade RNA transcribed from the corresponding endogenous genes.

Particularly preferred is the use of these RNA molecules in a therapeutic application of the RNAi technique, particularly in humans or in human cells. An RNAi technique particularly suited for mammalian cells makes use of double-stranded RNA oligonucleotides known as “small interfering RNA” (siRNA).

Therefore, according to a further preferred embodiment, the invention relates to the use of nucleic molecules comprising small interfering RNA with a sequence corresponding to any of the sequences given in table 3.

These siRNA molecules can be used for the therapeutic silencing of the expression of the genes of the invention comprising nucleic acid sequences of the invention, in mammalian cells, particularly in human cells, particularly for the therapy of Artherosclerosis.

The inhibition of a specific target gene in mammals is achieved by the introduction of an siRNA-molecule having a sequence that is specific (see above) for the target gene into the mammalian cell. The siRNAs comprise a first and a second RNA strand, both hybridized to each other, wherein the sequence of the first RNA strand is a fragment of one of the sequences of the invention and wherein the sequence of the second RNA strand is the antisense-strand of the first RNA strand. The siRNA-molecules may possess a characteristic 2- or 3-nucleotide 3′-overhanging sequence. Each strand of the siRNA molecule preferably has a length of 19 to 31 nucleotides.

The siRNAs can be introduced into the mammalian cell by any suitable known method of cell transfection, particularly lipofection, electroporation or microinjection. The RNA oligonucleotides can be generated and hybridized to each other in vitro or in vivo according to any of the known RNA synthesis methods.

In another embodiment, the invention relates to the use of a nucleic acid molecule as defined above, wherein the nucleic acid molecule is contained in at least one nucleic acid expression vector which is capable of producing a double-stranded RNA-molecule comprising a sense-RNA-stand and an antisense-RNA-strand under suitable conditions, wherein each RNA-strand, independently from the other, has a length of 19 to 31 nucleotides.

In this alternative method (also described in Tuschl, Nature Biotechnology, Vol. 20, pp. 446-448), vector systems capable of producing siRNAs instead of the siRNAs themselves are introduced into the mammalian cell for down-regulating gene expression. The preferred lengths of the RNA-strands produced by such vectors correspond to those preferred for siRNAs in general (see below).

microRNAs (miRNAs) are evolutionarily conserved small non-protein-coding RNA gene products that regulate gene expression at the post-transcriptional level. In animals, mature miRNAs are ˜22 nucleotides long and are generated from a primary transcript through sequential processing by nucleases belonging to the RNAseIII family.

An alternative to transfecting cells with chemically synthesized siRNAs are DNA-vector-mediated mechanisms to express substrates that can be converted into siRNA in vivo. In the first approach the sense and antisense strands of the siRNA are expressed from different, usually tandem promoters. Alternatively, short hairpin (sh)RNAs are expressed and processed by Dicer into siRNAs. In general, chemically synthesized short interfering (si)RNA sequences that are effective at silencing gene expression are also effective when generated from short hairpin (sh)RNAs. However, the length of the stem and the size and composition of the loop are important for the efficiency of silencing.

The coding sequence of interest may, if necessary, be operably linked to a suitable terminator or to a poly-adenylation sequence. In the case of RNA, particularly siRNA, “coding sequence” refers to the sequence encoding or corresponding to the relevant RNA strand or RNA strands.

Further, the vector may comprise a DNA sequence enabling the vector to replicate in the mammalian host cell. Examples of such a sequence—particularly when the host cell is a mammalian cell—is the SV40 origin of replication.

A number of vectors suitable for expression in mammalian cells are known in the art and several of them are commercially available. Some commercially available mammalian expression vectors which may be suitable include, but are not limited to, pMC1neo (Stratagene), pXT1 (Stratagene), pSG5 (Stratagene), pcDNAI (Invitrogen), EBO-pSV2-neo (ATCC 37593), pBPV-1(8-2) (ATCC 37110), pSV2-dhfr (ATCC 37146). Preferred are all suitable gene therapeutic vectors known in the art.

In a particularly preferred embodiment of the invention the vector is a retroviral vector. Retroviruses are RNA-viruses possessing a genome that after the infection of a cell, such as a human cell, is reversely transcribed in DNA and subsequently is integrated into the genome of the host cell. Retroviruses enter their host cell by receptor-mediated endocytosis. After the endocytosis into the cell the expression of the retroviral vector may be silenced to ensure that only a single cell is infected. The integration of the viral DNA into the genome is mediated by a virus-encoded protein called integrase, wherein the integration locus is not defined. Retroviral vectors are particularly appropriate for their use in gene therapeutic methods, since their transfer by receptor-mediated endocytosis into the host cell, also known to those skilled in the art as “retroviral transduction” is particularly efficient. A person skilled in the art also knows how to introduce such retroviral vectors into the host cell using so called “packaging cells”.

In another particularly preferred embodiment of the invention, the vector is an adenoviral vector or a derivative thereof. Adenoviral vectors comprise both replication-capable and replication-deficient vectors. The latter include vectors deficient in the E1 gene.

The recombinant vector is preferably introduced into the mammalian host cells by a suitable pharmaceutical carrier that allows transformation or transfection of the mammalian, in particular human cells. Preferred transformation/transfection techniques include, but are not limited to liposome-mediated transfection, virus-mediated transfection and calcium phosphate transfection.

In a preferred embodiment, the invention relates to the use of a vector system capable of producing siRNAs as defined above, wherein the nucleic acid corresponding to the siRNA is contained in at least one nucleic acid expression vector comprising a first expression cassette containing the nucleic acid corresponding to the sense-RNA-strand under the control of a first promoter and a second expression cassette containing the nucleic acid corresponding to the antisense-RNA-strand under the control of a second promoter.

In the above mentioned vector system, the vector comprises two individual promoters, wherein the first promoter controls the transcription of the sense-strand and the second promoter controls the transcription of the antisense strand (also described in Tuschl, Nature Biotechnology, Vol. 20, pp. 446-448). Finally the siRNA duplex is constituted by the hybridisation of the first and the second RNA-strand.

The promoter used in the aforementioned “expression cassettes” may be any DNA sequence which shows transcriptional activity in a host cell of choice, preferably in a mammalian host cell, particularly in a human host cell. The promoter may be derived from genes encoding proteins either homologous or heterologous to the host cell.

As a promoter in general every promoter known in the prior art can be used that allows the expression of the gene of interest under appropriate conditions in a mammalian host cell, in particular in a human host cell. Particularly promoters derived from RNA polymerase III transcription units, which normally encode the small nuclear RNAs (snRNAs) U6 or the human RNAse P RNA H1, can be used as promoters to express the therapeutic siRNAs. These particularly preferred promoters U6 and H1 RNA which are members of the type III class of Polymerase III promoters are—with the exception of the first transcribed nucleotide (+1 position)—only located upstream of the transcribed region.

In a preferred embodiment, the invention relates to the use of a vector system capable of producing siRNAs for the above identified nucleic acid sequences, wherein the sequence is contained in at least one nucleic acid expression vector comprising an expression cassette containing the sequence of the sense-RNA-strand and of the antisense-RNA-strand under the control of a promoter leading to a single-stranded RNA-molecule and wherein the single-stranded RNA-molecule is capable of forming a back-folded stem-loop-structure.

In this vector system (also described in Tuschl, Nature Biotechnology, Vol. 20, pp. 446-448), only a single RNA-strand is produced under the control of a single promoter, wherein the RNA strand comprises both the sense- and of the antisense-strand of the final double-stranded siRNA molecule. This structure leads to a back-folding of the RNA-strand by hybridisation of the complementary sense- and antisense-sequences under stem-loop formation. Finally the intracellular processing of this fold-back stem-loop-structure gives rise to siRNA.

In another preferred embodiment according to the present invention, the “nucleic acid expression vector” comprises an expression cassette containing the sequence of the sense-RNA-strand and of the antisense-RNA-strand both under the control of a single promoter leading to a single-stranded RNA-molecule. This single-stranded RNA-molecule is hereby capable to form a back-folded stem-loop-structure. These expressed “hairpin RNA-molecules” subsequently give rise to siRNAs after intracellular processing.

In a preferred embodiment of the invention the nucleic acid expression vector that gives rise to the expression of siRNAs according to the present invention is first introduced into therapeutic, non-toxic virus particles or virus-derived particles that are suitable for gene therapeutic applications and that can infect mammalian, in particular human target cells, such as packaging cells etc.

In a preferred embodiment, the first and the second RNA strand of the siRNA may have, independently from the other, a length of 19 to 25 nucleotides, more preferred of 20 to 25 nucleotides, and most preferred of 20 to 22 nucleotides.

In another preferred embodiment, the first and the second RNA strand of the siRNA may have, independently from the other, a length of 26 to 30 nucleotides, more preferred of 26 to 28 nucleotides, and most preferred of 27 nucleotides.

In another aspect, the invention relates to the use of isolated proteins or polypeptides comprising a sequence selected from the group consisting of
(a) a sequence as disclosed by the corresponding accession number in table 10;
(b) a sequence that exhibits a sequence identity with any of the sequences according to (a) of at least 90% over at least 100 residues,
(c) or functional variants of the sequences defined in (a) or (b),
   for the manufacture of a medicament for the treatment and/or prophylaxis of Cardiovascular diseases, preferably disorders of lipid metabolism and atherosclerosis.

Proteins, polypeptides and peptides can be introduced into the cells by various methods known in the art. For example, amphiphilic molecules may be membrane permeable and can enter cells directly. Membrane-bound proteins or polypeptides (usually lipophilic molecules or containing transmembrane domains) may insert directly into cell membranes and can thus exert their biological function. Other ways of introduction or intracellular uptake include microinjection, lipofection, receptor-mediated endocytosis, or the use of suitable carrier-molecules, particularly carrier-peptides. Suitable carrier-peptides include or can be derived from HIV-tat, antennapedia-related peptides (penetratins), galparan (transportan), polyarginine-containing peptides or polypeptides, Pep-1, herpes simplex virus VP-22 protein. Another possible introduction method is to introduce nucleic acid vectors capable of expressing such proteins, polypeptides or peptides.

Suitable methods to produce isolated polypeptides are known in the art. For example, such a method may comprise transferring the expression vector with an operably linked nucleic acid molecule encoding the polypeptide into a suitable host cell, cultivating said host cells under conditions which will permit the expression of said polypeptide or fragment thereof and, optionally, secretion of the expressed polypeptide into the culture medium. Depending on the cell-type different desired modifications, e.g. glycosylation, can be achieved.

The proteins, polypeptides and peptides may also be produced synthetically, e.g. by solid phase synthesis (Merrifield synthesis).

The polypeptides used in the invention may also include fusion polypeptides. In such fusion polypeptides another polypeptide may be fused at the N-terminus or the C-terminus of the polypeptide of interest or fragment thereof. A fusion polypeptide is produced by fusing a nucleic acid sequence (or a portion thereof) encoding another polypeptide to a nucleic acid sequence (or a portion thereof) of the present invention. Techniques for producing fusion polypeptides are known in the art and include ligating the coding sequences so that they are in frame and the expression of the fusion polypeptide is under control of the same promotor(s) and terminator.

Expression of the polypeptides of interest may also be performed using in vitro produced synthetic mRNA. Synthetic mRNA can be efficiently translated in various cell-free systems, including but not limited to, wheat germ extracts and reticulocyte extracts, as well as efficiently translated in cell based systems including, but not limited to, microinjection into frog oocytes, preferably Xenopus laevis oocytes.

Treatment and/or prophylaxis of Cardiovascular diseases, preferably disorders of lipid metabolism and atherosclerosis, using said isolated proteins or polypeptides, can be achieved by different ways familiar to the person skilled in the art: Overexpression of the protein or polypeptide may lead to suppression of the endogenous protein's biological function. By introducing deletions or other mutations, or by using suitable fragments, it is possible to generate sequences encoding dominant-negative peptides or polypeptides. Such dominant-negative peptides or polypeptides can inhibit the function of the corresponding endogenous protein. For example, functional variants or mutants can be generated which consist only of binding domains but are enzymatically inactive (i.e. partially lacking their biological function). Such dominant-negative molecules may interfere with the biological function of the endogenous proteins or polypeptides by binding to intracellular binding partners and thus blocking activation of the endogenous molecule.

In another aspect, the invention relates to the use of an antibody which is directed against at least one polypeptide comprising a sequence as defined above for the manufacture of a medicament for the treatment and/or prophylaxis of Cardiovascular diseases, preferably disorders of lipid metabolism and atherosclerosis.

The term “antibody” as used herein includes both polyclonal and monoclonal antibodies, as well as fragments thereof, such as Fv, Fab and F(ab)₂ fragments that are capable of binding antigen or hapten. The present invention also contemplates “humanized” hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art.

Antibodies specifically binding to proteins of the invention, or suitable fragments thereof, particularly in humanized form, may be used as therapeutic agents in a method for treating Cardiovascular diseases, preferably disorders of lipid metabolism and atherosclerosis. The use of said antibodies may also include the therapeutical inhibition of the above identified nucleic acid molecules or their corresponding polypeptides. In particular, this use may be directed to

Cardiovascular diseases, preferably disorders of lipid metabolism and atherosclerosis. The antibodies or fragments may be introduced into the body by any method known in the art. Delivery of antibodies, particularly of fragments, into live cells may be performed as described for peptides, polypeptides and proteins. If the antigen is extracellular or an extracellular domain, the antibody may exert its function by binding to this domain, without need for intracellular delivery.

Antibodies can be coupled covalently to a detectable label, such as a radiolabel, enzyme label, luminescent label, fluorescent label or the like, using linker technology established for this purpose. Labeling is particularly useful for diagnostic purposes (see below) or for monitoring the distribution of the antibody within the body or a neoplastic tumor, e.g. by computed tomography, PET (positron emission tomography), or SPECT (single photon emission computed tomography).

In another aspect, the invention relates to the use of an isolated nucleic acid molecule comprising a nucleic acid with a sequence selected from the group of sequences consisting of:
a) the nucleic acid sequences presented by the corresponding accession number in table 10;
b) nucleic acid sequences encoding polypeptides that exhibit a sequence identity with the protein encoded by a nucleic acid according to a) of at least 90% over at least 100 residues and/or which are detectable in a computer aided search using the BLAST sequence analysis programs with an e-value of at most 10⁻⁵,
c) sequences of nucleic acid molecules which are capable of hybridizing with the nucleic acid molecules with sequences corresponding to (a) or (b) under conditions of medium or high stringency,
d) the antisense-sequence of any of the sequences as defined in (a), (b) or (c),
e) functional variants of (a), (b), (c) or (d),
f) RNA sequences corresponding to any of the sequences as defined in (a), (b), (c), (d), or (e),
   for the manufacture of a medicament for the activation of Cardiovascular diseases, preferably disorders of lipid metabolism and atherosclerosis.

In another aspect, the invention relates to the use of a an isolated peptide or polypeptide comprising a peptide or polypeptide with a sequence selected from the group consisting of:
(a) a sequence as disclosed by the corresponding accession number in table 10;
(b) a sequence that exhibits a sequence identity with any of the sequences according to (a) of at least 90% over 100 residues.
(c) functional variants of the sequences defined in (a) or (b),
   for the manufacture of a medicament for the treatment and/or prophylaxis of Cardiovascular diseases, preferably disorders of lipid metabolism and atherosclerosis.

In another aspect, the invention relates to the use of an antibody which is directed against at least one peptide or polypeptide with a sequence as defined above for the manufacture of a medicament for the treatment and/or prevention of cardiovascular diseases, preferably disorders of lipid metabolism and atherosclerosis.

Expression of RNA or polypeptides may be achieved by introduction of genomic DNA or cDNA containing suitable promoters, preferably constitutive or homologous promoters. Alternatively, any suitable nucleic acid expression vector can be used. The encoded protein or polypeptide may be full-length or a fragment or peptide with a similar biological function.

The proteins, polypeptides or peptides may also be generated by any known in vivo or in vitro method and introduced directly into the cells.

It is known that suitable antibodies can be used to activate the biological function of target proteins they bind to. Activation may occur by inducing conformational changes upon binding to the target protein. Another possibility is that the antibody binds two or more target proteins and brings them into sufficiently close physical proximity to induce interaction of the target proteins. The latter mode of activation is particularly known for membrane-bound dimeric receptors.

With respect to the specific embodiments relating to the used nucleic acids, peptides, polypeptides, proteins, and antibodies the same applies as defined above for the other uses of the invention.

In another embodiment, the invention relates to a medicament containing an isolated nucleic acid molecule, peptide, polypeptide, or antibody selected from the group consisting of
a) nucleic acid molecules or nucleic acid expression vectors as defined above,
b) a peptide or polypeptide comprising a sequence as defined above,
c) an antibody directed against at least one peptide or polypeptide according to (b).

Preferably this isolated nucleic acid molecule is an RNA molecule and preferably is double-stranded. Particularly the isolated nucleic acid molecule is an siRNA molecule according to the present invention.

The following considerations for medicaments and their administration apply also to the medicaments of the invention as to the above disclosed uses.

The medicament preferably comprises additionally a suitable pharmaceutically acceptable carrier, preferably virus-particles or virus-derived particles that may harbour the viral vectors, transfection solutions comprising liposomes, particularly cationic liposomes, calcium phosphate etc. Preferably a carrier is used, which is capable of increasing the efficacy of the expression vector or virus particles containing the expression vector to enter the mammalian target cells. The medicament may additionally comprise other carrier substances, preferably starch, lactose, fats, stearin acid, alcohol, physiological NaCl-solutions or further additives, in particular stabilizers, preservatives, dyes and flavourings.

The medicaments may also comprise other suitable substances. For example, RNA or siRNA containing medicaments may contain substances which stabilize double-stranded RNA molecule and/or which enable the double-stranded RNA molecule or DNA expression vector to be transfected or to be injected into the human or animal cell.

Administration can be carried out by known methods, wherein a nucleic acid is introduced into a desired cell in vitro or in vivo. For therapeutic applications, the medicament may be in form of a solution, in particular an injectable solution, a cream, ointment, tablet, suspension, granulate or the like. The medicament may be administered in any suitable way, in particular by injection, by oral, nasal, rectal application. The medicament may particularly be administered parenteral, that means without entering the digestion apparatus, for example by subcutaneous injection. The medicament may also be injected intravenously in the form of solutions for infusions or injections. Other suitable administration forms may be direct administrations on the skin in the form of creams, ointments, sprays and other transdermal therapeutic substances or in the form of inhalative substances, such as nose sprays, aerosoles or in the form of microcapsules or implantates.

The optimal administration form and/or administration dosis for a medicament either comprising double-stranded RNA molecules with the above sequences or comprising nucleic acid vectors capable to express such double-stranded RNA molecules depend on the type and the progression of the disease to be treated.

In another embodiment of the invention, an activator or an inhibitor of a protein of the invention can be administered to a patient in need.

Preferably, the activator or inhibitor is administered in pharmaceutically effective amount. As used herein, a “pharmaceutically effective amount” of an activator or inhibitor is an amount effective to achieve the desired physiological result, either in cells treated in vitro or in a subject treated in vivo. Specifically, a pharmaceutically effective amount is an amount sufficient to positively influence, for some period of time, one or more clinically defined pathological effects associated with Cardiovascular diseases, preferably disorders of lipid metabolism and atherosclerosis. The pharmaceutically effective amount may vary depending on the specific activator or inhibitor selected, and is also dependent on a variety of factors and conditions related to the subject to be treated and the severity of the disease. For example, if the activator or inhibitor is to be administered in vivo, factors such as age, weight, sex, and general health of the patient as well as dose response curves and toxicity data obtained in pre-clinical animal tests would be among the factors to be considered. If the activator or inhibitor is to be contacted with cells in vitro, one would also design a variety of pre-clinical in vitro studies to assess parameters like uptake, half-life, dose, toxicity etc. The determination of a pharmaceutically effective amount for a given agent (activator or inhibitor) is well within the ability of those skilled in the art. Preferably, the activator or inhibitor is present in a concentration of 0.1 to 50% per weight of the pharmaceutical composition, more preferably 10 to 30%.

An inhibitor, activator, or drug according to the present invention may also be a “small molecule”. Small molecules are molecules which are not proteins, peptides antibodies or nucleic acids, and which exhibit a molecular weight of less than 5000 Da, preferably less than 2000 Da, more preferably less than 2000 Da, most preferably less than 500 Da. Such small molecules may be identified in high throughput procedures/screening assays starting from libraries. Such methods are known in the art. Suitable small molecules can also be designed or further modified by methods known as combinatorial chemistry.

In another aspect, the present invention relates to the use of an isolated nucleic acid molecule comprising a sequence as defined above or the use of a ligand binding specifically at least one polypeptide comprising a sequence as defined above for the in vitro diagnosis of Cardiovascular diseases, preferably disorders of lipid metabolism and atherosclerosis.

The diagnostic use of the above identified nucleic acid molecules and probes may include, but is not limited to the quantitative detection of expression of said target genes in biological probes (preferably, but not limited to tissue samples, cell extracts, body fluids, etc.), particularly by quantitative hybridization to the endogenous nucleic acid molecules comprising the above-characterized nucleic acid sequences (particularly cDNA, RNA)

The invention further relates to methods for diagnosis a pathological condition involving Atherosclerosis in a subject, said methods comprising the steps of: (a) determining the nucleic acid sequence of one of the target genes listed in Table 10 within the genomic DNA of said subject; (b) comparing the sequence from step (a) with the nucleic acid sequence obtained from a database and/or a healthy subject; and identifying any difference(s) related to the onset of Atherosclerosis.

Expression of the endogenous genes or their corresponding proteins can be analyzed in vitro in tissue samples, body fluids, and tissue and cell extracts. Expression analysis can be performed by any method known in the art, such as RNA in situ hybridization, PCR (including quantitative RT-PCR), and various serological or immunological assays which include, but are not limited to, precipitation, passive agglutination, enzyme-linked immunosorbent antibody (ELISA) technique and radioimmunoassay techniques.

The diagnostic use may also include the detection of mutations in endogenous genes corresponding to the above identified nucleic acid sequences.

Suitable nucleic acid probes may be synthesized by use of DNA synthesizers according to standard procedures or, preferably for long sequences, by use of PCR technology with a selected template sequence and selected primers. The probes may be labeled with any suitable label known to those skilled in the art, including radioactive and non-radioactive labels. Typical radioactive labels include ³²P, ¹²⁵I, ³⁵S, or the like. A probe labeled with a radioactive isotope can be constructed from a DNA template by a conventional nick translation reaction using a DNase and DNA polymerase. Non-radioactive labels include, for example, ligands such as biotin or thyroxin, or various luminescent or fluorescent compounds. The probe may also be labeled at both ends with different types of labels, for example with an isotopic label at one end and a biotin label at the other end. The labeled probe and sample can then be combined in a hybridization buffer solution and held at an appropriate temperature until annealing occurs. Such nucleic acid probes may also be used for other than diagnostic purposes, e.g. for the identification of further homologs or orthologs.

“Ligands” binding specifically to said polypeptides are known in the art. Such ligands include proteins or polypeptides, for example intracellular binding partners, antibodies, molecular affinity bodies, and small molecules. Specifically binding ligands can be identified by standard screening assays known in the art (see also below), for example by yeast two-hybrid screens and affinity chromatography. A specifically binding ligand does not need to exert another function such as inhibiting or activating the molecule with which it interacts.

In a preferred embodiment, the ligand is an antibody binding specifically at least one polypeptide comprising a sequence as defined above.

“Specific binding” according to the present invention means that the polypeptide to be identified (the target polypeptide) is bound with higher affinity than any other polypeptides present in the sample. Preferred is at least 3 times higher affinity, more preferred at least 10 times higher affinity, and most preferred at least 50 times higher affinity. Non-specific binding (“cross-reactivity”) may be tolerable if the target polypeptide can be identified unequivocally, e.g. by its size on a Western blot.

Preferably the specifically binding ligands can be labeled, e.g. with fluorescent labels, enzymes, molecular tags (e.g. GST, myc-tag or the like), radioactive isotopes, or with labeled substances, e.g. labeled secondary antibodies. For MRI (magnetic resonance imaging), the ligands may be chelated with gadolinium, superparamagnetic iron oxide or lanthanides. For PET (positron emission tomography) or SPECT (single photon emission computed tomography) commonly used isotopes include ¹¹C, ¹⁸F, ¹⁵O, ¹³N, ⁸⁶Y, ⁹⁰Y, and ¹⁶Co.

Diagnostic kits may comprise suitable isolated nucleic acid or amino acid sequences of the above identified genes or gene products, labelled or unlabelled, and/or specifically binding ligands (e.g. antibodies) thereto and auxiliary reagents as appropriate and known in the art. The assays may be liquid phase assays as well as solid phase assays (i.e. with one or more reagents immobilized on a support). The diagnostic kits may also include ligands directed towards other molecules indicative of the disease to be diagnosed.

In another aspect, the invention relates to the use of an isolated nucleic acid molecule or a nucleic acid expression vectors as defined above or of an antibody which is directed against at least one polypeptide comprising a sequence as defined above, in a screening assay for the identification and characterization of drugs that are useful in the treatment and/or prophylaxis of Cardiovascular diseases, preferably disorders of lipid metabolism and atherosclerosis.

“Screening assay” according to the present invention relates to assays which allow to identify substances, particularly potential drugs, useful in the treatment and/or prophylaxis of Cardiovascular diseases, preferably disorders of lipid metabolism and atherosclerosis, by screening libraries of substances. “Screening assay” according to the present invention also relates to assays to screen libraries for substances capable of binding to the nucleic acids, polypeptides, peptides or antibodies defined above. Suitable libraries may, for example, include small molecules, peptides, polypeptides or antibodies.

Suitable drugs include “interacting drugs”, i.e. drugs that bind to the polypeptides or nucleic acids identified above. Such interacting drugs may either inhibit or activate the molecule they are bound to. Examples for interacting substances are peptide nucleic acids comprising sequences identified above, antisense RNAs, siRNAs, ribozymes, aptamers, antibodies and molecular affinity bodies (CatchMabs, Netherlands). Such drugs may be used according to any aspect of the present invention, including use for the manufacture of medicaments and methods of treatment and/or prophylaxis of Cardiovascular diseases, preferably disorders of lipid metabolism and atherosclerosis. It is known that such interacting drugs can also be labeled and used as ligands for diagnosis of a disease associated Cardiovascular diseases, preferably disorders of lipid metabolism and atherosclerosis.

The term “expression vector” as used herein does not only relate to RNA or siRNA expressing vectors, but also to vectors expressing peptides, polypeptides or proteins. The transfer of the expression vector into the host cell or host organism hereby may be performed by all known transformation or transfection techniques, including, but not limited to calcium phosphate transformation, lipofection, microinjection. The expression vector may be any known vector that is suitable to allow the expression of the nucleic acid sequence as defined above. Preferred expression vectors possess expression cassettes comprising a promoter that allows an overexpression of the RNA, peptide or polypeptide as defined above. After the transfer of the expression vector into the host cell/host organism one part of the host cells or host organisms are cultured in the presence of at least one candidate of an inhibitor- or activator-molecule and under culture conditions that allow the expression, preferably the overexpression of the RNA, peptide or polypeptide as defined above. The other part of the transfected host cells are cultured under the same culture conditions, but in the absence of the candidate of an inhibitor- or activator-molecule.

In another preferred embodiment, the screening method for the identification and characterization of an interacting molecule useful in the treatment and/or prophylaxis of Cardiovascular diseases, preferably disorders of lipid metabolism and atherosclerosis from a library of test substances comprises the following steps:
a) recombinantly expressing a polypeptide encoded by a nucleic acid molecule sequence as defined above in a host cell,
b) isolating and optionally purifying the recombinantly expressed polypeptide of step (a),
c) optionally labelling of the test substances and/or labelling of the recombinantly expressed polypeptide,
d) immobilizing the recombinantly expressed polypeptide to a solid phase,
e) contacting of at least one test substance with the immobilized polypeptide,
f) optionally one or more washing steps, and
g) detecting the binding of the at least one test substance to the immobilized polypeptide at the solid phase.
h) performing a functional assay.
   Step a) includes the recombinant expression of the above identified polypeptide or of its derivative from a suitable expression system, in particular from cell-free translation, bacterial expression, or baculuvirus-based expression in insect cells.
   Step b) comprises the isolation and optionally the subsequent purification of said recombinantly expressed polypeptides with appropriate biochemical techniques that are familiar to a person skilled in the art.

Alternatively, these screening assays may also include the expression of derivatives of the above identified polypeptides which comprises the expression of said polypeptides as a fusion protein or as a modified protein, in particular as a protein bearing a “tag”-sequence. These “tag”-sequences consist of short nucleotide sequences that are ligated ‘in frame’ either to the N- or to the C-terminal end of the coding region of said target gene. Commonly used tags to label recombinantly expressed genes are the poly-Histidine-tag which encodes a homopolypeptide consisting merely of histidines, particularly six or more histidines, GST (glutathion S-transferase), c-myc, FLAG®, MBP (maltose binding protein), and GFP. In this context the term “polypeptide” does not merely comprise polypeptides with the nucleic acid sequences as listed in Table 10 their naturally occurring homologs, preferably orthologs, more preferably human orthologs, but also derivatives of these polypeptides, in particular fusion proteins or polypeptides comprising a tag-sequence.

These polypeptides, particularly those labelled by an appropriate tag-sequence (for instance a His-tag or GST-tag), may be purified by standard affinity chromatography protocols, in particular by using chromatography resins linked to anti-His-tag-antibodies or to anti-GST-antibodies which are both commercially available. Alternatively, His-tagged molecules may be purified by metal chelate affinity chromatography using Ni-ions. Alternatively to the use of ‘label-specific’ antibodies the purification may also involve the use of antibodies against said polypeptides. Screening assays that involve a purification step of the recombinantly expressed target genes as described above (step 2) are preferred embodiments of this aspect of the invention.

In an—optional—step c) the compounds tested for interaction may be labelled by incorporation of radioactive isotopes or by reaction with luminescent or fluorescent compounds. Alternatively or additionally also the recombinantly expressed polypeptide may be labelled.

In step d) the recombinantly expressed polypeptide is immobilized to a solid phase, particularly (but not limited) to a chromatography resin. The coupling to the solid phase is thereby preferably established by the generation of covalent bonds.

In step e) a candidate chemical compound that might be a potential interaction partner of the said recombinant polypeptide or a complex variety thereof (particularly a drug library) is brought into contact with the immobilized polypeptide.

In an—optional—step f) one or several washing steps may be performed. As a result just compounds that strongly interact with the immobilized polypeptide remain bound to the solid (immobilized) phase.

In step g) the interaction between the polypeptide and the specific compound is detected, in particular by monitoring the amount of label remaining associated with the solid phase over background levels.

Such interacting molecules may be used without functional characterization for diagnostic purposes as described above.

In another aspect, the invention relates to a method for the preparation of a pharmaceutical composition wherein an inhibitor or activator of cell cycle progression is identified according to any of the screening methods described above, synthesized in adequate amounts and formulated into a pharmaceutical composition.

Suitable methods to synthesize the inhibitor or activator molecules are known in the art. For example, peptides or polypeptides can be synthesized by recombinant expression (see also above), antibodies can be obtained from hybridoma cell lines or immunized animals. Small molecules can be synthesized according to any known organic synthesis methods.

Similarly, said inhibitor or activator may be provided by any of the screening methods described above and formulated into a pharmaceutical composition.

Another embodiment of the invention is the use of the screening methods of the invention in the field of cardiovascular diseases, preferably disorders of lipid metabolism and atherosclerosis.

The following examples illustrate the present invention without, however, limiting the same thereto.

Unless otherwise specified, the manipulations of nucleic acids and polypeptides/-proteins can be performed using standard methods of molecular biology and immunology (see, e.g. Maniatis et al. (1989), Molecular cloning: A laboratory manual, Cold Spring Harbour Lab., Cold Spring Harbour, N.Y.; Amusable, F. M. et al. (eds.) “Current protocols in Molecular Biology”. John Wiley and Sons, 1995; Tijssen, P., Practice and Theory of Enzyme Immunoassays, Elsevier Press, Amsterdam, Oxford, N.Y., 1985).

### EXAMPLES

### Example 1

### Generation of dsRNA Molecules for RNAi Experiments

siRNA of a given siRNA sequence were synthesized by Ambion, Inc. (Austin, Tex., USA), using standard methods known to the person skilled in the art of siRNA synthesis.

### Example 2

### Cell Seeding and Transfection of Cells

Huh human hepatoma cells, cultivated in RPMI (Gibco/Invitrogen) medium containing 10% FBS, 1% non-essential amino acid solution (Gibco/Invitrogen), 1% Penicillin/Streptomycin solution (Gibco/Invitrogen), 1% Glutamine (Gibco/Invitrogen) and 1% Hepes pH 8 (Gibco/Invitrogen), were treated with siRNAs at a final concentration of 100 nM using a lipofection based transfection protocol.

24 h before transfection, Huh cells were disattached from the flask by incubation with 3 ml Trypsine solution (Gibco/Invitrogen) for 5 min at 37° C. Cells were harvested by adding 10 ml of RPMI medium to the flask. 4000 cells/well were seeded in black, optical 96well plates (Costar/Corning) in a volume of 100 ul/well. To allow homogenous settling of the cells, important for an even intra well distribution of the cells, the plates were left for 30 min at RT before they were transferred to an incubator with 37° C. and 5% CO₂.

On the day of transfection, for each siRNA the transfection mix was prepared as follows: 4 μl of a 10 μM stock of siRNA was diluted with 64 μl of Opti-MEM (Invitrogen Inc.), and 1.6 μl Oligofectamine transfection reagent (Invitrogen) were diluted with 9.6 μl of Opti-MEM. For complex formation, both solutions were gently mixed and incubated for 20 min at RT. Culture medium was removed from the cells and 80 μl of fresh medium ([DMEM, Invitrogen) were added, followed by addition of 20 μl of transfection mix to each of replicate 3wells per siRNA. Cells were incubated at 37° C. for 4 hours and 50 μl of fresh medium, supplemented with 30% fetal calf serum were added. 24 h after addition of the transfection mix the complete medium described above, was replaced by RPMI medium, containing 2% Lipoprotein deficient serum (LPDS) instead of the 10% FBS.

As an internal control and for intra plate normalization each 96 well screening plate, transfected with 88 different sample siRNAs contained the following 8 control wells: 2 wells with siRNAs directed against HMGCR, 2 wells against SQLE, 3 wells with unspecific control siRNAs sharing no complete sequence homology with any coding sequence in the human transcriptome and 1 well without any siRNA.

The 3 replicate wells, assayed per siRNA were situated on 3 different screening plates (inter plate triplicates).

### Example 3

### Primary Screen

### Cell Staining and Fluorescence Microscopy Based Screening Readout

### Cell Staining:

For a primary readout, the expression level of the LDL receptor (LDLR) was measured by an indirect assay, quantifying the amount of available receptor by the amount of internalized LDL. To this end, 48 h after transfection the supernatant was replaced by pre-warmed fresh Lipoprotein deficient RPMI medium containing 2% LPDS and 3 ug/ml LDL, labelled with the lipid dye DiI (LDL-DiI), supplemented with 1 ug/ml Hoechst for staining of cell nuclei. After an incubation period of 60 min at 37° C. with this staining solution, cells were washed with phosphate buffered saline containing MgCL2 and CaCL2 (PBS+) and fixed with 4% PFA for 30 min at RT.

### Image Acquisition:

Cells were imaged using a fully automated fluorescence microscope from MDC (Molecular Devices Corporation, CA, USA). Per experimental well 6 fields with a dimension of approx. 2×1.5 mm were acquired using excitation/emission conditions, optimized to the spectral properties of the two chromophores, DiI and Hoechst.

### Image Analysis:

To quantify the degree of LDL-DiI uptake, each image acquired in the DiI channel was subjected to an automated image analysis algorithm, programmed using the MetaMorph image analysis software (Universal imaging/MDC). In this algorithm, an adaptive intensity threshold was used to define and measure the area covered by LDL-DiI labelled objects. For each image, this area was normalized to the fraction of total image area covered by cells (cell density).

The normalized LDL-DiI measurements and the cell density values derived from each of the 6 fields for a given well were averaged to obtain two data points (LDL-DiI and cell density) per experimental well. All experimental data points were normalized to the corresponding control data points taken from wells treated with non-template siRNA on the same plate. Finally, the plate-normalized LDL-DiI and cell density data points from corresponding wells on the 3 replicate plates were averaged to genearate a single mean value and standard deviation.

### Validation of Screening Method by Confirming Positive Control Genes

As an internal control and for intra plate normalization each 96 well screening plate, transfected with 88 different sample siRNAs contained the following 8 control wells: 2 wells with siRNAs directed against HMGCR, 2 wells against SQLE, 3 wells with unspecific control siRNAs sharing no complete sequence homology with any coding sequence in the human transcriptome and 1 well without any siRNA.

In addition to its function as positive control gene, SQLE was also part of the screened library, targeted by 3 different siRNAs. 2 of these 3 siRNAs, one of them being identical to the SQLE positive control siRNA, were confirmed as positive in the screen showing LDL-DiI uptake values of 348% and 522% of the corresponding unspecific control value. SiRNAs targeting HMGCR were not present in the screened siRNA library.

### Example 4

### Secondary Screen

Selection of siRNAs

All genes, for which at least one single siRNA showing an increase in LDL-DiI uptake of more then 300% as compared to the negative control had been found in pass1 were subjected to a second round of screening (pass2). To control for potential errors in the synthesis of the siRNAs used for pass1, all siRNAs used for pass2 were de nove synthesized. In addition to the siRNAs found positive in pass1, at least on additional siRNA with new sequence were tested for all genes found positive by only a single positive siRNA in pass 1.

### Assay

Cell cultivation, seeding and transfection conditions as well as the choice of positive and negative control siRNAs, was identical between pass1 and pass2.

Differing from the staining conditions, described above for pass1, the uptake of fluorescently labeled transferrin was included as additional readout in pass2 to control for differences in the activity of receptor mediated uptake in general. To that end the staining solution described for pass 1 was supplemented with the soluble iron binding protein transferrin coupled to the fluorophore alexa488 (invitrogen) in a final concentration of 50 ug/ml. The staining procedure, image acquisition and image analysis was performed as described for pass1 with the only difference that alexa488 staining was imaged in a third channel, optimized to the spectral properties of the fluorophore alexa488. The intensity Transferrin-alexa488 staining was analyzed by the same intensity threshold based algorithm as used for the quantification of the LDL-DiI image data. Final readouts of the pass2 analysis were LDL-DiI uptake, cell proliferation and transferrin-alexa488 uptake.

### Example 4

### Third Pass Screening

Selection of siRNAs

The primary positive criterion for the selection of genes for a third round of analysis (pass3) was the level and the robustness of the increase in LDL-DiI uptake measured in pass 1 and 2. Preferably only genes with at least 2 positive siRNAs were selected. Negative criteria were a strong increase in transferrin uptake as well as a decrease in cell proliferation.

### Assay

Cell cultivation, seeding and transfection conditions as well as the choice of positive and negative control siRNAs, was generally as described above for pass1 with the following differences:

Every siRNA, re-analyzed in pass3 was tested in a final concentration of 10 nM, 30 nM and 100 nM. The specific siRNAs were diluted with negative control siRNA solution such that the final total concentration of siRNA remained 100 nM.

In addition to the three wells, transfected with each siRNA and concentration for LDL-DiI and cell proliferation analysis another 3 wells were transfected for RT-PCR analysis of the knock down efficacy. Transfection for the functional assay and RT-PCR were done on separate experimental plates. For a better comparability all transfections were performed with the same transfection mix. To that end, the volume of the transfection mix generated for each siRNA and concentration as described above was doubled.

Final readouts of pass3 analysis were LDL-DiI uptake, cell proliferation and knockdown efficacy.

Validation of siRNA Efficacy by RT-PCR (qRT-PCR)

48-h after transfection total RNA was extracted from the cells using Invisorb 96 well kits (Invitek, Germany), following the protocol provided by the manufacturer. cDNA was synthesized using TaqMan RT reagents (Applied Biosystems, Foster City, Calif.) following the instructions provided by the manufacturer. Real-Time qPCR with gene-specific primers was performed in the following reaction mix
5.5 μl 2× SybrGreen PCR mix (ABgene, Surrey, UK)
3.0 μl cDNA
2.5 μl 2 μM primers
=11 μl total
in an ABI-7900-HT real-time PCR machine (Applied Biosystems) running the following program:
50° C. 2 min-95° C. 10 min-45 cycles (95° C. 15 sec-60° C. 1 min)-95° C. 15 sec-60° C. 15 sec−95° C. 15 sec (melting curve).

In addition to expression of the gene of interest, expression level of GAPDH as a housekeeper was determined for each sample in order to account for inter-sample variability. The degree of knockdown was determined by comparing the amplification level for the gene of interest, normalized through the level of GAPDH, between samples transfected with a specific siRNA and samples transfected with unspecific control siRNAs.

### Example 4

### Determination of the Expression Level in HepG2, Huh, Primary Hepatocytes, and Whole Liver Cells

The expression levels of targets of the invention were determined using standard methods known to the person skilled in the art. Whereas it is not necessary to perform additional expression profiling experiments in order to practise the invention, some experimental details relating to the expression profiling experiments are provided for information purposes:

Preparation of total RNA was carried out using Trizole (Invitrogen) according to the manufacturer's instruction. The RNA quality was checked by gel-run and the integrity of ribosomal RNA bands using “RNA 6000 Nano Chips” from Agilent Technologies. Sample preparation for hybridization was performed using “Once-Cycle cDNA Synthesis Kit” (Affymetrix) followed by “Gene Chip Expression 3′-Amplification for IVT Labeling Kit” (Affymetrix). Gene Chip Scanner 3000+equipment (Affymetrix) and human Gene Chips “HG-U133 Plus 2” (Affymetrix) were used for signal detection. Signals were analyzed primarily using GCOS software (Affymetrix) and subsequently with GeneData software.

Expression level data are shown in table 4.

### Example 5

### Screening for Compounds Useful in the Treatment and/or Prophylaxis of Atherosclerosis Using a Cell Based Assay

The screening method for the identification of agonists or antagonists of the human cysteinyl leukotriene receptor 2 (CysLTR2; NM_{—}020377) using a cell based assay will be taken as an example.

The recombinant CHO-K1(ATCC No.: CCL-61) screening cell line expresses constitutively the calcium sensitive photoprotein Aequorin. After reconstitution with its cofactor Coelenterazin and increasing intracellular calcium concentration Aequorin is able to emit light (Rizzuto R, Simpson A W, Brini M, Pozzan T.; Nature 358 (1992) 325-327). Additionally, after transfection with a recombinant expression plasmid containing the full length cDNA for human CysLTR2, the screening cell line is stably expressing the CysLTR2 protein (Heise et. al., JBC 275 (2000) 30531-30536). The CysLTR2 screening cell line is able to react on stimulation with known CysLTR2 agonists (i.e. Leukotriene D4 and Leukotriene C4) with an intracellular Ca⁺⁺ release and resulting luminescence can be measured with appropriate luminometer (Milligan G, Marshall F, Rees S, Trends in Pharmacological Sciences 17 (1996) 235-237). Preincubation with CysLTR2 antagonists diminish the Leukotriene D4 or Leukotriene C4 induced Ca⁺⁺ release and consequently the resulting luminescence.

Cells were seeded into 384 well cell culture plates and preincubated for 48 hours in culture medium (DMEM/F12 with Glutamax, Gibco Cat.# 61965-026; 10% Fetal Calf Serum, Gibco Cat.# 10270-106; 1.4 mM Natriumpyruvat, Gibco Cat.# 11360-039; 1.8 mM Natriumbicarbonate, Gibco Cat.# 25080-060; 10 mM HEPES, Gibco Cat.# 15290-026) under standard cell culture conditions (96% humidity, 5% v/v CO₂, 37° C.). Culture medium is replaced by Tyrode buffer (containing 140 mM NaCl, 5 mM KCl, 1 mM MgCl₂, 2 mM CaCl₂, 20 mM Glucose, 20 mM HEPES) plus Coelenterazin (50 μM) and incubation is continued for additional 3-4 hours. Reference agonists Leukotriene D4, Leukotriene C4 or putative agonists are added to the cells and luminescence is measured subsequently. For antagonist screening, 15 min preincubation with putative antagonists is allowed before Leukotriene D4 (3×100⁻⁸ M) stimulus.

### Example 6

### Screening for Compounds Useful in the Treatment and/or Prophylaxis of Atherosclerosis Using a Cell-Free Assay

The screening method for the identification of inhibitors of the human Phosphodiesterase 4B (PDE4B; NM_{—}002600) using a cell-free biochemical assay will be taken as an example. PDE4B (GenBank/EMBL Accession Number: NM_{—}002600, Obernolte et al. Gene. 1993 129, 239-247) was expressed in SD insect cells using the Bac-to-Bac™ baculovirus expression system. Cells were harvested 48 h after infection and suspended in lysis buffer (20 ml/1 l culture, 50 mM Tris-HCl, pH 7.4, 50 mM NaCl, 1 mM MgCl2, 1.5 mM EDTA, 10% Glycerin, 20 μL protease in-hibitor cocktail set III [CalBiochem, La Jolla, Calif. USA]). The cells were disrupted by sonication at 4° C. and cell debris were removed by centrifugation at 15,000×g at 4° C. for 30 minutes. The supernatant is designated PDE4B cell extract and is stored at −80° C.

For determination of the in vitro effect of test substances on the PDE4B reaction, test substances are dissolved in DMSO and serial dilutions in DMSO are performed. 2 μl of the diluted test compounds are placed in wells of microtiter plates (Isoplate; Wallac Inc., Atlanta, Ga.). 50 μl of a dilution of the PDE4B cell extract (see above) is added. The dilution of the PDE4B cell extract will be chosen that during the incubation with substrate the reaction kinetics is linear and less than 70% of the substrate is consumed (typical dilution 1:150 000; dilution buffer: 50 mM Tris/HCl pH 7.5, 8.3 mM MgCl2, 1.7 mM EDTA, 0.2% BSA). The substrate, [5′,8-3H] adenosine 3′,5′-cyclic phosphate (1 μCi/μl; Amersham Pharmacia Biotech., Piscataway, N.J.) is diluted 1:2000 in assay buffer (50 mM Tris/HCl pH 7.5, 8.3 mM MgCl2, 1.7 mM EDTA). The reaction starts by addition of 50 μl (0.025 μCi) of the diluted substrate and incubates at room temperature for 60 min. The reaction is stopped by addition of 25 μl of a suspension containing 18 mg/ml yttrium scintillation proximity beads in water (Amersham Pharmacia Biotech., Piscataway, N.J.). The microtiter plates are sealed, left at room temperature for 60 min, and are subsequently measured in a Microbeta scintillation counter (Wallac Inc., Atlanta, Ga.). IC50 values will be determined by plotting the substrate concentration against the percentage PDE4B inhibition.

TABLE 1

| Target | siRNA | | Proliferation | | |
|---|---|---|---|---|---|
| No. | ID | LDL-Dil Mean % | Mean % | Target Class(es) | Target symbol |
| 1 | 113613 | 724.0965615 | 107.8625974 | Protein kinase, Protease | ANKRD3 |
| 1 | 105742 | 557.2013279 | 129.7804577 | Protein kinase, Protease | ANKRD3 |
| 1 | 105202 | 426.5918541 | 119.7514464 | Protein kinase, Protease | ANKRD3 |
| 2 | 117853 | 579.7551381 | 125.5260757 | Other enzyme | HLCS |
| 2 | 117852 | 374.3721807 | 84.77067934 | Other enzyme | HLCS |
| 2 | 117851 | 348.0013938 | 110.6440668 | Other enzyme | HLCS |
| 3 | 117417 | 447.7431016 | 113.882518 | Membrane protein, Cytochrome P450 | SC4MOL |
| 3 | 117416 | 399.631074 | 108.1712372 | Membrane protein, Cytochrome P450 | SC4MOL |
| 3 | 117418 | 397.3576822 | 102.42365 | Membrane protein, Cytochrome P450 | SC4MOL |
| 4 | 42711 | 527.9275211 | 88.0695743 | Protease | CASP1 |
| 4 | 42626 | 365.2281846 | 105.920996 | Protease | CASP1 |
| 5 | 10418 | 413.9186256 | 93.80525184 | Other enzyme | CDC42 |
| 5 | 10505 | 332.8978976 | 102.4573283 | Other enzyme | CDC42 |
| 6 | 118135 | 361.0739928 | 86.00868898 | Ion channel, Other enzyme | ABCC2 |
| 6 | 116839 | 342.4148736 | 132.2113457 | Ion channel, Other enzyme | ABCC2 |
| 7 | 105039 | 937.1680485 | 122.6094906 | Protease | CTSE |
| 7 | 105041 | 328.0440766 | 113.0076858 | Protease | CTSE |
| 8 | 1147 | 501.3206562 | 107.3294904 | Protein kinase | FRK |
| 8 | 1243 | 377.3780055 | 110.0406079 | Protein kinase | FRK |
| 9 | 8225 | 395.3088334 | 105.3314627 | Other enzyme | HMBS |
| 9 | 117844 | 386.5308856 | 79.21133011 | Other enzyme | HMBS |
| 10 | 106087 | 551.4313155 | 88.44182963 | Transporter, Other enzyme | HSD17B4 |
| 10 | 106089 | 400.7479977 | 107.44149 | Transporter, Other enzyme | HSD17B4 |
| 11 | 121011 | 383.0979608 | 99.41485592 | Transporter | LCN2 |
| 11 | 121012 | 366.9633728 | 131.659559 | Transporter | LCN2 |
| 12 | 1766 | 723.8356377 | 107.0417947 | GPCR | OXTR |
| 12 | 1947 | 444.0141863 | 115.0667872 | GPCR | OXTR |
| 13 | 142836 | 377.9953683 | 127.8244045 | Phosphatase | PPP1R3C |
| 13 | 142834 | 339.4652831 | 105.2028745 | Phosphatase | PPP1R3C |
| 14 | 1547 | 478.2634431 | 133.7832329 | Protein kinase | MAPK9 |
| 14 | 1452 | 319.9391015 | 124.7111395 | Protein kinase | MAPK9 |
| 15 | 1699 | 499.9106861 | 111.7567033 | Protein kinase | MAP2K5 |
| 15 | 118252 | 403.7219125 | 105.1834443 | Protein kinase | MAP2K5 |
| 16 | 43916 | 322.0473129 | 131.2710631 | Other enzyme | TPI1 |
| 16 | 43820 | 321.2912134 | 119.4372173 | Other enzyme | TPI1 |
| 17 | 402 | 471.3653067 | 123.3723017 | Protein kinase | VRK1 |
| 17 | 401 | 386.3750915 | 134.9480071 | Protein kinase | VRK1 |
| 18 | 117695 | 457.7827807 | 107.5940504 | Ion channel | SLC30A2 |
| 18 | 117693 | 425.8395112 | 102.8000404 | Ion channel | SLC30A2 |
| 19 | 118261 | 747.4553015 | 106.336687 | Protein kinase | ULK1 |
| 19 | 118260 | 445.9651692 | 108.8989791 | Protein kinase | ULK1 |
| 20 | 5146 | 478.40008 | 117.5601535 | GPCR | GLP2R |
| 20 | 5237 | 451.8708414 | 104.3488796 | GPCR | GLP2R |
| 21 | 828 | 343.2595327 | 113.2848132 | Protein kinase | SNK |
| 21 | 829 | 340.2448823 | 102.9484122 | Protein kinase | SNK |
| 22 | 19104 | 538.6343597 | 77.27418771 | Protease | SPUVE |
| 22 | 19196 | 464.0190864 | 86.22550377 | Protease | SPUVE |
| 23 | 103354 | 389.1368559 | 101.8826824 | Protein kinase | PASK |
| 23 | 978 | 367.9530516 | 119.4915761 | Protein kinase | PASK |
| 24 | 2240 | 527.5919554 | 97.51493592 | GPCR | OR52A1 |
| 24 | 2061 | 397.4248924 | 112.9250133 | GPCR | OR52A1 |
| 25 | 20115 | 671.5714404 | 104.2916365 | Other | RGS17 |
| 25 | 20024 | 623.6859075 | 95.18970662 | Other | RGS17 |
| 26 | 118397 | 407.9391857 | 120.1308385 | Other enzyme | MYLIP |
| 26 | 118398 | 372.3127731 | 119.6270998 | Other enzyme | MYLIP |
| 27 | 104835 | 524.0586705 | 102.0993329 | Ion channel | PKD1L2 |
| 27 | 104830 | 339.3777111 | 115.0438781 | Ion channel | PKD1L2 |
| 28 | 119221 | 1232.307505 | 120.3077073 | Other enzyme | BPHL |
| 29 | 105141 | 1135.645376 | 111.8054786 | Protease | USP3 |
| 30 | 122236 | 1130.561627 | 109.6859813 | Other enzyme | NCE2 |
| 31 | 43781 | 1039.484616 | 117.1937166 | Ion channel | KCNK17 |
| 32 | 103636 | 1008.035233 | 80.27430588 | Protein kinase | WEE1 |
| 33 | 45922 | 995.1241621 | 107.4914643 | Ion channel | KCNE1 |
| 34 | 117371 | 961.5218898 | 104.0451117 | Membrane protein | RNUT1 |
| 35 | 111157 | 911.0548065 | 118.3141676 | Receptor, Transporter | M6PR |
| 36 | 38979 | 909.6821061 | 112.7311274 | Other enzyme | MGC39650 |
| 37 | 121933 | 894.7821748 | 109.4947078 | Metabolic kinase | FLJ22761 |
| 38 | 119829 | 883.6759642 | 125.6237574 | Other enzyme | PAPOLG |
| 39 | 19471 | 880.6891857 | 105.2230754 | Other enzyme | DNM1L |
| 40 | 120442 | 872.601332 | 113.0064783 | Other | PSMD14 |
| 41 | 105154 | 868.7484538 | 109.3678977 | Membrane protein, Protease | BACE |
| 42 | 6196 | 862.0651883 | 81.97030552 | GPCR | FKSG79 |
| 43 | 105753 | 815.426425 | 112.004123 | Extracellular Factor, Protease | LCN7 |
| 44 | 21895 | 783.7479458 | 92.43528375 | Other | STARD8 |
| 45 | 120445 | 771.9380665 | 107.8213079 | Metabolic kinase | RASGRP2 |
| 46 | 111807 | 757.3894801 | 123.5997027 | Protease | QPCT |
| 47 | 1721 | 750.0679293 | 124.7542444 | GPCR | ADORA1 |
| 48 | 1774 | 742.4680415 | 110.8603652 | GPCR | TACR1 |
| 49 | 117712 | 732.0427107 | 99.03670761 | Metabolic kinase | ABCA10 |
| 50 | 1795 | 717.9672016 | 111.0300467 | Other enzyme, GPCR | GPR56 |
| 51 | 117084 | 717.0543713 | 98.15556498 | Metabolic kinase | PRPSAP2 |
| 52 | 119926 | 702.3467999 | 112.1197297 | Other | SLC26A10 |
| 53 | 9067 | 701.3622509 | 101.2032536 | Other enzyme | ADH7 |
| 54 | 121179 | 694.363976 | 99.0917368 | Transporter | OBP2A |
| 55 | 38327 | 691.601585 | 110.4531546 | GPCR | MRGX1 |
| 56 | 111813 | 690.4898311 | 101.6885907 | Receptor | TNFRSF13B |
| 57 | 107569 | 685.2375591 | 113.0692617 | Other enzyme | TP53I3 |
| 58 | 10560 | 683.6891137 | 119.9488634 | Extracellular Factor, Protease | CPB2 |
| 59 | 10235 | 674.784399 | 103.4678804 | Other enzyme | ARFD1 |
| 60 | 104127 | 671.3189247 | 124.5246892 | Membrane protein, Protease | ADAM29 |
| 61 | 112077 | 669.0628066 | 106.5058056 | Membrane protein, Other enzyme | AGPAT3 |
| 62 | 105010 | 667.8419405 | 115.448212 | Protease | CTSK |
| 63 | 4154 | 666.2737834 | 115.6483854 | GPCR | GPR39 |
| 64 | 40980 | 665.0042604 | 115.2723859 | Protein kinase | TTBK |
| 65 | 120756 | 657.8375343 | 98.83391855 | Ion channel, Other enzyme | ATP2A3 |
| 66 | 1627 | 657.6377629 | 110.8290477 | Protein kinase | FYN |
| 67 | 122128 | 654.5962745 | 94.78045661 | Other | C20orf185 |
| 68 | 2207 | 652.1480701 | 108.8107556 | GPCR | HM74 |
| 69 | 4633 | 648.8978572 | 109.3070437 | GPCR | TRHR |
| 70 | 119952 | 643.060823 | 111.3827969 | Ion channel | SLC12A2 |
| 71 | 105325 | 639.0989022 | 117.6878074 | Protease | CPA3 |
| 72 | 112330 | 638.5537811 | 112.097435 | Membrane protein, Other enzyme | D4ST1 |
| 73 | 121576 | 637.0982295 | 111.5476447 | Other | APC2 |
| 74 | 117799 | 634.8557057 | 95.77884911 | Membrane protein, Transporter | MGC52019 |
| 75 | 104458 | 623.4163967 | 109.9925709 | Ion channel | VDAC2 |
| 76 | 112453 | 621.914958 | 104.8333388 | Other enzyme | OGT |
| 77 | 121337 | 620.1229465 | 76.02226238 | Other | CENPE |
| 78 | 110844 | 616.8572379 | 107.0996448 | Protein kinase | BCR |
| 79 | 119422 | 615.5313864 | 124.7454668 | Other | ARHGEF1 |
| 80 | 119276 | 611.4653566 | 116.6563534 | Other enzyme | VCP |
| 81 | 118817 | 607.1952205 | 114.8743124 | Other enzyme | MCCC1 |
| 82 | 118114 | 604.2286536 | 109.025425 | Other enzyme | FKBP7 |
| 83 | 118462 | 603.7820058 | 116.0990749 | Other enzyme | KIF13B |
| 84 | 46510 | 602.1553771 | 117.4146548 | GPCR | TG1019 |
| 85 | 119129 | 596.6163895 | 99.86760244 | Other enzyme | CRMP1 |
| 86 | 119399 | 596.5066921 | 108.1484036 | Membrane protein | ELOVL3 |
| 87 | 122166 | 596.0888651 | 123.6677082 | PDE, TF | APEX1 |
| 88 | 45063 | 595.5817158 | 108.7972263 | GPCR | GALR2 |
| 89 | 117601 | 591.648211 | 101.7554096 | Ion channel | SLC12A9 |
| 90 | 118446 | 586.7366596 | 87.23668446 | Other enzyme | KIF2C |
| 91 | 18240 | 585.1080767 | 150.6046065 | Other enzyme | FTCD |
| 92 | 104559 | 583.2891731 | 93.57598288 | Ion channel | CACNA2D2 |
| 93 | 1407 | 582.6120269 | 107.4681385 | Protein kinase | MAPK14 |
| 94 | 119077 | 580.3892176 | 107.480239 | Other enzyme | HEXA |
| 95 | 1371 | 579.6300222 | 118.6112653 | Metabolic kinase | SPHK1 |
| 96 | 106537 | 576.8755334 | 108.3728865 | Ion channel, Other enzyme | ATP5J |
| 97 | 120500 | 569.7068059 | 109.7162037 | Protease | POLG2 |
| 98 | 111654 | 569.0455244 | 89.29363902 | Receptor | CSF2RA |
| 99 | 118718 | 568.5187083 | 140.2249957 | Other | SERPINB9 |
| 100 | 120608 | 566.5601546 | 96.91618938 | Other enzyme | UBASH3A |
| 101 | 142253 | 565.0705142 | 96.60817895 | Other | PPP1R7 |
| 102 | 111081 | 561.6549265 | 105.5657572 | Protein kinase | HIPK1 |
| 103 | 103786 | 561.4171922 | 106.9513773 | Metabolic kinase | GUK1 |
| 104 | 121943 | 560.3439799 | 121.3027078 | Other enzyme | C2orf8 |
| 105 | 121806 | 558.8248481 | 102.018319 | Other | ITLN1 |
| 106 | 15527 | 555.5907735 | 113.5415583 | PDE | PDE1A |
| 107 | 143005 | 554.7628422 | 96.54027294 | Other | PKIA |
| 108 | 110607 | 554.5198986 | 113.1832064 | Receptor | GHR |
| 109 | 107834 | 553.9818333 | 109.131078 | Other enzyme | AASS |
| 110 | 121163 | 551.6493273 | 91.0952295 | | LOC339133 |
| 111 | 118522 | 547.5230683 | 96.41671994 | Other | KIAA0449 |
| 112 | 120179 | 540.9753304 | 113.9270416 | Other enzyme | UBE3A |
| 113 | 34999 | 538.7824646 | 105.9660181 | Membrane protein | FLJ14681 |
| 114 | 6091 | 535.7500463 | 117.4985747 | GPCR | GPR84 |
| 115 | 103829 | 534.428009 | 104.0677126 | | LOC374425 |
| 116 | 119396 | 529.5612528 | 129.9072277 | Other | ARHGEF7 |
| 117 | 8816 | 528.2010911 | 75.09671631 | Phosphatase | SMPD1 |
| 118 | 15339 | 523.459779 | 118.0882192 | Other | AKAP5 |
| 119 | 29459 | 521.6044998 | 124.3187155 | Protease, GPCR | FLJ21839 |
| 120 | 16059 | 520.3864804 | 104.3274648 | Other enzyme | PDHA2 |
| 121 | 104173 | 516.8720868 | 109.2701396 | Membrane protein, Protease | ADAM19 |
| 122 | 120611 | 514.8446941 | 98.9901288 | Other enzyme | RAB25 |
| 123 | 142929 | 514.641299 | 114.9158966 | Other enzyme | PRKAB1 |
| 124 | 35220 | 513.79528 | 109.7998872 | PDE | CNP |
| 125 | 119469 | 512.6228606 | 102.7033106 | Other | GCHFR |
| 126 | 121471 | 512.2218386 | 88.8904603 | Other | BCL10 |
| 127 | 122003 | 511.1386983 | 117.9098859 | Other enzyme | CTMP |
| 128 | 114058 | 501.7929708 | 108.376556 | Membrane protein | APP |
| 129 | 117031 | 499.8903038 | 128.6630551 | Other enzyme | GLRX |
| 130 | 110906 | 499.0068257 | 99.27448263 | Receptor | CXADR |
| 131 | 119517 | 496.4590532 | 107.397945 | Metabolic kinase | PRPS1L1 |
| 132 | 114048 | 496.432642 | 104.5733205 | Extracellular Factor | PROS1 |
| 133 | 809 | 496.0368444 | 109.2690557 | Receptor, Protein kinase | MERTK |
| 134 | 137195 | 492.7135544 | 97.82416987 | Membrane protein, Transporter | STX10 |
| 135 | 118341 | 489.8746301 | 106.0839193 | Other | Dlc2 |
| 136 | 46319 | 488.5824125 | 107.5095504 | TF, Other enzyme | KLP1 |
| 137 | 7204 | 488.1470659 | 118.6782488 | Ion channel | KCNN4 |
| 138 | 119081 | 487.2951811 | 117.7883959 | Other enzyme | MAN2B1 |
| 139 | 745 | 486.0636832 | 111.7504375 | Protein kinase | STK10 |
| 140 | 119216 | 483.9639174 | 115.4574626 | Membrane protein, Other enzyme | BCS1L |
| 141 | 117277 | 482.0554412 | 110.68092 | Ion channel | SLC22A14 |
| 142 | 14775 | 481.7919942 | 127.2075723 | Ion channel, Other enzyme | NDUFA1 |
| 143 | 119182 | 481.2601456 | 108.4484729 | Other | SCP2 |
| 144 | 1286 | 480.4428342 | 143.2857253 | Metabolic kinase | FLJ22055 |
| 145 | 1961 | 480.3311394 | 107.9689303 | GPCR | GPR1 |
| 146 | 119782 | 478.1125004 | 117.7041399 | Other enzyme | ADARB1 |
| 147 | 135366 | 477.8044802 | 101.0742182 | Receptor, Other enzyme | C20orf41 |
| 148 | 42362 | 477.3018528 | 116.0371527 | GPCR | OPN1LW |
| 149 | 12155 | 475.4036511 | 103.5565493 | Receptor | PVRL2 |
| 150 | 11 | 472.1435798 | 129.4741064 | Receptor, Protein kinase | ACVRL1 |
| 151 | 7881 | 470.7065238 | 105.5885503 | Protease | CAPN3 |
| 152 | 10428 | 470.1389417 | 103.1033828 | Other enzyme | ARF4 |
| 153 | 16123 | 468.9022491 | 112.8774241 | Other enzyme | HADHSC |
| 154 | 1049 | 468.8637745 | 129.5102382 | Protein kinase | CLK4 |
| 155 | 919 | 468.8403522 | 110.1488223 | Protein kinase | IKBKE |
| 156 | 121066 | 465.8888922 | 108.6829237 | Membrane protein, Transporter | PLSCR3 |
| 157 | 105169 | 465.65 | 109.56 | Protease | CAPN7 |
| 158 | 36311 | 461.5506217 | 101.7180979 | Other | RGS18 |
| 159 | 5884 | 460.9703523 | 100.2741634 | Extracellular Factor, GPCR | GPRC5D |
| 160 | 44940 | 460.1503308 | 117.9056609 | Extracellular Factor, Other enzyme | SOD3 |
| 161 | 1398 | 459.3250946 | 112.682103 | Protein kinase | KIS |
| 162 | 104626 | 459.0052531 | 106.500977 | Ion channel | SCN8A |
| 163 | 15563 | 458.1980207 | 99.48194892 | Other | CCM1 |
| 164 | 136772 | 455.6291794 | 112.5796027 | Protease | MAPK8IP3 |
| 165 | 46183 | 455.0369741 | 95.40698084 | Membrane protein, Other enzyme | DDX19 |
| 166 | 5377 | 454.3248519 | 102.7635827 | NHR | PPARD |
| 167 | 103491 | 453.9228137 | 104.860333 | Protein kinase | MAP3K6 |
| 168 | 117929 | 453.4469364 | 124.9105413 | Ion channel, Other enzyme | ATP5L |
| 169 | 110591 | 452.1013707 | 99.46085555 | Other enzyme | CPT2 |
| 170 | 103534 | 450.854997 | 99.55271092 | Metabolic kinase | UCK1 |
| 171 | 119066 | 448.5023269 | 116.4950259 | Other enzyme | PAFAH2 |
| 172 | 106403 | 447.7355841 | 103.1864783 | Other enzyme | ALDH7A1 |
| 173 | 121059 | 443.7782504 | 121.3182105 | Membrane protein, Other enzyme | NLGN3 |
| 174 | 121219 | 443.7626772 | 95.08540306 | Other enzyme | AGA |
| 175 | 117366 | 441.8016351 | 112.146955 | Receptor, Transporter, Other enzyme | ABCC9 |
| 176 | 105936 | 441.7006486 | 123.6696823 | Other enzyme | BCKDHB |
| 177 | 105919 | 441.4001365 | 91.29145842 | Other enzyme | ACYP2 |
| 178 | 103932 | 440.2236799 | 108.4298231 | Receptor, Protease | CRN |
| 179 | 43139 | 440.0553435 | 112.0568553 | GPCR | VN1R2 |
| 180 | 9108 | 439.4782496 | 97.36939074 | Other enzyme | GAD2 |
| 181 | 111592 | 439.2244568 | 94.54135852 | Membrane protein, Other enzyme | UST |
| 182 | 104388 | 438.7260273 | 102.5332305 | Ion channel | CLCN3 |
| 183 | 115931 | 437.8127994 | 135.1133409 | Membrane protein, Transporter | SLC7A8 |
| 184 | 30832 | 437.5023782 | 105.2856129 | Receptor | LENG4 |
| 185 | 105076 | 437.17 | 117.45 | Protease | USP13 |
| 186 | 44885 | 436.7502954 | 114.8286775 | Transporter | FABP6 |
| 187 | 103580 | 436.062396 | 108.1960182 | Protein kinase | MAP2K4 |
| 188 | 1555 | 435.5537573 | 109.605547 | Receptor, Protein kinase | EPHA5 |
| 189 | 105163 | 435.31 | 109.22 | Protease | USP25 |
| 190 | 105773 | 435.0740598 | 144.0864242 | Membrane protein, Other enzyme | GGTL3 |
| 191 | 37190 | 434.0342172 | 88.66505884 | ion channel | TPCN2 |
| 192 | 121953 | 430.5466199 | 105.0355231 | Other | NIPA2 |
| 193 | 9040 | 430.2437962 | 95.25449892 | Receptor | ITGAX |
| 194 | 119716 | 429.962131 | 117.0526368 | Membrane protein, Other enzyme | GCS1 |
| 195 | 1794 | 429.7748549 | 110.2235499 | GPCR | SMO |
| 196 | 115136 | 426.3537874 | 120.697878 | Membrane protein | BTNL3 |
| 197 | 116921 | 426.3489396 | 103.3179972 | Ion channel | SLC6A4 |
| 198 | 117213 | 424.9317649 | 114.6683856 | Ion channel, Other enzyme | ATP6V0B |
| 199 | 10426 | 424.6800256 | 100.6599002 | Other enzyme | ARF1 |
| 200 | 657 | 424.2803144 | 110.0707016 | Protein kinase | FER |
| 201 | 46002 | 424.0998283 | 106.3030606 | Other | CST4 |
| 202 | 105830 | 423.9794398 | 132.1654218 | Phosphatase | PPP1CC |
| 203 | 104815 | 422.7271262 | 108.4054772 | Ion channel | KCNK16 |
| 204 | 142280 | 422.4332159 | 117.4788526 | Other | PRKAR2B |
| 205 | 1940 | 422.2064234 | 111.9069403 | GPCR | HTR2C |
| 206 | 103397 | 421.7134033 | 103.2349516 | Receptor, Protein kinase | PRKCM |
| 207 | 117187 | 421.3769777 | 89.26506104 | Membrane protein, Other enzyme | AOC3 |
| 208 | 119405 | 419.7767205 | 115.1077039 | Other | RALGPS2 |
| 209 | 111208 | 419.5450302 | 111.3857744 | Receptor | PVRL1 |
| 210 | 118568 | 419.5080913 | 110.2725123 | Other enzyme | SUOX |
| 211 | 383 | 417.6612083 | 110.276135 | Protein kinase | TEC |
| 212 | 118038 | 417.4594611 | 98.91886222 | Other enzyme | NPL |
| 213 | 42454 | 417.4525613 | 136.8225457 | Membrane protein | BCL2L10 |
| 214 | 118423 | 417.3833267 | 113.9351218 | Other | KIF2 |
| 215 | 104231 | 415.5472125 | 122.6465741 | | ADAMTS6 |
| 216 | 121036 | 414.2913367 | 112.0586636 | Phosphatase | OBDPF |
| 217 | 5834 | 413.6532193 | 107.9929627 | Extracellular Factor, GPCR | GPRC5B |
| 218 | 114204 | 412.0637499 | 108.3105401 | Other enzyme | GLUL |
| 219 | 119258 | 411.550307 | 123.7394163 | Other enzyme | DPYSL4 |
| 220 | 111728 | 410.8622342 | 107.615019 | Receptor | LILRB5 |
| 221 | 119072 | 410.7692094 | 112.7932252 | Other enzyme | GALNS |
| 222 | 121053 | 410.6748168 | 125.8988577 | Other enzyme | FLJ10948 |
| 223 | 142803 | 408.4220781 | 111.9784737 | Other | PRKRIR |
| 224 | 117248 | 407.8600302 | 109.1391643 | Membrane protein, Other enzyme | PIGL |
| 225 | 111369 | 406.8963067 | 117.544814 | Receptor | TNFRSF14 |
| 226 | 118232 | 406.0702778 | 110.9422262 | Protein kinase, TF | ATM |
| 227 | 10238 | 404.4325747 | 102.5970025 | Other | ARF3 |
| 228 | 118663 | 404.1548174 | 110.3151909 | Protease | SERPINB2 |
| 229 | 13014 | 402.3581073 | 105.059737 | Other | VAV2 |
| 230 | 118922 | 401.7314201 | 125.7291744 | Membrane protein, Other enzyme | CA14 |
| 231 | 119792 | 401.5504069 | 112.8674237 | Extracellular Factor, Receptor | TRAP1 |
| 232 | 103447 | 401.4657662 | 113.3929041 | Protein kinase | CAMK1G |
| 233 | 23376 | 400.903468 | 129.3779453 | Protease | LAP3 |
| 234 | 17099 | 400.6668315 | 121.5410914 | Other enzyme | MDH2 |
| 235 | 138240 | 399.8410482 | 102.9862625 | Extracellular Factor, Other enzyme | PRKCABP |
| 236 | 9004 | 399.0331463 | 105.8619609 | Extracellular Factor | CRH |
| 237 | 1785 | 398.6434209 | 116.8090635 | GPCR | GPR3 |
| 238 | 107446 | 397.8207756 | 101.3664701 | Ion channel, Other enzyme | NDUFA10 |
| 239 | 108267 | 397.8059949 | 113.9873132 | Receptor | C1QR1 |
| 240 | 122036 | 397.8001642 | 113.3477815 | Other | FLJ32389 |
| 241 | 118553 | 397.6307055 | 105.0768078 | Extracellular Factor | SERPINA7 |
| 242 | 119612 | 397.6118587 | 98.7004085 | Other enzyme | DCTD |
| 243 | 121775 | 397.5898454 | 117.3560989 | Extracellular Factor | ANGPTL4 |
| 244 | 110854 | 397.3658168 | 82.32707254 | Membrane protein, Protein kinase | DCAMKL1 |
| 245 | 126062 | 396.7226181 | 116.1216962 | Phosphatase | FLJ23751 |
| 246 | 118792 | 396.7068231 | 121.4933783 | Other enzyme | BIA2 |
| 247 | 120597 | 396.6975195 | 138.6302845 | Other enzyme | HDAC8 |
| 248 | 119183 | 396.3604372 | 109.938439 | Other enzyme | UPP1 |
| 249 | 121277 | 395.9619555 | 100.6813608 | Other enzyme | ANG |
| 250 | 117497 | 395.0172017 | 96.78517027 | Ion channel | SLC39A2 |
| 251 | 1704 | 394.8804771 | 109.210148 | Metabolic kinase | PANK1 |
| 252 | 119905 | 394.0107872 | 145.6705418 | Membrane protein, Transporter | SLC25A11 |
| 253 | 121507 | 391.1060844 | 95.47181587 | Other enzyme | DDX21 |
| 254 | 121103 | 390.9844878 | 117.1793186 | Other enzyme | CACH-1 |
| 255 | 6501 | 390.7851321 | 110.5802459 | GPCR | ADRA1D |
| 256 | 43395 | 390.66731 | 115.7281563 | Membrane protein, Other enzyme | NLGN4Y |
| 257 | 1541 | 390.469141 | 109.3497418 | Protein kinase, GPCR | PDGFRB |
| 258 | 648 | 389.0628479 | 104.4644251 | Receptor, Protein kinase | EPHA1 |
| 259 | 22653 | 388.9007577 | 98.13047718 | Other | CENTD2 |
| 260 | 112041 | 388.5370327 | 114.1528932 | Other enzyme | AD-017 |
| 261 | 116939 | 388.4574861 | 107.1931501 | Other enzyme | ACLY |
| 262 | 111049 | 387.2108178 | 103.2891867 | Metabolic kinase | FUK |
| 263 | 120730 | 386.988569 | 124.866649 | Other | RHEBL1 |
| 264 | 1776 | 386.8180132 | 88.58584251 | GPCR | ADCYAP1R1 |
| 265 | 139134 | 386.3029721 | 118.0988203 | Metabolic kinase | PIP5K1B |
| 266 | 118865 | 386.072946 | 113.9593622 | Other | SERPINB11 |
| 267 | 119034 | 384.48802 | 120.2533328 | Other enzyme | GCH1 |
| 268 | 41802 | 384.3933062 | 114.8527283 | Membrane protein, Other enzyme | UGT2B11 |
| 269 | 115614 | 383.0489224 | 108.3278972 | TF | ATF1 |
| 270 | 120142 | 382.7535652 | 106.4879167 | Ion channel | SLC39A4 |
| 271 | 129420 | 382.5683087 | 101.9266991 | Metabolic kinase | PIK3AP1 |
| 272 | 35139 | 381.7248731 | 129.8871627 | Other enzyme | LDHL |
| 273 | 112237 | 381.5310991 | 121.4406138 | Other enzyme | AGXT2L1 |
| 274 | 118332 | 381.2599671 | 116.1870781 | Other | DNCLI1 |
| 275 | 202390 | 381.0330204 | 114.3109014 | Membrane protein, Other enzyme | HS3ST4 |
| 276 | 111442 | 379.6241977 | 116.4321259 | Membrane protein, Other enzyme | CHST2 |
| 277 | 119734 | 379.4911971 | 96.99996658 | Other enzyme | GNA13 |
| 278 | 46555 | 379.0545062 | 108.3805591 | Other | MGC30208 |
| 279 | 112493 | 378.5408605 | 89.61741639 | Membrane protein, Other enzyme | GALNT10 |
| 280 | 120542 | 378.1641943 | 103.8492052 | Other enzyme | NEDL1 |
| 281 | 143975 | 377.9499383 | 131.2863056 | Metabolic kinase | PIK3CD |
| 282 | 202509 | 377.8694154 | 97.95793669 | Protein kinase | KIAA0551 |
| 283 | 26615 | 376.8374238 | 103.2283291 | Other enzyme | RHOT1 |
| 284 | 2021 | 376.5797096 | 132.656305 | GPCR | MTNR1B |
| 285 | 1017 | 376.156958 | 114.0562439 | Protein kinase | FLJ10074 |
| 286 | 44902 | 375.5435041 | 106.7019829 | Other enzyme | GAPD |
| 287 | 121821 | 374.6192962 | 123.4205013 | GPCR | C20orf12 |
| 288 | 3573 | 373.9255838 | 112.1625485 | TF | TRIM16 |
| 289 | 111379 | 373.9111692 | 92.20755942 | Receptor | TNFRSF10C |
| 290 | 119725 | 373.7070491 | 123.9564273 | Other enzyme | RPC32 |
| 291 | 119012 | 373.7018113 | 113.7521602 | Other enzyme | ARSE |
| 292 | 11202 | 373.6632911 | 115.5299636 | Receptor | ITGB8 |
| 293 | 119352 | 372.7179192 | 113.6092933 | Other enzyme | DPYSL5 |
| 294 | 111028 | 372.5956763 | 104.5128705 | Protein kinase | MARK4 |
| 295 | 6242 | 372.0353982 | 101.495524 | GPCR | P2RY12 |
| 296 | 6829 | 371.9102091 | 113.1570297 | GPCR | GPR113 |
| 297 | 120986 | 370.5294104 | 94.75769169 | Other | PLIN |
| 298 | 282 | 370.3960758 | 116.4681362 | Metabolic kinase | PIK4CB |
| 299 | 119249 | 369.9428144 | 113.4634652 | Other | RAPGEF3 |
| 300 | 121002 | 369.8711654 | 116.7251919 | Transporter | RBP2 |
| 301 | 112098 | 369.5515149 | 128.5647748 | Membrane protein, Other enzyme | LOC57168 |
| 302 | 120006 | 369.3823539 | 107.6925684 | Ion channel | SLCO1B1 |
| 303 | 9145 | 369.3015495 | 102.517714 | Phosphatase | PLCB3 |
| 304 | 45672 | 369.0790566 | 108.3570787 | Other | ASB10 |
| 305 | 5997 | 368.5900838 | 116.6606331 | GPCR | MC3R |
| 306 | 5922 | 368.0630501 | 122.4474375 | NHR | NR2F2 |
| 307 | 112027 | 367.9815221 | 98.60310902 | Membrane protein, Other enzyme | LPAAT-e |
| 308 | 42079 | 367.8448019 | 100.7261083 | Ion channel | KCNJ4 |
| 309 | 104120 | 366.9358617 | 128.2650179 | Membrane protein, Protease | ADAM18 |
| 310 | 104465 | 366.3929912 | 113.6588387 | Ion channel, Other enzyme | KCNAB2 |
| 311 | 118241 | 365.9321943 | 106.4669235 | Metabolic kinase | NME3 |
| 312 | 12487 | 365.5328622 | 124.7024867 | Other enzyme | SMARCA3 |
| 313 | 139155 | 365.2402515 | 95.96747073 | Membrane protein, Transporter | STX7 |
| 314 | 7014 | 365.2343599 | 105.1510693 | Ion channel | CLCN5 |
| 315 | 107742 | 365.1722202 | 121.6315236 | Membrane protein, Other enzyme | HSD11B1 |
| 316 | 122070 | 363.9494025 | 97.98606684 | Other | ARHGEF19 |
| 317 | 119167 | 363.5067705 | 132.3645072 | Membrane protein, Other enzyme | LCT |
| 318 | 121082 | 363.4719077 | 123.18997 | Ion channel | SFXN1 |
| 319 | 34166 | 362.4141694 | 111.3714375 | Metabolic kinase | CARD11 |
| 320 | 36798 | 361.9815571 | 94.43917848 | Other enzyme | LYPLAL1 |
| 321 | 6764 | 361.5881078 | 116.6719888 | GPCR | MRGX2 |
| 322 | 112281 | 361.211568 | 104.7476966 | Receptor | HAVCR2 |
| 323 | 1359 | 360.6229671 | 109.7421686 | Protein kinase | DKFZp761P1010 |
| 324 | 120792 | 360.1322955 | 119.6152026 | Ion channel, Other enzyme | ATP6V1E1 |
| 325 | 120227 | 359.5034833 | 136.1347787 | Ion channel, Other enzyme | ATP2B1 |
| 326 | 117144 | 359.037843 | 84.88109411 | Other enzyme | UAP1 |
| 327 | 202463 | 358.8380192 | 105.8085832 | Metabolic kinase | LOC375133 |
| 328 | 326 | 358.400703 | 108.0356713 | Protein kinase | MAP2K1 |
| 329 | 121132 | 358.0920934 | 124.2705074 | Receptor | ITGA1 |
| 330 | 40762 | 357.9844049 | 129.9341867 | Protein kinase | SNF1LK |
| 331 | 106985 | 357.0935603 | 116.5846304 | Cytochrome P450 | SPR |
| 332 | 118634 | 357.0098956 | 112.4279362 | Other | CCNF |
| 333 | 1709 | 356.536362 | 114.2935848 | GPCR | AVPR2 |
| 334 | 119230 | 354.9303323 | 100.1501686 | Other | ARHGEF2 |
| 335 | 111644 | 354.8077797 | 119.2212102 | Membrane protein, Other enzyme | SIAT10 |
| 336 | 103331 | 354.3918817 | 107.4194451 | Receptor, Protein kinase | ERBB4 |
| 337 | 105105 | 353.6602154 | 122.3410109 | Protease | BAP1 |
| 338 | 6671 | 353.5108852 | 115.217319 | GPCR | CD97 |
| 339 | 117749 | 352.7265894 | 128.6629429 | Other enzyme | FLJ30473 |
| 340 | 104678 | 352.5167014 | 112.8467449 | Protein kinase, Ion channel | TRPM7 |
| 341 | 4236 | 352.3335634 | 110.0391798 | GPCR | P2RY1 |
| 342 | 119150 | 352.2135679 | 114.6078946 | Other enzyme | APOBEC1 |
| 343 | 120536 | 352.1285019 | 92.66377649 | Protease | USP34 |
| 344 | 4084 | 351.6370776 | 117.7994424 | GPCR | CNR1 |
| 345 | 8584 | 351.4439648 | 123.2153662 | Other enzyme | RAG1 |
| 346 | 118025 | 350.756329 | 106.4389061 | Other enzyme | FLJ21963 |
| 347 | 104025 | 350.4577675 | 107.774929 | Extracellular Factor, Protease | MMP13 |
| 348 | 142304 | 350.2300498 | 105.844301 | Protein kinase | MAPK3 |
| 349 | 119004 | 349.6278446 | 121.9397977 | Other enzyme | FLJ23322 |
| 350 | 112199 | 349.199572 | 122.4161424 | Membrane protein, Other enzyme | CHST5 |
| 351 | 140383 | 348.776124 | 139.6400449 | Membrane protein, Phosphatase | MIR16 |
| 352 | 43202 | 348.6681287 | 102.831723 | Other | LOC134285 |
| 353 | 118558 | 348.1809774 | 108.7933859 | Membrane protein, Cytochrome P450 | TYR |
| 354 | 122033 | 346.9686784 | 105.2816898 | Other | BIN1 |
| 355 | 110947 | 346.599087 | 100.2355326 | Receptor | GFRA3 |
| 356 | 122374 | 346.4990721 | 118.5241141 | Other | CALML3 |
| 357 | 121768 | 346.4657572 | 121.6190857 | Membrane protein | HMP19 |
| 358 | 110667 | 346.3715528 | 119.2857178 | Membrane protein, Other enzyme | UGT1A1 |
| 359 | 119683 | 346.0605729 | 116.9674912 | Other | SLC9A3R1 |
| 360 | 105368 | 345.9569033 | 115.4178217 | Membrane protein, Protease | MBTPS2 |
| 361 | 117476 | 345.8722247 | 105.0576544 | Ion channel | SLC30A4 |
| 362 | 8110 | 345.8696274 | 68.11996579 | Protease | PLG |
| 363 | 108254 | 345.5531839 | 104.001058 | Extracellular Factor, Receptor | PLA2R1 |
| 364 | 119254 | 345.1450824 | 112.4167696 | Other enzyme | POLD2 |
| 365 | 120528 | 345.139316 | 114.9820103 | Ion channel, Other enzyme | ATP2C1 |
| 366 | 114721 | 345.0744395 | 113.4585363 | TF, Protease | SUPT16H |
| 367 | 120404 | 344.600459 | 101.6414074 | Other enzyme | ARHI |
| 368 | 121560 | 344.3589134 | 88.96359657 | Other | ARPC4 |
| 369 | 8759 | 343.7750672 | 101.211582 | Extracellular Factor, Transporter | ORM1 |
| 370 | 121689 | 342.7532716 | 94.90392556 | Other | C4.4A |
| 371 | 116959 | 342.7449346 | 103.7426412 | Transporter | CLNS1A |
| 372 | 18269 | 342.5183985 | 120.5404591 | Membrane protein, Other enzyme | MAN1A2 |
| 373 | 4118 | 342.2340458 | 112.4560621 | Cytochrome P450 | CYP2F1 |
| 374 | 120313 | 342.1408384 | 109.1143318 | Other enzyme | UBE2E1 |
| 375 | 14778 | 342.0333685 | 103.8887697 | Ion channel, Other enzyme | NDUFB2 |
| 376 | 1554 | 340.9120072 | 146.1444041 | Protein kinase | CDKL1 |
| 377 | 120581 | 340.6051973 | 108.7843543 | Other enzyme | RRAGB |
| 378 | 135789 | 340.4662845 | 108.5254447 | Other | AKAP3 |
| 379 | 104313 | 340.3715661 | 136.4855116 | Extracellular Factor, Ion channel | GLRA1 |
| 380 | 5020 | 340.1495923 | 100.9024863 | GPCR | PNR |
| 381 | 103787 | 339.8383981 | 121.4124757 | Protein kinase | MAP3K11 |
| 382 | 38222 | 339.806784 | 120.1618953 | Receptor | GFRA4 |
| 383 | 119010 | 339.6060599 | 103.7237111 | Other enzyme | ARSB |
| 384 | 119464 | 339.4754061 | 100.5008021 | Other enzyme | TXNL |
| 385 | 111967 | 339.2763036 | 112.4119069 | Receptor | SH120 |
| 386 | 117597 | 339.238818 | 111.3546779 | Ion channel | SLC6A20 |
| 387 | 616 | 339.1783001 | 110.5612083 | Metabolic kinase | PIP5K2A |
| 388 | 104271 | 338.7413424 | 131.7387006 | Extracellular Factor, Protease | PLAT |
| 389 | 41648 | 338.7181328 | 115.2805567 | GPCR | GPR38 |
| 390 | 116760 | 338.1994712 | 104.0012206 | TF | CREB5 |
| 391 | 103742 | 338.1738446 | 95.33904505 | Protein kinase | NEK8 |
| 392 | 121625 | 338.1483829 | 117.8622144 | Other | FAF1 |
| 393 | 108793 | 337.9238774 | 121.4085775 | Membrane protein, Other enzyme | RDH11 |
| 394 | 46149 | 337.8978345 | 120.3236494 | Other enzyme | PIN4 |
| 395 | 121965 | 337.8789473 | 113.6213753 | Other | BBP |
| 396 | 104655 | 337.8013677 | 115.5912144 | Phosphatase | PTP4A1 |
| 397 | 3025 | 337.7404873 | 105.0254455 | TF | VAV1 |
| 398 | 23439 | 337.6708292 | 105.1774448 | Extracellular Factor, Other enzyme | PLA2G3 |
| 399 | 31620 | 337.1286426 | 124.1367317 | Other | FLJ22655 |
| 400 | 4069 | 336.8306941 | 105.5590406 | GPCR | BAI1 |
| 401 | 107669 | 336.303118 | 118.2304844 | Receptor | GFRA1 |
| 402 | 9248 | 336.0810213 | 122.0831997 | Cytochrome P450 | POR |
| 403 | 106198 | 335.9733958 | 105.6352148 | Other enzyme | ALDH3A1 |
| 404 | 112515 | 335.9637286 | 119.3357808 | Membrane protein | RARRES1 |
| 405 | 8537 | 335.3533595 | 121.335137 | Membrane protein, Transporter | AQP1 |
| 406 | 110610 | 335.3244422 | 106.7081378 | Extracellular Factor, Other enzyme | GPI |
| 407 | 43265 | 335.2808598 | 116.2065732 | Membrane protein, Phosphatase | PPAP2C |
| 408 | 180 | 335.1659998 | 114.9252844 | Protein kinase | CSNK1A1 |
| 409 | 117634 | 334.7917391 | 105.1004972 | Ion channel | SLC30A1 |
| 410 | 8947 | 334.6634975 | 94.95649933 | Receptor | ITGB6 |
| 411 | 10429 | 334.5855495 | 105.0652685 | Other enzyme | ARF4L |
| 412 | 107317 | 334.4278332 | 121.8454869 | Other enzyme | FASN |
| 413 | 121476 | 333.9429392 | 120.8680209 | Other enzyme | FEN1 |
| 414 | 126545 | 333.8186808 | 129.5423961 | Other | SPDY1 |
| 415 | 202300 | 333.4461042 | 111.6474763 | Phosphatase | DUSP7 |
| 416 | 105208 | 333.3722733 | 105.9736161 | Protease | KIAA1203 |
| 417 | 109375 | 332.6248847 | 111.0339775 | Receptor | IL22RA1 |
| 418 | 120071 | 332.0526699 | 104.9196346 | Ion channel, Other enzyme | ATP8B3 |
| 419 | 122067 | 332.006738 | 106.5738693 | Other | FLJ37300 |
| 420 | 18224 | 331.923628 | 104.6876436 | Other | IQGAP2 |
| 421 | 2057 | 331.888903 | 113.25982 | GPCR | OR1A2 |
| 422 | 6205 | 331.4027826 | 97.29546515 | Protease | CASP2 |
| 423 | 103432 | 331.0905347 | 111.3859232 | Protein kinase | STK18 |
| 424 | 107085 | 331.0174791 | 113.5678751 | Other enzyme | UGDH |
| 425 | 117546 | 330.8053804 | 116.4761789 | Other enzyme | DUOX1 |
| 426 | 41929 | 330.3190915 | 140.9052461 | NHR | NR2F6 |
| 427 | 112254 | 330.2324653 | 106.7015484 | Other enzyme | B3GNT5 |
| 428 | 105538 | 330.0716585 | 114.4822352 | Ion channel | KCNE2 |
| 429 | 444 | 329.6671336 | 111.1091926 | Protein kinase | MKNK1 |
| 430 | 6722 | 329.6243408 | 115.1625088 | Membrane protein | FLJ31819 |
| 431 | 40719 | 329.4969143 | 117.760941 | Protein kinase, Phosphatase | CAMK2G |
| 432 | 115262 | 329.2920103 | 120.0833852 | Other | ID2 |
| 433 | 106223 | 329.2569091 | 143.7439467 | Cytochrome P450 | CYP2C8 |
| 434 | 120151 | 328.5899481 | 119.5213398 | Ion channel | SLC6A18 |
| 435 | 105664 | 328.2937206 | 128.417486 | Protease | PRSS15 |
| 436 | 1020 | 328.0148328 | 89.51208851 | Protein kinase | FLJ11159 |
| 437 | 112308 | 327.8422314 | 115.5164776 | Extracellular Factor, Other enzyme | MGAT4B |
| 438 | 120484 | 327.5988635 | 115.9662965 | Other enzyme | SDS |
| 439 | 670 | 327.3947747 | 120.0872674 | Protein kinase | LCK |
| 440 | 1397 | 327.2921221 | 144.3640914 | Protein kinase | FLJ25006 |
| 441 | 104702 | 326.9211572 | 114.5827822 | Phosphatase | SYNJ1 |
| 442 | 1140 | 326.7712191 | 147.8017928 | Protein kinase | ALS2CR7 |
| 443 | 112418 | 326.2708208 | 124.3076331 | Membrane protein, Other enzyme | SIAT6 |
| 444 | 104693 | 326.0595643 | 123.7068081 | Ion channel | SCN3B |
| 445 | 8943 | 324.9305742 | 108.6926675 | Other enzyme | IMPDH1 |
| 446 | 107096 | 324.5453144 | 119.1902647 | Cytochrome P450 | YWHAZ |
| 447 | 2531 | 324.3124605 | 122.2897244 | Extracellular Factor, Protease | F2 |
| 448 | 118060 | 323.8821709 | 119.5157948 | Other enzyme | TRUB1 |
| 449 | 118590 | 323.302754 | 133.4437337 | Extracellular Factor | SERPINF2 |
| 450 | 118906 | 323.2258397 | 91.44527879 | Other enzyme | CA1 |
| 451 | 106243 | 321.8845718 | 87.98446503 | Membrane protein, Cytochrome P450 | CYP51A1 |
| 452 | 111874 | 320.7359898 | 99.33956661 | Other enzyme | SULT4A1 |
| 453 | 8193 | 320.6869655 | 116.0466428 | Other enzyme | PDHA1 |
| 454 | 2512 | 320.1814731 | 109.1446602 | Receptor, Transporter, Other enzyme | EBP |
| 455 | 16362 | 319.0943973 | 117.9639117 | Membrane protein, Protease | NAALADL1 |
| 456 | 14218 | 318.5137691 | 104.1842766 | Other enzyme | SDHA |
| 457 | 103493 | 318.4004706 | 107.8226635 | Protein kinase | MAP3K13 |
| 458 | 1176 | 317.5913147 | 69.8619217 | Protein kinase | ADCK1 |
| 459 | 111523 | 317.4037703 | 139.3022974 | Other enzyme | PCYT1B |
| 460 | 33700 | 316.7814948 | 111.129705 | GPCR | MASS1 |
| 461 | 2167 | 316.542118 | 111.6354846 | GPCR | RDS |
| 462 | 105854 | 316.2603175 | 119.0980681 | Other | PPP1R2 |
| 463 | 46281 | 316.2008308 | 110.8735838 | Receptor | FLJ10060 |
| 464 | 44894 | 315.4984566 | 117.0884463 | Protease | CTSC |
| 465 | 38041 | 314.8225353 | 111.1933054 | Other enzyme | B3GNT7 |
| 466 | 42278 | 314.3075428 | 111.7472547 | Membrane protein, Other enzyme | HS3ST3B1 |
| 467 | 112472 | 314.1907068 | 101.1194441 | Other enzyme | TRNT1 |

TABLE 2

| Target | siRNA | | RefSeq | Entrez | |
|---|---|---|---|---|---|
| No. | ID | Target symbol | accession | Gene ID | Target description |
| 1 | 113613 | ANKRD3 | NM_020639 | 54101 | Homo sapiens receptor-interacting serine-threonine |
| | | | | | kinase 4 (RIPK4), mRNA. |
| 1 | 105742 | ANKRD3 | NM_020639 | 54101 | Homo sapiens receptor-interacting serine-threonine |
| | | | | | kinase 4 (RIPK4), mRNA. |
| 1 | 105202 | ANKRD3 | NM_020639 | 54101 | Homo sapiens receptor-interacting serine-threonine |
| | | | | | kinase 4 (RIPK4), mRNA. |
| 2 | 117853 | HLCS | NM_000411 | 3141 | Homo sapiens holocarboxylase synthetase (biotin- |
| | | | | | [proprionyl-Coenzyme A-carboxylase (ATP- |
| | | | | | hydrolysing)] ligase) (HLCS), mRNA. |
| 2 | 117852 | HLCS | NM_000411 | 3141 | Homo sapiens holocarboxylase synthetase (biotin- |
| | | | | | [proprionyl-Coenzyme A-carboxylase (ATP- |
| | | | | | hydrolysing)] ligase) (HLCS), mRNA. |
| 2 | 117851 | HLCS | NM_000411 | 3141 | Homo sapiens holocarboxylase synthetase (biotin- |
| | | | | | [proprionyl-Coenzyme A-carboxylase (ATP- |
| | | | | | hydrolysing)] ligase) (HLCS), mRNA. |
| 3 | 117417 | SC4MOL | NM_006745 | 6307 | Homo sapiens sterol-C4-methyl oxidase-like |
| | | | | | (SC4MOL), mRNA. |
| 3 | 117416 | SC4MOL | NM_006745 | 6307 | Homo sapiens sterol-C4-methyl oxidase-like |
| | | | | | (SC4MOL), mRNA. |
| 3 | 117418 | SC4MOL | NM_006745 | 6307 | Homo sapiens sterol-C4-methyl oxidase-like |
| | | | | | (SC4MOL), mRNA. |
| 4 | 42711 | CASP1 | NM_033295* | 834 | Homo sapiens caspase 1, apoptosis-related cysteine |
| | | | | | protease (interleukin 1, beta, convertase) (CASP1), |
| | | | | | transcript variant epsilon, mRNA. |
| 4 | 42626 | CASP1 | NM_033295* | 834 | Homo sapiens caspase 1, apoptosis-related cysteine |
| | | | | | protease (interleukin 1, beta, convertase) (CASP1), |
| | | | | | transcript variant epsilon, mRNA. |
| 5 | 10418 | CDC42 | NM_001791* | 998 | Homo sapiens cell division cycle 42 (GTP binding |
| | | | | | protein, 25 kDa) (CDC42), transcript variant 1, mRNA. |
| 5 | 10505 | CDC42 | NM_001791* | 998 | Homo sapiens cell division cycle 42 (GTP binding |
| | | | | | protein, 25 kDa) (CDC42), transcript variant 1, mRNA. |
| 6 | 118135 | ABCC2 | NM_000392 | 1244 | Homo sapiens ATP-binding cassette, sub-family C |
| | | | | | (CFTR/MRP), member 2 (ABCC2), mRNA. |
| 6 | 116839 | ABCC2 | NM_000392 | 1244 | Homo sapiens ATP-binding cassette, sub-family C |
| | | | | | (CFTR/MRP), member 2 (ABCC2), mRNA. |
| 7 | 105039 | CTSE | NM_001910* | 1510 | Homo sapiens cathepsin E (CTSE), transcript variant |
| | | | | | 1, mRNA. |
| 7 | 105041 | CTSE | NM_001910* | 1510 | Homo sapiens cathepsin E (CTSE), transcript variant |
| | | | | | 1, mRNA. |
| 8 | 1147 | FRK | NM_002031 | 2444 | Homo sapiens fyn-related kinase (FRK), mRNA. |
| 8 | 1243 | FRK | NM_002031 | 2444 | Homo sapiens fyn-related kinase (FRK), mRNA. |
| 9 | 8225 | HMBS | NM_000190 | 3145 | Homo sapiens hydroxymethylbilane synthase (HMBS), |
| | | | | | mRNA. |
| 9 | 117844 | HMBS | NM_000190 | 3145 | Homo sapiens hydroxymethylbilane synthase (HMBS), |
| | | | | | mRNA. |
| 10 | 106087 | HSD17B4 | NM_000414 | 3295 | Homo sapiens hydroxysteroid (17-beta) |
| | | | | | dehydrogenase 4 (HSD17B4), mRNA. |
| 10 | 106089 | HSD17B4 | NM_000414 | 3295 | Homo sapiens hydroxysteroid (17-beta) |
| | | | | | dehydrogenase 4 (HSD17B4), mRNA. |
| 11 | 121011 | LCN2 | NM_005564 | 3934 | Homo sapiens lipocalin 2 (oncogene 24p3) (LCN2), |
| | | | | | mRNA. |
| 11 | 121012 | LCN2 | NM_005564 | 3934 | Homo sapiens lipocalin 2 (oncogene 24p3) (LCN2), |
| | | | | | mRNA. |
| 12 | 1766 | OXTR | NM_000916 | 5021 | Homo sapiens oxytocin receptor (OXTR), mRNA. |
| 12 | 1947 | OXTR | NM_000916 | 5021 | Homo sapiens oxytocin receptor (OXTR), mRNA. |
| 13 | 142836 | PPP1R3C | NM_005398 | 5507 | Homo sapiens protein phosphatase 1, regulatory |
| | | | | | (inhibitor) subunit 3C (PPP1R3C), mRNA. |
| 13 | 142834 | PPP1R3C | NM_005398 | 5507 | Homo sapiens protein phosphatase 1, regulatory |
| | | | | | (inhibitor) subunit 3C (PPP1R3C), mRNA. |
| 14 | 1547 | MAPK9 | NM_002752* | 5601 | Homo sapiens mitogen-activated protein kinase 9 |
| | | | | | (MAPK9), transcript variant 1, mRNA. |
| 14 | 1452 | MAPK9 | NM_002752* | 5601 | Homo sapiens mitogen-activated protein kinase 9 |
| | | | | | (MAPK9), transcript variant 1, mRNA. |
| 15 | 1699 | MAP2K5 | NM_145160* | 5607 | Homo sapiens mitogen-activated protein kinase kinase |
| | | | | | 5 (MAP2K5), transcript variant A, mRNA. |
| 15 | 118252 | MAP2K5 | NM_145160* | 5607 | Homo sapiens mitogen-activated protein kinase kinase |
| | | | | | 5 (MAP2K5), transcript variant A, mRNA. |
| 16 | 43916 | TPI1 | NM_000365 | 7167 | Homo sapiens triosephosphate isomerase 1 (TPI1), |
| | | | | | mRNA. |
| 16 | 43820 | TPI1 | NM_000365 | 7167 | Homo sapiens triosephosphate isomerase 1 (TPI1), |
| | | | | | mRNA. |
| 17 | 402 | VRK1 | NM_003384 | 7443 | Homo sapiens vaccinia related kinase 1 (VRK1), |
| | | | | | mRNA. |
| 17 | 401 | VRK1 | NM_003384 | 7443 | Homo sapiens vaccinia related kinase 1 (VRK1), |
| | | | | | mRNA. |
| 18 | 117695 | SLC30A2 | NM_032513 | 7780 | Homo sapiens solute carrier family 30 (zinc |
| | | | | | transporter), member 2 (SLC30A2), mRNA. |
| 18 | 117693 | SLC30A2 | NM_032513 | 7780 | Homo sapiens solute carrier family 30 (zinc |
| | | | | | transporter), member 2 (SLC30A2), mRNA. |
| 19 | 118261 | ULK1 | NM_003565 | 8408 | Homo sapiens unc-51-like kinase 1 (C. elegans) |
| | | | | | (ULK1), mRNA. |
| 19 | 118260 | ULK1 | NM_003565 | 8408 | Homo sapiens unc-51-like kinase 1 (C. elegans) |
| | | | | | (ULK1), mRNA. |
| 20 | 5146 | GLP2R | NM_004246 | 9340 | Homo sapiens glucagon-like peptide 2 receptor |
| | | | | | (GLP2R), mRNA. |
| 20 | 5237 | GLP2R | NM_004246 | 9340 | Homo sapiens glucagon-like peptide 2 receptor |
| | | | | | (GLP2R), mRNA. |
| 21 | 828 | SNK | NM_006622 | 10769 | Homo sapiens polo-like kinase 2 (Drosophila) (PLK2), |
| | | | | | mRNA. |
| 21 | 829 | SNK | NM_006622 | 10769 | Homo sapiens polo-like kinase 2 (Drosophila) (PLK2), |
| | | | | | mRNA. |
| 22 | 19104 | SPUVE | NM_007173 | 11098 | Homo sapiens protease, serine, 23 (PRSS23), mRNA. |
| 22 | 19196 | SPUVE | NM_007173 | 11098 | Homo sapiens protease, serine, 23 (PRSS23), mRNA. |
| 23 | 103354 | PASK | NM_015148 | 23178 | Homo sapiens PAS domain containing |
| | | | | | serine/threonine kinase (PASK), mRNA. |
| 23 | 978 | PASK | NM_015148 | 23178 | Homo sapiens PAS domain containing |
| | | | | | serine/threonine kinase (PASK), mRNA. |
| 24 | 2240 | OR52A1 | NM_012375 | 23538 | Homo sapiens olfactory receptor, family 52, subfamily |
| | | | | | A, member 1 (OR52A1), mRNA. |
| 24 | 2061 | OR52A1 | NM_012375 | 23538 | Homo sapiens olfactory receptor, family 52, subfamily |
| | | | | | A, member 1 (OR52A1), mRNA. |
| 25 | 20115 | RGS17 | NM_012419 | 26575 | Homo sapiens regulator of G-protein signalling 17 |
| | | | | | (RGS17), mRNA. |
| 25 | 20024 | RGS17 | NM_012419 | 26575 | Homo sapiens regulator of G-protein signalling 17 |
| | | | | | (RGS17), mRNA. |
| 26 | 118397 | MYLIP | NM_013262 | 29116 | Homo sapiens myosin regulatory light chain interacting |
| | | | | | protein (MYLIP), mRNA. |
| 26 | 118398 | MYLIP | NM_013262 | 29116 | Homo sapiens myosin regulatory light chain interacting |
| | | | | | protein (MYLIP), mRNA. |
| 27 | 104835 | PKD1L2 | NM_182740* | 114780 | Homo sapiens polycystic kidney disease 1-like 2 |
| | | | | | (PKD1L2), transcript variant 2, mRNA. |
| 27 | 104830 | PKD1L2 | NM_182740* | 114780 | Homo sapiens polycystic kidney disease 1-like 2 |
| | | | | | (PKD1L2), transcript variant 2, mRNA. |
| 28 | 119221 | BPHL | NM_004332 | 670 | Homo sapiens biphenyl hydrolase-like (serine |
| | | | | | hydrolase; breast epithelial mucin-associated antigen) |
| | | | | | (BPHL), mRNA. |
| 29 | 105141 | USP3 | NM_006537 | 9960 | Homo sapiens ubiquitin specific protease 3 (USP3), |
| | | | | | mRNA. |
| 30 | 122236 | NCE2 | NM_080678 | 140739 | Homo sapiens NEDD8-conjugating enzyme (NCE2), |
| | | | | | mRNA. |
| 31 | 43781 | KCNK17 | NM_031460 | 89822 | Homo sapiens potassium channel, subfamily K, |
| | | | | | member 17 (KCNK17), mRNA. |
| 32 | 103636 | WEE1 | NM_003390 | 7465 | Homo sapiens WEE1 homolog (S. pombe) (WEE1), |
| | | | | | mRNA. |
| 33 | 45922 | KCNE1 | NM_000219 | 3753 | Homo sapiens potassium voltage-gated channel, Isk- |
| | | | | | related family, member 1 (KCNE1), mRNA. |
| 34 | 117371 | RNUT1 | NM_005701 | 10073 | Homo sapiens RNA, U transporter 1 (RNUT1), mRNA. |
| 35 | 111157 | M6PR | NM_002355 | 4074 | Homo sapiens mannose-6-phosphate receptor (cation |
| | | | | | dependent) (M6PR), mRNA. |
| 36 | 38979 | MGC39650 | NM_152465 | 147011 | Homo sapiens hypothetical protein MGC39650 |
| | | | | | (MGC39650), mRNA. |
| 37 | 121933 | FLJ22761 | NM_025130 | 80201 | Homo sapiens hypothetical protein FLJ22761 |
| | | | | | (FLJ22761), mRNA. |
| 38 | 119829 | PAPOLG | NM_022894 | 64895 | Homo sapiens poly(A) polymerase gamma (PAPOLG), |
| | | | | | mRNA. |
| 39 | 19471 | DNM1L | NM_012062* | 10059 | Homo sapiens dynamin 1-like (DNM1L), transcript |
| | | | | | variant 1, mRNA. |
| 40 | 120442 | PSMD14 | NM_005805 | 10213 | Homo sapiens proteasome (prosome, macropain) 26S |
| | | | | | subunit, non-ATPase, 14 (PSMD14), mRNA. |
| 41 | 105154 | BACE | NM_138973* | 23621 | Homo sapiens beta-site APP-cleaving enzyme 1 |
| | | | | | (BACE1), transcript variant d, mRNA. |
| 42 | 6196 | FKSG79 | NM_032553 | 84636 | Homo sapiens G protein-coupled receptor 174 |
| | | | | | (GPR174), mRNA. |
| 43 | 105753 | LCN7 | NM_022164 | 64129 | Homo sapiens lipocalin 7 (LCN7), mRNA. |
| 44 | 21895 | STARD8 | NM_014725 | 9754 | Homo sapiens START domain containing 8 (STARD8), |
| | | | | | mRNA. |
| 45 | 120445 | RASGRP2 | NM_153819* | 10235 | Homo sapiens RAS guanyl releasing protein 2 (calcium |
| | | | | | and DAG-regulated) (RASGRP2), transcript variant 2, |
| | | | | | mRNA. |
| 46 | 111807 | QPCT | NM_012413 | 25797 | Homo sapiens glutaminyl-peptide cyclotransferase |
| | | | | | (glutaminyl cyclase) (QPCT), mRNA. |
| 47 | 1721 | ADORA1 | NM_000674 | 134 | Homo sapiens adenosine A1 receptor (ADORA1), |
| | | | | | mRNA. |
| 48 | 1774 | TACR1 | NM_001058* | 6869 | Homo sapiens tachykinin receptor 1 (TACR1), |
| | | | | | transcript variant long, mRNA. |
| 49 | 117712 | ABCA10 | NM_080282 | 10349 | Homo sapiens ATP-binding cassette, sub-family A |
| | | | | | (ABC1), member 10 (ABCA10), mRNA. |
| 50 | 1795 | GPR56 | NM_005682* | 9289 | Homo sapiens G protein-coupled receptor 56 (GPR56), |
| | | | | | transcript variant 1, mRNA. |
| 51 | 117084 | PRPSAP2 | NM_002767 | 5636 | Homo sapiens phosphoribosyl pyrophosphate |
| | | | | | synthetase-associated protein 2 (PRPSAP2), mRNA. |
| 52 | 119926 | SLC26A10 | NM_133489 | 65012 | Homo sapiens solute carrier family 26, member 10 |
| | | | | | (SLC26A10), mRNA. |
| 53 | 9067 | ADH7 | NM_000673 | 131 | Homo sapiens alcohol dehydrogenase 7 (class IV), mu |
| | | | | | or sigma polypeptide (ADH7), mRNA. |
| 54 | 121179 | OBP2A | NM_014582 | 29991 | Homo sapiens odorant binding protein 2A (OBP2A), |
| | | | | | mRNA. |
| 55 | 38327 | MRGX1 | NM_147199 | 259249 | Homo sapiens G protein-coupled receptor MRGX1 |
| | | | | | (MRGX1), mRNA. |
| 56 | 111813 | TNFRSF13B | NM_012452 | 23495 | Homo sapiens tumor necrosis factor receptor |
| | | | | | superfamily, member 13B (TNFRSF13B), mRNA. |
| 57 | 107569 | TP53I3 | NM_004881* | 9540 | Homo sapiens tumor protein p53 inducible protein 3 |
| | | | | | (TP53I3), transcript variant 1, mRNA. |
| 58 | 10560 | CPB2 | NM_016413* | 1361 | Homo sapiens carboxypeptidase B2 (plasma, |
| | | | | | carboxypeptidase U) (CPB2), transcript variant 2, |
| | | | | | mRNA. |
| 59 | 10235 | ARFD1 | NM_033228* | 373 | Homo sapiens tripartite motif-containing 23 (TRIM23), |
| | | | | | transcript variant gamma, mRNA. |
| 60 | 104127 | ADAM29 | NM_021780* | 11086 | Homo sapiens a disintegrin and metalloproteinase |
| | | | | | domain 29 (ADAM29), transcript variant 2, mRNA. |
| 61 | 112077 | AGPAT3 | NM_020132 | 56894 | Homo sapiens 1-acylglycerol-3-phosphate O- |
| | | | | | acyltransferase 3 (AGPAT3), mRNA. |
| 62 | 105010 | CTSK | NM_000396 | 1513 | Homo sapiens cathepsin K (pycnodysostosis) (CTSK), |
| | | | | | mRNA. |
| 63 | 4154 | GPR39 | NM_001508 | 2863 | Homo sapiens G protein-coupled receptor 39 (GPR39), |
| | | | | | mRNA. |
| 64 | 40980 | TTBK | NM_173500 | 146057 | Homo sapiens tau tubulin kinase 2 (TTBK2), mRNA. |
| 65 | 120756 | ATP2A3 | NM_174957* | 489 | Homo sapiens ATPase, Ca++ transporting, ubiquitous |
| | | | | | (ATP2A3), transcript variant 6, mRNA. |
| 66 | 1627 | FYN | NM_002037* | 2534 | Homo sapiens FYN oncogene related to SRC, FGR, |
| | | | | | YES (FYN), transcript variant 1, mRNA. |
| 67 | 122128 | C20orf185 | NM_182658 | 359710 | Homo sapiens chromosome 20 open reading frame |
| | | | | | 185 (C20orf185), mRNA. |
| 68 | 2207 | HM74 | NM_006018 | 8843 | Homo sapiens G protein-coupled receptor 109B |
| | | | | | (GPR109B), mRNA. |
| 69 | 4633 | TRHR | NM_003301 | 7201 | Homo sapiens thyrotropin-releasing hormone receptor |
| | | | | | (TRHR), mRNA. |
| 70 | 119952 | SLC12A2 | NM_001046 | 6558 | Homo sapiens solute carrier family 12 |
| | | | | | (sodium/potassium/chloride transporters), member 2 |
| | | | | | (SLC12A2), mRNA. |
| 71 | 105325 | CPA3 | NM_001870 | 1359 | Homo sapiens carboxypeptidase A3 (mast cell) |
| | | | | | (CPA3), mRNA. |
| 72 | 112330 | D4ST1 | NM_130468 | 113189 | Homo sapiens dermatan 4 sulfotransferase 1 (D4ST1), |
| | | | | | mRNA. |
| 73 | 121576 | APC2 | NM_005883 | 10297 | Homo sapiens adenomatosis polyposis coli 2 (APC2), |
| | | | | | mRNA. |
| 74 | 117799 | MGC52019 | NM_178498 | 159963 | Homo sapiens solute carrier family 5 (sodium/glucose |
| | | | | | cotransporter), member 12 (SLC5A12), mRNA. |
| 75 | 104458 | VDAC2 | NM_003375 | 7417 | Homo sapiens voltage-dependent anion channel 2 |
| | | | | | (VDAC2), mRNA. |
| 76 | 112453 | OGT | NM_181672* | 8473 | Homo sapiens O-linked N-acetylglucosamino (GlcNAc) |
| | | | | | transferase (UDP-N-acetylglucosamine:polypeptide-N- |
| | | | | | acetylglucosaminyl transferase) (OGT), transcript |
| | | | | | variant 1, mRNA. |
| 77 | 121337 | CENPE | NM_001813 | 1062 | Homo sapiens centromere protein E, 312 kDa |
| | | | | | (CENPE), mRNA. |
| 78 | 110844 | BCR | NM_021574* | 613 | Homo sapiens breakpoint cluster region (BCR), |
| | | | | | transcript variant 2, mRNA. |
| 79 | 119422 | ARHGEF1 | NM_004706* | 9138 | Homo sapiens Rho guanine nucleotide exchange |
| | | | | | factor (GEF) 1 (ARHGEF1), transcript variant 2, |
| | | | | | mRNA. |
| 80 | 119276 | VCP | NM_007126 | 7415 | Homo sapiens valosin-containing protein (VCP), |
| | | | | | mRNA. |
| 81 | 118817 | MCCC1 | NM_020166 | 56922 | Homo sapiens methylcrotonoyl-Coenzyme A |
| | | | | | carboxylase 1 (alpha) (MCCC1), mRNA. |
| 82 | 118114 | FKBP7 | NM_181342* | 51661 | Homo sapiens FK506 binding protein 7 (FKBP7), |
| | | | | | transcript variant 2, mRNA. |
| 83 | 118462 | KIF13B | NM_015254 | 23303 | Homo sapiens kinesin family member 13B (KIF13B), |
| | | | | | mRNA. |
| 84 | 46510 | TG1019 | NM_148962 | 165140 | Homo sapiens oxoeicosanoid (OXE) receptor 1 |
| | | | | | (OXER1), mRNA. |
| 85 | 119129 | CRMP1 | NM_001313 | 1400 | Homo sapiens collapsin response mediator protein 1 |
| | | | | | (CRMP1), mRNA. |
| 86 | 119399 | ELOVL3 | NM_152310 | 83401 | Homo sapiens elongation of very long chain fatty acids |
| | | | | | (FEN1/Elo2, SUR4/Elo3, yeast)-like 3 (ELOVL3), |
| | | | | | mRNA. |
| 87 | 122166 | APEX1 | NM_001641* | 328 | Homo sapiens APEX nuclease (multifunctional DNA |
| | | | | | repair enzyme) 1 (APEX1), transcript variant 1, mRNA. |
| 88 | 45063 | GALR2 | NM_003857 | 8811 | Homo sapiens galanin receptor 2 (GALR2), mRNA. |
| 89 | 117601 | SLC12A9 | NM_020246 | 56996 | Homo sapiens solute carrier family 12 |
| | | | | | (potassium/chloride transporters), member 9 |
| | | | | | (SLC12A9), mRNA. |
| 90 | 118446 | KIF2C | NM_006845 | 11004 | Homo sapiens kinesin family member 2C (KIF2C), |
| | | | | | mRNA. |
| 91 | 18240 | FTCD | NM_006657* | 10841 | Homo sapiens formiminotransferase cyclodeaminase |
| | | | | | (FTCD), transcript variant B, mRNA. |
| 92 | 104559 | CACNA2D2 | NM_006030 | 9254 | Homo sapiens calcium channel, voltage-dependent, |
| | | | | | alpha 2/delta subunit 2 (CACNA2D2), mRNA. |
| 93 | 1407 | MAPK14 | NM_139012* | 1432 | Homo sapiens mitogen-activated protein kinase 14 |
| | | | | | (MAPK14), transcript variant 2, mRNA. |
| 94 | 119077 | HEXA | NM_000520 | 3073 | Homo sapiens hexosaminidase A (alpha polypeptide) |
| | | | | | (HEXA), mRNA. |
| 95 | 1371 | SPHK1 | NM_021972* | 8877 | Homo sapiens sphingosine kinase 1 (SPHK1), mRNA. |
| 96 | 106537 | ATP5J | NM_001003696* | 522 | Homo sapiens ATP synthase, H+ transporting, |
| | | | | | mitochondrial F0 complex, subunit F6 (ATP5J), nuclear |
| | | | | | gene encoding mitochondrial protein, transcript variant |
| | | | | | 3, mRNA. |
| 97 | 120500 | POLG2 | NM_007215 | 11232 | Homo sapiens polymerase (DNA directed), gamma 2, |
| | | | | | accessory subunit (POLG2), mRNA. |
| 98 | 111654 | CSF2RA | NM_172245* | 1438 | Homo sapiens colony stimulating factor 2 receptor, |
| | | | | | alpha, low-affinity (granulocyte-macrophage) |
| | | | | | (CSF2RA), transcript variant 2, mRNA. |
| 99 | 118718 | SERPINB9 | NM_004155 | 5272 | Homo sapiens serine (or cysteine) proteinase inhibitor, |
| | | | | | clade B (ovalbumin), member 9 (SERPINB9), mRNA. |
| 100 | 120608 | UBASH3A | NM_001001895* | 53347 | Homo sapiens ubiquitin associated and SH3 domain |
| | | | | | containing, A (UBASH3A), transcript variant 2, mRNA. |
| 101 | 142253 | PPP1R7 | NM_002712 | 5510 | Homo sapiens protein phosphatase 1, regulatory |
| | | | | | subunit 7 (PPP1R7), mRNA. |
| 102 | 111081 | HIPK1 | NM_198268* | 204851 | Homo sapiens homeodomain interacting protein kinase |
| | | | | | 1 (HIPK1), transcript variant 1, mRNA. |
| 103 | 103786 | GUK1 | NM_000858 | 2987 | Homo sapiens guanylate kinase 1 (GUK1), mRNA. |
| 104 | 121943 | C2orf8 | NM_025264 | 80745 | Homo sapiens THUMP domain containing 2 |
| | | | | | (THUMPD2), mRNA. |
| 105 | 121806 | ITLN1 | NM_017625 | 55600 | Homo sapiens intelectin 1 (galactofuranose binding) |
| | | | | | (ITLN1), mRNA. |
| 106 | 15527 | PDE1A | NM_001003683* | 5136 | Homo sapiens phosphodiesterase 1A, calmodulin- |
| | | | | | dependent (PDE1A), transcript variant 2, mRNA. |
| 107 | 143005 | PKIA | NM_006823* | 5569 | Homo sapiens protein kinase (cAMP-dependent, |
| | | | | | catalytic) inhibitor alpha (PKIA), transcript variant 1, |
| | | | | | mRNA. |
| 108 | 110607 | GHR | NM_000163 | 2690 | Homo sapiens growth hormone receptor (GHR), |
| | | | | | mRNA. |
| 109 | 107834 | AASS | NM_005763 | 10157 | Homo sapiens aminoadipate-semialdehyde synthase |
| | | | | | (AASS), mRNA. |
| 110 | 121163 | LOC339133 | | 339133 | |
| 111 | 118522 | KIAA0449 | | 23046 | |
| 112 | 120179 | UBE3A | NM_130839* | 7337 | Homo sapiens ubiquitin protein ligase E3A (human |
| | | | | | papilloma virus E6-associated protein, Angelman |
| | | | | | syndrome) (UBE3A), transcript variant 3, mRNA. |
| 113 | 34999 | FLJ14681 | NM_032824 | 84910 | Homo sapiens hypothetical protein FLJ14681 |
| | | | | | (FLJ14681), mRNA. |
| 114 | 6091 | GPR84 | NM_020370 | 53831 | Homo sapiens G protein-coupled receptor 84 (GPR84), |
| | | | | | mRNA. |
| 115 | 103829 | LOC374425 | | 374425 | |
| 116 | 119396 | ARHGEF7 | NM_145735* | 8874 | Homo sapiens Rho guanine nucleotide exchange |
| | | | | | factor (GEF) 7 (ARHGEF7), transcript variant 2, |
| | | | | | mRNA. |
| 117 | 8816 | SMPD1 | NM_000543 | 6609 | Homo sapiens sphingomyelin phosphodiesterase 1, |
| | | | | | acid lysosomal (acid sphingomyelinase) (SMPD1), |
| | | | | | mRNA. |
| 118 | 15339 | AKAP5 | NM_004857 | 9495 | Homo sapiens A kinase (PRKA) anchor protein 5 |
| | | | | | (AKAP5), mRNA. |
| 119 | 29459 | FLJ21839 | NM_021831 | 60509 | Homo sapiens hypothetical protein FLJ21839 |
| | | | | | (FLJ21839), mRNA. |
| 120 | 16059 | PDHA2 | NM_005390 | 5161 | Homo sapiens pyruvate dehydrogenase (lipoamide) |
| | | | | | alpha 2 (PDHA2), mRNA. |
| 121 | 104173 | ADAM19 | NM_033274* | 8728 | Homo sapiens a disintegrin and metalloproteinase |
| | | | | | domain 19 (meltrin beta) (ADAM19), transcript variant |
| | | | | | 2, mRNA. |
| 122 | 120611 | RAB25 | NM_020387 | 57111 | Homo sapiens RAB25, member RAS oncogene family |
| | | | | | (RAB25), mRNA. |
| 123 | 142929 | PRKAB1 | NM_006253 | 5564 | Homo sapiens protein kinase, AMP-activated, beta 1 |
| | | | | | non-catalytic subunit (PRKAB1), mRNA. |
| 124 | 35220 | CNP | NM_033133 | 1267 | Homo sapiens 2,3-cyclic nucleotide 3 |
| | | | | | phosphodiesterase (CNP), mRNA. |
| 125 | 119469 | GCHFR | NM_005258 | 2644 | Homo sapiens GTP cyclohydrolase I feedback |
| | | | | | regulator (GCHFR), mRNA. |
| 126 | 121471 | BCL10 | NM_003921 | 8915 | Homo sapiens B-cell CLL/lymphoma 10 (BCL10), |
| | | | | | mRNA. |
| 127 | 122003 | CTMP | NM_053055* | 117145 | Homo sapiens C-terminal modulator protein (CTMP), |
| | | | | | transcript variant 1, mRNA. |
| 128 | 114058 | APP | NM_201414* | 351 | Homo sapiens amyloid beta (A4) precursor protein |
| | | | | | (protease nexin-II, Alzheimer disease) (APP), transcript |
| | | | | | variant 3, mRNA. |
| 129 | 117031 | GLRX | NM_002064 | 2745 | Homo sapiens glutaredoxin (thioltransferase) (GLRX), |
| | | | | | mRNA. |
| 130 | 110906 | CXADR | NM_001338 | 1525 | Homo sapiens coxsackie virus and adenovirus |
| | | | | | receptor (CXADR), mRNA. |
| 131 | 119517 | PRPS1L1 | NM_175886 | 221823 | Homo sapiens phosphoribosyl pyrophosphate |
| | | | | | synthetase 1-like 1 (PRPS1L1), mRNA. |
| 132 | 114048 | PROS1 | NM_000313 | 5627 | Homo sapiens protein S (alpha) (PROS1), mRNA. |
| 133 | 809 | MERTK | NM_006343 | 10461 | Homo sapiens c-mer proto-oncogene tyrosine kinase |
| | | | | | (MERTK), mRNA. |
| 134 | 137195 | STX10 | NM_003765 | 8677 | Homo sapiens syntaxin 10 (STX10), mRNA. |
| 135 | 118341 | Dlc2 | NM_080677 | 140735 | Homo sapiens dynein light chain 2 (Dlc2), mRNA. |
| 136 | 46319 | KLP1 | NM_020378 | 57106 | Homo sapiens K562 cell-derived leucine-zipper-like |
| | | | | | protein 1 (KLP1), mRNA. |
| 137 | 7204 | KCNN4 | NM_002250 | 3783 | Homo sapiens potassium intermediate/small |
| | | | | | conductance calcium-activated channel, subfamily N, |
| | | | | | member 4 (KCNN4), mRNA. |
| 138 | 119081 | MAN2B1 | NM_000528 | 4125 | Homo sapiens mannosidase, alpha, class 2B, member |
| | | | | | 1 (MAN2B1), mRNA. |
| 139 | 745 | STK10 | NM_005990 | 6793 | Homo sapiens serine/threonine kinase 10 (STK10), |
| | | | | | mRNA. |
| 140 | 119216 | BCS1L | NM_004328 | 617 | Homo sapiens BCS1-like (yeast) (BCS1L), mRNA. |
| 141 | 117277 | SLC22A14 | NM_004803 | 9389 | Homo sapiens solute carrier family 22 (organic cation |
| | | | | | transporter), member 14 (SLC22A14), mRNA. |
| 142 | 14775 | NDUFA1 | NM_004541 | 4694 | Homo sapiens NADH dehydrogenase (ubiquinone) 1 |
| | | | | | alpha subcomplex, 1, 7.5 kDa (NDUFA1), nuclear gene |
| | | | | | encoding mitochondrial protein, mRNA. |
| 143 | 119182 | SCP2 | NM_002979 | 6342 | Homo sapiens sterol carrier protein 2 (SCP2), mRNA. |
| 144 | 1286 | FLJ22055 | NM_024779 | 79837 | Homo sapiens phosphatidylinositol-4-phosphate 5- |
| | | | | | kinase, type II, gamma (PIP5K2C), mRNA. |
| 145 | 1961 | GPR1 | NM_005279 | 2825 | Homo sapiens G protein-coupled receptor 1 (GPR1), |
| | | | | | mRNA. |
| 146 | 119782 | ADARB1 | NM_015833* | 104 | Homo sapiens adenosine deaminase, RNA-specific, |
| | | | | | B1 (RED1 homolog rat) (ADARB1), transcript variant |
| | | | | | DRABA2b, mRNA. |
| 147 | 135366 | C20orf41 | NM_032957* | 51750 | Homo sapiens chromosome 20 open reading frame 41 |
| | | | | | (C20orf41), transcript variant 2, mRNA. |
| 148 | 42362 | OPN1LW | NM_020061 | 5956 | Homo sapiens opsin 1 (cone pigments), long-wave- |
| | | | | | sensitive (color blindness, protan) (OPN1LW), mRNA. |
| 149 | 12155 | PVRL2 | NM_002856 | 5819 | Homo sapiens poliovirus receptor-related 2 |
| | | | | | (herpesvirus entry mediator B) (PVRL2), mRNA. |
| 150 | 11 | ACVRL1 | NM_000020 | 94 | Homo sapiens activin A receptor type II-like 1 |
| | | | | | (ACVRL1), mRNA. |
| 151 | 7881 | CAPN3 | NM_212467* | 825 | Homo sapiens calpain 3, (p94) (CAPN3), transcript |
| | | | | | variant 9, mRNA. |
| 152 | 10428 | ARF4 | NM_001660 | 378 | Homo sapiens ADP-ribosylation factor 4 (ARF4), |
| | | | | | mRNA. |
| 153 | 16123 | HADHSC | NM_005327 | 3033 | Homo sapiens L-3-hydroxyacyl-Coenzyme A |
| | | | | | dehydrogenase, short chain (HADHSC), mRNA. |
| 154 | 1049 | CLK4 | NM_020666 | 57396 | Homo sapiens CDC-like kinase 4 (CLK4), mRNA. |
| 155 | 919 | IKBKE | NM_014002 | 9641 | Homo sapiens inhibitor of kappa light polypeptide gene |
| | | | | | enhancer in B-cells, kinase epsilon (IKBKE), mRNA. |
| 156 | 121066 | PLSCR3 | NM_020360 | 57048 | Homo sapiens phospholipid scramblase 3 (PLSCR3), |
| | | | | | mRNA. |
| 157 | 105169 | CAPN7 | NM_014296 | 23473 | Homo sapiens calpain 3, (p94) (CAPN3), transcript |
| | | | | | variant 9, mRNA. |
| 158 | 36311 | RGS18 | NM_130782 | 64407 | Homo sapiens regulator of G-protein signalling 18 |
| | | | | | (RGS18), mRNA. |
| 159 | 5884 | GPRC5D | NM_018654 | 55507 | Homo sapiens G protein-coupled receptor, family C, |
| | | | | | group 5, member D (GPRC5D), mRNA. |
| 160 | 44940 | SOD3 | NM_003102 | 6649 | Homo sapiens superoxide dismutase 3, extracellular |
| | | | | | (SOD3), mRNA. |
| 161 | 1398 | KIS | NM_144624* | 127933 | Homo sapiens kinase interacting with leukemia- |
| | | | | | associated gene (stathmin) (KIS), mRNA. |
| 162 | 104626 | SCN8A | NM_014191 | 6334 | Homo sapiens sodium channel, voltage gated, type |
| | | | | | VIII, alpha (SCN8A), mRNA. |
| 163 | 15563 | CCM1 | NM_194456* | 889 | Homo sapiens cerebral cavernous malformations 1 |
| | | | | | (CCM1), transcript variant 1, mRNA. |
| 164 | 136772 | MAPK8IP3 | NM_015133* | 23162 | Homo sapiens mitogen-activated protein kinase 8 |
| | | | | | interacting protein 3 (MAPK8IP3), transcript variant 1, |
| | | | | | mRNA. |
| 165 | 46183 | DDX19 | NM_007242 | 11269 | Homo sapiens DEAD (Asp-Glu-Ala-As) box |
| | | | | | polypeptide 19 (DDX19), mRNA. |
| 166 | 5377 | PPARD | NM_006238* | 5467 | Homo sapiens peroxisome proliferative activated |
| | | | | | receptor, delta (PPARD), transcript variant 1, mRNA. |
| 167 | 103491 | MAP3K6 | NM_004672 | 9064 | Homo sapiens mitogen-activated protein kinase kinase |
| | | | | | kinase 6 (MAP3K6), mRNA. |
| 168 | 117929 | ATP5L | NM_006476 | 10632 | Homo sapiens ATP synthase, H+ transporting, |
| | | | | | mitochondrial F0 complex, subunit g (ATP5L), nuclear |
| | | | | | gene encoding mitochondrial protein, mRNA. |
| 169 | 110591 | CPT2 | NM_000098 | 1376 | Homo sapiens carnitine palmitoyltransferase II (CPT2), |
| | | | | | nuclear gene encoding mitochondrial protein, mRNA. |
| 170 | 103534 | UCK1 | NM_031432 | 83549 | Homo sapiens uridine-cytidine kinase 1 (UCK1), |
| | | | | | mRNA. |
| 171 | 119066 | PAFAH2 | NM_000437 | 5051 | Homo sapiens platelet-activating factor acetylhydrolase |
| | | | | | 2, 40 kDa (PAFAH2), mRNA. |
| 172 | 106403 | ALDH7A1 | NM_001182 | 501 | Homo sapiens aldehyde dehydrogenase 7 family, |
| | | | | | member A1 (ALDH7A1), mRNA. |
| 173 | 121059 | NLGN3 | NM_018977 | 54413 | Homo sapiens neuroligin 3 (NLGN3), mRNA. |
| 174 | 121219 | AGA | NM_000027 | 175 | Homo sapiens aspartylglucosaminidase (AGA), mRNA. |
| 175 | 117366 | ABCC9 | NM_020297* | 10060 | Homo sapiens ATP-binding cassette, sub-family C |
| | | | | | (CFTR/MRP), member 9 (ABCC9), transcript variant |
| | | | | | SUR2B, mRNA. |
| 176 | 105936 | BCKDHB | NM_183050* | 594 | Homo sapiens branched chain keto acid |
| | | | | | dehydrogenase E1, beta polypeptide (maple syrup |
| | | | | | urine disease) (BCKDHB), nuclear gene encoding |
| | | | | | mitochondrial protein, transcript variant 1, mRNA. |
| 177 | 105919 | ACYP2 | NM_138448 | 98 | Homo sapiens acylphosphatase 2, muscle type |
| | | | | | (ACYP2), mRNA. |
| 178 | 103932 | CRN | NM_006587 | 10699 | Homo sapiens corin, serine protease (CORIN), mRNA. |
| 179 | 43139 | VN1R2 | NM_173856 | 317701 | Homo sapiens vomeronasal 1 receptor 2 (VN1R2), |
| | | | | | mRNA. |
| 180 | 9108 | GAD2 | NM_000818 | 2572 | Homo sapiens glutamate decarboxylase 2 (pancreatic |
| | | | | | islets and brain, 65 kDa) (GAD2), mRNA. |
| 181 | 111592 | UST | NM_005715 | 10090 | Homo sapiens uronyl-2-sulfotransferase (UST), mRNA. |
| 182 | 104388 | CLCN3 | NM_001829* | 1182 | Homo sapiens chloride channel 3 (CLCNa3), mRNA. |
| 183 | 115931 | SLC7A8 | NM_012244* | 23428 | Homo sapiens solute carrier family 7 (cationic amino |
| | | | | | acid transporter, y+ system), member 8 (SLC7A8), |
| | | | | | transcript variant 1, mRNA. |
| 184 | 30832 | LENG4 | NM_024298 | 79143 | Homo sapiens leukocyte receptor cluster (LRC) |
| | | | | | member 4 (LENG4), mRNA. |
| 185 | 105076 | USP13 | NM_003940 | 8975 | Homo sapiens ubiquitin specific protease 13 |
| | | | | | (isopeptidase T-3) (USP13), mRNA. |
| 186 | 44885 | FABP6 | NM_001445 | 2172 | Homo sapiens fatty acid binding protein 6, ileal |
| | | | | | (gastrotropin) (FABP6), mRNA. |
| 187 | 103580 | MAP2K4 | NM_003010 | 6416 | Homo sapiens mitogen-activated protein kinase kinase |
| | | | | | 4 (MAP2K4), mRNA. |
| 188 | 1555 | EPHA5 | NM_182472* | 2044 | Homo sapiens EphA5 (EPHA5), transcript variant 2, |
| | | | | | mRNA. |
| 189 | 105163 | USP25 | NM_013396 | 29761 | Homo sapiens ubiquitin specific protease 25 (USP25), |
| | | | | | mRNA. |
| 190 | 105773 | GGTL3 | NM_052830* | 2686 | Homo sapiens gamma-glutamyltransferase-like 3 |
| | | | | | (GGTL3), transcript variant 1, mRNA. |
| 191 | 37190 | TPCN2 | NM_139075 | 219931 | Homo sapiens two pore segment channel 2 (TPCN2), |
| | | | | | mRNA. |
| 192 | 121953 | NIPA2 | NM_030922 | 81614 | Homo sapiens non-imprinted in Prader-Willi/Angelman |
| | | | | | syndrome 2 (NIPA2), mRNA. |
| 193 | 9040 | ITGAX | NM_000887 | 3687 | Homo sapiens integrin, alpha X (antigen CD11C |
| | | | | | (p150), alpha polypeptide) (ITGAX), mRNA. |
| 194 | 119716 | GCS1 | NM_006302 | 7841 | Homo sapiens glucosidase I (GCS1), mRNA. |
| 195 | 1794 | SMO | NM_005631 | 6608 | Homo sapiens smoothened homolog (Drosophila) |
| | | | | | (SMO), mRNA. |
| 196 | 115136 | BTNL3 | NM_197975* | 10917 | Homo sapiens butyrophilin-like 3 (BTNL3), transcript |
| | | | | | variant 1, mRNA. |
| 197 | 116921 | SLC6A4 | NM_001045 | 6532 | Homo sapiens solute carrier family 6 (neurotransmitter |
| | | | | | transporter, serotonin), member 4 (SLC6A4), mRNA. |
| 198 | 117213 | ATP6V0B | NM_004047 | 533 | Homo sapiens ATPase, H+ transporting, lysosomal |
| | | | | | 21 kDa, V0 subunit c (ATP6V0B), mRNA. |
| 199 | 10426 | ARF1 | NM_001658 | 375 | Homo sapiens ADP-ribosylation factor 1 (ARF1), |
| | | | | | mRNA. |
| 200 | 657 | FER | NM_005246 | 2241 | Homo sapiens fer (fps/fes related) tyrosine kinase |
| | | | | | (phosphoprotein NCP94) (FER), mRNA. |
| 201 | 46002 | CST4 | NM_001899 | 1472 | Homo sapiens cystatin S (CST4), mRNA. |
| 202 | 105830 | PPP1CC | NM_002710 | 5501 | Homo sapiens protein phosphatase 1, catalytic |
| | | | | | subunit, gamma isoform (PPP1CC), mRNA. |
| 203 | 104815 | KCNK16 | NM_032115 | 83795 | Homo sapiens potassium channel, subfamily K, |
| | | | | | member 16 (KCNK16), mRNA. |
| 204 | 142280 | PRKAR2B | NM_002736 | 5577 | Homo sapiens protein kinase, cAMP-dependent, |
| | | | | | regulatory, type II, beta (PRKAR2B), mRNA. |
| 205 | 1940 | HTR2C | NM_000868 | 3358 | Homo sapiens 5-hydroxytryptamine (serotonin) |
| | | | | | receptor 2C (HTR2C), mRNA. |
| 206 | 103397 | PRKCM | NM_002742 | 5587 | Homo sapiens protein kinase C, mu (PRKCM), mRNA. |
| 207 | 117187 | AOC3 | NM_003734 | 8639 | Homo sapiens amine oxidase, copper containing 3 |
| | | | | | (vascular adhesion protein 1) (AOC3), mRNA. |
| 208 | 119405 | RALGPS2 | NM_018037* | 55103 | Homo sapiens Ral GEF with PH domain and SH3 |
| | | | | | binding motif 2 (RALGPS2), mRNA. |
| 209 | 111208 | PVRL1 | NM_203285* | 5818 | Homo sapiens poliovirus receptor-related 1 |
| | | | | | (herpesvirus entry mediator C; nectin) (PVRL1), |
| | | | | | transcript variant 2, mRNA. |
| 210 | 118568 | SUOX | NM_000456 | 6821 | Homo sapiens sulfite oxidase (SUOX), nuclear gene |
| | | | | | encoding mitochondrial protein, mRNA. |
| 211 | 383 | TEC | NM_003215 | 7006 | Homo sapiens tec protein tyrosine kinase (TEC), |
| | | | | | mRNA. |
| 212 | 118038 | NPL | NM_030769 | 80896 | Homo sapiens N-acetylneuraminate pyruvate lyase |
| | | | | | (dihydrodipicolinate synthase) (NPL), mRNA. |
| 213 | 42454 | BCL2L10 | NM_020396 | 10017 | Homo sapiens BCL2-like 10 (apoptosis facilitator) |
| | | | | | (BCL2L10), mRNA. |
| 214 | 118423 | KIF2 | NM_004520 | 3796 | Homo sapiens kinesin heavy chain member 2 (KIF2), |
| | | | | | mRNA. |
| 215 | 104231 | ADAMTS6 | NM_197941 | 345667 | Homo sapiens similar to ADAMTS-10 precursor (A |
| | | | | | disintegrin and metalloproteinase with thrombospondin |
| | | | | | motifs 10) (ADAM-TS 10) (ADAM-TS10) (LOC345667), |
| | | | | | mRNA. |
| 216 | 121036 | OBDPF | NM_017711 | 54857 | Homo sapiens glycerophosphodiester |
| | | | | | phosphodiesterase domain containing 2 (GDPD2), |
| | | | | | mRNA. |
| 217 | 5834 | GPRC5B | NM_016235 | 51704 | Homo sapiens G protein-coupled receptor, family C, |
| | | | | | group 5, member B (GPRC5B), mRNA. |
| 218 | 114204 | GLUL | NM_002065 | 2752 | Homo sapiens glutamate-ammonia ligase (glutamine |
| | | | | | synthase) (GLUL), mRNA. |
| 219 | 119258 | DPYSL4 | NM_006426 | 10570 | Homo sapiens dihydropyrimidinase-like 4 (DPYSL4), |
| | | | | | mRNA. |
| 220 | 111728 | LILRB5 | NM_006840 | 10990 | Homo sapiens leukocyte immunoglobulin-like receptor, |
| | | | | | subfamily B (with TM and ITIM domains), member 5 |
| | | | | | (LILRB5), mRNA. |
| 221 | 119072 | GALNS | NM_000512 | 2588 | Homo sapiens galactosamine (N-acetyl)-6-sulfate |
| | | | | | sulfatase (Morquio syndrome, mucopolysaccharidosis |
| | | | | | type IVA) (GALNS), mRNA. |
| 222 | 121053 | FLJ10948 | NM_018281 | 55268 | Homo sapiens hypothetical protein FLJ10948 |
| | | | | | (FLJ10948), mRNA. |
| 223 | 142803 | PRKRIR | NM_004705 | 5612 | Homo sapiens protein-kinase, interferon-inducible |
| | | | | | double stranded RNA dependent inhibitor, repressor of |
| | | | | | (P58 repressor) (PRKRIR), mRNA. |
| 224 | 117248 | PIGL | NM_004278 | 9487 | Homo sapiens phosphatidylinositol glycan, class L |
| | | | | | (PIGL), mRNA. |
| 225 | 111369 | TNFRSF14 | NM_003820 | 8764 | Homo sapiens tumor necrosis factor receptor |
| | | | | | superfamily, member 14 (herpesvirus entry mediator) |
| | | | | | (TNFRSF14), mRNA. |
| 226 | 118232 | ATM | NM_000051* | 472 | Homo sapiens ataxia telangiectasia mutated (includes |
| | | | | | complementation groups A, C and D) (ATM), transcript |
| | | | | | variant 1, mRNA. |
| 227 | 10238 | ARF3 | NM_001659 | 377 | Homo sapiens ADP-ribosylation factor 3 (ARF3), |
| | | | | | mRNA. |
| 228 | 118663 | SERPINB2 | NM_002575 | 5055 | Homo sapiens serine (or cysteine) proteinase inhibitor, |
| | | | | | clade B (ovalbumin), member 2 (SERPINB2), mRNA. |
| 229 | 13014 | VAV2 | NM_003371 | 7410 | Homo sapiens vav 2 oncogene (VAV2), mRNA. |
| 230 | 118922 | CA14 | NM_012113 | 23632 | Homo sapiens carbonic anhydrase XIV (CA14), |
| | | | | | mRNA. |
| 231 | 119792 | TRAP1 | NM_016292 | 10131 | Homo sapiens TNF receptor-associated protein 1 |
| | | | | | (TRAP1), mRNA. |
| 232 | 103447 | CAMK1G | NM_020439 | 57172 | Homo sapiens calcium/calmodulin-dependent protein |
| | | | | | kinase IG (CAMK1G), mRNA. |
| 233 | 23376 | LAP3 | NM_015907 | 51056 | Homo sapiens leucine aminopeptidase 3 (LAP3), |
| | | | | | mRNA. |
| 234 | 17099 | MDH2 | NM_005918 | 4191 | Homo sapiens malate dehydrogenase 2, NAD |
| | | | | | (mitochondrial) (MDH2), mRNA. |
| 235 | 138240 | PRKCABP | NM_012407 | 9463 | Homo sapiens protein kinase C, alpha binding protein |
| | | | | | (PRKCABP), mRNA. |
| 236 | 9004 | CRH | NM_000756 | 1392 | Homo sapiens corticotropin releasing hormone (CRH), |
| | | | | | mRNA. |
| 237 | 1785 | GPR3 | NM_005281 | 2827 | Homo sapiens G protein-coupled receptor 3 (GPR3), |
| | | | | | mRNA. |
| 238 | 107446 | NDUFA10 | NM_004544 | 4705 | Homo sapiens NADH dehydrogenase (ubiquinone) 1 |
| | | | | | alpha subcomplex, 10, 42 kDa (NDUFA10), nuclear |
| | | | | | gene encoding mitochondrial protein, mRNA. |
| 239 | 108267 | C1QR1 | NM_012072 | 22918 | Homo sapiens complement component 1, q |
| | | | | | subcomponent, receptor 1 (C1QR1), mRNA. |
| 240 | 122036 | FLJ32389 | NM_144617 | 126393 | Homo sapiens heat shock protein, alpha-crystallin- |
| | | | | | related, B6 (HSPB6), mRNA. |
| 241 | 118553 | SERPINA7 | NM_000354 | 6906 | Homo sapiens serine (or cysteine) proteinase inhibitor, |
| | | | | | clade A (alpha-1 antiproteinase, antitrypsin), member 7 |
| | | | | | (SERPINA7), mRNA. |
| 242 | 119612 | DCTD | NM_001921 | 1635 | Homo sapiens dCMP deaminase (DCTD), mRNA. |
| 243 | 121775 | ANGPTL4 | NM_139314* | 51129 | Homo sapiens angiopoietin-like 4 (ANGPTL4), |
| | | | | | transcript variant 1, mRNA. |
| 244 | 110854 | DCAMKL1 | NM_004734 | 9201 | Homo sapiens doublecortin and CaM kinase-like 1 |
| | | | | | (DCAMKL1), mRNA. |
| 245 | 126062 | FLJ23751 | NM_152282 | 92370 | Homo sapiens hypothetical protein FLJ23751 |
| | | | | | (FLJ23751), mRNA. |
| 246 | 118792 | BIA2 | NM_015431 | 25893 | Homo sapiens BIA2 (BIA2), mRNA. |
| 247 | 120597 | HDAC8 | NM_018486 | 55869 | Homo sapiens histone deacetylase 8 (HDAC8), mRNA. |
| 248 | 119183 | UPP1 | NM_181597* | 7378 | Homo sapiens uridine phosphorylase 1 (UPP1), |
| | | | | | transcript variant 2, mRNA. |
| 249 | 121277 | ANG | NM_001145 | 283 | Homo sapiens angiogenin, ribonuclease, RNase A |
| | | | | | family, 5 (ANG), mRNA. |
| 250 | 117497 | SLC39A2 | NM_014579 | 29986 | Homo sapiens solute carrier family 39 (zinc |
| | | | | | transporter), member 2 (SLC39A2), mRNA. |
| 251 | 1704 | PANK1 | NM_148977* | 53354 | Homo sapiens pantothenate kinase 1 (PANK1), |
| | | | | | transcript variant alpha, mRNA. |
| 252 | 119905 | SLC25A11 | NM_003562 | 8402 | Homo sapiens solute carrier family 25 (mitochondrial |
| | | | | | carrier; oxoglutarate carrier), member 11 (SLC25A11), |
| | | | | | mRNA. |
| 253 | 121507 | DDX21 | NM_004728 | 9188 | Homo sapiens DEAD (Asp-Glu-Ala-Asp) box |
| | | | | | polypeptide 21 (DDX21), mRNA. |
| 254 | 121103 | CACH-1 | NM_130767 | 134526 | Homo sapiens cytosolic acetyl-CoA hydrolase (CACH- |
| | | | | | 1), mRNA. |
| 255 | 6501 | ADRA1D | NM_000678 | 146 | Homo sapiens adrenergic, alpha-1D-, receptor |
| | | | | | (ADRA1D), mRNA. |
| 256 | 43395 | NLGN4Y | NM_014893 | 22829 | Homo sapiens neuroligin 4, Y-linked (NLGN4Y), |
| | | | | | mRNA. |
| 257 | 1541 | PDGFRB | NM_002609 | 5159 | Homo sapiens platelet-derived growth factor receptor, |
| | | | | | beta polypeptide (PDGFRB), mRNA. |
| 258 | 648 | EPHA1 | NM_005232 | 2041 | Homo sapiens EphA1 (EPHA1), mRNA. |
| 259 | 22653 | CENTD2 | NM_139181* | 116985 | Homo sapiens centaurin, delta 2 (CENTD2), transcript |
| | | | | | variant 1, mRNA. |
| 260 | 112041 | AD-017 | NM_018446* | 55830 | Homo sapiens glycosyltransferase AD-017 (AD-017), |
| | | | | | transcript variant 2, mRNA. |
| 261 | 116939 | ACLY | NM_198830* | 47 | Homo sapiens ATP citrate lyase (ACLY), transcript |
| | | | | | variant 2, mRNA. |
| 262 | 111049 | FUK | NM_145059 | 197258 | Homo sapiens fucokinase (FUK), mRNA. |
| 263 | 120730 | RHEBL1 | NM_144593 | 121268 | Homo sapiens Ras homolog enriched in brain like 1 |
| | | | | | (RHEBL1), mRNA. |
| 264 | 1776 | ADCYAP1R1 | NM_001118 | 117 | Homo sapiens adenylate cyclase activating |
| | | | | | polypeptide 1 (pituitary) receptor type I (ADCYAP1R1), |
| | | | | | mRNA. |
| 265 | 139134 | PIP5K1B | NM_003558 | 8395 | Homo sapiens phosphatidylinositol-4-phosphate 5- |
| | | | | | kinase, type I, beta (PIP5K1B), mRNA. |
| 266 | 118865 | SERPINB11 | NM_080475 | 89778 | Homo sapiens serine (or cysteine) proteinase inhibitor, |
| | | | | | clade B (ovalbumin), member 11 (SERPINB11), |
| | | | | | mRNA. |
| 267 | 119034 | GCH1 | NM_000161 | 2643 | Homo sapiens GTP cyclohydrolase 1 (dopa-responsive |
| | | | | | dystonia) (GCH1), mRNA. |
| 268 | 41802 | UGT2B11 | NM_001073 | 10720 | Homo sapiens UDP glycosyltransferase 2 family, |
| | | | | | polypeptide B11 (UGT2B11), mRNA. |
| 269 | 115614 | ATF1 | NM_005171 | 466 | Homo sapiens activating transcription factor 1 (ATF1), |
| | | | | | mRNA. |
| 270 | 120142 | SLC39A4 | NM_017767* | 55630 | Homo sapiens solute carrier family 39 (zinc |
| | | | | | transporter), member 4 (SLC39A4), mRNA. |
| 271 | 129420 | PIK3AP1 | NM_152309 | 118788 | Homo sapiens phosphoinositide-3-kinase adaptor |
| | | | | | protein 1 (PIK3AP1), mRNA. |
| 272 | 35139 | LDHL | NM_033195 | 92483 | Homo sapiens lactate dehydrogenase A-like 6B |
| | | | | | (LDHAL6B), mRNA. |
| 273 | 112237 | AGXT2L1 | NM_031279 | 64850 | Homo sapiens alanine-glyoxylate aminotransferase 2- |
| | | | | | like 1 (AGXT2L1), mRNA. |
| 274 | 118332 | DNCLI1 | NM_016141 | 51143 | Homo sapiens dynein, cytoplasmic, light intermediate |
| | | | | | polypeptide 1 (DNCLI1), mRNA. |
| 275 | 202390 | HS3ST4 | NM_006040 | 9951 | Homo sapiens heparan sulfate (glucosamine) 3-O- |
| | | | | | sulfotransferase 4 (HS3ST4), mRNA. |
| 276 | 111442 | CHST2 | NM_004267 | 9435 | Homo sapiens carbohydrate (N-acetylglucosamine-6- |
| | | | | | O) sulfotransferase 2 (CHST2), mRNA. |
| 277 | 119734 | GNA13 | NM_006572 | 10672 | Homo sapiens guanine nucleotide binding protein (G |
| | | | | | protein), alpha 13 (GNA13), mRNA. |
| 278 | 46555 | MGC30208 | NM_173804 | 255043 | Homo sapiens hypothetical protein MGC30208 |
| | | | | | (MGC30208), mRNA. |
| 279 | 112493 | GALNT10 | NM_198321* | 55568 | Homo sapiens UDP-N-acetyl-alpha-D- |
| | | | | | galactosamine:polypeptide N- |
| | | | | | acetylgalactosaminyltransferase 10 (GalNAc-T10) |
| | | | | | (GALNT10), transcript variant 1, mRNA. |
| 280 | 120542 | NEDL1 | NM_015052 | 23072 | Homo sapiens NEDD4-like ubiquitin-protein ligase 1 |
| | | | | | (NEDL1), mRNA. |
| 281 | 143975 | PIK3CD | NM_005026 | 5293 | Homo sapiens phosphoinositide-3-kinase, catalytic, |
| | | | | | delta polypeptide (PIK3CD), mRNA. |
| 282 | 202509 | KIAA0551 | XM_039796 | 23043 | PREDICTED: Homo sapiens TRAF2 and NCK |
| | | | | | interacting kinase (TNIK), mRNA. |
| 283 | 26615 | RHOT1 | NM_018307 | 55288 | Homo sapiens ras homolog gene family, member T1 |
| | | | | | (RHOT1), mRNA. |
| 284 | 2021 | MTNR1B | NM_005959 | 4544 | Homo sapiens melatonin receptor 1B (MTNR1B), |
| | | | | | mRNA. |
| 285 | 1017 | FLJ10074 | NM_017988 | 55681 | Homo sapiens hypothetical protein FLJ10074 |
| | | | | | (FLJ10074), mRNA. |
| 286 | 44902 | GAPD | NM_002046 | 2597 | Homo sapiens glyceraldehyde-3-phosphate |
| | | | | | dehydrogenase (GAPD), mRNA. |
| 287 | 121821 | C20orf12 | NM_018152 | 55184 | Homo sapiens chromosome 20 open reading frame 12 |
| | | | | | (C20orf12), mRNA. |
| 288 | 3573 | TRIM16 | NM_006470 | 10626 | Homo sapiens tripartite motif-containing 16 (TRIM16), |
| | | | | | mRNA. |
| 289 | 111379 | TNFRSF10C | NM_003841 | 8794 | Homo sapiens tumor necrosis factor receptor |
| | | | | | superfamily, member 10c, decoy without an |
| | | | | | intracellular domain (TNFRSF10C), mRNA. |
| 290 | 119725 | RPC32 | NM_006467 | 10622 | Homo sapiens polymerase (RNA) III (DNA directed) |
| | | | | | polypeptide G (32 kD) (POLR3G), mRNA. |
| 291 | 119012 | ARSE | NM_000047 | 415 | Homo sapiens arylsulfatase E (chondrodysplasia |
| | | | | | punctata 1) (ARSE), mRNA. |
| 292 | 11202 | ITGB8 | NM_002214 | 3696 | Homo sapiens integrin, beta 8 (ITGB8), mRNA. |
| 293 | 119352 | DPYSL5 | NM_020134 | 56896 | Homo sapiens dihydropyrimidinase-like 5 (DPYSL5), |
| | | | | | mRNA. |
| 294 | 111028 | MARK4 | NM_031417 | 57787 | Homo sapiens MAP/microtubule affinity-regulating |
| | | | | | kinase 4 (MARK4), mRNA. |
| 295 | 6242 | P2RY12 | NM_022788* | 64805 | Homo sapiens purinergic receptor P2Y, G-protein |
| | | | | | coupled, 12 (P2RY12), transcript variant 1, mRNA. |
| 296 | 6829 | GPR113 | NM_153835 | 165082 | Homo sapiens G protein-coupled receptor 113 |
| | | | | | (GPR113), mRNA. |
| 297 | 120986 | PLIN | NM_002666 | 5346 | Homo sapiens perilipin (PLIN), mRNA. |
| 298 | 282 | PIK4CB | NM_002651 | 5298 | Homo sapiens phosphatidylinositol 4-kinase, catalytic, |
| | | | | | beta polypeptide (PIK4CB), mRNA. |
| 299 | 119249 | RAPGEF3 | NM_006105 | 10411 | Homo sapiens Rap guanine nucleotide exchange |
| | | | | | factor (GEF) 3 (RAPGEF3), mRNA. |
| 300 | 121002 | RBP2 | NM_004164 | 5948 | Homo sapiens retinol binding protein 2, cellular |
| | | | | | (RBP2), mRNA. |
| 301 | 112098 | LOC57168 | NM_020437 | 57168 | Homo sapiens similar to aspartate beta hydroxylase |
| | | | | | (ASPH) (LOC57168), mRNA. |
| 302 | 120006 | SLCO1B1 | NM_006446 | 10599 | Homo sapiens solute carrier organic anion transporter |
| | | | | | family, member 1B1 (SLCO1B1), mRNA. |
| 303 | 9145 | PLCB3 | NM_000932 | 5331 | Homo sapiens phospholipase C, beta 3 |
| | | | | | (phosphatidylinositol-specific) (PLCB3), mRNA. |
| 304 | 45672 | ASB10 | NM_080871 | 136371 | Homo sapiens ankyrin repeat and SOCS box- |
| | | | | | containing 10 (ASB10), mRNA. |
| 305 | 5997 | MC3R | NM_019888 | 4159 | Homo sapiens melanocortin 3 receptor (MC3R), |
| | | | | | mRNA. |
| 306 | 5922 | NR2F2 | NM_021005 | 7026 | Homo sapiens nuclear receptor subfamily 2, group F, |
| | | | | | member 2 (NR2F2), mRNA. |
| 307 | 112027 | LPAAT-e | NM_018361 | 55326 | Homo sapiens acid acyltransferase-epsilon (LPAAT-e), |
| | | | | | mRNA. |
| 308 | 42079 | KCNJ4 | NM_152868* | 3761 | Homo sapiens potassium inwardly-rectifying channel, |
| | | | | | subfamily J, member 4 (KCNJ4), transcript variant 1, |
| | | | | | mRNA. |
| 309 | 104120 | ADAM18 | NM_014237 | 8749 | Homo sapiens a disintegrin and metalioproteinase |
| | | | | | domain 18 (ADAM18), mRNA. |
| 310 | 104465 | KCNAB2 | NM_003636* | 8514 | Homo sapiens potassium voltage-gated channel, |
| | | | | | shaker-related subfamily, beta member 2 (KCNAB2), |
| | | | | | transcript variant 1, mRNA. |
| 311 | 118241 | NME3 | NM_002513 | 4832 | Homo sapiens non-metastatic cells 3, protein |
| | | | | | expressed in (NME3), mRNA. |
| 312 | 12487 | SMARCA3 | NM_139048* | 6596 | Homo sapiens SWI/SNF related, matrix associated, |
| | | | | | actin dependent regulator of chromatin, subfamily a, |
| | | | | | member 3 (SMARCA3), transcript variant 2, mRNA. |
| 313 | 139155 | STX7 | NM_003569 | 8417 | Homo sapiens syntaxin 7 (STX7), mRNA. |
| 314 | 7014 | CLCN5 | NM_000084 | 1184 | Homo sapiens chloride channel 5 (nephrolithiasis 2, X- |
| | | | | | linked, Dent disease) (CLCN5), mRNA. |
| 315 | 107742 | HSD11B1 | NM_181755* | 3290 | Homo sapiens hydroxysteroid (11-beta) |
| | | | | | dehydrogenase 1 (HSD11B1), transcript variant 2, |
| | | | | | mRNA. |
| 316 | 122070 | ARHGEF19 | NM_153213 | 128272 | Homo sapiens Rho guanine nucleotide exchange |
| | | | | | factor (GEF) 19 (ARHGEF19), mRNA. |
| 317 | 119167 | LCT | NM_002299 | 3938 | Homo sapiens lactase (LCT), mRNA. |
| 318 | 121082 | SFXN1 | NM_022754 | 94081 | Homo sapiens sideroflexin 1 (SFXN1), mRNA. |
| 319 | 34166 | CARD11 | NM_032415 | 84433 | Homo sapiens caspase recruitment domain family, |
| | | | | | member 11 (CARD11), mRNA. |
| 320 | 36798 | LYPLAL1 | NM_138794 | 127018 | Homo sapiens lysophospholipase-like 1 (LYPLAL1), |
| | | | | | mRNA. |
| 321 | 6764 | MRGX2 | NM_054030 | 117194 | Homo sapiens G protein-coupled receptor MRGX2 |
| | | | | | (MRGX2), mRNA. |
| 322 | 112281 | HAVCR2 | NM_032782 | 84868 | Homo sapiens hepatitis A virus cellular receptor 2 |
| | | | | | (HAVCR2), mRNA. |
| 323 | 1359 | DKFZp761P1010 | NM_018423 | 55359 | Homo sapiens protein kinase STYK1 (STYK1), mRNA. |
| 324 | 120792 | ATP6V1E1 | NM_001696 | 529 | Homo sapiens ATPase, H+ transporting, lysosomal |
| | | | | | 31 kDa, V1 subunit E isoform 1 (ATP6V1E1), mRNA. |
| 325 | 120227 | ATP2B1 | NM_001001323* | 490 | Homo sapiens ATPase, Ca++ transporting, plasma |
| | | | | | membrane 1 (ATP2B1), transcript variant 1, mRNA. |
| 326 | 117144 | UAP1 | NM_003115 | 6675 | Homo sapiens UDP-N-acteylglucosamine |
| | | | | | pyrophosphorylase 1 (UAP1), mRNA. |
| 327 | 202463 | LOC375133 | NM_199345 | 375133 | Homo sapiens similar to phosphatidylinositol 4-kinase |
| | | | | | alpha (LOC375133), mRNA. |
| 328 | 326 | MAP2K1 | NM_002755 | 5604 | Homo sapiens mitogen-activated protein kinase kinase |
| | | | | | 1 (MAP2K1), mRNA. |
| 329 | 121132 | ITGA1 | NM_181501 | 3672 | Homo sapiens integrin, alpha 1 (ITGA1), mRNA. |
| 330 | 40762 | SNF1LK | NM_173354 | 150094 | Homo sapiens SNF1-like kinase (SNF1LK), mRNA. |
| 331 | 106985 | SPR | NM_003124 | 6697 | Homo sapiens sepiapterin reductase (7,8- |
| | | | | | dihydrobiopterin:NADP+ oxidoreductase) (SPR), |
| | | | | | mRNA. |
| 332 | 118634 | CCNF | NM_001761 | 899 | Homo sapiens cyclin F (CCNF), mRNA. |
| 333 | 1709 | AVPR2 | NM_000054 | 554 | Homo sapiens arginine vasopressin receptor 2 |
| | | | | | (nephrogenic diabetes insipidus) (AVPR2), mRNA. |
| 334 | 119230 | ARHGEF2 | NM_004723 | 9181 | Homo sapiens rho/rac guanine nucleotide exchange |
| | | | | | factor (GEF) 2 (ARHGEF2), mRNA. |
| 335 | 111644 | SIAT10 | NM_006100 | 10402 | Homo sapiens sialyltransferase 10 (alpha-2,3- |
| | | | | | sialyltransferase VI) (SIAT10), mRNA. |
| 336 | 103331 | ERBB4 | NM_005235 | 2066 | Homo sapiens v-erb-a erythroblastic leukemia viral |
| | | | | | oncogene homolog 4 (avian) (ERBB4), mRNA. |
| 337 | 105105 | BAP1 | NM_004656 | 8314 | Homo sapiens BRCA1 associated protein-1 (ubiquitin |
| | | | | | carboxy-terminal hydrolase) (BAP1), mRNA. |
| 338 | 6671 | CD97 | NM_001784* | 976 | Homo sapiens CD97 antigen (CD97), transcript variant |
| | | | | | 2, mRNA. |
| 339 | 117749 | FLJ30473 | NM_144704 | 150209 | Homo sapiens hypothetical protein FLJ30473 |
| | | | | | (FLJ30473), mRNA. |
| 340 | 104678 | TRPM7 | NM_017672 | 54822 | Homo sapiens transient receptor potential cation |
| | | | | | channel, subfamily M, member 7 (TRPM7), mRNA. |
| 341 | 4236 | P2RY1 | NM_002563 | 5028 | Homo sapiens purinergic receptor P2Y, G-protein |
| | | | | | coupled, 1 (P2RY1), mRNA. |
| 342 | 119150 | APOBEC1 | NM_005889* | 339 | Homo sapiens apolipoprotein B mRNA editing enzyme, |
| | | | | | catalytic polypeptide 1 (APOBEC1), transcript variant |
| | | | | | 2, mRNA. |
| 343 | 120536 | USP34 | NM_014709 | 9736 | Homo sapiens ubiquitin specific protease 34 (USP34), |
| | | | | | mRNA. |
| 344 | 4084 | CNR1 | NM_016083* | 1268 | Homo sapiens cannabinoid receptor 1 (brain) (CNR1), |
| | | | | | transcript variant 1, mRNA. |
| 345 | 8584 | RAG1 | NM_000448 | 5896 | Homo sapiens recombination activating gene 1 |
| | | | | | (RAG1), mRNA. |
| 346 | 118025 | FLJ21963 | NM_024560 | 79611 | Homo sapiens FLJ21963 protein (FLJ21963), mRNA. |
| 347 | 104025 | MMP13 | NM_002427 | 4322 | Homo sapiens matrix metalloproteinase 13 |
| | | | | | (collagenase 3) (MMP13), mRNA. |
| 348 | 142304 | MAPK3 | NM_002746 | 5595 | Homo sapiens mitogen-activated protein kinase 3 |
| | | | | | (MAPK3), mRNA. |
| 349 | 119004 | FLJ23322 | NM_024955 | 80020 | Homo sapiens hypothetical protein FLJ23322 |
| | | | | | (FLJ23322), mRNA. |
| 350 | 112199 | CHST5 | NM_012126* | 23563 | Homo sapiens carbohydrate (N-acetylglucosamine 6- |
| | | | | | O) sulfotransferase 5 (CHST5), mRNA. |
| 351 | 140383 | MIR16 | NM_016641 | 51573 | Homo sapiens membrane interacting protein of RGS16 |
| | | | | | (MIR16), mRNA. |
| 352 | 43202 | LOC134285 | NM_173490 | 134285 | Homo sapiens hypothetical protein LOC134285 |
| | | | | | (LOC134285), mRNA. |
| 353 | 118558 | TYR | NM_000372 | 7299 | Homo sapiens tyrosinase (oculocutaneous albinism IA) |
| | | | | | (TYR), mRNA. |
| 354 | 122033 | BIN1 | NM_139348* | 274 | Homo sapiens bridging integrator 1 (BIN1), transcript |
| | | | | | variant 6, mRNA. |
| 355 | 110947 | GFRA3 | NM_001496 | 2676 | Homo sapiens GDNF family receptor alpha 3 (GFRA3), |
| | | | | | mRNA. |
| 356 | 122374 | CALML3 | NM_005185 | 810 | Homo sapiens calmodulin-like 3 (CALML3), mRNA. |
| 357 | 121768 | HMP19 | NM_015980 | 51617 | Homo sapiens HMP19 protein (HMP19), mRNA. |
| 358 | 110667 | UGT1A1 | NM_000463 | 54658 | Homo sapiens UDP glycosyltransferase 1 family, |
| | | | | | polypeptide A1 (UGT1A1), mRNA. |
| 359 | 119683 | SLC9A3R1 | NM_004252 | 9368 | Homo sapiens solute carrier family 9 (sodium/hydrogen |
| | | | | | exchanger), isoform 3 regulator 1 (SLC9A3R1), mRNA. |
| 360 | 105368 | MBTPS2 | NM_015884 | 51360 | Homo sapiens membrane-bound transcription factor |
| | | | | | protease, site 2 (MBTPS2), mRNA. |
| 361 | 117476 | SLC30A4 | NM_013309 | 7782 | Homo sapiens solute carrier family 30 (zinc |
| | | | | | transporter), member 4 (SLC30A4), mRNA. |
| 362 | 8110 | PLG | NM_000301 | 5340 | Homo sapiens plasminogen (PLG), mRNA. |
| 363 | 108254 | PLA2R1 | NM_007366 | 22925 | Homo sapiens phospholipase A2 receptor 1, 180 kDa |
| | | | | | (PLA2R1), mRNA. |
| 364 | 119254 | POLD2 | NM_006230 | 5425 | Homo sapiens polymerase (DNA directed), delta 2, |
| | | | | | regulatory subunit 50 kDa (POLD2), mRNA. |
| 365 | 120528 | ATP2C1 | NM_001001485* | 27032 | Homo sapiens ATPase, Ca++ transporting, type 2C, |
| | | | | | member 1 (ATP2C1), transcript variant 3, mRNA. |
| 366 | 114721 | SUPT16H | NM_007192 | 11198 | Homo sapiens suppressor of Ty 16 homolog (S. cerevisiae) |
| | | | | | (SUPT16H), mRNA. |
| 367 | 120404 | ARHI | NM_004675 | 9077 | Homo sapiens ras homolog gene family, member I |
| | | | | | (ARHI), mRNA. |
| 368 | 121560 | ARPC4 | NM_005718 | 10093 | Homo sapiens actin related protein 2/3 complex, |
| | | | | | subunit 4, 20 kDa (ARPC4), mRNA. |
| 369 | 8759 | ORM1 | NM_000607 | 5004 | Homo sapiens orosomucoid 1 (ORM1), mRNA. |
| 370 | 121689 | C4.4A | NM_014400 | 27076 | Homo sapiens GPI-anchored metastasis-associated |
| | | | | | protein homolog (C4.4A), mRNA. |
| 371 | 116959 | CLNS1A | NM_001293 | 1207 | Homo sapiens chloride channel, nucleotide-sensitive, |
| | | | | | 1A (CLNS1A), mRNA. |
| 372 | 18269 | MAN1A2 | NM_006699 | 10905 | Homo sapiens mannosidase, alpha, class 1A, member |
| | | | | | 2 (MAN1A2), mRNA. |
| 373 | 4118 | CYP2F1 | NM_000774 | 1572 | Homo sapiens cytochrome P450, family 2, subfamily F, |
| | | | | | polypeptide 1 (CYP2F1), mRNA. |
| 374 | 120313 | UBE2E1 | NM_003341* | 7324 | Homo sapiens ubiquitin-conjugating enzyme E2E 1 |
| | | | | | (UBC4/5 homolog, yeast) (UBE2E1), transcript variant |
| | | | | | 1, mRNA. |
| 375 | 14778 | NDUFB2 | NM_004546 | 4708 | Homo sapiens NADH dehydrogenase (ubiquinone) 1 |
| | | | | | beta subcomplex, 2, 8 kDa (NDUFB2), nuclear gene |
| | | | | | encoding mitochondrial protein, mRNA. |
| 376 | 1554 | CDKL1 | NM_004196 | 8814 | Homo sapiens cyclin-dependent kinase-like 1 (CDC2- |
| | | | | | related kinase) (CDKL1), mRNA. |
| 377 | 120581 | RRAGB | NM_016656* | 10325 | Homo sapiens Ras-related GTP binding B (RRAGB), |
| | | | | | transcript variant RAGBI, mRNA. |
| 378 | 135789 | AKAP3 | NM_006422 | 10566 | Homo sapiens A kinase (PRKA) anchor protein 3 |
| | | | | | (AKAP3), mRNA. |
| 379 | 104313 | GLRA1 | NM_000171 | 2741 | Homo sapiens glycine receptor, alpha 1 (startle |
| | | | | | disease/hyperekplexia, stiff man syndrome) (GLRA1), |
| | | | | | mRNA. |
| 380 | 5020 | PNR | NM_003967 | 9038 | Homo sapiens putative neurotransmitter receptor |
| | | | | | (PNR), mRNA. |
| 381 | 103787 | MAP3K11 | NM_002419 | 4296 | Homo sapiens mitogen-activated protein kinase kinase |
| | | | | | kinase 11 (MAP3K11), mRNA. |
| 382 | 38222 | GFRA4 | NM_022139* | 64096 | Homo sapiens GDNF family receptor alpha 4 (GFRA4), |
| | | | | | transcript variant 1, mRNA. |
| 383 | 119010 | ARSB | NM_000046* | 411 | Homo sapiens arylsulfatase B (ARSB), transcript |
| | | | | | variant 1, mRNA. |
| 384 | 119464 | TXNL | NM_004786 | 9352 | Homo sapiens thioredoxin-like 1 (TXNL1), mRNA. |
| 385 | 111967 | SH120 | NM_016334 | 51463 | Homo sapiens G protein-coupled receptor 89 (GPR89), |
| | | | | | mRNA. |
| 386 | 117597 | SLC6A20 | NM_022405* | 54716 | Homo sapiens solute carrier family 6 (neurotransmitter |
| | | | | | transporter), member 20 (SLC6A20), transcript variant |
| | | | | | 2, mRNA. |
| 387 | 616 | PIP5K2A | NM_005028 | 5305 | Homo sapiens phosphatidylinositol-4-phosphate 5- |
| | | | | | kinase, type II, alpha (PIP5K2A), mRNA. |
| 388 | 104271 | PLAT | NM_033011* | 5327 | Homo sapiens plasminogen activator, tissue (PLAT), |
| | | | | | transcript variant 3, mRNA. |
| 389 | 41648 | GPR38 | NM_001507 | 2862 | Homo sapiens motilin receptor (MLNR), mRNA. |
| 390 | 116760 | CREB5 | NM_182899* | 9586 | Homo sapiens cAMP responsive element binding |
| | | | | | protein 5 (CREB5), mRNA. |
| 391 | 103742 | NEK8 | NM_178170 | 284086 | Homo sapiens NIMA (never in mitosis gene a)-related |
| | | | | | kinase 8 (NEK8), mRNA. |
| 392 | 121625 | FAF1 | NM_131917* | 11124 | Homo sapiens Fas (TNFRSF6) associated factor 1 |
| | | | | | (FAF1), transcript variant 2, mRNA. |
| 393 | 108793 | RDH11 | NM_016026 | 51109 | Homo sapiens retinol dehydrogenase 11 (all-trans and |
| | | | | | 9-cis) (RDH11), mRNA. |
| 394 | 46149 | PIN4 | NM_006223 | 5303 | Homo sapiens protein (peptidyl-prolyl cis/trans |
| | | | | | isomerase) NIMA-interacting, 4 (parvulin) (PIN4), |
| | | | | | mRNA. |
| 395 | 121965 | BBP | NM_032027 | 83941 | Homo sapiens beta-amyloid binding protein precursor |
| | | | | | (BBP), mRNA. |
| 396 | 104655 | PTP4A1 | NM_003463 | 7803 | Homo sapiens protein tyrosine phosphatase type IVA, |
| | | | | | member 1 (PTP4A1), mRNA. |
| 397 | 3025 | VAV1 | NM_005428 | 7409 | Homo sapiens vav 1 oncogene (VAV1), mRNA. |
| 398 | 23439 | PLA2G3 | NM_015715 | 50487 | Homo sapiens phospholipase A2, group III (PLA2G3), |
| | | | | | mRNA. |
| 399 | 31620 | FLJ22655 | NM_024730 | 79785 | Homo sapiens hypothetical protein FLJ22655 |
| | | | | | (FLJ22655), mRNA. |
| 400 | 4069 | BAI1 | NM_001702 | 575 | Homo sapiens brain-specific angiogenesis inhibitor 1 |
| | | | | | (BAI1), mRNA. |
| 401 | 107669 | GFRA1 | NM_145793* | 2674 | Homo sapiens GDNF family receptor alpha 1 (GFRA1), |
| | | | | | transcript variant 2, mRNA. |
| 402 | 9248 | POR | NM_000941 | 5447 | Homo sapiens P450 (cytochrome) oxidoreductase |
| | | | | | (POR), mRNA. |
| 403 | 106198 | ALDH3A1 | NM_000691 | 218 | Homo sapiens aldehyde dehydrogenase 3 family, |
| | | | | | memberA1 (ALDM3A1), mRNA. |
| 404 | 112515 | RARRES1 | NM_206963* | 5918 | Homo sapiens retinoic acid receptor responder |
| | | | | | (tazarotene induced) 1 (RARRES1), transcript variant |
| | | | | | 1, mRNA. |
| 405 | 8537 | AQP1 | NM_198098* | 358 | Homo sapiens aquaporin 1 (channel-forming integral |
| | | | | | protein, 28 kDa) (AQP1), transcript variant 1, mRNA. |
| 406 | 110610 | GPI | NM_000175 | 2821 | Homo sapiens glucose phosphate isomerase (GPI), |
| | | | | | mRNA. |
| 407 | 43265 | PPAP2C | NM_177526* | 8612 | Homo sapiens phosphatidic acid phosphatase type 2C |
| | | | | | (PPAP2C), transcript variant 2, mRNA. |
| 408 | 180 | CSNK1A1 | NM_001892 | 1452 | Homo sapiens casein kinase 1, alpha 1 (CSNK1A1), |
| | | | | | mRNA. |
| 409 | 117634 | SLC30A1 | NM_021194 | 7779 | Homo sapiens solute carrier family 30 (zinc |
| | | | | | transporter), member 1 (SLC30A1), mRNA. |
| 410 | 8947 | ITGB6 | NM_000888 | 3694 | Homo sapiens integrin, beta 6 (ITGB6), mRNA. |
| 411 | 10429 | ARF4L | NM_001661 | 379 | Homo sapiens ADP-ribosylation factor 4-like (ARF4L), |
| | | | | | mRNA. |
| 412 | 107317 | FASN | NM_004104 | 2194 | Homo sapiens fatty acid synthase (FASN), mRNA. |
| 413 | 121476 | FEN1 | NM_004111 | 2237 | Homo sapiens flap structure-specific endonuclease 1 |
| | | | | | (FEN1), mRNA. |
| 414 | 126545 | SPDY1 | NM_182756 | 245711 | Homo sapiens speedy homolog 1 (Drosophila) |
| | | | | | (SPDY1), mRNA. |
| 415 | 202300 | DUSP7 | NM_001947 | 1849 | Homo sapiens dual specificity phosphatase 7 |
| | | | | | (DUSP7), mRNA. |
| 416 | 105208 | KIAA1203 | NM_020718 | 57478 | Homo sapiens ubiquitin specific protease 31 (USP31), |
| | | | | | mRNA. |
| 417 | 109375 | IL22RA1 | NM_021258 | 58985 | Homo sapiens interleukin 22 receptor, alpha 1 |
| | | | | | (IL22RA1), mRNA. |
| 418 | 120071 | ATP8B3 | NM_138813 | 148229 | Homo sapiens ATPase, Class I, type 8B, member 3 |
| | | | | | (ATP8B3), mRNA. |
| 419 | 122067 | FLJ37300 | NM_153209 | 124602 | Homo sapiens hypothetical protein FLJ37300 |
| | | | | | (FLJ37300), mRNA. |
| 420 | 18224 | IQGAP2 | NM_006633 | 10788 | Homo sapiens IQ motif containing GTPase activating |
| | | | | | protein 2 (IQGAP2), mRNA. |
| 421 | 2057 | OR1A2 | NM_012352 | 26189 | Homo sapiens olfactory receptor, family 1, subfamily A, |
| | | | | | member 2 (OR1A2), mRNA. |
| 422 | 6205 | CASP2 | NM_032982* | 835 | Homo sapiens caspase 2, apoptosis-related cysteine |
| | | | | | protease (neural precursor cell expressed, |
| | | | | | developmentally down-regulated 2) (CASP2), transcript |
| | | | | | variant 1, mRNA. |
| 423 | 103432 | STK18 | NM_014264 | 10733 | Homo sapiens polo-like kinase 4 (Drosophila) (PLK4), |
| | | | | | mRNA. |
| 424 | 107085 | UGDH | NM_003359 | 7358 | Homo sapiens UDP-glucose dehydrogenase (UGDH), |
| | | | | | mRNA. |
| 425 | 117546 | DUOX1 | NM_017434* | 53905 | Homo sapiens dual oxidase 1 (DUOX1), transcript |
| | | | | | variant 1, mRNA. |
| 426 | 41929 | NR2F6 | NM_005234 | 2063 | Homo sapiens nuclear receptor subfamily 2, group F, |
| | | | | | member 6 (NR2F6), mRNA. |
| 427 | 112254 | B3GNT5 | NM_032047 | 84002 | Homo sapiens UDP-GlcNAc:betaGal beta-1,3-N- |
| | | | | | acetylglucosaminyltransferase 5 (B3GNT5), mRNA. |
| 428 | 105538 | KCNE2 | NM_172201 | 9992 | Homo sapiens potassium voltage-gated channel, Isk- |
| | | | | | related family, member 2 (KCNE2), mRNA. |
| 429 | 444 | MKNK1 | NM_003684* | 8569 | Homo sapiens MAP kinase interacting serine/threonine |
| | | | | | kinase 1 (MKNK1), mRNA. |
| 430 | 6722 | FLJ31819 | NM_152529 | 151556 | Homo sapiens G protein-coupled receptor 155 |
| | | | | | (GPR155), mRNA. |
| 431 | 40719 | CAMK2G | NM_172170* | 818 | Homo sapiens calcium/calmodulin-dependent protein |
| | | | | | kinase (CaM kinase) II gamma (CAMK2G), transcript |
| | | | | | variant 3, mRNA. |
| 432 | 115262 | ID2 | NM_002166 | 3398 | Homo sapiens inhibitor of DNA binding 2, dominant |
| | | | | | negative helix-loop-helix protein (ID2), mRNA. |
| 433 | 106223 | CYP2C8 | NM_000770* | 1558 | Homo sapiens cytochrome P450, family 2, subfamily |
| | | | | | C, polypeptide 8 (CYP2C8), transcript variant Hp1-1, |
| | | | | | mRNA. |
| 434 | 120151 | SLC6A18 | NM_182632 | 348932 | Homo sapiens solute carrier family 6 (neurotransmitter |
| | | | | | transporter), member 18 (SLC6A18), mRNA. |
| 435 | 105664 | PRSS15 | NM_004793 | 9361 | Homo sapiens protease, serine, 15 (PRSS15), nuclear |
| | | | | | gene encoding mitochondrial protein, mRNA. |
| 436 | 1020 | FLJ11159 | NM_018343 | 55781 | Homo sapiens RIO kinase 2 (yeast) (RIOK2), mRNA. |
| 437 | 112308 | MGAT4B | NM_014275* | 11282 | Homo sapiens mannosyl (alpha-1,3-)-glycoprotein |
| | | | | | beta-1,4-N-acetylglucosaminyltransferase, isoenzyme |
| | | | | | B (MGAT4B), transcript variant 1, mRNA. |
| 438 | 120484 | SDS | NM_006843 | 10993 | Homo sapiens serine dehydratase (SDS), mRNA. |
| 439 | 670 | LCK | NM_005356 | 3932 | Homo sapiens lymphocyte-specific protein tyrosine |
| | | | | | kinase (LCK), mRNA. |
| 440 | 1397 | FLJ25006 | NM_144610 | 124923 | Homo sapiens hypothetical protein FLJ25006 |
| | | | | | (FLJ25006), mRNA. |
| 441 | 104702 | SYNJ1 | NM_003895* | 8867 | Homo sapiens synaptojanin 1 (SYNJ1), transcript |
| | | | | | variant 1, mRNA. |
| 442 | 1140 | ALS2CR7 | NM_139158 | 65061 | Homo sapiens amyotrophic lateral sclerosis 2 (juvenile) |
| | | | | | chromosome region, candidate 7 (ALS2CR7), mRNA. |
| 443 | 112418 | SIAT6 | NM_174963* | 6487 | Homo sapiens sialyltransferase 6 (N- |
| | | | | | acetyllacosaminide alpha 2,3-sialyltransferase) |
| | | | | | (SIAT6), transcript variant 1, mRNA. |
| 444 | 104693 | SCN3B | NM_018400 | 55800 | Homo sapiens sodium channel, voltage-gated, type III, |
| | | | | | beta (SCN3B), mRNA. |
| 445 | 8943 | IMPDH1 | NM_000883* | 3614 | Homo sapiens IMP (inosine monophosphate) |
| | | | | | dehydrogenase 1 (IMPDH1), transcript variant 1, |
| | | | | | mRNA. |
| 446 | 107096 | YWHAZ | NM_003406* | 7534 | Homo sapiens tyrosine 3-monooxygenase/tryptophan |
| | | | | | 5-monooxygenase activation protein, zeta polypeptide |
| | | | | | (YWHAZ), transcript variant 1, mRNA. |
| 447 | 2531 | F2 | NM_000506 | 2147 | Homo sapiens coagulation factor II (thrombin) (F2), |
| | | | | | mRNA. |
| 448 | 118060 | TRUB1 | NM_139169 | 142940 | Homo sapiens TruB pseudouridine (psi) synthase |
| | | | | | homolog 1 (E. coli) (TRUB1), mRNA. |
| 449 | 118590 | SERPINF2 | NM_000934 | 5345 | Homo sapiens serine (or cysteine) proteinase inhibitor, |
| | | | | | clade F (alpha-2 antiplasmin, pigment epithelium |
| | | | | | derived factor), member 2 (SERPINF2), mRNA. |
| 450 | 118906 | CA1 | NM_001738 | 759 | Homo sapiens carbonic anhydrase I (CA1), mRNA. |
| 451 | 106243 | CYP51A1 | NM_000786 | 1595 | Homo sapiens cytochrome P450, family 51, subfamily |
| | | | | | A, polypeptide 1 (CYP51A1), mRNA. |
| 452 | 111874 | SULT4A1 | NM_014351* | 25830 | Homo sapiens sulfotransferase family 4A, member 1 |
| | | | | | (SULT4A1), transcript variant 1, mRNA. |
| 453 | 8193 | PDHA1 | NM_000284 | 5160 | Homo sapiens pyruvate dehydrogenase (lipoamide) |
| | | | | | alpha 1 (PDHA1), mRNA. |
| 454 | 2512 | EBP | NM_006579 | 10682 | Homo sapiens emopamil binding protein (sterol |
| | | | | | isomerase) (EBP), mRNA. |
| 455 | 16362 | NAALADL1 | NM_005468 | 10004 | Homo sapiens N-acetylated alpha-linked acidic |
| | | | | | dipeptidase-like 1 (NAALADL1), mRNA. |
| 456 | 14218 | SDHA | NM_004168 | 6389 | Homo sapiens succinate dehydrogenase complex, |
| | | | | | subunit A, flavoprotein (Fp) (SDHA), nuclear gene |
| | | | | | encoding mitochondrial protein, mRNA. |
| 457 | 103493 | MAP3K13 | NM_004721 | 9175 | Homo sapiens mitogen-activated protein kinase kinase |
| | | | | | kinase 13 (MAP3K13), mRNA. |
| 458 | 1176 | ADCK1 | NM_020421 | 57143 | Homo sapiens aarF domain containing kinase 1 |
| | | | | | (ADCK1), mRNA. |
| 459 | 111523 | PCYT1B | NM_004845 | 9468 | Homo sapiens phosphate cytidylyltransferase 1, |
| | | | | | choline, beta isoform (PCYT1B), mRNA. |
| 460 | 33700 | MASS1 | NM_000322 | 84059 | Homo sapiens monogenic, audiogenic seizure |
| | | | | | susceptibility 1 homolog (mouse) (MASS1), mRNA. |
| 461 | 2167 | RDS | NM_000322 | 5961 | Homo sapiens retinal degeneration, slow (RDS), |
| | | | | | mRNA. |
| 462 | 105854 | PPP1R2 | NM_006241 | 5504 | Homo sapiens protein phosphatase 1, regulatory |
| | | | | | (inhibitor) subunit 2 (PPP1R2), mRNA. |
| 463 | 46281 | FLJ10060 | NM_017986 | 55065 | Homo sapiens G protein-coupled receptor 172B |
| | | | | | (GPR172B), mRNA. |
| 464 | 44894 | CTSC | NM_001814* | 1075 | Homo sapiens cathepsin C (CTSC), transcript variant |
| | | | | | 1, mRNA. |
| 465 | 38041 | B3GNT7 | NM_145236 | 93010 | Homo sapiens UDP-GlcNAc:betaGal beta-1,3-N- |
| | | | | | acetylglucosaminyltransferase 7 (B3GNT7), mRNA. |
| 466 | 42278 | HS3ST3B1 | NM_006041 | 9953 | Homo sapiens heparan sulfate (glucosamine) 3-O- |
| | | | | | sulfotransferase 3B1 (HS3ST3B1), mRNA. |
| 467 | 112472 | TRNT1 | NM_182916* | 51095 | Homo sapiens tRNA nucleotidyl transferase, CCA- |
| | | | | | adding, 1 (TRNT1), mRNA. |

TABLE 3

| | | |
|---|---|---|
| Target No. | siRNA ID | siRNA sense sequence (21-mer) |

| | | |
|---|---|---|
| 1 | 113613 | CCAAGCACGAUGUAUACAGTT |
| 1 | 105742 | GCGCAUGGAGCUUUUGGAATT |
| 1 | 105202 | GGAGAAUAUCGUGCGCAUCTT |
| 2 | 117853 | CGAAAGUUAACAAAACUCCTT |
| 2 | 117852 | GGACGGUAUGGAGCAUGUUTT |
| 2 | 117851 | GCCUGAACCUUCUCUUGAGTT |
| 3 | 117417 | CGUAAACCUUCCUGAAAGATT |
| 3 | 117416 | CCAUUCGUUUAUUAGAAACTT |
| 3 | 117418 | CCUUUAAUUACCUUCCUAGTT |
| 4 | 42711 | GAAUAUGCCUGUUCCUGUGTT |
| 4 | 42626 | GACUCAUUGAACAUAUGCATT |
| 5 | 10418 | GGAUUAUGACAGAUUACGATT |
| 5 | 10505 | GGCUGUCAAGUAUGUGGAGTT |
| 6 | 118135 | GCAGACAAUGGUGUGUACATT |
| 6 | 116839 | GCAGGUAUUCGUUGGUUUUTT |
| 7 | 105039 | GGCCCAUAUAUUGCAUUUATT |
| 7 | 105041 | GGAGGCAGAUAAUGCUGGUTT |
| 8 | 1147 | GGCAGACAAGUCAACCGUGTT |
| 8 | 1243 | GGCAUGGCCACUACUUUGUTT |
| 9 | 8225 | GGAAUGCAUGUAUGCUGUGTT |
| 9 | 117844 | CCAAUCCUACUAAUAAACCTT |
| 10 | 106087 | GGAAUAUAUGGCAACUUUGTT |
| 10 | 106089 | GGGCACACUACACUAUUAATT |
| 11 | 121011 | CGAGUUACCUCGUCCGAGUTT |
| 11 | 121012 | GCAUGCUAUGGUGUUCUUCTT |
| 12 | 1766 | GGUGCACAUCUUCUCUCUGTT |
| 12 | 1947 | GGUACCUAUCAGUUUGUAUTT |
| 13 | 142836 | CCGAUUACUUAAGUUUCCGTT |
| 13 | 142834 | CGACGACAUUUUGUGAAUATT |
| 14 | 1547 | GGUUAUUCACAUGGAGCUGTT |
| 14 | 1452 | GGGAUUGUUUGUGCUGCAUTT |
| 15 | 1699 | GGUUAAGCUGUGUGAUUUUTT |
| 15 | 118252 | GCCCUCCAAUAUGCUAGUATT |
| 16 | 43916 | GAGAAGGCAUGUCUUUGGGTT |
| 16 | 43820 | GAGAGAAGGCAUGUCUUUGTT |
| 17 | 402 | GGAGGCUUUGGCUGUAUAUTT |
| 17 | 401 | GGAAUGGAAAGUAGGAUUATT |
| 18 | 117695 | CCAUCCUGAGAGAUGUGAUTT |
| 18 | 117693 | GCCAGAAUACAAGUAUGUATT |
| 19 | 118261 | CCCAAGCACUUUAUGCAUATT |
| 19 | 118260 | GCGAAUUUUGUGUGAUUUCTT |
| 20 | 5146 | GGCAUGUCUGAGAGACUUATT |
| 20 | 5237 | GGAACCUUCUGGCAUAUUUTT |
| 21 | 828 | GGUAUACAAUGCCGUCCUCTT |
| 21 | 829 | GGACUUUGGAACUGUGAAUTT |
| 22 | 19104 | GGCCAAGCAAUAUCUGUCUTT |
| 22 | 19196 | GGGUCUUCAGGAAAGUCUCTT |
| 23 | 103354 | GGACCAGCAAAUCACUGCCTT |
| 23 | 978 | GGUCUGAACCAGUGGAUGUTT |
| 24 | 2240 | GGAUUUGACCUCACAUUCATT |
| 24 | 2061 | GGGUAUAGAGUCAGGCAUCTT |
| 25 | 20115 | GGUUUUCCAUGGUUAAGUUTT |
| 25 | 20024 | GGAAGUCUUGUCCUGGUCUTT |
| 26 | 118397 | GGAGCAGACUAGGCAUAUCTT |
| 26 | 118398 | GCAGACUAGGCAUAUCUUUTT |
| 27 | 104835 | GGCGAUAUGGAGGUGUACATT |
| 27 | 104830 | GGAGGCCAUUUGGUCUUCATT |
| 28 | 119221 | CGGUUUCAUGCCGACUUCATT |
| 29 | 105141 | GGCAGCCAGACUGCAUUUATT |
| 30 | 122236 | GUUGUCUGCUUACCUUAACTT |
| 31 | 43781 | GCUCUAAGGAAGACUUCAATT |
| 32 | 103636 | GGCUGGAUGGAUGCAUUUATT |
| 33 | 45922 | GCUACAUCCGCUCCAAGAATT |
| 34 | 117371 | CCAUUCUAGAUUGCAUUUATT |
| 35 | 111157 | GCGUGGCAAAGUCCAAGAUTT |
| 36 | 38979 | GGUGAUCAAGAAGGUAAAGTT |
| 37 | 121933 | GGAAUCAUUGCAUGCUUAATT |
| 38 | 119829 | GCCUUGAUAUAAGGUGUAUTT |
| 39 | 19471 | GGAAAUAAUAAGGGAGUAATT |
| 40 | 120442 | GGCAUUAAUUCAUGGACUATT |
| 41 | 105154 | GGCAGUCUCUGGUAUACACTT |
| 42 | 6196 | GGUCUCAUAGGGAAUAUAUTT |
| 43 | 105753 | GUGGCAGUGUGACCAAGAATT |
| 44 | 21895 | GGAAAGUAAGUGCUGGUCUTT |
| 45 | 120445 | GGCUCUGCUAGACCAAGAATT |
| 46 | 111807 | GGUGGUUCGAAAGACUUCATT |
| 47 | 1721 | GGUCAUCAGCAUGGAGUACTT |
| 48 | 1774 | GGACAGUGACGAACUAUUUTT |
| 49 | 117712 | GCGAGUUUUACCUGAUAAATT |
| 50 | 1795 | GGGAAGACUUUCGCUUCUGTT |
| 51 | 117084 | GCUCCUGAUCAUGGUGUAUTT |
| 52 | 119926 | GGAGAAAAGGAGACUUCAATT |
| 53 | 9067 | GGCUAUUUGCCAGCAUAUATT |
| 54 | 121179 | CGGACGACUACGUCUUUUATT |
| 55 | 38327 | GGGAAGUCUUAUUUUGUCATT |
| 56 | 111813 | CCCUAAGCAAUGUGCAUACTT |
| 57 | 107569 | GGUCUAGGGACAAUAAGUATT |
| 58 | 10560 | GGCAUCCUGAUAUGCUUACTT |
| 59 | 10235 | GGUGCUAGAGUGUGGAGUUTT |
| 60 | 104127 | GGACAAAGUGUUUGCUUGATT |
| 61 | 112077 | CGGAGUGUACACUGUUCACTT |
| 62 | 105010 | GGAAGAGAGUUGUAUGUACTT |
| 63 | 4154 | GGCUGAUUGUUGUGACAUUTT |
| 64 | 40980 | GGAAAGACCAUGUUUGUAGTT |
| 65 | 120756 | CCUGGUGGUUUGUGUAUGATT |
| 66 | 1627 | GGAAGUUUACUGGAUUUCUTT |
| 67 | 122128 | CCAAAGUGGGCAUGCAUUGTT |
| 68 | 2207 | GGCAUGAUUAAACAAGGCATT |
| 69 | 4633 | GGUACAUAGCAAUCUGUCATT |
| 70 | 119952 | CCGUGGUGGAGUUGCUUAATT |
| 71 | 105325 | GAAAAAAUCGUUCCAAGAATT |
| 72 | 112330 | GGAUGUGCUGCCUAAGUAUTT |
| 73 | 121576 | CGUGUAAAUAGUGGUAAAUTT |
| 74 | 117799 | CGAUUCUCUACACAGGAGUTT |
| 75 | 104458 | GGAUCAAUUUAUCAGAAAGTT |
| 76 | 112453 | GCAUUAUCGACAUGCAUUGTT |
| 77 | 121337 | CCAAUCAUCGAUUCUGCCATT |
| 78 | 110844 | GGAUCCAACGACCAAGAACTT |
| 79 | 119422 | CCGAUCACAAAGCCUUCUATT |
| 60 | 119276 | GGUAUACCUUAAGCCGUACTT |
| 81 | 118817 | GCCAAAAAACUGGGUGUACTT |
| 82 | 118114 | CGAUGAACUAUAGCAUAUUTT |
| 83 | 118462 | GCCGAAGGUGUUUGCUUAUTT |
| 84 | 46510 | GACUCCAGAACCUUCUCUCTT |
| 85 | 119129 | CGAUGACCAAUCCCUUUAUTT |
| 66 | 119399 | GCAAGGUCAUAGAACUCGGTT |
| 87 | 122166 | GAAAGGAUUAGAUUGGGUATT |
| 88 | 45063 | GCAUCCUGACGGUUGAUGUTT |
| 89 | 117601 | GCUUCACUUGUGACAGGACTT |
| 90 | 118446 | GCAACUUGUUUUGCAUAUGTT |
| 91 | 18240 | GGAAGUUCCUCAUUGCUUUTT |
| 92 | 104559 | GGAGUCCUAUGACUAUCAGTT |
| 93 | 1407 | GGUGGAUGUAUAGCAUUUUTT |
| 94 | 119077 | GGAGAUGAGGUUGAUUUCATT |
| 95 | 1371 | GGGCAGGCAUAUGGAGUAUTT |
| 96 | 106537 | GGACCUGUUGAUGCUAGUUTT |
| 97 | 120500 | GCAGAUACGAAAUGGUGUUTT |
| 98 | 111654 | CGAAUGUUCGUGCACAUUUTT |
| 99 | 118718 | CCCUUAAAGCUCACUUCAATT |
| 100 | 120608 | GCACGAGACGCACUUUUAUTT |
| 101 | 142253 | GGAGCUACAGAGUCUUCGATT |
| 102 | 111081 | GGCAGACCGAAGAGAAUACTT |
| 103 | 103786 | GAACGGCAAAGAUUACUACTT |
| 104 | 121943 | CCAUUGUAUUGUUGCUUAGTT |
| 105 | 121806 | CGAAUGUCCUAGUGCAUUUTT |
| 106 | 15527 | GGAAUACUAAGAUGCUUGGTT |
| 107 | 143005 | GCACUGUUUUUAGCAUUACTT |
| 108 | 110607 | CCUGAGCGAGAGACUUUUUTT |
| 109 | 107834 | GGGUCUAUAGAGUUUAUGATT |
| 110 | 121163 | GGUGCAAUGCGACUAUCACTT |
| 111 | 118522 | CCUAUGACUUUGUCUUCGATT |
| 112 | 120179 | CGAAUGAGUUUUGUGCUUGTT |
| 113 | 34999 | GGUUCUAUAGUCCUUUUAATT |
| 114 | 6091 | GGUAAUAGGAGAAUAGGUGTT |
| 115 | 103829 | GGAAAUGACUACAGAGCUGTT |
| 116 | 119396 | CGUACCUACGGCCAUUGCATT |
| 117 | 8816 | GGAGACAAAGUGCAUAUAATT |
| 118 | 15339 | GGAAAAGGCAUCCAUGCUUTT |
| 119 | 29459 | GGCAAGAGGAUAUUCUUCUTT |
| 120 | 16059 | GGAUGAUGCUGACUGUUCGTT |
| 121 | 104173 | GGUGGUGGAAUGUGUCUCUTT |
| 122 | 120611 | GGUCUUCAUGCCCUAUCACTT |
| 123 | 142929 | CCUACAUUCUCGAUUUUUCTT |
| 124 | 35220 | GGCCUUCAAGAAGGAGCUGTT |
| 125 | 119469 | GCCUUGGGAAACAACUUUUTT |
| 126 | 121471 | GCAUACUUCUAGGAUAGCUTT |
| 127 | 122003 | GGAAGUCAUUCUUAAGGACTT |
| 128 | 114058 | GGCAGUUAUCCAGCAUUUCTT |
| 129 | 117031 | GCAUAGUUGGUCUUGGUGUTT |
| 130 | 110906 | GCAUCUAUCAGAUUAAGUUTT |
| 131 | 119517 | CGACAGUCUAAUGGAGCUUTT |
| 132 | 114048 | CCAAACAGGGUAAUCUUGATT |
| 133 | 809 | GGGAAGAAGCCAAGCCUUATT |
| 134 | 137195 | CCAUCGGUAUAGUGGAAGCTT |
| 135 | 118341 | CGAGACAAAGCACUUCAUCTT |
| 136 | 46319 | GCAAAGACCUGUGAAGCUATT |
| 137 | 7204 | GGAACUGGCAUUGGACUCATT |
| 138 | 119081 | CCGUGGACCAGUACUUUUATT |
| 139 | 745 | GGUUUACAAGGCCAAGAAUTT |
| 140 | 119216 | GCUAUGCCUGGUUGCUUAGTT |
| 141 | 117277 | GCAUCAUGUCGGCCUUCUUTT |
| 142 | 14775 | GGGUUGCUCAUUUUGGGUATT |
| 143 | 119182 | CCUCCUGAUCAAUAAGUAUTT |
| 144 | 1286 | GGUCAACAAUCACCUUUUCTT |
| 145 | 1961 | GGAACUCAGAAACCAAGAATT |
| 146 | 119782 | GGACUCAAGUAUGACUUCCTT |
| 147 | 135366 | CCAAGGUCCUGGAAUGUCUTT |
| 148 | 42362 | GCAUUGUGAACCAGGUCUCTT |
| 149 | 12155 | GGAUGUGCGAGUUCAAGUGTT |
| 150 | 11 | GGAGCACCUGAUUCCUUUCTT |
| 151 | 7881 | GGAACAGCAACAAUUCCGGTT |
| 152 | 10428 | GGAUUUGGCAAAUGCUAUGTT |
| 153 | 16123 | GGGAAUUGAGGAAAGCCUUTT |
| 154 | 1049 | GGAAAAGAUCCAGGAGUAUTT |
| 155 | 919 | GGUACUGGUGAUGGAGUACTT |
| 156 | 121066 | CCAAAGACGGUGGAUAUAGTT |
| 157 | 105169 | GGAAAUCUAGUGGUGACUATT |
| 158 | 36311 | GGCUUUUACCAGAUUUCUUTT |
| 159 | 5884 | GGUAUGAUGUUUGUGAAUATT |
| 160 | 44940 | GUGGAUCCGAGACAUGUACTT |
| 161 | 1398 | GGCAAUCAGGAUGUAAAGUTT |
| 162 | 104626 | GGAAAGACGUAACAGGAGATT |
| 163 | 15563 | GGAACGACAGUGGGUAGAUTT |
| 164 | 136772 | CCCACAUAUCUGCUCUGUATT |
| 165 | 46183 | GAUACCAAUGGUGCUGUUGTT |
| 166 | 5377 | GGCCUUCUCCAAGCACAUCTT |
| 167 | 103491 | GGUGGGUAUGUACAAGGUCTT |
| 168 | 117929 | GGUCACAAUUUCUCUUGAUTT |
| 169 | 110591 | CCUCAUUAUCGCCAAGGAUTT |
| 170 | 103534 | GGACAGUACAAUUUUGACCTT |
| 171 | 119066 | GCGAGUGUUUACGGGUGUUTT |
| 172 | 106403 | GGUCUACUUGUACUAUCAATT |
| 173 | 121059 | GCGAGAAUAUUGCCUUCUUTT |
| 174 | 121219 | GCAUGAUCAGUCCUGAUUGTT |
| 175 | 117366 | CCUUUGCACUAUAUACAUCTT |
| 176 | 105936 | GGCCAAAGGACUUCUUUUGTT |
| 177 | 105919 | GCCCUAGUUCUCGCAUUGATT |
| 178 | 103932 | GGAAGCAUCCAUCAGCUGGTT |
| 179 | 43139 | GUCUCCUGCUUUGUAUCCATT |
| 180 | 9108 | GGCACAGGUGUAAAUAUAGTT |
| 181 | 111592 | GGUUAUUUUACAUCAUUCCTT |
| 182 | 104388 | GGAUGGCUAGUAGUAACACTT |
| 183 | 115931 | GCCCAAGUGUUUCAGUGACTT |
| 184 | 30832 | GGCGGCUUCCUUGGAGUAUTT |
| 185 | 105076 | GGAUGAACUGAUCGCUUAUTT |
| 186 | 44885 | GUUCACUGUUGGCAAGGAATT |
| 187 | 103580 | GGAGCUUAUGGUUcUGUCATT |
| 188 | 1555 | GGUCAGAGAUGUAGGACCUTT |
| 189 | 105163 | GGUAAUCAUGUUACUAACCTT |
| 190 | 105773 | GCAGCCUUGUGUUUGGGUATT |
| 191 | 37190 | GGUGUUUCUGGAUGCAUAUTT |
| 192 | 121953 | GCUCUCAGCGUGCUAGUAATT |
| 193 | 9040 | GGACAUUGUGUUCCUGAUCTT |
| 194 | 119716 | GGAAAUCAAGCCCUGCCAATT |
| 195 | 1794 | GGUGCAGAACAUCAAGUUCTT |
| 196 | 115136 | CCCUAUAUCCAGCAUGCGATT |
| 197 | 116921 | CCGAAAUGGAUGCAUUUCATT |
| 198 | 117213 | GCUGUGUCCCUUAGCCUUUTT |
| 199 | 10426 | GGGUCCGAUUUGCCAUCGATT |
| 200 | 657 | GGCUCACCAUGAUGAUUAATT |
| 201 | 46002 | GCUCCAAGGAGGAGAAUAGTT |
| 202 | 105830 | GCCUAUCCUACUAGAACUUTT |
| 203 | 104815 | GGCUCCUCUUACCUCCCAATT |
| 204 | 142280 | GCUUUAUUGAGUCACUGCCTT |
| 205 | 1940 | GGUGAUGAAUUUACCAUCATT |
| 208 | 103397 | GGGUGUGGUCUGAAUUACCTT |
| 207 | 117187 | GCAGCUCAAGUUAGCAUUUTT |
| 208 | 119405 | CCAGAUGAGCUUUCAAGUUTT |
| 209 | 111208 | CCAAUUGGAUAGAGGGUACTT |
| 210 | 118568 | CCAUCAAGGGCUAUGCAUGTT |
| 211 | 383 | GGUUGUUCAUGAUGCUAACTT |
| 212 | 118038 | CCAAAGAUAUCGUGAUUAATT |
| 213 | 42454 | GGCUUUUCUGUCAUGCUUGTT |
| 214 | 118423 | CCUGGAGAGCAUCUUUUCATT |
| 215 | 104231 | GGAGGUGGUCUGUAAAAGGTT |
| 216 | 121036 | CCAGCAAGUGCGACUGUAUTT |
| 217 | 5834 | GGAAAUUUGGAAAUCCUAGTT |
| 218 | 114204 | CCCUAACAAGCUGGUGUUATT |
| 219 | 119258 | CCUAAGCUUCCAUGUAGCCTT |
| 220 | 111728 | GGAAAAACCCUCAGGUGUGTT |
| 221 | 119072 | GCAAGAUUGUCGGCAAGUGTT |
| 222 | 121053 | CGAGGGUUGGGUUUGCUUUTT |
| 223 | 142803 | CCUUUGACUAAUAGGAGUUTT |
| 224 | 117248 | GCAAUCACAUUGCUCUGUATT |
| 225 | 111369 | CCACUGACCCACAGACUCUTT |
| 226 | 118232 | GCACUGACCUCUGUGACUUTT |
| 227 | 10238 | GGAAAGACCACCAUCCUAUTT |
| 228 | 118663 | GCUUCCGGGAAGAAUAUAUTT |
| 229 | 13014 | GGAACAGCGAGCUGUUUGATT |
| 230 | 118922 | GGCAUAAAUUCCUUCUCAGTT |
| 231 | 119792 | GGCCGAGACAAAGAAGCUUTT |
| 232 | 103447 | GGUCUUGUCGGCAGUGAAATT |
| 233 | 23376 | GGGAAGACUCGAACCUUUUTT |
| 234 | 17099 | GGUUGUGAUGUGGUAGUUATT |
| 235 | 138240 | CCUCCUACGGGCCUUUUAUTT |
| 236 | 9004 | GGUGUUUAUAGUGGUGUUUTT |
| 237 | 1785 | GGAUGUGCAGAAAGUGCUGTT |
| 238 | 107446 | GGACAAUCGCACUUUAUACTT |
| 239 | 108267 | GGUAUUUUCUACGGGUGUUTT |
| 240 | 122036 | CCAAACUGUACAGACUCUCTT |
| 241 | 118553 | GCCAAGCAGGAGAUUAACATT |
| 242 | 119612 | CCGAUGUGAAAGGCUGUAGTT |
| 243 | 121775 | CGAAAGACGGUGACUCUUGTT |
| 244 | 110854 | GGAAUGUGUAGAAAGAUCGTT |
| 245 | 126062 | CCAGUUUUAGAUGACUCUUTT |
| 246 | 118792 | CCUUGGAUUACAUAAGGAUTT |
| 247 | 120597 | CGGGCCAGUAUGGUGCAUUTT |
| 248 | 119183 | CCGCAUGGGAGAUGUUCUTT |
| 249 | 121277 | GGGCCCAAAGAAAGAGCUATT |
| 250 | 117497 | GCAGGGUUCAUGCAUAUGATT |
| 251 | 1704 | GGUGUCAGAUAGUAAUAUCTT |
| 252 | 119905 | GCAGUUCUUACUGGACUCATT |
| 253 | 121507 | GCAUCUGGCUAUUAAGUGCTT |
| 254 | 121103 | GGUCAACUUUCAAUUACUGTT |
| 255 | 6501 | GGUGCCCAGAACUCUUUUCTT |
| 256 | 43395 | CAACAACAACUACAAGGAGTT |
| 257 | 1541 | GGACAGAAGCUACAUCUGCTT |
| 258 | 648 | GGAGACCUUCAACCUUCUGTT |
| 259 | 22653 | GGGAUCUUACAUCUAUCAGTT |
| 260 | 112041 | CGACAGAAUAUAACUAACCTT |
| 261 | 116939 | GGAGUUCUAUGUCUGCAUCTT |
| 262 | 111049 | GGCAUUCCCUAUGGCUUAGTT |
| 263 | 120730 | GGACUUCACGGGUAUGGUUTT |
| 264 | 1776 | GGAUUAUUACUACCUGUCATT |
| 265 | 139134 | CCUCUAAUAGAACUGUCUATT |
| 266 | 118865 | GCAAUAUUUAAGCUGUUCUTT |
| 267 | 119034 | GCAACACACAUGUGUAUGGTT |
| 268 | 41802 | GAUAGAUAGGACAACUUCATT |
| 269 | 115614 | GCCUUACAGUUGGCAAGUCTT |
| 270 | 120142 | CGUGAUGGCUGCAUAUGGATT |
| 271 | 129420 | GGUUAUAGUGUGAGACUUGTT |
| 272 | 35139 | GGGAGAAACGCGCCUUAAUTT |
| 273 | 112237 | GCCAACGACUUAGCCUUACTT |
| 274 | 118332 | GCACUUAUUUACACUUCAGTT |
| 275 | 202390 | CCAGGAAUUGUUCUAGUAATT |
| 276 | 111442 | GCCUUUCGUGGUAUCUGCATT |
| 277 | 119734 | GCAACGUGAUCAAAGGUAUTT |
| 278 | 46555 | GCUCCUCAUCACACUGUCATT |
| 279 | 112493 | GCCUUUACUCUGAGGAUAATT |
| 280 | 120542 | CCCAACCAUAAUGGUAAAATT |
| 281 | 143975 | CGACUUUCGCGCCAAGAUGTT |
| 282 | 202509 | CCGGAAUAUUGCUACAUACTT |
| 283 | 26615 | GGCUAAUGUCAUCUGUAUATT |
| 284 | 2021 | GGUAAUUUGUUCUUGGUGATT |
| 285 | 1017 | GGAAGUUCGUGAACAUGUATT |
| 286 | 44902 | GGCUGAGAACGGGAAGCUUTT |
| 287 | 121821 | GCACGGAGAUAAUGAGAAUTT |
| 288 | 3573 | GGUACUAUUUUGAGGUGGATT |
| 289 | 111379 | CGGGAUUUAUUCAGCCUUGTT |
| 290 | 119725 | GGCAACCUAUUAGGCAUGATT |
| 291 | 119012 | GGCUAUGCCACUGGACUCATT |
| 292 | 11202 | GGAUUUCAUUUCAGGUGGATT |
| 293 | 119352 | GGACCUGUACAUGCUUCGATT |
| 294 | 111028 | GGUCACAAGUUGCCAUCUATT |
| 295 | 6242 | GGACCACUGAGAACUUUUGTT |
| 296 | 6829 | GGUGAGUACAUGAGCUGCUTT |
| 297 | 120986 | CCCUAGUCUUCGAAAUGUUTT |
| 298 | 282 | GGCCUGCCAGGAGGUGUUGTT |
| 299 | 119249 | GGAACCGGUAUACAGUGAUTT |
| 300 | 121002 | GGCAUCUGGGUGGGUUUUATT |
| 301 | 112098 | GCGCAUUCCUUUGAUUGGUTT |
| 302 | 120006 | GGAGCUAGAUUCAUAUCCUTT |
| 303 | 9145 | GGUCUGGUCUGAGGAGCUATT |
| 304 | 45672 | GCUGGCAGAUCUGCUUCUATT |
| 305 | 5997 | GGUACGUCACCAUCUUUUATT |
| 306 | 5922 | GGCCAUAGUCCUGUUCACCTT |
| 307 | 112027 | GGGUUAAAAUGGCUGCCAUTT |
| 308 | 42079 | GGUGGACUACUCACGUUUUTT |
| 309 | 104120 | GGGAAAGGGAUAUGUAAUATT |
| 310 | 104465 | GGCACUGGUUAGGAAGGAUTT |
| 311 | 118241 | GGUUGGCAAGAACCUGAUUTT |
| 312 | 12487 | GGUCAUGUGGUUGGACUACTT |
| 313 | 139155 | GCUAUUGUAUAAAGGAUGGTT |
| 314 | 7014 | GGGAGCCUUUGCCUACAUATT |
| 315 | 107742 | GGGAUUUUGGGACUGUUCUTT |
| 316 | 122070 | GCUAGGGAAGUUUGCCGUUTT |
| 317 | 119167 | CCUAUGACUUUUUUGGGUUTT |
| 318 | 121082 | CGAGCCAAUCAUUUCUUCATT |
| 319 | 34166 | GGAUGAAGAUGAAGUGCUUTT |
| 320 | 36798 | GGUGAUUCUGGACAAGGAUTT |
| 321 | 6764 | GGACAUUGCUGAGGUGGAUTT |
| 322 | 112281 | GGUGAAAGCAUAACUUUUUTT |
| 323 | 1359 | GGGCUAGAAAAGAAUGUAATT |
| 324 | 120792 | GAGCAAGAGAUGACCUUAUTT |
| 325 | 120227 | GCCAUAGUAUCAUUGGGCCTT |
| 326 | 117144 | CGAUAGGAAUAGCUUUUAUTT |
| 327 | 202463 | GCUCUGACCAAGUGGAGAUTT |
| 328 | 326 | GGAGCUAGAGCUUGAUGAGTT |
| 329 | 121132 | GCUAUAACCGAGGAAAUUUTT |
| 330 | 40762 | GGAAAACUGUGAACUUUCUTT |
| 331 | 106985 | GGUUAUGGGUAUUGGUGUCTT |
| 332 | 118634 | GCGCAGCUGUCUUUAGCCATT |
| 333 | 1709 | GGACACUUCAUCGUGAGGATT |
| 334 | 119230 | GGUAAUCUACAGUGAGCUGTT |
| 335 | 111644 | GGACAACCUUCCGACUUUUTT |
| 336 | 103331 | GGAUCGAUAUGCCUUGGCATT |
| 337 | 105105 | GGCCCAUAAUAGCCAUGCCTT |
| 338 | 6671 | GGUCACCAUCCAGAAUGUCTT |
| 339 | 117749 | GCUCACAUGCAGUAGACUUTT |
| 340 | 104678 | GGUACUAAUGCAUCUGCAUTT |
| 341 | 4236 | GGUUCAUCUUUCAUGUGAATT |
| 342 | 119150 | CCUUGUUAACAGUGGAGUATT |
| 343 | 120536 | GCCUAGAUCUUGCAUUUAATT |
| 344 | 4084 | GGCCUUCCUACCACUUCAUTT |
| 345 | 8584 | GGAGAAAAAGAUGUCUUUUTT |
| 346 | 118025 | GGAAGCAUUCAAGCAUUUATT |
| 347 | 104025 | GGUUGAUGCGGAGGUGUUUTT |
| 348 | 142304 | CCGGAUGUUAACCUUUAACTT |
| 349 | 119004 | GCCAUCUUAGCAACUUUCUTT |
| 350 | 112199 | GCAGGUCCCUACUAUCAACTT |
| 351 | 140383 | CCUCACUUCUAGACUUUCATT |
| 352 | 43202 | GCAUUGGGAUAUUAAGUAGTT |
| 353 | 118558 | CCUUUACGGCGUAAUCCUGTT |
| 354 | 122033 | CGGUCUGUGUGCUGUUUGATT |
| 355 | 110947 | GCCUAUGGUAGCUGGACUUTT |
| 356 | 122374 | GAAGCAGAGCUGACCUUAGTT |
| 357 | 121768 | CCCAUAGUGAAAUGUGCUGTT |
| 358 | 110667 | GGAUGUGAAAGAGUCUUUUTT |
| 359 | 119683 | GCAAGAAAAACGAACUCUUTT |
| 360 | 105368 | GCAUUUGUUGAUCUGUUCATT |
| 361 | 117476 | GCUGUGCAAAGAACUAUCCTT |
| 362 | 8110 | GGAAGUAUAGAAGAAUGUGTT |
| 363 | 108254 | GGUACGCUGUUAAGUAUUATT |
| 364 | 119254 | CGUGUCAAAGCUGGUUACGTT |
| 365 | 120528 | CCAGAGAUUUGUUUUAUGATT |
| 366 | 114721 | GGAAUUAAGACAUGGUGUGTT |
| 367 | 120404 | GCCAAGACCGAUGUGAAUGTT |
| 368 | 121560 | GCUCCUGUUACAACCUGUGTT |
| 369 | 8759 | GGGACACCAAGACCUACAUTT |
| 370 | 121689 | GCGAUCAUGUCUACAAGGGTT |
| 371 | 116959 | CCUCUUAGGUUGUAUCCUUTT |
| 372 | 18269 | GGGCUACCUUGAGACUUUCTT |
| 373 | 4118 | GGCAGAUGUGGCAUGUCUUTT |
| 374 | 120313 | GGGUGGGAGUUGGUAAAGATT |
| 375 | 14778 | GGUGAGAAUUUCAAGGAUUTT |
| 376 | 1554 | GGAAAAUCGGAUGUGGAUCTT |
| 377 | 120581 | CCCUCUAUAAGGCUUGGUCTT |
| 378 | 135789 | GCCUCAGUAAGAUAGCAUCTT |
| 379 | 104313 | GGUAGCAGAUGGACUAACUTT |
| 380 | 5020 | GGCACUGAAACUCACACUGTT |
| 381 | 103787 | GGCUUUGGCAAGGUGUACATT |
| 382 | 38222 | GGCAUCUUGGUUGUAAGUCTT |
| 383 | 119010 | GGGCUAUAGCCCUCAUAACTT |
| 384 | 119464 | GCUGCCACCAACAAUAUAUTT |
| 385 | 111967 | GCAAUAUCCGACUACUGCATT |
| 386 | 117597 | CCAACACUUUUGACCUUCATT |
| 387 | 616 | GGUGGACAAUCACCUUUUUTT |
| 388 | 104271 | GGAAAGACGGAUUGCAUUATT |
| 389 | 41648 | GUACUUUAACAUCGUCGCUTT |
| 390 | 116760 | CCUUUUCUGUAUAUAGCCATT |
| 391 | 103742 | GGAGCCUCUGCUGAGUAUATT |
| 392 | 121625 | GCUAUCAAUGGUGUAAUACTT |
| 393 | 108793 | GGAGCUCGAGUAUAUUUAGTT |
| 394 | 46149 | GAAAAGAUAUUGGAUGCUCTT |
| 395 | 121965 | GCCUAAUUGAUUUCAUUCUTT |
| 396 | 104655 | GGAAAUGCAGCCUAGUCUUTT |
| 397 | 3025 | GGAAGAUUAUUCUGAAUACTT |
| 398 | 23439 | GGUUGAUGUAACCUUUUAATT |
| 399 | 31620 | GGUGGAAAUGAUGUUUAUCTT |
| 400 | 4069 | GGACAACUUUGGUGCUGUGTT |
| 401 | 107669 | GGCUUUGGGAUAUGCUGUATT |
| 402 | 9248 | GGAGUCCAACAAGAAGCACTT |
| 403 | 106198 | GGAUCUGUACCCAGUAAUCTT |
| 404 | 112515 | CCAUCAAUGUAACUUGUACTT |
| 405 | 8537 | GGAAAAUGACUUGUAAGGUTT |
| 406 | 110610 | CCAACCAUGGGCAUAUCCUTT |
| 407 | 43265 | GACCUGGCCAAGUACAUGATT |
| 408 | 180 | GGAAGUGGCAGUGAAGCUATT |
| 409 | 117634 | CCUAUCCAUUACUUAAGGATT |
| 410 | 8947 | GGUGCAGAAACCUGUGAAGTT |
| 411 | 10429 | GGACUGUGGAGACGUAAAUTT |
| 412 | 107317 | GGUAUGCGACGGGAAAGUATT |
| 413 | 121476 | GCUACUUUGGCCGUAAGGUTT |
| 414 | 126545 | GGAGGUUAUGGCCAUUGCATT |
| 415 | 202300 | GUUCACCUACAAGGAGAUCTT |
| 416 | 105208 | GGCAAAUGUUCUCACUGCATT |
| 417 | 109375 | GGAGUUUCAGACCCUAUCCTT |
| 418 | 120071 | GGGAUAUGCAAACCUCAUCTT |
| 419 | 122067 | GGACUCAGACACAGGUGAUTT |
| 420 | 18224 | GGCGGCAGAACAUUGCUUATT |
| 421 | 2057 | GGCAGACAGCUAUACCUUGTT |
| 422 | 6205 | GGACAUCAUCACCUUGGAATT |
| 423 | 103432 | GGGAUGUUGUAUCUUCAUUTT |
| 424 | 107085 | GGUAGCAACAGCGAUUGGATT |
| 425 | 117546 | GCUAUGCAGAUGGCGUGUATT |
| 426 | 41929 | GCAUUACGGUGUCUUCACCTT |
| 427 | 112254 | CCAAUCGAUAAUCACAUUGTT |
| 428 | 105538 | GACGGGAACACUCCAAUGATT |
| 429 | 444 | GGAAAGCAAUCACUUCUCATT |
| 430 | 6722 | GGCUUUUUCCAGCCUUACUTT |
| 431 | 40719 | GGAGAUCAUUAAGAUUACATT |
| 432 | 115262 | GCGGUGUUCAUGAUUUCUUTT |
| 433 | 106223 | GGACAUUCCCACUAUUAUGTT |
| 434 | 120151 | GGGAAGGUGAUUUACUUCATT |
| 435 | 105664 | GAGAUGACAGCAAUGAGUCTT |
| 436 | 1020 | GGACAACAAUUUGCAUUAATT |
| 437 | 112308 | CGUAAGUCCACAUAUACUUTT |
| 436 | 120484 | GGCUAUGAAUUGGACCUUUTT |
| 439 | 670 | GGAGUUCAAUAAAUGUCUGTT |
| 440 | 1397 | GGGAAAACGGCACCUUUUCTT |
| 441 | 104702 | GGCAAUCAAGGGUACAUACTT |
| 442 | 1140 | GGAUCUGAGGCAGGGUUUUTT |
| 443 | 112418 | CCAAGUACGCAAACUUUUCTT |
| 444 | 104693 | GGAGUGAUUAGUUCGGGUUTT |
| 445 | 8943 | GGAUUCAUAGACUUCAUAGTT |
| 446 | 107096 | GGAAAAAUGAAUUGCUUGGTT |
| 447 | 2531 | GGUAACUGUGCUGAGGGUCTT |
| 448 | 118060 | GGGUCCAAGAGAUAUACUGTT |
| 449 | 118590 | GCUCCUUAAGGCUCUUUUGTT |
| 450 | 118906 | CCUAUUAGUGUCUCCUACATT |
| 451 | 106243 | GGUCACAUUUAUGUGGAAGTT |
| 452 | 111874 | CGUGCUUUUUCUCAAGUAUTT |
| 453 | 8193 | GGAUGGGCUCAAAUACUACTT |
| 454 | 2512 | GGAAAUAAAAGAUCUUGACTT |
| 455 | 16362 | GGCUCCUACUACGAGUAUUTT |
| 456 | 14218 | GGGUUUAAUACAGCAUGUGTT |
| 457 | 103493 | GGCAUAUUCCACUGAUUACTT |
| 458 | 1176 | GGUCCACAAGGCAGUGCUGTT |
| 459 | 111523 | CGAAGUUAUCAGAGAUGCUTT |
| 460 | 33700 | GGAGAAAUGACCUCAUUUUTT |
| 461 | 2167 | GGAAUGAUCCAUACUGAAATT |
| 462 | 105854 | GACAUCUUAGCCAGGAAAUTT |
| 463 | 46281 | GCUGCCUGUGGUGGUAAAATT |
| 464 | 44894 | GAAGCUGGAUACAGCAUAUTT |
| 465 | 38041 | GGAAAGACAACAAAUACUATT |
| 466 | 42278 | GUUAGCUUCAUAAUCUGUUTT |
| 467 | 112472 | GCCUAUCAAGACUUCAUUATT |

TABLE 4

| | | Expr. in | | Expr. in | | Expr. in | |
|---|---|---|---|---|---|---|---|
| Target | siRNA | HepG2 cells | | Huh cells | | primary Hepatocytes | |
| No. | ID | AffyID | AvgExp | AffyID | AvgExp | AffyID | AvgExp |
| 1 | 113613 | 221215_s_at | 331 | 221215_s_at | 1314 | 221215_s_at | 1453 |
| 1 | 105742 | 221215_s_at | 331 | 221215_s_at | 1314 | 221215_s_at | 1453 |
| 1 | 105202 | 221215_s_at | 331 | 221215_s_at | 1314 | 221215_s_at | 1453 |
| 2 | 117853 | 207833_s_at | 111 | 209399_at | 254 | 207833_s_at | 246 |
| 2 | 117852 | 207833_s_at | 111 | 209399_at | 254 | 207833_s_at | 246 |
| 2 | 117851 | 207833_s_at | 111 | 209399_at | 254 | 207833_s_at | 246 |
| 3 | 117417 | 209146_at | 16655 | 209146_at | 20005 | 209146_at | 17401 |
| 3 | 117416 | 209146_at | 16655 | 209146_at | 20005 | 209146_at | 17401 |
| 3 | 117418 | 209146_at | 16655 | 209146_at | 20005 | 209146_at | 17401 |
| 4 | 42711 | 209970_x_at | 1411 | 211366_x_at | 155 | 211366_x_at | 1281 |
| 4 | 42626 | 209970_x_at | 1411 | 211366_x_at | 155 | 211366_x_at | 1281 |
| 5 | 10418 | 208727_s_at | 5287 | 208727_s_at | 16227 | 208727_s_at | 6132 |
| 5 | 10505 | 208727_s_at | 5287 | 208727_s_at | 16227 | 208727_s_at | 6132 |
| 6 | 118135 | 206155_at | 690 | 206155_at | 655 | 206155_at | 4275 |
| 6 | 116839 | 206155_at | 690 | 206155_at | 655 | 206155_at | 4275 |
| 7 | 105039 | 205927_s_at | 139 | 205927_s_at | 815 | 205927_s_at | 167 |
| 7 | 105041 | 205927_s_at | 139 | 205927_s_at | 815 | 205927_s_at | 167 |
| 8 | 1147 | 207178_s_at | 64 | 207178_s_at | 209 | 207178_s_at | 348 |
| 8 | 1243 | 207178_s_at | 64 | 207178_s_at | 209 | 207178_s_at | 348 |
| 9 | 8225 | 203040_s_at | 1156 | 203040_s_at | 1002 | 203040_s_at | 1214 |
| 9 | 117844 | 203040_s_at | 1156 | 203040_s_at | 1002 | 203040_s_at | 1214 |
| 10 | 106087 | 201413_at | 6700 | 201413_at | 12772 | 201413_at | 4948 |
| 10 | 106089 | 201413_at | 6700 | 201413_at | 12772 | 201413_at | 4948 |
| 11 | 121011 | 212531_at | 51 | 212531_at | 33 | 212531_at | 1215 |
| 11 | 121012 | 212531_at | 51 | 212531_at | 33 | 212531_at | 1215 |
| 12 | 1766 | 206825_at | 108 | 206825_at | 168 | 206825_at | 81 |
| 12 | 1947 | 206825_at | 108 | 206825_at | 168 | 206825_at | 81 |
| 13 | 142836 | 204284_at | 909 | 204284_at | 1704 | 204284_at | 6326 |
| 13 | 142834 | 204284_at | 909 | 204284_at | 1704 | 204284_at | 6326 |
| 14 | 1547 | 203218_at | 2388 | 203218_at | 2503 | 203218_at | 927 |
| 14 | 1452 | 203218_at | 2388 | 203218_at | 2503 | 203218_at | 927 |
| 15 | 1699 | 211370_s_at | 315 | 211370_s_at | 439 | 204756_at | 209 |
| 15 | 118252 | 211370_s_at | 315 | 211370_s_at | 439 | 204756_at | 209 |
| 16 | 43916 | | | | | | |
| 16 | 43820 | | | | | | |
| 17 | 402 | 203856_at | 1496 | 203856_at | 2144 | 203856_at | 511 |
| 17 | 401 | 203856_at | 1496 | 203856_at | 2144 | 203856_at | 511 |
| 18 | 117695 | 230084_at | 91 | 230084_at | 36 | 230084_at | 249 |
| 18 | 117693 | 230084_at | 91 | 230084_at | 36 | 230084_at | 249 |
| 19 | 118261 | 209333_at | 171 | 209333_at | 242 | 209333_at | 194 |
| 19 | 118260 | 209333_at | 171 | 209333_at | 242 | 209333_at | 194 |
| 20 | 5146 | 221312_at | 15 | 221312_at | 17 | 221312_at | 10 |
| 20 | 5237 | 221312_at | 15 | 221312_at | 17 | 221312_at | 10 |
| 21 | 828 | 201939_at | 168 | 201939_at | 218 | 201939_at | 8886 |
| 21 | 829 | 201939_at | 168 | 201939_at | 218 | 201939_at | 8886 |
| 22 | 19104 | 202458_at | 91 | 202458_at | 538 | 202458_at | 669 |
| 22 | 19196 | 202458_at | 91 | 202458_at | 538 | 202458_at | 669 |
| 23 | 103354 | 216945_x_at | 282 | 213534_s_at | 185 | 216945_x_at | 35 |
| 23 | 978 | 216945_x_at | 282 | 213534_s_at | 185 | 216945_x_at | 35 |
| 24 | 2240 | 221329_at | 31 | 221329_at | 40 | 221329_at | 85 |
| 24 | 2061 | 221329_at | 31 | 221329_at | 40 | 221329_at | 85 |
| 25 | 20115 | 220334_at | 52 | 220334_at | 33 | 220334_at | 43 |
| 25 | 20024 | 220334_at | 52 | 220334_at | 33 | 220334_at | 43 |
| 26 | 118397 | 228098_s_at | 1559 | 228098_s_at | 505 | 228098_s_at | 911 |
| 26 | 118398 | 228098_s_at | 1559 | 228098_s_at | 505 | 228098_s_at | 911 |
| 27 | 104835 | 1559261_a_at | 156 | 1559261_a_at | 104 | 1559261_a_at | 158 |
| 27 | 104830 | 1559261_a_at | 156 | 1559261_a_at | 104 | 1559261_a_at | 158 |
| 28 | 119221 | 205750_at | 260 | 205750_at | 853 | 205750_at | 413 |
| 29 | 105141 | 221654_s_at | 5010 | 221654_s_at | 3066 | 221654_s_at | 3157 |
| 30 | 122236 | 225783_at | 4314 | 225787_at | 1528 | 225783_at | 3676 |
| 31 | 43781 | 224049_at | 126 | 224049_at | 65 | 224049_at | 177 |
| 32 | 103636 | 212533_at | 2413 | 212533_at | 3573 | 212533_at | 6944 |
| 33 | 45922 | 236407_at | 153 | 208514_at | 45 | 236407_at | 353 |
| 34 | 117371 | 207438_s_at | 1946 | 207438_s_at | 1674 | 207438_s_at | 847 |
| 35 | 111157 | 200901_s_at | 5755 | 200901_s_at | 3087 | 200900_s_at | 1971 |
| 36 | 38979 | 232647_at | 94 | 232647_at | 141 | 232647_at | 118 |
| 37 | 121933 | 227614_at | 139 | 227614_at | 44 | 227614_at | 660 |
| 38 | 119829 | 222839_s_at | 631 | 222839_s_at | 476 | 224427_s_at | 559 |
| 39 | 19471 | 203105_s_at | 1802 | 203105_s_at | 980 | 203105_s_at | 1867 |
| 40 | 120442 | 212296_at | 8661 | 212296_at | 14932 | 212296_at | 9585 |
| 41 | 105154 | 222462_s_at | 507 | 222462_s_at | 726 | 222462_s_at | 578 |
| 42 | 6196 | 224285_at | 30 | 224285_at | 25 | 224285_at | 26 |
| 43 | 105753 | 219058_x_at | 74 | 219058_x_at | 156 | 219058_x_at | 101 |
| 44 | 21895 | 206868_at | 207 | 206868_at | 108 | 206868_at | 154 |
| 45 | 120445 | 214369_s_at | 262 | 214369_s_at | 120 | 214369_s_at | 266 |
| 46 | 111807 | 205174_s_at | 1889 | 205174_s_at | 31 | 205174_s_at | 135 |
| 47 | 1721 | 216220_s_at | 127 | 216220_s_at | 121 | 216220_s_at | 170 |
| 48 | 1774 | 208048_at | 60 | 208048_at | 28 | 208048_at | 50 |
| 49 | 117712 | | | | | | |
| 50 | 1795 | 212070_at | 395 | 212070_at | 69 | 212070_at | 187 |
| 51 | 117084 | 203537_at | 1771 | 203537_at | 1683 | 203537_at | 1339 |
| 52 | 119926 | 214951_at | 19 | 214951_at | 55 | 214951_at | 26 |
| 53 | 9067 | 210505_at | 52 | 210505_at | 30 | 210505_at | 53 |
| 54 | 121179 | 234436_x_at | 30 | 234436_x_at | 21 | 234841_x_at | 24 |
| 55 | 38327 | | | | | | |
| 56 | 111813 | 207641_at | 82 | 207641_at | 122 | 207641_at | 150 |
| 57 | 107569 | 210609_s_at | 836 | 210609_s_at | 993 | 210609_s_at | 1914 |
| 58 | 10560 | 206651_s_at | 3972 | 206651_s_at | 2798 | 206651_s_at | 23261 |
| 59 | 10235 | 204732_s_at | 330 | 204732_s_at | 400 | 210995_s_at | 504 |
| 60 | 104127 | 221337_s_at | 10 | 221337_s_at | 35 | 221337_s_at | 34 |
| 61 | 112077 | 225440_at | 981 | 223184_s_at | 1081 | 225440_at | 1232 |
| 62 | 105010 | 202450_s_at | 253 | 202450_s_at | 251 | 202450_s_at | 203 |
| 63 | 4154 | 229105_at | 86 | 229105_at | 559 | 229105_at | 683 |
| 64 | 40980 | 213922_at | 127 | 213922_at | 120 | 1554293_at | 130 |
| 65 | 120756 | 213036_x_at | 220 | 207521_s_at | 31 | 207521_s_at | 66 |
| 66 | 1627 | 210105_s_at | 1206 | 210105_s_at | 472 | 210105_s_at | 283 |
| 67 | 122128 | | | | | | |
| 68 | 2207 | | | | | | |
| 69 | 4633 | 211438_at | 7 | 211438_at | 11 | 211438_at | 32 |
| 70 | 119952 | 225835_at | 899 | 225835_at | 2939 | 204404_at | 447 |
| 71 | 105325 | 205624_at | 36 | 205624_at | 21 | 205624_at | 16 |
| 72 | 112330 | 226314_at | 444 | 226314_at | 340 | 226314_at | 93 |
| 73 | 121576 | 205320_at | 150 | 205320_at | 138 | 205320_at | 205 |
| 74 | 117799 | 231424_at | 23 | 231424_at | 179 | 214389_at | 29 |
| 75 | 104458 | 211662_s_at | 14795 | 211662_s_at | 10012 | 211662_s_at | 25082 |
| 76 | 112453 | 209240_at | 2917 | 209240_at | 3035 | 209240_at | 1612 |
| 77 | 121337 | 205046_at | 489 | 205046_at | 1193 | 205046_at | 9 |
| 78 | 110844 | 202315_s_at | 345 | 226602_s_at | 477 | 202315_s_at | 388 |
| 79 | 119422 | 1560445_x_at | 212 | 1560445_x_at | 177 | 1560445_x_at | 171 |
| 80 | 119276 | 208649_s_at | 3384 | 208649_s_at | 6397 | 208649_s_at | 4972 |
| 81 | 118817 | 218440_at | 1546 | 218440_at | 2214 | 218440_at | 1212 |
| 82 | 118114 | 224002_s_at | 509 | 224002_s_at | 1031 | 224002_s_at | 460 |
| 83 | 118462 | 202962_at | 265 | 202962_at | 724 | 202962_at | 582 |
| 84 | 46510 | 1553222_at | 153 | 1553222_at | 145 | 1553222_at | 68 |
| 85 | 119129 | 202517_at | 100 | 202517_at | 38 | 202517_at | 66 |
| 86 | 119399 | 234513_at | 24 | 234513_at | 27 | 234513_at | 27 |
| 87 | 122166 | 210027_s_at | 6714 | 210027_s_at | 5325 | 210027_s_at | 7170 |
| 88 | 45063 | 211226_at | 53 | 211226_at | 83 | 211226_at | 96 |
| 89 | 117601 | 220371_s_at | 249 | 220371_s_at | 238 | 220371_s_at | 168 |
| 90 | 118446 | 209408_at | 1715 | 209408_at | 2483 | 209408_at | 112 |
| 91 | 18240 | 224300_x_at | 183 | 224300_x_at | 416 | 223702_x_at | 1248 |
| 92 | 104559 | 204811_s_at | 169 | 204811_s_at | 200 | 204811_s_at | 303 |
| 93 | 1407 | 202530_at | 2293 | 202530_at | 2001 | 211561_x_at | 1358 |
| 94 | 119077 | 201765_s_at | 2783 | 201765_s_at | 1993 | 201765_s_at | 1371 |
| 95 | 1371 | 219257_s_at | 1030 | 219257_s_at | 394 | 219257_s_at | 508 |
| 96 | 106537 | 202325_s_at | 17972 | 202325_s_at | 19536 | 202325_s_at | 16304 |
| 97 | 120500 | 205811_at | 605 | 205811_at | 852 | 205811_at | 839 |
| 98 | 111654 | 207085_x_at | 147 | 211286_x_at | 47 | 211287_x_at | 131 |
| 99 | 118718 | 209723_at | 596 | 209723_at | 3656 | 209723_at | 910 |
| 100 | 120608 | 220418_at | 16 | 220418_at | 22 | 220418_at | 8 |
| 101 | 142253 | 213465_s_at | 4176 | 213465_s_at | 1986 | 213465_s_at | 2635 |
| 102 | 111081 | 212293_at | 2245 | 212293_at | 2410 | 212293_at | 1664 |
| 103 | 103786 | 200075_s_at | 2999 | 200075_s_at | 2534 | 200075_s_at | 3897 |
| 104 | 121943 | 219248_at | 819 | 219248_at | 569 | 219248_at | 654 |
| 105 | 121806 | 223597_at | 103 | 223597_at | 65 | 223597_at | 70 |
| 106 | 15527 | 233547_x_at | 38 | 233547_x_at | 34 | 208396_s_at | 37 |
| 107 | 143005 | 204612_at | 26 | 204612_at | 1000 | 204612_at | 54 |
| 108 | 110607 | 205498_at | 11 | 205498_at | 217 | 205498_at | 1373 |
| 109 | 107834 | 210852_s_at | 121 | 214829_at | 254 | 214829_at | 131 |
| 110 | 121163 | | | | | | |
| 111 | 118522 | 204411_at | 193 | 204411_at | 92 | 204411_at | 39 |
| 112 | 120179 | 211285_s_at | 4332 | 211285_s_at | 3412 | 211285_s_at | 4322 |
| 113 | 34999 | 225411_at | 5315 | 225411_at | 1649 | 225411_at | 583 |
| 114 | 6091 | 223767_at | 797 | 223767_at | 30 | 223767_at | 10 |
| 115 | 103829 | | | | | | |
| 116 | 119396 | 202548_s_at | 1872 | 202548_s_at | 827 | 202548_s_at | 1083 |
| 117 | 8816 | 209420_s_at | 328 | 209420_s_at | 296 | 209420_s_at | 561 |
| 118 | 15339 | 207800_at | 26 | 207800_at | 18 | 207800_at | 32 |
| 119 | 29459 | 218480_at | 498 | 218480_at | 368 | 231857_s_at | 133 |
| 120 | 16059 | 214518_at | 93 | 214518_at | 126 | 214518_at | 187 |
| 121 | 104173 | 209765_at | 139 | 221128_at | 69 | 209765_at | 260 |
| 122 | 120611 | 218186_at | 38 | 218186_at | 13 | 218186_at | 26 |
| 123 | 142929 | 201834_at | 543 | 201834_at | 744 | 201835_s_at | 760 |
| 124 | 35220 | 208912_s_at | 1547 | 208912_s_at | 1273 | 208912_s_at | 1142 |
| 125 | 119469 | 204867_at | 1286 | 204867_at | 1727 | 204867_at | 733 |
| 126 | 121471 | 205263_at | 2961 | 205263_at | 3849 | 205263_at | 2914 |
| 127 | 122003 | 229253_at | 786 | 229253_at | 954 | 229253_at | 911 |
| 128 | 114058 | 200602_at | 2782 | 214953_s_at | 4350 | 200602_at | 2082 |
| 129 | 117031 | 206662_at | 9085 | 206662_at | 2432 | 206662_at | 11092 |
| 130 | 110906 | 203917_at | 6573 | 203917_at | 8611 | 203917_at | 4681 |
| 131 | 119517 | | | | | | |
| 132 | 114048 | | | | | | |
| 133 | 809 | 206028_s_at | 3023 | 206028_s_at | 543 | 211913_s_at | 326 |
| 134 | 137195 | 212625_at | 1388 | 212625_at | 1037 | 212625_at | 554 |
| 135 | 118341 | 229106_at | 223 | 229106_at | 168 | 229106_at | 451 |
| 136 | 46319 | 223284_at | 281 | 223284_at | 104 | 223284_at | 56 |
| 137 | 7204 | 204401_at | 2183 | 204401_at | 16 | 204401_at | 63 |
| 138 | 119081 | 209166_s_at | 3051 | 209166_s_at | 743 | 209166_s_at | 257 |
| 139 | 745 | 40420_at | 1233 | 40420_at | 159 | 40420_at | 522 |
| 140 | 119216 | 207618_s_at | 689 | 207618_s_at | 626 | 207618_s_at | 1792 |
| 141 | 117277 | 207408_at | 162 | 207408_at | 165 | 207408_at | 201 |
| 142 | 14775 | 202298_at | 19882 | 202298_at | 9707 | 202298_at | 12823 |
| 143 | 119182 | 211733_x_at | 9927 | 201339_s_at | 15103 | 211733_x_at | 9050 |
| 144 | 1286 | 218942_at | 935 | 218942_at | 1495 | 218942_at | 952 |
| 145 | 1961 | 214605_x_at | 66 | 214605_x_at | 50 | 214605_x_at | 43 |
| 146 | 119782 | 203865_s_at | 751 | 203865_s_at | 203 | 203865_s_at | 148 |
| 147 | 135366 | 206092_x_at | 179 | 206092_x_at | 167 | 206092_x_at | 111 |
| 148 | 42362 | 221327_s_at | 34 | 221327_s_at | 31 | 221327_s_at | 84 |
| 149 | 12155 | 225418_at | 1120 | 225418_at | 1413 | 203149_at | 1037 |
| 150 | 11 | 210838_s_at | 57 | 210838_s_at | 53 | 210838_s_at | 31 |
| 151 | 7881 | 210944_s_at | 250 | 210944_s_at | 103 | 210944_s_at | 273 |
| 152 | 10428 | 201097_s_at | 12304 | 201097_s_at | 11471 | 201097_s_at | 19811 |
| 153 | 16123 | 201036_s_at | 2233 | 201036_s_at | 2352 | 201036_s_at | 3584 |
| 154 | 1049 | 210346_s_at | 1129 | 210346_s_at | 506 | 210346_s_at | 1149 |
| 155 | 919 | 214398_s_at | 639 | 204549_at | 163 | 214398_s_at | 180 |
| 156 | 121066 | 56197_at | 1567 | 56197_at | 946 | 56197_at | 1354 |
| 157 | 105169 | 203356_at | 1488 | 203356_at | 7040 | 203356_at | 1403 |
| 158 | 36311 | 223809_at | 222 | 223809_at | 45 | 223809_at | 57 |
| 159 | 5884 | 221297_at | 47 | 221297_at | 79 | 221297_at | 119 |
| 160 | 44940 | 205236_x_at | 128 | 205236_x_at | 125 | 205236_x_at | 61 |
| 161 | 1398 | 224691_at | 9910 | 224691_at | 7244 | 224691_at | 6110 |
| 162 | 104626 | 1561820_at | 103 | 1561820_at | 73 | 1561820_at | 124 |
| 163 | 15563 | 216713_at | 877 | 216713_at | 1296 | 34031_i_at | 990 |
| 164 | 136772 | 213178_s_at | 279 | 213178_s_at | 215 | 213178_s_at | 251 |
| 165 | 46183 | | | | | | |
| 166 | 5377 | 37152_at | 8976 | 37152_at | 1215 | 37152_at | 3151 |
| 167 | 103491 | 1552631_a_at | 112 | 219278_at | 75 | 1552631_a_at | 97 |
| 168 | 117929 | 208746_x_at | 21945 | 208746_x_at | 14947 | 210453_x_at | 14839 |
| 169 | 110591 | 204264_at | 1945 | 204264_at | 1752 | 204264_at | 661 |
| 170 | 103534 | 223141_at | 516 | 223141_at | 565 | 223141_at | 777 |
| 171 | 119066 | 205232_s_at | 335 | 205232_s_at | 349 | 205232_s_at | 743 |
| 172 | 106403 | 208950_s_at | 1587 | 208950_s_at | 4917 | 208950_s_at | 1784 |
| 173 | 121059 | 219726_at | 92 | 219726_at | 64 | 219726_at | 27 |
| 174 | 121219 | | | | | | |
| 175 | 117366 | 208462_s_at | 40 | 208462_s_at | 49 | 208562_s_at | 151 |
| 176 | 105936 | 210653_s_at | 485 | 210653_s_at | 530 | 210653_s_at | 465 |
| 177 | 105919 | 206833_s_at | 901 | 206833_s_at | 1029 | 206833_s_at | 967 |
| 178 | 103932 | 239260_at | 40 | 220356_at | 27 | 239261_s_at | 54 |
| 179 | 43139 | 1553549_at | 31 | 1553549_at | 24 | 1553549_at | 34 |
| 180 | 9108 | 206780_at | 73 | 206780_at | 94 | 206780_at | 67 |
| 181 | 111592 | 205139_s_at | 87 | 205139_s_at | 42 | 205139_s_at | 70 |
| 182 | 104388 | 201734_at | 1886 | 201734_at | 2375 | 201735_s_at | 1535 |
| 183 | 115931 | 216092_s_at | 446 | 216092_s_at | 154 | 216092_s_at | 280 |
| 184 | 30832 | 209179_s_at | 399 | 209179_s_at | 371 | 209179_s_at | 320 |
| 185 | 105076 | 205356_at | 1738 | 205356_at | 998 | 205356_at | 599 |
| 186 | 44885 | 210445_at | 59 | 210445_at | 62 | 210445_at | 45 |
| 187 | 103580 | 203266_s_at | 642 | 203266_s_at | 978 | 203266_s_at | 1360 |
| 188 | 1555 | 215664_s_at | 17 | 237939_at | 307 | 237939_at | 26 |
| 189 | 105163 | 220419_s_at | 1776 | 220419_s_at | 2185 | 220419_s_at | 2778 |
| 190 | 105773 | 226469_s_at | 91 | 226470_at | 158 | 229788_s_at | 61 |
| 191 | 37190 | 229250_at | 307 | 229250_at | 353 | 229251_s_at | 358 |
| 192 | 121953 | 212129_at | 5571 | 212129_at | 4127 | 212129_at | 4333 |
| 193 | 9040 | 210184_at | 975 | 1563003_at | 43 | 1563003_at | 58 |
| 194 | 119716 | 210627_s_at | 546 | 210627_s_at | 775 | 210627_s_at | 501 |
| 195 | 1794 | 218629_at | 119 | 218629_at | 229 | 218629_at | 177 |
| 196 | 115136 | 217207_s_at | 197 | 217207_s_at | 134 | 217207_s_at | 230 |
| 197 | 116921 | 207519_at | 137 | 207519_at | 206 | 207519_at | 45 |
| 198 | 117213 | 200078_s_at | 13495 | 200078_s_at | 5732 | 200078_s_at | 7471 |
| 199 | 10426 | 200065_s_at | 13052 | 200065_s_at | 10059 | 200065_s_at | 10196 |
| 200 | 657 | 206412_at | 383 | 206412_at | 260 | 206412_at | 239 |
| 201 | 46002 | 206994_at | 77 | 206994_at | 79 | 206994_at | 72 |
| 202 | 105830 | 200726_at | 19179 | 200726_at | 16496 | 200726_at | 9290 |
| 203 | 104815 | 234554_at | 20 | 234554_at | 30 | 234554_at | 89 |
| 204 | 142280 | 203680_at | 303 | 203680_at | 1362 | 203680_at | 40 |
| 205 | 1940 | 211479_s_at | 39 | 207307_at | 38 | 211479_s_at | 60 |
| 206 | 103397 | 205880_at | 140 | 205680_at | 116 | 217705_at | 31 |
| 207 | 117187 | 204894_s_at | 683 | 204894_s_at | 187 | 204894_s_at | 328 |
| 208 | 119405 | 227224_at | 601 | 227224_at | 2949 | 227224_at | 936 |
| 209 | 111208 | 211846_s_at | 55 | 208455_at | 48 | 208455_at | 68 |
| 210 | 118568 | 204067_at | 502 | 204067_at | 660 | 204067_at | 429 |
| 211 | 383 | 206301_at | 14 | 206301_at | 22 | 206301_at | 59 |
| 212 | 118038 | 221210_s_at | 2274 | 223405_at | 36 | 223405_at | 612 |
| 213 | 42454 | 221320_at | 67 | 221320_at | 77 | 236491_at | 185 |
| 214 | 118423 | 203087_s_at | 1764 | 203087_s_at | 2058 | 203087_s_at | 1001 |
| 215 | 104231 | 237411_at | 201 | 237411_at | 354 | 1570351_at | 114 |
| 216 | 121036 | 220291_at | 30 | 220291_at | 71 | 220291_at | 33 |
| 217 | 5834 | 203631_s_at | 58 | 203632_s_at | 859 | 203632_s_at | 566 |
| 218 | 114204 | 215001_s_at | 17454 | 215001_s_at | 14848 | 215001_s_at | 11147 |
| 219 | 119258 | 205493_s_at | 128 | 205493_s_at | 376 | 205492_s_at | 120 |
| 220 | 111728 | 206856_at | 10 | 206856_at | 9 | 206856_at | 13 |
| 221 | 119072 | 206335_at | 892 | 206335_at | 666 | 206335_at | 308 |
| 222 | 121053 | 218552_at | 707 | 218552_at | 932 | 218552_at | 4149 |
| 223 | 142803 | 209323_at | 2444 | 209323_at | 2027 | 209323_at | 2860 |
| 224 | 117248 | 213889_at | 685 | 213889_at | 562 | 213889_at | 747 |
| 225 | 111369 | 209354_at | 564 | 209354_at | 186 | 209354_at | 520 |
| 226 | 118232 | 212672_at | 1417 | 212672_at | 939 | 210858_x_at | 265 |
| 227 | 10238 | 200011_s_at | 3446 | 200011_s_at | 2073 | 200734_s_at | 2101 |
| 228 | 118663 | 204614_at | 260 | 204614_at | 13 | 204614_at | 76 |
| 229 | 13014 | 226063_at | 496 | 226063_at | 884 | 226063_at | 1181 |
| 230 | 118922 | 219464_at | 151 | 219464_at | 108 | 219464_at | 121 |
| 231 | 119792 | 201391_at | 2041 | 201391_at | 2119 | 201391_at | 2717 |
| 232 | 103447 | 217128_s_at | 84 | 217128_s_at | 79 | 217128_s_at | 143 |
| 233 | 23376 | 217933_s_at | 7272 | 217933_s_at | 8327 | 217933_s_at | 6105 |
| 234 | 17099 | 209036_s_at | 9784 | 209036_s_at | 10620 | 209036_s_at | 13667 |
| 235 | 138240 | 204746_s_at | 121 | 204746_s_at | 118 | 204746_s_at | 176 |
| 236 | 9004 | 205629_s_at | 38 | 205629_s_at | 42 | 205629_s_at | 73 |
| 237 | 1785 | 214613_at | 37 | 214613_at | 16 | 214613_at | 22 |
| 238 | 107446 | 217860_at | 4455 | 217860_at | 2574 | 217860_at | 3804 |
| 239 | 108267 | 202878_s_at | 1326 | 202878_s_at | 52 | 202878_s_at | 441 |
| 240 | 122036 | 226700_at | 86 | 226700_at | 56 | 226700_at | 128 |
| 241 | 118553 | 206386_at | 3756 | 206386_at | 260 | 206386_at | 5243 |
| 242 | 119612 | 201571_s_at | 1735 | 210137_s_at | 503 | 210137_s_at | 2446 |
| 243 | 121775 | 221009_s_at | 123 | 221009_s_at | 65 | 221009_s_at | 9529 |
| 244 | 110854 | 205399_at | 22 | 205399_at | 22 | 205399_at | 30 |
| 245 | 126062 | 226925_at | 794 | 226925_at | 1420 | 226925_at | 381 |
| 246 | 118792 | 215047_at | 64 | 215047_at | 17 | 215047_at | 11 |
| 247 | 120597 | 223345_at | 562 | 223345_at | 312 | 223909_s_at | 384 |
| 248 | 119183 | 203234_at | 3913 | 203234_at | 304 | 203234_at | 4234 |
| 249 | 121277 | 205141_at | 2433 | 205141_at | 675 | 205141_at | 9942 |
| 250 | 117497 | 220413_at | 79 | 220413_at | 31 | 220413_at | 62 |
| 251 | 1704 | 226649_at | 1117 | 226649_at | 2012 | 226649_at | 684 |
| 252 | 119905 | 207088_s_at | 1012 | 207088_s_at | 719 | 207088_s_at | 1485 |
| 253 | 121507 | 224654_at | 7484 | 224654_at | 8956 | 224654_at | 13951 |
| 254 | 121103 | 238160_at | 24 | 238160_at | 99 | 238160_at | 416 |
| 255 | 6501 | 210961_s_at | 73 | 210961_s_at | 32 | 210961_s_at | 63 |
| 256 | 43395 | 207703_at | 101 | 1554125_a_at | 36 | 1554125_a_at | 33 |
| 257 | 1541 | 202273_at | 131 | 202273_at | 108 | 202273_at | 191 |
| 258 | 648 | 205977_s_at | 112 | 205977_s_at | 157 | 205977_s_at | 92 |
| 259 | 22653 | 34206_at | 544 | 34206_at | 386 | 34206_at | 398 |
| 260 | 112041 | 218146_at | 3290 | 218146_at | 1717 | 218146_at | 2189 |
| 261 | 116939 | 201128_s_at | 7740 | 201128_s_at | 10360 | 201128_s_at | 3752 |
| 262 | 111049 | 226072_at | 230 | 226072_at | 405 | 226072_at | 116 |
| 263 | 120730 | 1553713_a_at | 96 | 1553713_a_at | 85 | 1553713_a_at | 45 |
| 264 | 1776 | 207151_at | 100 | 207151_at | 72 | 207151_at | 96 |
| 265 | 139134 | 205632_s_at | 336 | 205632_s_at | 1079 | 205632_s_at | 40 |
| 266 | 118865 | 1552463_at | 40 | 1552463_at | 43 | 1552463_at | 53 |
| 267 | 119034 | 204224_s_at | 1342 | 204224_s_at | 4014 | 204224_s_at | 8906 |
| 268 | 41802 | | | | | | |
| 269 | 115614 | 222103_at | 3418 | 222103_at | 2368 | 222103_at | 3426 |
| 270 | 120142 | 219215_s_at | 966 | 219215_s_at | 90 | 219215_s_at | 400 |
| 271 | 129420 | 226459_at | 6078 | 226459_at | 1329 | 226459_at | 2520 |
| 272 | 35139 | 210712_at | 48 | 210712_at | 18 | 210712_at | 25 |
| 273 | 112237 | 221008_s_at | 97 | 221008_s_at | 31 | 221008_s_at | 2784 |
| 274 | 118332 | 222479_s_at | 1695 | 222479_s_at | 5068 | 222479_s_at | 2723 |
| 275 | 202390 | 228206_at | 51 | 228206_at | 17 | 228206_at | 72 |
| 276 | 111442 | 203921_at | 607 | 203921_at | 47 | 203921_at | 203 |
| 277 | 119734 | 224761_at | 10651 | 224761_at | 8145 | 224761_at | 19421 |
| 278 | 46555 | 229736_at | 122 | 229736_at | 253 | 229736_at | 245 |
| 279 | 112493 | 207357_s_at | 177 | 212256_at | 201 | 212256_at | 210 |
| 280 | 120542 | 215584_at | 162 | 215584_at | 114 | 215584_at | 139 |
| 281 | 143975 | 203879_at | 2234 | 203879_at | 161 | 203879_at | 201 |
| 282 | 202509 | 213107_at | 217 | 213107_at | 1581 | 213107_at | 460 |
| 283 | 26615 | 218323_at | 3614 | 218323_at | 2478 | 218323_at | 1000 |
| 284 | 2021 | 208516_at | 34 | 208516_at | 8 | 208516_at | 15 |
| 285 | 1017 | 224960_at | 4065 | 224960_at | 6242 | 224960_at | 3403 |
| 286 | 44902 | 212581_x_at | 51985 | 212581_x_at | 45533 | 212581_x_at | 46383 |
| 287 | 121821 | 219951_s_at | 243 | 219951_s_at | 149 | 219951_s_at | 59 |
| 288 | 3573 | 204341_at | 291 | 204341_at | 237 | 204341_at | 1105 |
| 289 | 111379 | 211163_s_at | 61 | 206222_at | 34 | 211163_s_at | 100 |
| 290 | 119725 | 206653_at | 195 | 206653_at | 316 | 206653_at | 308 |
| 291 | 119012 | 205894_at | 321 | 205994_at | 876 | 205894_at | 1389 |
| 292 | 11202 | 211488_s_at | 71 | 211488_s_at | 67 | 242982_x_at | 40 |
| 293 | 119352 | 224100_s_at | 99 | 224100_s_at | 97 | 224100_s_at | 98 |
| 294 | 111028 | 55065_at | 1146 | 55065_at | 1142 | 55065_at | 392 |
| 295 | 6242 | 235885_at | 29 | 224102_at | 27 | 235885_at | 29 |
| 296 | 6829 | 1553016_at | 56 | 1553016_at | 39 | 1553016_at | 111 |
| 297 | 120986 | 205913_at | 23 | 205913_at | 15 | 205913_at | 153 |
| 298 | 282 | 206138_s_at | 1437 | 206138_s_at | 1128 | 206138_s_at | 1004 |
| 299 | 119249 | 210051_at | 40 | 210051_at | 44 | 210051_at | 134 |
| 300 | 121002 | 231734_at | 59 | 231734_at | 1544 | 231734_at | 44 |
| 301 | 112098 | 227014_at | 106 | 227015_at | 61 | 227014_at | 63 |
| 302 | 120006 | 210366_at | 62 | 210366_at | 76 | 210366_at | 2617 |
| 303 | 9145 | 213384_x_at | 418 | 213384_x_at | 426 | 213384_x_at | 402 |
| 304 | 45672 | 1564537_a_at | 29 | 1564537_a_at | 18 | 1564536_at | 22 |
| 305 | 5997 | 221442_at | 28 | 221442_at | 22 | 221442_at | 65 |
| 306 | 5922 | 209120_at | 1366 | 209120_at | 2925 | 209120_at | 1772 |
| 307 | 112027 | 218096_at | 2927 | 218096_at | 4692 | 218096_at | 2642 |
| 308 | 42079 | 211451_s_at | 21 | 211451_s_at | 38 | 208359_s_at | 18 |
| 309 | 104120 | 207597_at | 56 | 207597_at | 37 | 207597_at | 103 |
| 310 | 104465 | 203402_at | 641 | 203402_at | 89 | 203402_at | 35 |
| 311 | 118241 | 204862_s_at | 393 | 204862_s_at | 350 | 204862_s_at | 494 |
| 312 | 12487 | | | | | | |
| 313 | 139155 | 203457_at | 384 | 203457_at | 309 | 203457_at | 293 |
| 314 | 7014 | 206704_at | 218 | 206704_at | 472 | 206704_at | 170 |
| 315 | 107742 | 205404_at | 1132 | 205404_at | 31 | 205404_at | 28211 |
| 316 | 122070 | 226857_at | 25 | 226857_at | 44 | 226857_at | 16 |
| 317 | 119167 | 206945_at | 95 | 206945_at | 579 | 206945_at | 116 |
| 318 | 121082 | 218392_x_at | 1012 | 230069_at | 1511 | 218392_x_at | 1122 |
| 319 | 34166 | 223514_at | 42 | 223514_at | 28 | 1562368_at | 52 |
| 320 | 36798 | 226851_at | 5701 | 226851_at | 4411 | 226851_at | 3646 |
| 321 | 6764 | 1553889_at | 10 | 1553889_at | 10 | 1553889_at | 11 |
| 322 | 112281 | 235458_at | 1627 | 1554285_at | 86 | 235458_at | 250 |
| 323 | 1359 | 221696_s_at | 50 | 221696_s_at | 73 | 221696_s_at | 36 |
| 324 | 120792 | 208678_at | 5405 | 208678_at | 4778 | 208678_at | 3658 |
| 325 | 120227 | 215716_s_at | 2499 | 215716_s_at | 4375 | 215716_s_at | 1298 |
| 326 | 117144 | 209340_at | 4289 | 209340_at | 4274 | 209340_at | 6062 |
| 327 | 202463 | | | | | | |
| 328 | 326 | 202670_at | 2777 | 202670_at | 3561 | 202670_at | 3948 |
| 329 | 121132 | 226731_at | 164 | 226731_at | 1184 | 226731_at | 197 |
| 330 | 40762 | 208078_s_at | 851 | 208078_s_at | 1514 | 208078_s_at | 1302 |
| 331 | 106985 | 203458_at | 743 | 203458_at | 1415 | 203458_at | 1981 |
| 332 | 118634 | 204826_at | 371 | 204826_at | 502 | 204826_at | 110 |
| 333 | 1709 | 208108_s_at | 40 | 208108_s_at | 47 | 208108_s_at | 51 |
| 334 | 119230 | 209435_s_at | 5965 | 209435_s_at | 1077 | 209435_s_at | 1421 |
| 335 | 111644 | 210942_s_at | 1874 | 213355_at | 726 | 213355_at | 962 |
| 336 | 103331 | 206794_at | 34 | 206794_at | 42 | 206794_at | 44 |
| 337 | 105105 | 201419_at | 578 | 201419_at | 448 | 201419_at | 469 |
| 338 | 6671 | 202910_s_at | 1509 | 202910_s_at | 449 | 202910_s_at | 98 |
| 339 | 117749 | 244084_at | 67 | 244084_at | 59 | 244084_at | 26 |
| 340 | 104678 | 1565886_at | 189 | 1565886_at | 153 | 223323_x_at | 257 |
| 341 | 4236 | 207455_at | 52 | 207455_at | 46 | 207455_at | 61 |
| 342 | 119150 | 207158_at | 62 | 207158_at | 103 | 207158_at | 131 |
| 343 | 120536 | 212066_s_at | 2987 | 212066_s_at | 2546 | 212066_s_at | 2097 |
| 344 | 4084 | 213436_at | 215 | 213436_at | 36 | 213436_at | 28 |
| 345 | 8584 | 206591_at | 54 | 206591_at | 97 | 206591_at | 83 |
| 346 | 118025 | 229222_at | 1138 | 229222_at | 1396 | 229222_at | 535 |
| 347 | 104025 | 205959_at | 8 | 205959_at | 6 | 205959_at | 11 |
| 348 | 142304 | 212046_x_at | 704 | 212046_x_at | 691 | 212046_x_at | 218 |
| 349 | 119004 | 231846_at | 197 | 231846_at | 291 | 231846_at | 160 |
| 350 | 112199 | 219182_at | 249 | 219182_at | 132 | 221164_x_at | 125 |
| 351 | 140383 | 226214_at | 2687 | 226214_at | 3411 | 202593_s_at | 2754 |
| 352 | 43202 | 240770_at | 23 | 240770_at | 24 | 240770_at | 190 |
| 353 | 118558 | 206630_at | 138 | 206630_at | 120 | 206630_at | 175 |
| 354 | 122033 | 210201_x_at | 288 | 210201_x_at | 1209 | 210201_x_at | 656 |
| 355 | 110947 | 229936_at | 54 | 229936_at | 29 | 229936_at | 55 |
| 356 | 122374 | 210020_x_at | 108 | 210020_x_at | 92 | 210020_x_at | 161 |
| 357 | 121768 | | | | | | |
| 358 | 110667 | | | | | | |
| 359 | 119683 | 201349_at | 1028 | 201349_at | 3895 | 201349_at | 3727 |
| 360 | 105368 | 206473_at | 252 | 206473_at | 123 | 206473_at | 465 |
| 361 | 117476 | 207362_at | 14 | 207362_at | 12 | 207362_at | 8 |
| 362 | 8110 | 209977_at | 32 | 209977_at | 57 | 209977_at | 12240 |
| 363 | 108254 | 240039_at | 79 | 240039_at | 83 | 240039_at | 153 |
| 364 | 119254 | 201115_at | 1417 | 201115_at | 1515 | 201115_at | 1513 |
| 365 | 120528 | 212255_s_at | 1375 | 212255_s_at | 1248 | 212255_s_at | 922 |
| 366 | 114721 | 217815_at | 2794 | 217815_at | 3006 | 217815_at | 2134 |
| 367 | 120404 | 215506_s_at | 33 | 215506_s_at | 24 | 215506_s_at | 300 |
| 368 | 121560 | 211672_s_at | 2730 | 211672_s_at | 1515 | 211672_s_at | 1540 |
| 369 | 8759 | | | | | | |
| 370 | 121689 | 204952_at | 12 | 204952_at | 13 | 204952_at | 26 |
| 371 | 116959 | 209143_s_at | 5524 | 209143_s_at | 4904 | 209143_a_at | 5191 |
| 372 | 18269 | 217922_at | 1702 | 217922_at | 3222 | 217922_at | 1151 |
| 373 | 4118 | 207913_at | 22 | 207913_at | 23 | 207913_at | 21 |
| 374 | 120313 | 212519_at | 9619 | 212519_at | 7238 | 212519_at | 5318 |
| 375 | 14778 | 218200_s_at | 9899 | 218200_s_at | 8827 | 218200_s_at | 10816 |
| 376 | 1554 | 235512_at | 66 | 235512_at | 88 | 235512_at | 259 |
| 377 | 120581 | 205540_s_at | 135 | 205540_s_at | 57 | 205540_s_at | 58 |
| 378 | 135789 | 207344_at | 41 | 207344_at | 20 | 207344_at | 38 |
| 379 | 104313 | 207972_at | 14 | 207972_at | 10 | 207972_at | 28 |
| 380 | 5020 | 221459_at | 27 | 221459_at | 20 | 221459_at | 23 |
| 381 | 103787 | 203652_at | 586 | 203652_at | 705 | 203652_at | 455 |
| 382 | 38222 | 221199_at | 16 | 234868_s_at | 14 | 221199_at | 21 |
| 383 | 119010 | 232197_x_at | 947 | 232197_x_at | 220 | 1554030_at | 179 |
| 384 | 119464 | 201588_at | 10693 | 201588_at | 9916 | 201588_at | 16536 |
| 385 | 111967 | 223531_x_at | 1293 | 225463_x_at | 2133 | 223531_x_at | 1385 |
| 386 | 117597 | 234291_s_at | 23 | 234291_s_at | 64 | 234291_s_at | 79 |
| 387 | 616 | 205570_at | 216 | 205570_at | 234 | 205570_at | 136 |
| 388 | 104271 | 201860_s_at | 56 | 201860_s_at | 19 | 201860_s_at | 65 |
| 389 | 41648 | 221365_at | 16 | 221365_at | 9 | 221365_at | 32 |
| 390 | 116760 | 205931_s_at | 100 | 205931_s_at | 143 | 205931_s_at | 166 |
| 391 | 103742 | 1557172_x_at | 269 | 1557172_x_at | 315 | 1557172_x_at | 245 |
| 392 | 121625 | 224217_s_at | 1896 | 224217_s_at | 2305 | 218080_x_at | 1094 |
| 393 | 108793 | 217776_at | 7701 | 217776_at | 8105 | 217776_at | 7355 |
| 394 | 46149 | 214224_s_at | 3320 | 214224_s_at | 3818 | 204571_x_at | 4719 |
| 395 | 121965 | 213883_s_at | 5749 | 213883_s_at | 5402 | 213883_s_at | 3728 |
| 396 | 104655 | 200733_s_at | 8461 | 200732_s_at | 10330 | 200730_s_at | 18714 |
| 397 | 3025 | 206219_s_at | 1091 | 206219_s_at | 11 | 206219_s_at | 61 |
| 398 | 23439 | 220780_at | 64 | 220780_at | 32 | 220780_at | 17 |
| 399 | 31620 | 220276_at | 6 | 220276_at | 7 | 220276_at | 21 |
| 400 | 4069 | 206083_at | 56 | 206083_at | 58 | 206083_at | 73 |
| 401 | 107669 | 205696_s_at | 20 | 205696_s_at | 34 | 205696_s_at | 137 |
| 402 | 9248 | 208928_at | 870 | 208928_at | 758 | 208928_at | 4551 |
| 403 | 106198 | 205623_at | 58 | 205623_at | 93 | 205623_at | 84 |
| 404 | 112515 | 221872_at | 99 | 206391_at | 21 | 221872_at | 214 |
| 405 | 8537 | 207542_s_at | 72 | 207542_s_at | 27 | 207542_s_at | 91 |
| 406 | 110610 | 208308_s_at | 6852 | 208308_s_at | 3502 | 208308_s_at | 4380 |
| 407 | 43265 | 209529_at | 92 | 209529_at | 38 | 209529_at | 110 |
| 408 | 180 | 213860_x_at | 11037 | 213860_x_at | 4720 | 213860_x_at | 10843 |
| 409 | 117634 | 212907_at | 6906 | 212907_at | 4832 | 212907_at | 11682 |
| 410 | 8947 | 226535_at | 18 | 226535_at | 60 | 226535_at | 175 |
| 411 | 10429 | 203586_s_at | 569 | 203586_s_at | 423 | 203586_s_at | 675 |
| 412 | 107317 | 212218_s_at | 513 | 212218_s_at | 868 | 212218_s_at | 293 |
| 413 | 121476 | 204767_s_at | 2911 | 204767_s_at | 8349 | 204767_s_at | 848 |
| 414 | 126545 | 238262_at | 32 | 238262_at | 44 | 238262_at | 12 |
| 415 | 202300 | 213848_at | 5887 | 213848_at | 651 | 213848_at | 349 |
| 416 | 105208 | 226035_at | 1506 | 226035_at | 1291 | 226035_at | 1847 |
| 417 | 109375 | 220056_at | 209 | 220056_at | 95 | 220056_at | 196 |
| 418 | 120071 | 239457_at | 363 | 1554704_at | 245 | 1554704_at | 276 |
| 419 | 122067 | 1553314_a_at | 10 | 1553314_a_at | 38 | 1553314_a_at | 41 |
| 420 | 18224 | 203474_at | 2493 | 203474_at | 5981 | 203474_at | 1416 |
| 421 | 2057 | 221445_at | 30 | 221445_at | 20 | 221445_at | 25 |
| 422 | 6205 | 226032_at | 1926 | 226032_at | 1989 | 226032_at | 592 |
| 423 | 103432 | 204887_s_at | 611 | 204887_s_at | 675 | 204887_s_at | 31 |
| 424 | 107085 | 203343_at | 5668 | 203343_at | 7340 | 203343_at | 20563 |
| 425 | 117546 | 215800_at | 95 | 1565795_at | 90 | 215800_at | 94 |
| 426 | 41929 | 209262_s_at | 1091 | 209262_s_at | 5127 | 209262_s_at | 1358 |
| 427 | 112254 | 225612_s_at | 992 | 225612_s_at | 2540 | 225612_s_at | 1950 |
| 428 | 105538 | 221095_s_at | 15 | 221095_s_at | 32 | 221095_s_at | 33 |
| 429 | 444 | 209467_s_at | 946 | 209467_s_at | 1271 | 209467_s_at | 929 |
| 430 | 6722 | 244509_at | 70 | 244509_at | 68 | 244509_at | 178 |
| 431 | 40719 | 212757_s_at | 305 | 212757_s_at | 622 | 212757_s_at | 355 |
| 432 | 115262 | 201565_s_at | 13660 | 201565_s_at | 15882 | 201565_s_at | 12247 |
| 433 | 106223 | 208147_s_at | 31 | 208147_s_at | 258 | 208147_s_at | 16267 |
| 434 | 120151 | 238215_at | 35 | 238215_at | 45 | 238215_at | 32 |
| 435 | 105664 | 209017_s_at | 2420 | 209017_s_at | 1368 | 209017_s_at | 2473 |
| 436 | 1020 | 218535_s_at | 1192 | 218535_s_at | 611 | 218535_s_at | 2151 |
| 437 | 112308 | 224598_at | 6304 | 224598_at | 3980 | 224598_at | 10583 |
| 438 | 120484 | 205695_at | 791 | 205695_at | 124 | 205695_at | 6095 |
| 439 | 670 | 204891_s_at | 56 | 204890_s_at | 14 | 204891_s_at | 23 |
| 440 | 1397 | 244547_at | 122 | 244547_at | 211 | 244547_at | 74 |
| 441 | 104702 | 212990_at | 835 | 212990_at | 834 | 212990_at | 408 |
| 442 | 1140 | 1554826_at | 110 | 1554826_at | 52 | 1554826_at | 36 |
| 443 | 112418 | 225905_s_at | 256 | 225905_s_at | 186 | 1555181_a_at | 292 |
| 444 | 104693 | 204722_at | 14 | 204722_at | 32 | 204722_at | 23 |
| 445 | 8943 | 204169_at | 482 | 204169_at | 397 | 204169_at | 152 |
| 446 | 107096 | 200638_s_at | 11640 | 200639_s_at | 9507 | 200639_s_at | 13084 |
| 447 | 2531 | 205754_at | 1978 | 205754_at | 4537 | 205754_at | 12014 |
| 448 | 118060 | 226339_at | 2165 | 226339_at | 3185 | 226339_at | 2266 |
| 449 | 118590 | 205075_at | 214 | 205075_at | 584 | 205075_at | 1538 |
| 450 | 118906 | 205950_s_at | 95 | 205950_s_at | 85 | 205950_s_at | 115 |
| 451 | 106243 | 202314_at | 6354 | 202314_at | 7921 | 216607_s_at | 4948 |
| 452 | 111874 | 219425_at | 43 | 219425_at | 15 | 219425_at | 23 |
| 453 | 8193 | 200980_s_at | 4052 | 200980_s_at | 5058 | 200980_s_at | 4410 |
| 454 | 2512 | 202735_at | 3488 | 202735_at | 17315 | 213787_s_at | 3559 |
| 455 | 16362 | 228424_at | 136 | 228424_at | 111 | 228424_at | 131 |
| 456 | 14218 | 222021_x_at | 4167 | 222021_x_at | 3075 | 222021_x_at | 5033 |
| 457 | 103493 | 206249_at | 110 | 206249_at | 408 | 206249_at | 264 |
| 458 | 1176 | 227482_at | 232 | 227482_at | 161 | 227482_at | 220 |
| 459 | 111523 | 232553_at | 67 | 232553_at | 56 | 210456_at | 53 |
| 460 | 33700 | 223582_at | 178 | 223582_at | 491 | 223582_at | 407 |
| 461 | 2167 | 206625_at | 40 | 206625_at | 55 | 206625_at | 86 |
| 462 | 105854 | 202166_s_at | 3145 | 202166_s_at | 2777 | 202166_s_at | 3761 |
| 463 | 46281 | 220756_s_at | 47 | 220756_s_at | 16 | 220756_s_at | 19 |
| 464 | 44894 | 201487_at | 6223 | 201487_at | 9803 | 201487_at | 1880 |
| 465 | 38041 | 1555962_at | 215 | 1555962_at | 320 | 1555963_x_at | 193 |
| 466 | 42278 | 227361_at | 2094 | 227361_at | 768 | 227361_at | 2349 |
| 467 | 112472 | 222754_at | 2172 | 222754_at | 2734 | 222754_at | 1804 |

TABLE 5

| Target | Target | | | Transferrin | Transferrin | Transferrin | Transferrin |
|---|---|---|---|---|---|---|---|
| No | Symbol | Gene ID | siRNA ID | Run1 Mean % | Run1 SD % | Runs Mean % | Run2 SD % |
| 2 | HLCS | 3141 | 117851 | 146.8 | 47.4 | | |
| 2 | HLCS | 3141 | 117852 | 164.4 | 33.1 | | |
| 2 | HLCS | 3141 | 117853 | 113.3 | 6.5 | | |
| 3 | SC4MOL | 6307 | 117416 | 178.7 | 13.0 | | |
| 3 | SC4MOL | 6307 | 117417 | 184.0 | 17.9 | | |
| 3 | SC4MOL | 6307 | 117418 | 109.6 | 26.3 | | |
| 9 | HMBS | 3145 | 8225 | 260.9 | 18.6 | | |
| 9 | HMBS | 3145 | 117844 | 239.0 | 9.7 | | |
| 10 | HSD17B4 | 3295 | 106087 | 451.0 | 83.7 | | |
| 10 | HSD17B4 | 3295 | 106089 | 178.8 | 18.3 | 208.0 | 18.2 |
| 11 | LCN2 | 3934 | 121011 | 43.9 | 7.0 | | |
| 11 | LCN2 | 3934 | 121012 | 54.6 | 14.4 | | |
| 13 | PPP1R3C | 5507 | 142834 | 107.6 | 12.5 | | |
| 13 | PPP1R3C | 5507 | 142836 | 104.7 | 18.5 | | |
| 16 | TPI1 | 7167 | 43820 | 108.4 | 10.7 | | |
| 16 | TPI1 | 7167 | 43916 | 204.2 | 36.6 | | |
| 18 | SLC30A2 | 7780 | 117693 | 172.2 | 25.4 | | |
| 18 | SLC30A2 | 7780 | 117695 | 316.7 | 51.7 | | |
| 19 | ULK1 | 8408 | 118260 | 126.3 | 27.9 | | |
| 19 | ULK1 | 8408 | 118261 | 333.2 | 56.7 | | |
| 22 | SPUVE | 11098 | 19104 | 130.0 | 11.1 | 142.7 | 2.7 |
| 22 | SPUVE | 11098 | 19196 | 176.5 | 18.5 | 123.6 | 4.4 |
| 22 | SPUVE | 11098 | 212941 | 79.5 | 13.3 | 74.8 | 2.4 |
| 22 | SPUVE | 11098 | 212942 | 221.5 | 53.8 | | |
| 26 | MYLIP | 29116 | 118397 | 134.7 | 26.9 | | |
| 26 | MYLIP | 29116 | 118398 | 98.0 | 10.8 | | |
| 27 | PKD1L2 | 114780 | 104830 | 159.8 | 27.5 | 168.3 | 17.1 |
| 27 | PKD1L2 | 114780 | 104835 | 250.0 | 50.2 | | |
| 28 | BPHL | 670 | 119221 | 230.3 | 16.3 | | |
| 28 | BPHL | 670 | 214767 | 145.0 | 4.8 | | |
| 29 | USP3 | 9960 | 105141 | 478.2 | 66.2 | | |
| 29 | USP3 | 9960 | 214567 | 83.3 | 20.0 | | |
| 31 | KCNK17 | 89822 | 43781 | 375.9 | 21.1 | 346.4 | 3.1 |
| 31 | KCNK17 | 89822 | 212814 | 51.8 | 3.4 | | |
| 31 | KCNK17 | 89822 | 212815 | 70.7 | 17.3 | | |
| 32 | WEE1 | 7465 | 212739 | 199.1 | 56.6 | 295.1 | 20.4 |
| 34 | RNUT1 | 10073 | 117371 | 328.2 | 25.7 | | |
| 34 | RNUT1 | 10073 | 214780 | 72.8 | 6.7 | | |
| 35 | M6PR | 4074 | 111157 | 133.3 | 16.5 | | |
| 35 | M6PR | 4074 | 214744 | 85.5 | 7.0 | | |
| 36 | MGC39650 | 147011 | 38979 | 320.9 | 46.8 | | |
| 36 | MGC39650 | 147011 | 214871 | 76.8 | 15.5 | | |
| 37 | FLJ22761 | 80201 | 121933 | 308.7 | 45.1 | | |
| 37 | FLJ22761 | 80201 | 214839 | 183.5 | 26.0 | | |
| 38 | PAPOLG | 64895 | 119829 | 52.3 | 1.9 | | |
| 38 | PAPOLG | 64895 | 214280 | 272.9 | 26.9 | 190.1 | 3.7 |
| 39 | DNM1L | 10059 | 19471 | 151.2 | 27.2 | | |
| 39 | DNM1L | 10059 | 214799 | 104.7 | 12.7 | | |
| 43 | LCN7 | 64129 | 105753 | 197.2 | 25.5 | | |
| 43 | LCN7 | 64129 | 214577 | 52.2 | 10.8 | | |
| 45 | RASGRP2 | 10235 | 120445 | 175.9 | 13.8 | | |
| 45 | RASGRP2 | 10235 | 214874 | 73.7 | 8.9 | | |
| 51 | PRPSAP2 | 5636 | 117084 | 54.0 | 6.8 | | |
| 51 | PRPSAP2 | 5636 | 214750 | 117.4 | 9.9 | | |
| 52 | SLC26A10 | 65012 | 119926 | 248.0 | 13.3 | | |
| 52 | SLC26A10 | 65012 | 214589 | 181.1 | 12.6 | | |
| 56 | TNFRSF13B | 23495 | 111813 | 248.5 | 28.7 | | |
| 56 | TNFRSF13B | 23495 | 214802 | 105.0 | 21.4 | | |
| 62 | CTSK | 1513 | 105010 | 460.6 | 63.8 | | |
| 62 | CTSK | 1513 | 214550 | 108.4 | 6.9 | | |
| 72 | D4ST-1 | 113189 | 112330 | 247.0 | 28.3 | 262.6 | 18.6 |
| 72 | D4ST-1 | 113189 | 214285 | 137.6 | 12.9 | 233.0 | 21.2 |
| 73 | APCL | 10297 | 121576 | 194.5 | 25.8 | | |
| 73 | APCL | 10297 | 214783 | 172.5 | 12.8 | | |
| 79 | ARHGEF1 | 9138 | 119422 | 217.2 | 35.9 | | |
| 79 | ARHGEF1 | 9138 | 214561 | 162.0 | 29.3 | | |
| 81 | MCCC1 | 56922 | 118817 | 48.0 | 2.0 | | |
| 81 | MCCC1 | 56922 | 214276 | 57.6 | 16.2 | 81.5 | 10.0 |
| 88 | GALR2 | 8811 | 212858 | 125.8 | 23.7 | 143.6 | 28.3 |
| 117 | SMPD1 | 6609 | 8816 | 112.3 | 4.2 | | |
| 117 | SMPD1 | 6609 | 212695 | 136.7 | 10.0 | | |
| 143 | SCP2 | 6342 | 119182 | 191.9 | 30.7 | | |
| 143 | SCP2 | 6342 | 214903 | 125.4 | 29.7 | | |
| 163 | CCM1 | 889 | 15563 | 87.6 | 18.2 | | |
| 163 | CCM1 | 889 | 214883 | 213.3 | 51.5 | | |
| 171 | PAFAH2 | 5051 | 119066 | 70.8 | 8.2 | | |
| 171 | PAFAH2 | 5051 | 214710 | 98.7 | 18.0 | | |
| 180 | GAD2 | 2572 | 9108 | 165.2 | 19.8 | | |
| 180 | GAD2 | 2572 | 214716 | 285.5 | 18.1 | | |
| 205 | HTR2C | 3358 | 144642 | 87.8 | 2.7 | | |
| 205 | HTR2C | 3358 | 212705 | 127.3 | 7.6 | | |
| 215 | LOC345667 | 11174 | 212853 | 161.3 | 35.3 | | |
| 237 | GPR3 | 2827 | 1785 | 70.9 | 19.6 | 56.7 | 10.5 |
| 237 | GPR3 | 2827 | 212766 | 87.6 | 13.8 | | |
| 240 | FLJ32389 | 126393 | 122036 | 194.9 | 22.0 | | |
| 240 | FLJ32389 | 126393 | 214864 | 110.7 | 20.1 | | |
| 241 | SERPINA7 | 6906 | 118553 | 182.3 | 10.3 | | |
| 241 | SERPINA7 | 6906 | 214548 | 337.9 | 11.6 | | |
| 242 | DCTD | 1635 | 119612 | 440.5 | 32.2 | | |
| 242 | DCTD | 1635 | 214555 | 152.9 | 17.2 | | |
| 261 | ACLY | 47 | 116939 | 106.8 | 3.2 | | |
| 261 | ACLY | 47 | 214886 | 92.2 | 13.1 | | |
| 273 | AGXT2L1 | 64850 | 112237 | 60.3 | 9.8 | | |
| 273 | AGXT2L1 | 64850 | 214281 | 102.0 | 16.9 | 147.6 | 35.4 |
| 291 | ARSE | 415 | 119012 | 185.2 | 31.3 | | |
| 291 | ARSE | 415 | 214706 | 353.7 | 43.1 | | |
| 292 | ITGB8 | 3696 | 11202 | 95.6 | 16.5 | | |
| 292 | ITGB8 | 3696 | 214742 | 225.5 | 8.7 | | |
| 306 | NR2F2 | 7026 | 5922 | 167.0 | 32.8 | 168.5 | 44.3 |
| 306 | NR2F2 | 7026 | 212809 | 49.5 | 7.4 | | |
| 309 | ADAM18 | 8749 | 212787 | 269.8 | 19.3 | 224.5 | 21.0 |
| 334 | ARHGEF2 | 9181 | 119230 | 340.3 | 107.3 | | |
| 334 | ARHGEF2 | 9181 | 214562 | 279.4 | 26.5 | | |
| 352 | PRP2 | 134285 | 43202 | 186.8 | 28.5 | | |
| 352 | LOC134285 | 134285 | 128215 | 98.5 | 24.2 | | |
| 352 | LOC134285 | 134285 | 212841 | 43.0 | 1.5 | | |
| 363 | PLA2R1 | 22925 | 108254 | 173.7 | 27.0 | | |
| 363 | PLA2R1 | 22925 | 214723 | 215.6 | 31.0 | | |
| 390 | CREB5 | 9586 | 116760 | 63.1 | 5.7 | | |
| 390 | CREB5 | 9586 | 214882 | 197.1 | 14.8 | | |
| 413 | FEN1 | 2237 | 121476 | 107.0 | 28.6 | | |
| 413 | FEN1 | 2237 | 214763 | 116.1 | 13.0 | | |
| 435 | PRSS15 | 9361 | 105664 | 141.2 | 5.6 | 128.9 | 5.1 |
| 435 | PRSS15 | 9361 | 212757 | 93.3 | 13.2 | | |
| 435 | PRSS15 | 9361 | 212758 | 88.9 | 10.6 | | |
| 441 | SYNJ1 | 8867 | 104702 | 388.9 | 7.4 | 354.9 | 53.3 |
| 441 | SYNJ1 | 8867 | 212746 | 10.7 | 1.1 | | |
| 441 | SYNJ1 | 8867 | 212747 | 129.0 | 12.2 | | |
| 459 | PCYT1B | 9468 | 111523 | 146.8 | 16.0 | | |
| 459 | PCYT1B | 9468 | 214260 | 183.3 | 27.0 | 197.7 | 14.8 |
| 486 | FLJ21736 | 79984 | 119838 | 275.3 | 13.8 | | |
| 486 | FLJ21736 | 79984 | 235619 | 216.7 | 51.0 | | |
| 489 | GPR10 | 2834 | 103837 | 141.9 | 10.0 | | |
| 489 | GPR10 | 2834 | 235614 | 171.2 | 8.9 | | |
| 495 | KIR2DS1 | 3806 | 212646 | 224.1 | 17.0 | | |
| 495 | KIR2DS1 | 3806 | 235634 | 140.4 | 10.0 | | |
| 496 | KIR2DS3 | 3808 | 213159 | 214.4 | 25.8 | | |
| 496 | KIR2DS3 | 3808 | 235633 | 87.8 | 12.8 | | |
| 498 | LOC135896 | 135896 | 213242 | 259.7 | 7.7 | | |
| 498 | LOC135896 | 135896 | 235629 | 49.9 | 7.0 | | |
| 506 | MOXD1 | 26002 | 111923 | 193.5 | 17.0 | | |
| 506 | MOXD1 | 26002 | 235615 | 178.9 | 27.0 | | |
| 507 | MPN2 | 339501 | 113991 | 45.7 | 5.8 | | |
| 507 | MPN2 | 339501 | 235626 | 302.7 | 27.3 | | |
| 514 | PEPD | 5184 | 105302 | 313.2 | 67.0 | 353.2 | 64.4 |
| 514 | PEPD | 5184 | 212688 | 213.9 | 14.9 | | |

TABLE 6

| Target | Target | Gene | siRNA | siRNA | LDL-Dil run1 | LDL-Dil | LDL-Dil run2 | LDL-Dil run2 | LDL-Dil run3 | LDL-Dil run3 |
|---|---|---|---|---|---|---|---|---|---|---|
| No | Symbol | Id | ID | conc. | Mean % | run1 SD % | Mean % | SD % | Mean % | SD % |
| 2 | HLCS | 3141 | 117852 | 10 | 414.5 | 93.1 | 451 | 99.6 | 126.3 | 22.6 |
| 2 | HLCS | 3141 | 117852 | 30 | 591.2 | 39.7 | 631.6 | 62.1 | 233 | 47.9 |
| 2 | HLCS | 3141 | 117852 | 100 | 502.9 | 112.1 | 720.9 | 143 | 266.9 | 17.7 |
| 2 | HLCS | 3141 | 117853 | 10 | 379.4 | 81.1 | 421.3 | 106.9 | 363.3 | 37 |
| 2 | HLCS | 3141 | 117853 | 30 | 590.7 | 78 | 572.4 | 60.8 | 418.6 | 42.8 |
| 2 | HLCS | 3141 | 117853 | 100 | 737.3 | 36.3 | 930.2 | 178.1 | 787.2 | 64.5 |
| 3 | SC4MOL | 6307 | 117417 | 10 | 658.1 | 147.8 | 612.5 | 115.7 | 421.4 | 17.9 |
| 3 | SC4MOL | 6307 | 117417 | 30 | 918.8 | 50.2 | 509.2 | 148.3 | 179.9 | 9 |
| 3 | SC4MOL | 6307 | 117417 | 100 | 1341.9 | 148.3 | 857.9 | 190.6 | 436.9 | 155.6 |
| 3 | SC4MOL | 6307 | 117418 | 10 | 420.1 | 66.8 | 596.2 | 63.5 | 258.1 | 55.6 |
| 3 | SC4MOL | 6307 | 117418 | 30 | 703.9 | 6.8 | 670.7 | 26.5 | 164.6 | 3.6 |
| 3 | SC4MOL | 6307 | 117418 | 100 | 912.2 | 96.2 | 786 | 22.6 | 351.9 | 38.9 |
| 4 | CASP1 | 834 | 42626 | 10 | 300.7 | 27 | 210.1 | 64.5 | 242.6 | 74.3 |
| 4 | CASP1 | 834 | 42626 | 30 | 275.3 | 70.1 | 234.8 | 13.6 | 274.5 | 30.8 |
| 4 | CASP1 | 834 | 42626 | 100 | 343.6 | 87 | 388.3 | 39 | 452.7 | 128 |
| 4 | CASP1 | 834 | 42711 | 10 | 336.7 | 70.2 | 351.8 | 54.1 | 360.3 | 127.5 |
| 4 | CASP1 | 834 | 42711 | 30 | 642.9 | 45.3 | 377.6 | 51.6 | 469.1 | 74.9 |
| 4 | CASP1 | 834 | 42711 | 100 | 860.1 | 70.8 | 623.9 | 70.3 | 469.7 | 118.1 |
| 7 | CTSE | 1510 | 105039 | 10 | 521.4 | 15.8 | 318.4 | 63.9 | 500.9 | 99.9 |
| 7 | CTSE | 1510 | 105039 | 30 | 647 | 131.3 | 674.6 | 106.7 | 631.5 | 36.3 |
| 7 | CTSE | 1510 | 105039 | 100 | 695.2 | 29.7 | 1494.5 | 191.2 | 832.3 | 122.9 |
| 7 | CTSE | 1510 | 105041 | 10 | 148.4 | 8 | 164.1 | 27.1 | 184.4 | 61.9 |
| 7 | CTSE | 1510 | 105041 | 30 | 198.1 | 9.5 | 208.5 | 37.5 | 179.9 | 41.2 |
| 7 | CTSE | 1510 | 105041 | 100 | 229.2 | 67 | 354.7 | 68.4 | 231.7 | 13.2 |
| 8 | FRK | 2444 | 1147 | 10 | 282.1 | 87.5 | 255.4 | 78.7 | 221 | 26.4 |
| 8 | FRK | 2444 | 1147 | 30 | 321.9 | 37.3 | 350.1 | 12.7 | 256 | 25.5 |
| 8 | FRK | 2444 | 1147 | 100 | 508.8 | 22.1 | 431.5 | 33.7 | 270.3 | 52.8 |
| 8 | FRK | 2444 | 1243 | 10 | 268.4 | 90.6 | 275.7 | 75.3 | 241.2 | 21.5 |
| 8 | FRK | 2444 | 1243 | 30 | 267.4 | 7.4 | 268.5 | 12.6 | 279.1 | 24.8 |
| 8 | FRK | 2444 | 1243 | 100 | 310.3 | 17.7 | 497.1 | 60.1 | 485.5 | 130.5 |
| 9 | HMBS | 3145 | 8225 | 10 | 304.7 | 63.7 | 291.6 | 64.1 | 287.2 | 24.9 |
| 9 | HMBS | 3145 | 8225 | 30 | 448.8 | 7.8 | 368.2 | 78.1 | 287.4 | 39.1 |
| 9 | HMBS | 3145 | 8225 | 100 | 562.9 | 122.7 | 530.8 | 93.9 | 413.3 | 32.8 |
| 9 | HMBS | 3145 | 117844 | 10 | 194 | 27.8 | 199.3 | 26.1 | 166.2 | 21 |
| 9 | HMBS | 3145 | 117844 | 30 | 204.8 | 7.6 | 187.5 | 30.6 | 171.1 | 16.9 |
| 9 | HMBS | 3145 | 117844 | 100 | 152.4 | 21 | 253.1 | 37.2 | 185.9 | 52.3 |
| 10 | HSD17B4 | 3295 | 106087 | 10 | 397 | 56.5 | 390.6 | 127.4 | 261.1 | 38.3 |
| 10 | HSD17B4 | 3295 | 106087 | 30 | 479.9 | 47.8 | 502.6 | 98.4 | 320.6 | 23.9 |
| 10 | HSD17B4 | 3295 | 106087 | 100 | 552.6 | 33.8 | 682.4 | 115.5 | 403.2 | 50 |
| 10 | HSD17B4 | 3295 | 106089 | 10 | 239.4 | 53 | 171.9 | 47.3 | 127.2 | 23.8 |
| 10 | HSD17B4 | 3295 | 106089 | 30 | 380.8 | 18.4 | 243.2 | 29.9 | 168 | 33.2 |
| 10 | HSD17B4 | 3295 | 106089 | 100 | 288 | 35.5 | 272.1 | 11.8 | 176.4 | 22.6 |
| 12 | OXTR | 5021 | 1766 | 10 | 605.7 | 38.7 | 316.3 | 40.2 | 233.3 | 41.1 |
| 12 | OXTR | 5021 | 1766 | 30 | 703 | 42.5 | 389.3 | 61.8 | 238.6 | 16.5 |
| 12 | OXTR | 5021 | 1766 | 100 | 720.8 | 80.2 | 596.9 | 114.3 | 253.2 | 42.9 |
| 12 | OXTR | 5021 | 1947 | 10 | 432.6 | 55.5 | 176.9 | 51.4 | 241.6 | 65.8 |
| 12 | OXTR | 5021 | 1947 | 30 | 271.8 | 6.5 | 208.7 | 16 | 260.3 | 51.7 |
| 12 | OXTR | 5021 | 1947 | 100 | 354.6 | 96.8 | 409.4 | 60.1 | 373.2 | 89 |
| 16 | TPI1 | 7167 | 43820 | 10 | 230.6 | 100.9 | 243 | 11.4 | 173.2 | 13.7 |
| 16 | TPI1 | 7167 | 43820 | 30 | 326.7 | 39.3 | 336.8 | 56.6 | 240.9 | 20.8 |
| 16 | TPI1 | 7167 | 43820 | 100 | 276.9 | 51.3 | 569.4 | 95.5 | 255 | 79.5 |
| 16 | TPI1 | 7167 | 43916 | 10 | 590.4 | 629.2 | 381.4 | 37.9 | 273 | 12 |
| 16 | TPI1 | 7167 | 43916 | 30 | 700.7 | 40.5 | 546 | 123.3 | 354.1 | 37.7 |
| 16 | TPI1 | 7167 | 43916 | 100 | 754.8 | 79.3 | 969.1 | 187.7 | 398.4 | 48.5 |
| 18 | SLC30A2 | 7780 | 117693 | 10 | 261.6 | 40.1 | 218.4 | 29.1 | 145.4 | 30.8 |
| 18 | SLC30A2 | 7780 | 117693 | 30 | 362.7 | 12.5 | 309 | 59.2 | 185.2 | 25.2 |
| 18 | SLC30A2 | 7780 | 117693 | 100 | 420.8 | 37.8 | 562.7 | 62.5 | 312.5 | 67.9 |
| 18 | SLC30A2 | 7780 | 117695 | 10 | 581.1 | 50.6 | 305.6 | 36.4 | 311 | 19.8 |
| 18 | SLC30A2 | 7780 | 117695 | 30 | 622 | 92.2 | 399.7 | 46.6 | 365.5 | 23.4 |
| 18 | SLC30A2 | 7780 | 117695 | 100 | 665.6 | 39 | 680.6 | 136.2 | 553.5 | 28.2 |
| 19 | ULK1 | 8408 | 118260 | 10 | 283.6 | 98.5 | 251.3 | 28.1 | 219.3 | 5.6 |
| 19 | ULK1 | 8408 | 118260 | 30 | 356.4 | 39.6 | 346.2 | 71.5 | 269.1 | 32.8 |
| 19 | ULK1 | 8408 | 118260 | 100 | 428.9 | 44.8 | 471.6 | 54.1 | 319.7 | 29.1 |
| 19 | ULK1 | 8408 | 118261 | 10 | 336.4 | 110.4 | 475.9 | 48.1 | 134.5 | 6.2 |
| 19 | ULK1 | 8408 | 118261 | 30 | 750 | 96 | 715.4 | 172 | 444.1 | 95 |
| 19 | ULK1 | 8408 | 118261 | 100 | 1009.5 | 162.2 | 1610.5 | 116 | 1007.2 | 271 |
| 22 | SPUVE | 11098 | 19104 | 10 | 357.4 | 47.1 | 323.6 | 82.7 | 314.4 | 46.3 |
| 22 | SPUVE | 11098 | 19104 | 30 | 757.4 | 42.5 | 598.2 | 91.7 | 412.9 | 53.1 |
| 22 | SPUVE | 11098 | 19104 | 100 | 713 | 51 | 1405 | 174.5 | 495.9 | 51.7 |
| 22 | SPUVE | 11098 | 19196 | 10 | 209.1 | 72.2 | 201.2 | 57.3 | 255.7 | 17 |
| 22 | SPUVE | 11098 | 19196 | 30 | 351.9 | 32.4 | 242.2 | 49.3 | 147.2 | 20.5 |
| 22 | SPUVE | 11098 | 19196 | 100 | 350.6 | 35.1 | 430.5 | 48.4 | 451.8 | 18.7 |
| 27 | PKD1L2 | 114780 | 104830 | 10 | 203.9 | 24.1 | 192.5 | 35.9 | 126.6 | 18.3 |
| 27 | PKD1L2 | 114780 | 104830 | 30 | 274 | 51.9 | 199.8 | 31.6 | 179.7 | 21.6 |
| 27 | PKD1L2 | 114780 | 104830 | 100 | 249.7 | 24.6 | 305.4 | 19.6 | 255.4 | 31.3 |
| 27 | PKD1L2 | 114780 | 104835 | 10 | 383.9 | 45.3 | 526.7 | 46.2 | 538.4 | 68 |
| 27 | PKD1L2 | 114780 | 104835 | 30 | 517.1 | 35.4 | 660.5 | 114.7 | 489.6 | 28.8 |
| 27 | PKD1L2 | 114780 | 104835 | 100 | 716.7 | 115.9 | 964.3 | 127.2 | 1060.5 | 161.7 |
| 39 | DNM1L | 10059 | 19471 | 10 | 280.6 | 61.3 | 424 | 18.7 | 359 | 48 |
| 39 | DNM1L | 10059 | 19471 | 30 | 484.8 | 63 | 541.7 | 78.7 | 403.4 | 66.5 |
| 39 | DNM1L | 10059 | 19471 | 100 | 725.2 | 41.8 | 802.2 | 68.3 | 539.9 | 40.2 |
| 39 | DNM1L | 10059 | 214799 | 10 | 378.6 | 39.7 | 460.4 | 86.7 | 291.9 | 31.1 |
| 39 | DNM1L | 10059 | 214799 | 30 | 428.8 | 57 | 515.3 | 25.3 | 332.7 | 17 |
| 39 | DNM1L | 10059 | 214799 | 100 | 454.6 | 57.4 | 615.1 | 245.1 | 385.2 | 45.7 |
| 88 | GALR2 | 8811 | 44971 | 10 | 996.6 | 184.4 | 520.3 | 73.8 | 466.7 | 101 |
| 88 | GALR2 | 8811 | 44971 | 30 | 891.6 | 91.1 | 606.4 | 126.9 | 439.5 | 43.2 |
| 88 | GALR2 | 8811 | 44971 | 100 | 1077.3 | 28.1 | 1020 | 150.9 | 723.4 | 98.9 |
| 88 | GALR2 | 8811 | 45063 | 10 | 67.2 | 16.1 | 103.6 | 2.7 | 98.6 | 57.3 |
| 88 | GALR2 | 8811 | 45063 | 30 | 77.8 | 17.1 | 110.3 | 33.3 | 91 | 12.1 |
| 88 | GALR2 | 8811 | 45063 | 100 | 63.4 | 9.9 | 122.8 | 28.6 | 113.4 | 7.6 |
| 143 | SCP2 | 6342 | 117110 | 10 | | | | | 398 | 81.2 |
| 143 | SCP2 | 6342 | 117110 | 30 | | | | | 169.3 | 21.6 |
| 143 | SCP2 | 6342 | 117110 | 100 | | | | | 152.6 | 40.4 |
| 143 | SCP2 | 6342 | 119182 | 10 | 689.2 | 495.8 | 432.8 | 50.8 | 294.2 | 74.8 |
| 143 | SCP2 | 6342 | 119182 | 30 | 1117.7 | 310.7 | 648.3 | 78.3 | 399.5 | 67.5 |
| 143 | SCP2 | 6342 | 119182 | 100 | 958 | 112.2 | 1365.2 | 233.3 | 521.1 | 101.1 |
| 171 | PAFAH2 | 5051 | 119066 | 10 | 277.3 | 83.4 | 315.2 | 48.9 | 270.7 | 33.2 |
| 171 | PAFAH2 | 5051 | 119066 | 30 | 464 | 26.4 | 368.9 | 33.5 | 309.9 | 16.9 |
| 171 | PAFAH2 | 5051 | 119066 | 100 | 612.7 | 11.6 | 559.8 | 132 | 382.7 | 103.9 |
| 171 | PAFAH2 | 5051 | 214710 | 10 | 284.8 | 65.8 | 336.5 | 70.8 | 235 | 27.4 |
| 171 | PAFAH2 | 5051 | 214710 | 30 | 415.4 | 45.3 | 398.6 | 75.8 | 230.2 | 36 |
| 171 | PAFAH2 | 5051 | 214710 | 100 | 322.9 | 27.6 | 541.8 | 36 | 332 | 24.5 |
| 180 | GAD2 | 2572 | 9108 | 10 | 302.3 | 49.7 | 266.4 | 65.3 | 282.5 | 35.8 |
| 180 | GAD2 | 2572 | 9108 | 30 | 490.1 | 51.1 | 356 | 54.7 | 380.3 | 60.6 |
| 180 | GAD2 | 2572 | 9108 | 100 | 527.8 | 29.3 | 212.2 | 40 | 521.8 | 67.1 |
| 180 | GAD2 | 2572 | 214716 | 10 | 309.9 | 104.4 | 294.8 | 45.6 | 129 | 15.1 |
| 180 | GAD2 | 2572 | 214716 | 30 | 415.6 | 87.9 | 273.3 | 16 | 275.8 | 43.4 |
| 180 | GAD2 | 2572 | 214716 | 100 | 434.7 | 17.8 | 386.5 | 108.7 | 278.6 | 91.8 |
| 205 | HTR2C | 3358 | 1852 | 10 | 454.3 | 107.4 | 447.3 | 10.3 | 429.1 | 74.9 |
| 205 | HTR2C | 3358 | 1852 | 30 | 565.3 | 137.7 | 596.6 | 92.7 | 568 | 44.1 |
| 205 | HTR2C | 3358 | 1852 | 100 | 437.6 | 75.9 | 1158.7 | 227.7 | 774.3 | 237.8 |
| 205 | HTR2C | 3358 | 1940 | 10 | 86.8 | 22.7 | 136.7 | 11.5 | 124.1 | 38.9 |
| 205 | HTR2C | 3358 | 1940 | 30 | 105.4 | 17.1 | 112.6 | 12.9 | 155.5 | 21.6 |
| 205 | HTR2C | 3358 | 1940 | 100 | 83.9 | 15.1 | 156.3 | 21.1 | 199.1 | 60.9 |
| 215 | LOC345667 | 345667 | 104231 | 10 | 301.2 | 53.3 | 317.5 | 38.1 | 302.1 | 93.8 |
| 215 | LOC345667 | 345667 | 104231 | 30 | 353.7 | 24 | 377.5 | 36.2 | 309 | 49.5 |
| 215 | LOC345667 | 345667 | 104231 | 100 | 490.6 | 33.8 | 635.2 | 35.6 | 461.4 | 36 |
| 215 | LOC345667 | 345667 | 212853 | 10 | 436.6 | 140.7 | 489.6 | 74.1 | 371.3 | 4.2 |
| 215 | LOC345667 | 345667 | 212853 | 30 | 649.3 | 82.1 | 580.8 | 82.2 | 523.5 | 97.5 |
| 215 | LOC345667 | 345667 | 212853 | 100 | 952.9 | 107.5 | 873.4 | 112.1 | 932.2 | 144.8 |
| 237 | GPR3 | 2827 | 1785 | 10 | 803.5 | 125.7 | 389.1 | 66.8 | 329 | 76.1 |
| 237 | GPR3 | 2827 | 1785 | 30 | 1236.3 | 235.5 | 577.6 | 78.8 | 346.8 | 26.3 |
| 237 | GPR3 | 2827 | 1785 | 100 | 1205.3 | 182 | 1253.8 | 182.6 | 490.4 | 32.5 |
| 237 | GPR3 | 2827 | 212766 | 10 | 151.3 | 38.7 | 154.2 | 26.8 | 123.1 | 4.5 |
| 237 | GPR3 | 2827 | 212766 | 30 | 185.3 | 57.4 | 171.4 | 18.5 | 180.3 | 4.5 |
| 237 | GPR3 | 2827 | 212766 | 100 | 137.5 | 40.8 | 152.3 | 53.3 | 191.1 | 39.3 |
| 242 | DCTD | 1635 | 119612 | 10 | 420.5 | 55 | 469.3 | 154.6 | 125.7 | 11.9 |
| 242 | DCTD | 1635 | 119512 | 30 | 779.7 | 68 | 568.5 | 84.9 | 428.5 | 107 |
| 242 | DCTD | 1635 | 119612 | 100 | 846.4 | 93.1 | 1253.4 | 63.9 | 593.4 | 84.1 |
| 242 | DCTD | 1635 | 214555 | 10 | 243.8 | 68.8 | 222.4 | 36.5 | 189.4 | 37.2 |
| 242 | DCTD | 1635 | 214555 | 30 | 281.3 | 38.2 | 242.3 | 20.6 | 240.6 | 30.8 |
| 242 | DCTD | 1635 | 214555 | 100 | 321.5 | 71.7 | 406 | 79.3 | 353 | 7.5 |
| 261 | ACLY | 47 | 116939 | 10 | 455.5 | 44.1 | 675.6 | 114 | 366.8 | 82.7 |
| 261 | ACLY | 47 | 116939 | 30 | 552.2 | 212.1 | 763.5 | 18.7 | 474.8 | 48.6 |
| 261 | ACLY | 47 | 116939 | 100 | 845.5 | 113.3 | 1174.6 | 41 | 957.5 | 179 |
| 261 | ACLY | 47 | 116940 | 10 | | | | | 291.9 | 16.8 |
| 261 | ACLY | 47 | 116940 | 30 | | | | | 351.9 | 4.6 |
| 261 | ACLY | 47 | 116940 | 100 | | | | | 448.9 | 62.3 |
| 273 | AGXT2L1 | 64850 | 112236 | 10 | | | | | 232.7 | 60.7 |
| 273 | AGXT2L1 | 64850 | 112236 | 30 | | | | | 249.9 | 26.5 |
| 273 | AGXT2L1 | 64850 | 112236 | 100 | | | | | 283.1 | 52.9 |
| 273 | AGXT2L1 | 64850 | 112237 | 10 | 347.6 | 251.7 | 352.3 | 29.2 | 327.5 | 45.7 |
| 273 | AGXT2L1 | 64850 | 112237 | 30 | 505.2 | 87.6 | 415.2 | 51.4 | 326.4 | 29 |
| 273 | AGXT2L1 | 64850 | 112237 | 100 | 513.9 | 67.3 | 524.4 | 90.5 | 448.5 | 60.9 |
| 291 | ARSE | 415 | 119012 | 10 | 226.3 | 41.4 | 241.9 | 35.3 | 243 | 24.8 |
| 291 | ARSE | 415 | 119012 | 30 | 497.1 | 20.2 | 355.5 | 43.9 | 175.4 | 12.2 |
| 291 | ARSE | 415 | 119012 | 100 | 501.9 | 23.4 | 468.1 | 52.1 | 364 | 107.9 |
| 291 | ARSE | 415 | 214706 | 10 | 252.7 | 63.8 | 330.9 | 48 | 327.3 | 43.2 |
| 291 | ARSE | 415 | 214706 | 30 | 458.6 | 92.1 | 297.7 | 36.4 | 370.2 | 59.7 |
| 291 | ARSE | 415 | 214706 | 100 | 439 | 55.5 | 583.1 | 37.9 | 539.5 | 173.4 |
| 306 | NR2F2 | 7026 | 5922 | 10 | 286.8 | 46 | 251.8 | 35.5 | 262.3 | 23.5 |
| 306 | NR2F2 | 7026 | 5922 | 30 | 452.2 | 40.5 | 402.6 | 34.1 | 299.8 | 47.5 |
| 306 | NR2F2 | 7026 | 5922 | 100 | 557.1 | 54.2 | 753.1 | 132.2 | 415.2 | 55.7 |
| 306 | NR2F2 | 7026 | 45374 | 10 | 298.6 | 69.2 | 339.9 | 73.2 | 356.6 | 87.2 |
| 306 | NR2F2 | 7026 | 45374 | 30 | 485.8 | 28.6 | 481.3 | 32.3 | 413.6 | 36.8 |
| 306 | NR2F2 | 7026 | 45374 | 100 | 640.1 | 69.3 | 672.3 | 97.4 | 655.9 | 208 |
| 309 | ADAM18 | 8749 | 104120 | 10 | 205 | 37.9 | 197.6 | 29.9 | 262 | 8.6 |
| 309 | ADAM18 | 8749 | 104120 | 30 | 257.9 | 63.4 | 333.5 | 38.5 | 331.6 | 71.6 |
| 309 | ADAM18 | 8749 | 104120 | 100 | 281.2 | 56.9 | 626.9 | 119.8 | 537.8 | 63.6 |
| 309 | ADAM18 | 8749 | 212787 | 10 | 203 | 21.7 | 239 | 20.1 | 224.3 | 29.3 |
| 309 | ADAM18 | 8749 | 212787 | 30 | 306.9 | 32 | 320.7 | 16.3 | 265.8 | 39.3 |
| 309 | ADAM18 | 8749 | 212787 | 100 | 302.7 | 11.5 | 618.5 | 94.2 | 339 | 21.1 |
| 334 | ARHGEF2 | 9181 | 119230 | 10 | 548.2 | 9.1 | 284.9 | 16.5 | 216.2 | 21.9 |
| 334 | ARHGEF2 | 9181 | 119230 | 30 | 660.5 | 171.5 | 382 | 77.7 | 167.7 | 21.6 |
| 334 | ARHGEF2 | 9181 | 119230 | 100 | 702.6 | 97.1 | 551.9 | 145.2 | 195.4 | 38.2 |
| 334 | ARHGEF2 | 9181 | 214562 | 10 | 408.2 | 81 | 271.4 | 29.7 | 185 | 40 |
| 334 | ARHGEF2 | 9181 | 214562 | 30 | 470.5 | 63.7 | 325.5 | 57 | 230.2 | 41.4 |
| 334 | ARHGEF2 | 9181 | 214562 | 100 | 507.9 | 109.7 | 592 | 67.3 | 263.6 | 48.5 |
| 435 | PRSS15 | 9361 | 105664 | 10 | 274.8 | 92.1 | 266.8 | 53.4 | 284.1 | 11.1 |
| 435 | PRSS15 | 9361 | 105664 | 30 | 438.6 | 6.5 | 326.8 | 12.5 | 279.5 | 23.6 |
| 435 | PRSS15 | 9361 | 105664 | 100 | 435.9 | 84.6 | 480 | 75 | 367.1 | 120.5 |
| 435 | PRSS15 | 9361 | 212758 | 10 | 148.4 | 12.3 | 195.8 | 50.6 | 184.8 | 19 |
| 435 | PRSS15 | 9361 | 212758 | 30 | 241.1 | 44.1 | 226 | 46.9 | 263.2 | 21.9 |
| 435 | PRSS15 | 9361 | 212758 | 100 | 236.4 | 30.4 | 344.9 | 52.1 | 285.8 | 41.9 |
| 459 | PCYT1B | 9468 | 111523 | 10 | 293.2 | 57 | 238.5 | 49.4 | 429.8 | 56.8 |
| 459 | PCYT1B | 9468 | 111523 | 30 | 478.6 | 30 | 284.3 | 28.5 | 295.3 | 32.5 |
| 459 | PCYT1B | 9468 | 111523 | 100 | 439.9 | 24.8 | 529.4 | 101.8 | 555.2 | 147.4 |
| 459 | PCYT1B | 9468 | 214260 | 10 | 282.2 | 21.6 | 334.1 | 75.7 | 325.7 | 18.2 |
| 459 | PCYT1B | 9468 | 214260 | 30 | 429 | 35.2 | 397.2 | 24.2 | 299.9 | 39.5 |
| 459 | PCYT1B | 9468 | 214260 | 100 | 542.7 | 8.9 | 584.9 | 60 | 495.8 | 117.1 |
| 486 | FLJ21736 | 79984 | 119838 | 10 | | | | | 233 | 32.4 |
| 486 | FLJ21736 | 79984 | 119838 | 30 | | | | | 393.4 | 72.6 |
| 486 | FLJ21736 | 79984 | 119838 | 100 | | | | | 525.6 | 153 |
| 486 | FLJ21736 | 79984 | 235619 | 10 | | | | | 247.9 | 6.5 |
| 486 | FLJ21736 | 79984 | 235619 | 30 | | | | | 354.5 | 137 |
| 486 | FLJ21736 | 79984 | 235619 | 100 | | | | | 358.7 | 38.5 |
| 495 | KIR2DS1 | 3806 | 212646 | 10 | | | | | 602 | 48.9 |
| 495 | KIR2DS1 | 3806 | 212646 | 30 | | | | | 878.6 | 201.5 |
| 495 | KIR2DS1 | 3806 | 212646 | 100 | | | | | 974.8 | 205.1 |
| 495 | KIR2DS1 | 3806 | 235634 | 10 | | | | | 223.8 | 25.5 |
| 495 | KIR2DS1 | 3806 | 235634 | 30 | | | | | 303.2 | 46.3 |
| 495 | KIR2DS1 | 3806 | 235634 | 100 | | | | | 259.7 | 17.5 |
| 496 | KIR2DS3 | 3808 | 213159 | 10 | | | | | 760.9 | 110.6 |
| 496 | KIR2DS3 | 3808 | 213159 | 30 | | | | | 1006.6 | 156.5 |
| 496 | KIR2DS3 | 3808 | 213159 | 100 | | | | | 909.4 | 190.6 |
| 496 | KIR2DS3 | 3808 | 235633 | 10 | | | | | 177 | 42.2 |
| 496 | KIR2DS3 | 3808 | 235633 | 30 | | | | | 191.7 | 64.8 |
| 496 | KIR2DS3 | 3808 | 235633 | 100 | | | | | 178.8 | 35.4 |
| 506 | MOXD1 | 26002 | 111923 | 10 | | | | | 383.4 | 57.9 |
| 506 | MOXD1 | 26002 | 111923 | 30 | | | | | 486.8 | 106.6 |
| 506 | MOXD1 | 26002 | 111923 | 100 | | | | | 749.4 | 363.5 |
| 506 | MOXD1 | 26002 | 235615 | 10 | | | | | 191.5 | 36.4 |
| 506 | MOXD1 | 26002 | 235615 | 30 | | | | | 237.6 | 108 |
| 506 | MOXD1 | 26002 | 235615 | 100 | | | | | 303.6 | 22.6 |
| 514 | PEPD | 5184 | 105302 | 10 | 388 | 31.1 | 263.6 | 10.2 | 178.4 | 39.1 |
| 514 | PEPD | 5184 | 105302 | 30 | 441.3 | 124.9 | 293.3 | 67.3 | 220.3 | 70.9 |
| 514 | PEPD | 5184 | 105302 | 100 | 466.7 | 10.5 | 327.4 | 70.3 | 326.2 | 33.3 |
| 514 | PEPD | 5184 | 212688 | 10 | 234.7 | 113.3 | 239.4 | 42.8 | 172 | 9.9 |
| 514 | PEPD | 5184 | 212688 | 30 | 320.8 | 70.2 | 381.4 | 49.1 | 264.6 | 14.5 |
| 514 | PEPD | 5184 | 212688 | 100 | 281.9 | 30.1 | 664.8 | 157.3 | 316.6 | 41.4 |

TABLE 7

| Target | Target | Gene | siRNA | | Proliferation | Proliferation | Proliferation | Proliferation | Proliferation | Proliferation |
|---|---|---|---|---|---|---|---|---|---|---|
| No | Symbol | Id | ID | siRNA conc. | run1 Mean % | run1 SD % | run2 Mean % | run2 SD % | run3 Mean % | run3 SD % |
| 2 | HLCS | 3141 | 117852 | 10 | 89.1 | 10.6 | 86.4 | 3.3 | 105.1 | 2.5 |
| 2 | HLCS | 3141 | 117852 | 30 | 73.5 | 1 | 88.3 | 3.1 | 106.3 | 2.6 |
| 2 | HLCS | 3141 | 117852 | 100 | 84.9 | 9.8 | 74.8 | 6.5 | 105.9 | 2.2 |
| 2 | HLCS | 3141 | 117853 | 10 | 136.3 | 22.1 | 108.8 | 2.7 | 102.2 | 0.7 |
| 2 | HLCS | 3141 | 117853 | 30 | 112.2 | 5.8 | 114.5 | 1.6 | 105.4 | 1 |
| 2 | HLCS | 3141 | 117853 | 100 | 116.1 | 14.2 | 119.3 | 2.4 | 103.1 | 0.6 |
| 3 | SC4MOL | 6307 | 117417 | 10 | 97 | 29.7 | 107.2 | 2.6 | 99.5 | 3.6 |
| 3 | SC4MOL | 6307 | 117417 | 30 | 98.8 | 1.2 | 103.7 | 5.5 | 101.5 | 2.7 |
| 3 | SC4MOL | 6307 | 117417 | 100 | 114.8 | 7.1 | 110.1 | 1.5 | 99.6 | 0.7 |
| 3 | SC4MOL | 6307 | 117418 | 10 | 85.8 | 14.3 | 103.1 | 6.7 | 96.7 | 2.4 |
| 3 | SC4MOL | 6307 | 117418 | 30 | 98.6 | 1 | 104.5 | 2 | 101.7 | 1.2 |
| 3 | SC4MOL | 6307 | 117418 | 100 | 110.7 | 11 | 106.6 | 3.2 | 96.1 | 1.8 |
| 4 | CASP1 | 834 | 42626 | 10 | 101.2 | 49.9 | 110.6 | 5.1 | 97 | 9.7 |
| 4 | CASP1 | 834 | 42626 | 30 | 98.8 | 5.3 | 108.2 | 3.8 | 99.6 | 4.3 |
| 4 | CASP1 | 834 | 42626 | 100 | 120.7 | 3.3 | 109.2 | 3.8 | 100 | 2.3 |
| 4 | CASP1 | 834 | 42711 | 10 | 73.9 | 31.3 | 102.5 | 6.4 | 105 | 7.4 |
| 4 | CASP1 | 834 | 42711 | 30 | 83 | 2.4 | 105 | 5 | 97 | 2.7 |
| 4 | CASP1 | 834 | 42711 | 100 | 107 | 5.6 | 99.9 | 5.9 | 102.1 | 4.7 |
| 7 | CTSE | 1510 | 105039 | 10 | 88.4 | 50.9 | 105.4 | 0.5 | 104 | 4.8 |
| 7 | CTSE | 1510 | 105039 | 30 | 103.4 | 4.1 | 97 | 2.8 | 102.3 | 7.4 |
| 7 | CTSE | 1510 | 105039 | 100 | 86.5 | 5 | 97.3 | 1.7 | 103.5 | 3.3 |
| 7 | CTSE | 1510 | 105041 | 10 | 117.5 | 21 | 104.8 | 2.7 | 100 | 4.1 |
| 7 | CTSE | 1510 | 105041 | 30 | 105.3 | 4.3 | 106 | 5.4 | 106.1 | 5 |
| 7 | CTSE | 1510 | 105041 | 100 | 118.2 | 12.3 | 106.5 | 1.8 | 99.9 | 4.6 |
| 8 | FRK | 2444 | 1147 | 10 | 91 | 32.6 | 101.1 | 4.7 | 99.9 | 2 |
| 8 | FRK | 2444 | 1147 | 30 | 89.5 | 1.5 | 102.9 | 5.9 | 96.2 | 2.2 |
| 8 | ERK | 2444 | 1147 | 100 | 110.7 | 2.9 | 100.1 | 1.9 | 98 | 1 |
| 8 | FRK | 2444 | 1243 | 10 | 134.5 | 25 | 111.7 | 7.1 | 109 | 2.2 |
| 8 | FRK | 2444 | 1243 | 30 | 106.5 | 8.3 | 115 | 5 | 105.8 | 1.3 |
| 8 | FRK | 2444 | 1243 | 100 | 122.8 | 10.8 | 119.6 | 7.4 | 105.6 | 1.4 |
| 9 | HMBS | 3145 | 8225 | 10 | 97.8 | 23.9 | 103.8 | 1.7 | 95.3 | 1.6 |
| 9 | HMBS | 3145 | 8225 | 30 | 84.7 | 8 | 103.3 | 1.8 | 95.4 | 0.4 |
| 9 | HMBS | 3145 | 8225 | 100 | 101.9 | 7.1 | 105.4 | 5.5 | 94.5 | 1.5 |
| 9 | HMBS | 3145 | 117844 | 10 | 99 | 26.1 | 101.7 | 1.6 | 100.5 | 2 |
| 9 | HMBS | 3145 | 117844 | 30 | 95.1 | 0.9 | 100.6 | 1.4 | 101.6 | 2.8 |
| 9 | HMBS | 3145 | 117844 | 100 | 107.2 | 5.7 | 105.8 | 4.3 | 98.6 | 1.4 |
| 10 | HSD17B4 | 3295 | 106087 | 10 | 73 | 11.1 | 104.7 | 2.9 | 99.1 | 3 |
| 10 | HSD17B4 | 3295 | 106087 | 30 | 93.7 | 1.8 | 105.7 | 3.2 | 95.9 | 0.4 |
| 10 | HSD17B4 | 3295 | 106087 | 100 | 97.2 | 13.9 | 103.7 | 2.6 | 96.8 | 2.9 |
| 10 | HSD17B4 | 3295 | 106089 | 10 | 83.5 | 22.4 | 100 | 1.8 | 114.3 | 1.6 |
| 10 | HSD17B4 | 3295 | 106089 | 30 | 91.8 | 4.3 | 102.8 | 4.3 | 101.3 | 0.9 |
| 10 | HSD17B4 | 3295 | 106089 | 100 | 108.2 | 13.2 | 104.9 | 4.7 | 106.8 | 2.2 |
| 12 | OXTR | 5021 | 1766 | 10 | 133.6 | 20.8 | 114.8 | 7.1 | 110.7 | 8.6 |
| 12 | OXTR | 5021 | 1766 | 30 | 102.8 | 1.9 | 108.1 | 5.1 | 106.1 | 6.8 |
| 12 | OXTR | 5021 | 1766 | 100 | 101 | 16.9 | 96.9 | 9.4 | 108.3 | 6 |
| 12 | OXTR | 5021 | 1947 | 10 | 87.2 | 35.8 | 104.4 | 4.1 | 114.8 | 6.6 |
| 12 | OXTR | 5021 | 1947 | 30 | 99.5 | 10.1 | 107.5 | 6.1 | 106.8 | 4.4 |
| 12 | OXTR | 5021 | 1947 | 100 | 113.5 | 14.2 | 111.3 | 1.8 | 110.1 | 4.4 |
| 16 | TPI1 | 7167 | 43820 | 10 | 100.2 | 27.7 | 109.9 | 2.7 | 98.9 | 0.5 |
| 16 | TPI1 | 7167 | 43820 | 30 | 102.2 | 3.7 | 105.6 | 3.8 | 97.6 | 1.8 |
| 16 | TPI1 | 7167 | 43820 | 100 | 124.7 | 5.5 | 102 | 1.8 | 97.1 | 2.9 |
| 16 | TPI1 | 7167 | 43916 | 10 | 108.9 | 25.7 | 112.1 | 4.1 | 97.2 | 1.7 |
| 16 | TPI1 | 7167 | 43916 | 30 | 105.3 | 2.2 | 106.4 | 4.1 | 96.1 | 3.7 |
| 16 | TPI1 | 7167 | 43916 | 100 | 124.7 | 4.9 | 101.8 | 3.4 | 96.5 | 2.1 |
| 18 | SLC30A2 | 7780 | 117693 | 10 | 115.3 | 21.7 | 107.7 | 3.7 | 103.6 | 2.4 |
| 18 | SLC30A2 | 7780 | 117693 | 30 | 88.2 | 10.2 | 112.2 | 1.4 | 103.8 | 0.5 |
| 18 | SLC30A2 | 7780 | 117693 | 100 | 113.4 | 11.2 | 111.8 | 1.9 | 101.2 | 1.9 |
| 18 | SLC30A2 | 7780 | 117695 | 10 | 115.3 | 21.7 | 108.5 | 6.1 | 94.9 | 1.5 |
| 18 | SLC30A2 | 7780 | 117695 | 30 | 103.2 | 3.6 | 104.8 | 3.2 | 91.3 | 1.9 |
| 18 | SLC30A2 | 7780 | 117695 | 100 | 113.4 | 11.2 | 100.7 | 2.8 | 87.3 | 2.5 |
| 19 | ULK1 | 8408 | 118260 | 10 | 76.1 | 41.8 | 115 | 4.8 | 108.6 | 1.3 |
| 19 | ULK1 | 8408 | 118260 | 30 | 108.9 | 7 | 117.3 | 3.1 | 105.2 | 0.7 |
| 19 | ULK1 | 8408 | 118260 | 100 | 114.2 | 0.9 | 119.6 | 7.3 | 105.9 | 1.3 |
| 19 | ULK1 | 8408 | 118261 | 10 | 86.2 | 39.5 | 110 | 5.3 | 103.6 | 2.5 |
| 19 | ULK1 | 8408 | 118261 | 30 | 79.3 | 29.9 | 111 | 3.2 | 103.1 | 1.1 |
| 19 | ULK1 | 8408 | 118261 | 100 | 90.9 | 11.1 | 107.2 | 2.5 | 102.8 | 1.5 |
| 22 | SPUVE | 11098 | 19104 | 10 | 104.9 | 26.2 | 112.5 | 2.3 | 100.6 | 1.3 |
| 22 | SPUVE | 11098 | 19104 | 30 | 104.3 | 3.8 | 114.7 | 0.9 | 99.6 | 3.5 |
| 22 | SPUVE | 11098 | 19104 | 100 | 115.4 | 10.3 | 115.3 | 2.1 | 101.1 | 2.1 |
| 22 | SPUVE | 11098 | 19196 | 10 | 119 | 11.3 | 114.3 | 4.9 | 105.8 | 2.6 |
| 22 | SPUVE | 11098 | 19196 | 30 | 103.9 | 13.7 | 117.7 | 2 | 100.9 | 1.9 |
| 22 | SPUVE | 11098 | 19196 | 100 | 110.3 | 17.8 | 121.9 | 4.9 | 102.6 | 3.2 |
| 27 | PKD1L2 | 114780 | 104830 | 10 | 100.9 | 45.8 | 108.8 | 5.5 | 102.6 | 1 |
| 27 | PKD1L2 | 114780 | 104830 | 30 | 102.4 | 1.9 | 113.9 | 3.2 | 99.3 | 0.8 |
| 27 | PKD1L2 | 114780 | 104830 | 100 | 108.9 | 8.1 | 116.6 | 4.7 | 99.4 | 0.3 |
| 27 | PKD1L2 | 114780 | 104835 | 10 | 88.4 | 50.9 | 106.7 | 2.2 | 100.4 | 2 |
| 27 | PKD1L2 | 114780 | 104835 | 30 | 91.3 | 9.7 | 103.4 | 3 | 99.5 | 1.6 |
| 27 | PKD1L2 | 114780 | 104835 | 100 | 86.5 | 5 | 105.2 | 1 | 98.7 | 1.3 |
| 39 | DNM1L | 10059 | 19471 | 10 | 100.2 | 27.7 | 107.4 | 5.4 | 113.1 | 2.6 |
| 39 | DNM1L | 10059 | 19471 | 30 | 107.7 | 1.5 | 111 | 7.3 | 108.6 | 1.3 |
| 39 | DNM1L | 10059 | 19471 | 100 | 124.7 | 5.5 | 113.2 | 4.2 | 111.6 | 2.7 |
| 39 | DNM1L | 10059 | 214799 | 10 | 94.6 | 20.6 | 99.4 | 6.5 | 97 | 1.1 |
| 39 | DNM1L | 10059 | 214799 | 30 | 95.8 | 5.6 | 103.3 | 1.4 | 93.9 | 2.1 |
| 39 | DNM1L | 10059 | 214799 | 100 | 105.5 | 12.2 | 100.2 | 6.8 | 94.5 | 1.5 |
| 88 | GALR2 | 8811 | 44971 | 10 | 117.1 | 23.6 | 98 | 2.2 | 89 | 6.7 |
| 88 | GALR2 | 8811 | 44971 | 30 | 99.5 | 2.6 | 96.5 | 1.6 | 90 | 3.4 |
| 88 | GALR2 | 8811 | 44971 | 100 | 98.8 | 19.5 | 88.3 | 7.2 | 91.2 | 3.1 |
| 88 | GALR2 | 8811 | 45063 | 10 | 76.1 | 41.8 | 111.4 | 3.3 | 105.8 | 10.6 |
| 88 | GALR2 | 8811 | 45063 | 30 | 99.2 | 1.2 | 109.5 | 3.8 | 95 | 3.9 |
| 88 | GALR2 | 8811 | 45063 | 100 | 114.2 | 0.9 | 106.1 | 2.9 | 98.3 | 7.9 |
| 143 | SCP2 | 6342 | 117110 | 10 | | | | | 106.2 | 1.2 |
| 143 | SCP2 | 6342 | 117110 | 30 | | | | | 105.3 | 1.6 |
| 143 | SCP2 | 6342 | 117110 | 100 | | | | | 109 | 1.8 |
| 143 | SCP2 | 6342 | 119182 | 10 | 101.2 | 49.9 | 112 | 3.9 | 98.4 | 1.1 |
| 143 | SCP2 | 6342 | 119182 | 30 | 99.5 | 1.8 | 107 | 5.7 | 99.3 | 3.1 |
| 143 | SCP2 | 6342 | 119182 | 100 | 120.7 | 3.3 | 105.6 | 2.2 | 96.5 | 1.8 |
| 171 | PAFAH2 | 5051 | 119066 | 10 | 131.7 | 27.8 | 103.7 | 5.1 | 102.6 | 1.8 |
| 171 | PAFAH2 | 5051 | 119066 | 30 | 100.1 | 8.9 | 101.5 | 6.5 | 99.9 | 0.8 |
| 171 | PAFAH2 | 5051 | 119066 | 100 | 121.2 | 8.7 | 113.5 | 4.6 | 102.7 | 2.8 |
| 171 | PAFAH2 | 5051 | 214710 | 10 | 110.2 | 34.7 | 105.6 | 2.9 | 104.2 | 1.9 |
| 171 | PAFAH2 | 5051 | 214710 | 30 | 97.3 | 2.4 | 108.7 | 3.9 | 102.1 | 0.7 |
| 171 | PAFAH2 | 5051 | 214710 | 100 | 116.8 | 5.8 | 108 | 3.3 | 100.5 | 0.9 |
| 180 | GAD2 | 2572 | 9108 | 10 | 114.9 | 23.7 | 103.1 | 0.3 | 104.6 | 0.6 |
| 180 | GAD2 | 2572 | 9108 | 30 | 82 | 11.4 | 102.2 | 5.3 | 101.7 | 2.2 |
| 180 | GAD2 | 2572 | 9108 | 100 | 98 | 13 | 117.5 | 5.5 | 102 | 0.5 |
| 180 | GAD2 | 2572 | 214716 | 10 | 84.1 | 47.7 | 99.5 | 6 | 105.4 | 2.1 |
| 180 | GAD2 | 2572 | 214716 | 30 | 84.4 | 5.3 | 95.1 | 2.4 | 101.4 | 1.3 |
| 180 | GAD2 | 2572 | 214716 | 100 | 109 | 4.3 | 97.2 | 5.6 | 99.7 | 2.2 |
| 205 | HTR2C | 3358 | 1852 | 10 | 73.6 | 33.8 | 102.5 | 2.1 | 106.5 | 5.2 |
| 205 | HTR2C | 3358 | 1852 | 30 | 104.3 | 1.8 | 100.4 | 7 | 97 | 6.9 |
| 205 | HTR2C | 3358 | 1852 | 100 | 108.4 | 5.1 | 99.3 | 1.4 | 99.5 | 2 |
| 205 | HTR2C | 3358 | 1940 | 10 | 109.4 | 39.8 | 100.6 | 5.2 | 97.7 | 7.4 |
| 205 | HTR2C | 3358 | 1940 | 30 | 99.2 | 3.1 | 93.4 | 4.8 | 96.4 | 6.2 |
| 205 | HTR2C | 3358 | 1940 | 100 | 123.1 | 7.6 | 92 | 3 | 100.7 | 2.8 |
| 215 | LOC345667 | 345667 | 104231 | 10 | 94.5 | 35.5 | 103.8 | 1.5 | 100.5 | 7.2 |
| 215 | LOC345667 | 345667 | 104231 | 30 | 98.6 | 2.2 | 104.9 | 4.5 | 94.2 | 5.2 |
| 215 | LOC345667 | 345667 | 104231 | 100 | 119.8 | 5.9 | 106.8 | 4.6 | 99.2 | 3.5 |
| 215 | LOC345667 | 345667 | 212853 | 10 | 73.6 | 33.8 | 107 | 5 | 107.7 | 1.7 |
| 215 | LOC345667 | 345667 | 212853 | 30 | 100.1 | 6.7 | 111 | 1.4 | 104.9 | 1.9 |
| 215 | LOC345667 | 345667 | 212853 | 100 | 108.4 | 5.1 | 112.3 | 4.7 | 107 | 1.8 |
| 237 | GPR3 | 2827 | 1785 | 10 | 96.6 | 25.4 | 102.9 | 4.4 | 103.5 | 0.6 |
| 237 | GPR3 | 2827 | 1785 | 30 | 101.2 | 0.3 | 97.8 | 4 | 100.4 | 2.6 |
| 237 | GPR3 | 2627 | 1785 | 100 | 106.2 | 6.8 | 95.3 | 5 | 98.8 | 1.8 |
| 237 | GPR3 | 2827 | 212766 | 10 | 87 | 8.2 | 100.4 | 3.9 | 107.1 | 2 |
| 237 | GPR3 | 2827 | 212766 | 30 | 99.3 | 2.4 | 104.3 | 2.2 | 103.3 | 2 |
| 237 | GPR3 | 2827 | 212766 | 100 | 112.2 | 5.5 | 102.3 | 2 | 103.1 | 2.3 |
| 242 | DCTD | 1635 | 119612 | 10 | 96.6 | 25.4 | 99.6 | 7.7 | 103.4 | 0.9 |
| 242 | DCTD | 1635 | 119612 | 30 | 99.9 | 3.2 | 102.5 | 7.8 | 96.1 | 1.4 |
| 242 | DCTD | 1635 | 119612 | 100 | 106.2 | 6.8 | 108.7 | 8.2 | 94.7 | 1.4 |
| 242 | DCTD | 1635 | 214555 | 10 | 109.4 | 39.8 | 111.4 | 4.3 | 104.5 | 1.8 |
| 242 | DCTD | 1635 | 214555 | 30 | 113.9 | 3.3 | 115.3 | 3.2 | 100.5 | 1.9 |
| 242 | DCTD | 1635 | 214555 | 100 | 123.1 | 7.6 | 117.6 | 4.3 | 98.6 | 2.2 |
| 261 | ACLY | 47 | 116939 | 10 | 111.7 | 21 | 107.4 | 2.8 | 101.9 | 0.8 |
| 261 | ACLY | 47 | 116939 | 30 | 102 | 8.1 | 109.6 | 2.4 | 100.8 | 1.7 |
| 261 | ACLY | 47 | 116939 | 100 | 107.1 | 17 | 111.7 | 3.3 | 98.4 | 1 |
| 261 | ACLY | 47 | 116940 | 10 | | | | | 99.8 | 1.2 |
| 261 | ACLY | 47 | 116940 | 30 | | | | | 97.3 | 2.2 |
| 261 | ACLY | 47 | 116940 | 100 | | | | | 98.4 | 2.3 |
| 273 | AGXT2L1 | 64850 | 112236 | 10 | | | | | 106.7 | 1.2 |
| 273 | AGXT2L1 | 64850 | 112236 | 30 | | | | | 102.2 | 0.2 |
| 273 | AGXT2L1 | 64850 | 112236 | 100 | | | | | 104.3 | 2.2 |
| 273 | AGXT2L1 | 64850 | 112237 | 10 | 73 | 11.1 | 103.7 | 6.5 | 99.9 | 2.8 |
| 273 | AGXT2L1 | 64850 | 112237 | 30 | 104.9 | 1.9 | 110.3 | 3.2 | 98.2 | 1.3 |
| 273 | AGXT2L1 | 64850 | 112237 | 100 | 97.2 | 13.9 | 105.6 | 1.4 | 99.1 | 0.2 |
| 291 | ARSE | 415 | 119012 | 10 | 141 | 29.1 | 106.2 | 2.5 | 104.3 | 1.7 |
| 291 | ARSE | 415 | 119012 | 30 | 94.2 | 4.2 | 109.6 | 2.4 | 99.8 | 1.1 |
| 291 | ARSE | 415 | 119012 | 100 | 115.2 | 15.1 | 116.1 | 3.3 | 100.7 | 1.2 |
| 291 | ARSE | 415 | 214706 | 10 | 117.3 | 24.2 | 96 | 4.8 | 104.8 | 1.5 |
| 291 | ARSE | 415 | 214706 | 30 | 73.6 | 8 | 95.6 | 4.7 | 96.6 | 0.7 |
| 291 | ARSE | 415 | 214706 | 100 | 72.1 | 8.8 | 86.4 | 4.5 | 95.7 | 0.8 |
| 306 | NR2F2 | 7026 | 5922 | 10 | 120.8 | 24.1 | 109.2 | 0.8 | 108.3 | 0.4 |
| 306 | NR2F2 | 7026 | 5922 | 30 | 99.1 | 6.6 | 110 | 1.8 | 102.1 | 1 |
| 306 | NR2F2 | 7026 | 5922 | 100 | 107.1 | 12.2 | 113.4 | 7.9 | 104.5 | 0.9 |
| 306 | NR2F2 | 7026 | 45374 | 10 | 86 | 18.2 | 112.1 | 4 | 109.8 | 6.4 |
| 306 | NR2F2 | 7026 | 45374 | 30 | 112.1 | 6 | 114.1 | 2.2 | 105.9 | 7.2 |
| 306 | NR2F2 | 7026 | 45374 | 100 | 129.2 | 9.4 | 116 | 2.1 | 104.4 | 8.1 |
| 309 | ADAM18 | 8749 | 104120 | 10 | 111.7 | 21 | 114.8 | 9.2 | 102.8 | 9.8 |
| 309 | ADAM18 | 8749 | 104120 | 30 | 102.1 | 1.8 | 106.2 | 3.3 | 97.3 | 9.7 |
| 309 | ADAM18 | 8749 | 104120 | 100 | 107.1 | 17 | 97.3 | 5.6 | 98.6 | 2.1 |
| 309 | ADAM18 | 8749 | 212787 | 10 | 90.2 | 28.8 | 90.9 | 1.9 | 102.3 | 0.7 |
| 309 | ADAM18 | 8749 | 212787 | 30 | 98.5 | 1.2 | 99.4 | 2.1 | 101.7 | 1.6 |
| 309 | ADAM18 | 8749 | 212787 | 100 | 98.4 | 8.8 | 99.5 | 4.6 | 100.6 | 1.4 |
| 334 | ARHGEF2 | 9181 | 119230 | 10 | 104.9 | 26.2 | 97.2 | 7.9 | 108 | 2.7 |
| 334 | ARHGEF2 | 9181 | 119230 | 30 | 99.1 | 3.4 | 93.1 | 7.7 | 100.3 | 0.2 |
| 334 | ARHGEF2 | 9181 | 119230 | 100 | 115.4 | 10.3 | 92 | 1.8 | 106 | 1.6 |
| 334 | ARHGEF2 | 9181 | 214562 | 10 | 74.4 | 39.6 | 105.8 | 2.4 | 97.9 | 1.9 |
| 334 | ARHGEF2 | 9181 | 214562 | 30 | 101.4 | 2.5 | 97.1 | 3.1 | 97.4 | 4 |
| 334 | ARHGEF2 | 9181 | 214562 | 100 | 83.5 | 3.5 | 93.9 | 1.8 | 96.5 | 2.4 |
| 435 | PRSS15 | 9361 | 105664 | 10 | 130.9 | 14.3 | 106.4 | 8.7 | 106.7 | 0.6 |
| 435 | PRSS15 | 9361 | 105664 | 30 | 96.1 | 7.7 | 114.5 | 2.9 | 102.9 | 1.5 |
| 435 | PRSS15 | 9361 | 105664 | 100 | 110.9 | 7.8 | 112.8 | 6.1 | 104 | 1.4 |
| 435 | PRSS15 | 9361 | 212758 | 10 | 140.1 | 28.8 | 115.5 | 3.6 | 100.4 | 0.8 |
| 435 | PRSS15 | 9361 | 212758 | 30 | 113.8 | 6.1 | 115.8 | 4.9 | 104.2 | 2.6 |
| 435 | PRSS15 | 9361 | 212758 | 100 | 131.2 | 14.1 | 121.6 | 3.2 | 107.7 | 2.1 |
| 459 | PCYT1B | 9468 | 111523 | 10 | 107.8 | 34.4 | 116.3 | 1.7 | 101.8 | 1.4 |
| 459 | PCYT1B | 9468 | 111523 | 30 | 113.3 | 8.7 | 115.9 | 1.7 | 106.4 | 1.7 |
| 459 | PCYT1B | 9468 | 111523 | 100 | 107.3 | 11.8 | 119.2 | 1.3 | 106.1 | 1.4 |
| 459 | PCYT1B | 9468 | 214260 | 10 | 133.6 | 20.8 | 108.6 | 4.5 | 104 | 2.6 |
| 459 | PCYT1B | 9468 | 214260 | 30 | 103.6 | 14.1 | 109.2 | 3.6 | 102.9 | 1.6 |
| 459 | PCYT1B | 9468 | 214260 | 100 | 101 | 16.9 | 109.2 | 3.4 | 102.3 | 2.2 |
| 486 | FLJ21736 | 79984 | 119838 | 10 | | | | | 129.8 | 12.4 |
| 486 | FLJ21736 | 79984 | 119838 | 30 | | | | | 158.8 | 28.4 |
| 486 | FLJ21736 | 79984 | 119838 | 100 | | | | | 165.3 | 25.2 |
| 486 | FLJ21736 | 79984 | 235619 | 10 | | | | | 129.1 | 23.3 |
| 486 | FLJ21736 | 79984 | 235619 | 30 | | | | | 170.4 | 2.5 |
| 486 | FLJ21736 | 79984 | 235619 | 100 | | | | | 146.9 | 23 |
| 495 | KIR2DS1 | 3806 | 212646 | 10 | | | | | 126.8 | 7 |
| 495 | KIR2DS1 | 3806 | 212646 | 30 | | | | | 137.9 | 16.6 |
| 495 | KIR2DS1 | 3806 | 212646 | 100 | | | | | 132.8 | 16.9 |
| 495 | KIR2DS1 | 3806 | 235634 | 10 | | | | | 117.5 | 13.2 |
| 495 | KIR2DS1 | 3806 | 235634 | 30 | | | | | 137.5 | 30.8 |
| 495 | KIR2DS1 | 3806 | 235634 | 100 | | | | | 143 | 5 |
| 496 | KIR2DS3 | 3808 | 213159 | 10 | | | | | 127.9 | 8.4 |
| 496 | KIR2DS3 | 3808 | 213159 | 30 | | | | | 150.6 | 4 |
| 496 | KIR2DS3 | 3808 | 213159 | 100 | | | | | 155.8 | 26.2 |
| 496 | KIR2DS3 | 3808 | 235633 | 10 | | | | | 122.2 | 19.5 |
| 496 | KIR2DS3 | 3808 | 235633 | 30 | | | | | 116.3 | 13.4 |
| 496 | KIR2DS3 | 3808 | 235633 | 100 | | | | | 130 | 12.8 |
| 506 | MOXD1 | 26002 | 111923 | 10 | | | | | 125.3 | 6.4 |
| 506 | MOXD1 | 26002 | 111923 | 30 | | | | | 152.1 | 20.1 |
| 506 | MOXD1 | 26002 | 111923 | 100 | | | | | 142 | 19.8 |
| 506 | MOXD1 | 26002 | 235615 | 10 | | | | | 134.9 | 7.5 |
| 506 | MOXD1 | 26002 | 235615 | 30 | | | | | 145 | 10.9 |
| 506 | MOXD1 | 26002 | 235615 | 100 | | | | | 137.7 | 12.5 |
| 514 | PEPD | 5184 | 105302 | 10 | 84.1 | 47.7 | 117.1 | 4 | 108.6 | 1.5 |
| 514 | PEPD | 5184 | 105302 | 30 | 105 | 1.7 | 114.9 | 1.2 | 104.9 | 2.4 |
| 514 | PEPD | 5184 | 105302 | 100 | 109 | 4.3 | 113 | 2.8 | 104.3 | 2 |
| 514 | PEPD | 5184 | 212688 | 10 | 96.5 | 43 | 109.8 | 6.8 | 99.9 | 1.6 |
| 514 | PEPD | 5184 | 212688 | 30 | 102.8 | 1.1 | 104.7 | 3.8 | 96.9 | 2.7 |
| 514 | PEPD | 5184 | 212688 | 100 | 121.7 | 6.4 | 96.5 | 4.5 | 98.5 | 0.8 |

TABLE 8

| | Target | | | | % mRNA |
|---|---|---|---|---|---|
| Target No | Symbol | Gene Id | siRNA ID | siRNA conc. | Mean |
| 2 | HLCS | 3141 | 117852 | 10 | 26.6 |
| 2 | HLCS | 3141 | 117852 | 30 | 20.3 |
| 2 | HLCS | 3141 | 117852 | 100 | 13.2 |
| 2 | HLCS | 3141 | 117853 | 10 | 66 |
| 2 | HLCS | 3141 | 117853 | 30 | 54.7 |
| 2 | HLCS | 3141 | 117853 | 100 | 50.6 |
| 3 | SC4MOL | 6307 | 117417 | 10 | 34.4 |
| 3 | SC4MOL | 6307 | 117417 | 30 | 10.8 |
| 3 | SC4MOL | 6307 | 117417 | 100 | 8.3 |
| 3 | SC4MOL | 6307 | 117418 | 10 | 43.5 |
| 3 | SC4MOL | 6307 | 117418 | 30 | 23.4 |
| 3 | SC4MOL | 6307 | 117418 | 100 | 23.4 |
| 4 | CASP1 | 834 | 42626 | 10 | |
| 4 | CASP1 | 834 | 42626 | 30 | |
| 4 | CASP1 | 834 | 42626 | 100 | |
| 4 | CASP1 | 834 | 42711 | 10 | |
| 4 | CASP1 | 834 | 42711 | 30 | |
| 4 | CASP1 | 834 | 42711 | 100 | |
| 7 | CTSE | 1510 | 105039 | 10 | 11.5 |
| 7 | CTSE | 1510 | 105039 | 30 | 26.6 |
| 7 | CTSE | 1510 | 105039 | 100 | 8.2 |
| 7 | CTSE | 1510 | 105041 | 10 | 60.5 |
| 7 | CTSE | 1510 | 105041 | 30 | 62.7 |
| 7 | CTSE | 1510 | 105041 | 100 | 20.8 |
| 8 | FRK | 2444 | 1147 | 10 | 27.7 |
| 8 | FRK | 2444 | 1147 | 30 | 24.4 |
| 8 | FRK | 2444 | 1147 | 100 | 22.4 |
| 8 | FRK | 2444 | 1243 | 10 | 87.2 |
| 8 | FRK | 2444 | 1243 | 30 | 70.6 |
| 8 | FRK | 2444 | 1243 | 100 | 69.2 |
| 9 | HMBS | 3145 | 8225 | 10 | |
| 9 | HMBS | 3145 | 8225 | 30 | 37.4 |
| 9 | HMBS | 3145 | 8225 | 100 | 31.9 |
| 9 | HMBS | 3145 | 117844 | 10 | 27.3 |
| 9 | HMBS | 3145 | 117844 | 30 | 15.6 |
| 9 | HMBS | 3145 | 117844 | 100 | 12.5 |
| 10 | HSD17B4 | 3295 | 106087 | 10 | 10.9 |
| 10 | HSD17B4 | 3295 | 106087 | 30 | 9.1 |
| 10 | HSD17B4 | 3295 | 106087 | 100 | 11.8 |
| 10 | HSD17B4 | 3295 | 106089 | 10 | 39.5 |
| 10 | HSD17B4 | 3295 | 106089 | 30 | 23.5 |
| 10 | HSD17B4 | 3295 | 106089 | 100 | 13.3 |
| 12 | OXTR | 5021 | 1766 | 10 | 36.9 |
| 12 | OXTR | 5021 | 1766 | 30 | 61.7 |
| 12 | OXTR | 5021 | 1766 | 100 | 9.9 |
| 12 | OXTR | 5021 | 1947 | 10 | |
| 12 | OXTR | 5021 | 1947 | 30 | |
| 12 | OXTR | 5021 | 1947 | 100 | |
| 16 | TPI1 | 7167 | 43820 | 10 | 8.7 |
| 16 | TPI1 | 7167 | 43820 | 30 | 12.5 |
| 16 | TPI1 | 7167 | 43820 | 100 | 21.8 |
| 16 | TPI1 | 7167 | 43916 | 10 | 18 |
| 16 | TPI1 | 7167 | 43916 | 30 | 28.5 |
| 16 | TPI1 | 7167 | 43916 | 100 | 10.7 |
| 18 | SLC30A2 | 7780 | 117693 | 10 | |
| 18 | SLC30A2 | 7780 | 117693 | 30 | |
| 18 | SLC30A2 | 7780 | 117693 | 100 | |
| 18 | SLC30A2 | 7780 | 117695 | 10 | |
| 18 | SLC30A2 | 7780 | 117695 | 30 | |
| 18 | SLC30A2 | 7780 | 117695 | 100 | |
| 19 | ULK1 | 8408 | 118260 | 10 | 23.6 |
| 19 | ULK1 | 8408 | 118260 | 30 | 130.9 |
| 19 | ULK1 | 8408 | 118260 | 100 | 162.7 |
| 19 | ULK1 | 8408 | 118261 | 10 | 17.5 |
| 19 | ULK1 | 8408 | 118261 | 30 | 24 |
| 19 | ULK1 | 8408 | 118261 | 100 | 38.6 |
| 22 | SPUVE | 11098 | 19104 | 10 | 9.7 |
| 22 | SPUVE | 11098 | 19104 | 30 | 28.9 |
| 22 | SPUVE | 11098 | 19104 | 100 | 12 |
| 22 | SPUVE | 11098 | 19196 | 10 | 30 |
| 22 | SPUVE | 11098 | 19196 | 30 | 20 |
| 22 | SPUVE | 11098 | 19196 | 100 | 17.4 |
| 27 | PKD1L2 | 114780 | 104830 | 10 | |
| 27 | PKD1L2 | 114780 | 104830 | 30 | |
| 27 | PKD1L2 | 114780 | 104830 | 100 | |
| 27 | PKD1L2 | 114780 | 104835 | 10 | |
| 27 | PKD1L2 | 114780 | 104835 | 30 | |
| 27 | PKD1L2 | 114780 | 104835 | 100 | |
| 39 | DNM1L | 10059 | 19471 | 10 | 44 |
| 39 | DNM1L | 10059 | 19471 | 30 | 29.9 |
| 39 | DNM1L | 10059 | 19471 | 100 | 10.2 |
| 39 | DNM1L | 10059 | 214799 | 10 | 19.4 |
| 39 | DNM1L | 10059 | 214799 | 30 | 22.4 |
| 39 | DNM1L | 10059 | 214799 | 100 | 21.4 |
| 88 | GALR2 | 8811 | 44971 | 10 | |
| 88 | GALR2 | 8811 | 44971 | 30 | |
| 88 | GALR2 | 8811 | 44971 | 100 | |
| 88 | GALR2 | 8811 | 45063 | 10 | |
| 88 | GALR2 | 8811 | 45063 | 30 | |
| 88 | GALR2 | 8811 | 45063 | 100 | |
| 143 | SCP2 | 6342 | 117110 | 10 | 21.5 |
| 143 | SCP2 | 6342 | 117110 | 30 | 15.3 |
| 143 | SCP2 | 6342 | 117110 | 100 | 4.5 |
| 143 | SCP2 | 6342 | 119182 | 10 | 25.7 |
| 143 | SCP2 | 6342 | 119182 | 30 | 47.1 |
| 143 | SCP2 | 6342 | 119182 | 100 | 16.9 |
| 171 | PAFAH2 | 5051 | 119066 | 10 | 26.1 |
| 171 | PAFAH2 | 5051 | 119066 | 30 | 18.9 |
| 171 | PAFAH2 | 5051 | 119066 | 100 | 15.2 |
| 171 | PAFAH2 | 5051 | 214710 | 10 | 14.4 |
| 171 | PAFAH2 | 5051 | 214710 | 30 | 9.3 |
| 171 | PAFAH2 | 5051 | 214710 | 100 | 11.2 |
| 180 | GAD2 | 2572 | 9108 | 10 | |
| 180 | GAD2 | 2572 | 9108 | 30 | |
| 180 | GAD2 | 2572 | 9108 | 100 | |
| 180 | GAD2 | 2572 | 214716 | 10 | |
| 180 | GAD2 | 2572 | 214716 | 30 | |
| 180 | GAD2 | 2572 | 214716 | 100 | |
| 205 | HTR2C | 3358 | 1852 | 10 | |
| 205 | HTR2C | 3358 | 1852 | 30 | |
| 205 | HTR2C | 3358 | 1852 | 100 | |
| 205 | HTR2C | 3358 | 1940 | 10 | |
| 205 | HTR2C | 3358 | 1940 | 30 | |
| 205 | HTR2C | 3358 | 1940 | 100 | |
| 215 | LOC345667 | 345667 | 104231 | 10 | |
| 215 | LOC345667 | 345667 | 104231 | 30 | |
| 215 | LOC345667 | 345667 | 104231 | 100 | |
| 215 | LOC345667 | 345667 | 212853 | 10 | 66.6 |
| 215 | LOC345667 | 345667 | 212853 | 30 | 64.8 |
| 215 | LOC345667 | 345667 | 212853 | 100 | 56.7 |
| 237 | GPR3 | 2827 | 1785 | 10 | 60.4 |
| 237 | GPR3 | 2827 | 1785 | 30 | 165.8 |
| 237 | GPR3 | 2827 | 1785 | 100 | 82.7 |
| 237 | GPR3 | 2827 | 212766 | 10 | 20.2 |
| 237 | GPR3 | 2827 | 212766 | 30 | 89.3 |
| 237 | GPR3 | 2827 | 212766 | 100 | 85.9 |
| 242 | DCTD | 1635 | 119612 | 10 | 43 |
| 242 | DCTD | 1635 | 119612 | 30 | 37.5 |
| 242 | DCTD | 1635 | 119612 | 100 | 33.9 |
| 242 | DCTD | 1635 | 214555 | 10 | 13.5 |
| 242 | DCTD | 1635 | 214555 | 30 | 11.7 |
| 242 | DCTD | 1635 | 214555 | 100 | 9.6 |
| 261 | ACLY | 47 | 116939 | 10 | 318.9 |
| 261 | ACLY | 47 | 116939 | 30 | |
| 261 | ACLY | 47 | 116939 | 100 | 13.6 |
| 261 | ACLY | 47 | 116940 | 10 | 38.3 |
| 261 | ACLY | 47 | 116940 | 30 | 30.2 |
| 261 | ACLY | 47 | 116940 | 100 | 33.7 |
| 273 | AGXT2L1 | 64850 | 112236 | 10 | |
| 273 | AGXT2L1 | 64850 | 112236 | 30 | |
| 273 | AGXT2L1 | 64850 | 112236 | 100 | |
| 273 | AGXT2L1 | 64850 | 112237 | 10 | |
| 273 | AGXT2L1 | 64850 | 112237 | 30 | |
| 273 | AGXT2L1 | 64850 | 112237 | 100 | |
| 291 | ARSE | 415 | 119012 | 10 | 17.9 |
| 291 | ARSE | 415 | 119012 | 30 | 18.3 |
| 291 | ARSE | 415 | 119012 | 100 | 11 |
| 291 | ARSE | 415 | 214706 | 10 | 17.7 |
| 291 | ARSE | 415 | 214706 | 30 | 19.3 |
| 291 | ARSE | 415 | 214706 | 100 | 32.9 |
| 306 | NR2F2 | 7026 | 5922 | 10 | 61.8 |
| 306 | NR2F2 | 7026 | 5922 | 30 | 44.5 |
| 306 | NR2F2 | 7026 | 5922 | 100 | 27.2 |
| 306 | NR2F2 | 7026 | 45374 | 10 | 74.3 |
| 306 | NR2F2 | 7026 | 45374 | 30 | 51.9 |
| 306 | NR2F2 | 7026 | 45374 | 100 | 40 |
| 309 | ADAM18 | 8749 | 104120 | 10 | |
| 309 | ADAM18 | 8749 | 104120 | 30 | |
| 309 | ADAM18 | 8749 | 104120 | 100 | |
| 309 | ADAM18 | 8749 | 212787 | 10 | 107.7 |
| 309 | ADAM18 | 8749 | 212787 | 30 | 94.1 |
| 309 | ADAM18 | 8749 | 212787 | 100 | 75 |
| 334 | ARHGEF2 | 9181 | 119230 | 10 | 25.8 |
| 334 | ARHGEF2 | 9181 | 119230 | 30 | 32.4 |
| 334 | ARHGEF2 | 9181 | 119230 | 100 | 40.9 |
| 334 | ARHGEF2 | 9181 | 214562 | 10 | 49.7 |
| 334 | ARHGEF2 | 9181 | 214562 | 30 | 96.7 |
| 334 | ARHGEF2 | 9181 | 214562 | 100 | 21 |
| 435 | PRSS15 | 9361 | 105664 | 10 | 24.6 |
| 435 | PRSS15 | 9361 | 105664 | 30 | 16.2 |
| 435 | PRSS15 | 9361 | 105664 | 100 | 14.8 |
| 435 | PRSS15 | 9361 | 212758 | 10 | 1.6 |
| 435 | PRSS15 | 9361 | 212758 | 30 | 7.3 |
| 435 | PRSS15 | 9361 | 212758 | 100 | 17.9 |
| 459 | PCYT1B | 9468 | 111523 | 10 | 115 |
| 459 | PCYT1B | 9468 | 111523 | 30 | 31.5 |
| 459 | PCYT1B | 9468 | 111523 | 100 | 12.8 |
| 459 | PCYT1B | 9468 | 214260 | 10 | 12.5 |
| 459 | PCYT1B | 9468 | 214260 | 30 | 10.7 |
| 459 | PCYT1B | 9468 | 214260 | 100 | 9.8 |
| 486 | FLJ21736 | 79984 | 119838 | 10 | 29.3 |
| 486 | FLJ21736 | 79984 | 119838 | 30 | 12.8 |
| 486 | FLJ21736 | 79984 | 119838 | 100 | |
| 486 | FLJ21736 | 79984 | 235619 | 10 | 44.1 |
| 486 | FLJ21736 | 79984 | 235619 | 30 | 26.7 |
| 486 | FLJ21736 | 79984 | 235619 | 100 | |
| 495 | KIR2DS1 | 3806 | 212646 | 10 | |
| 495 | KIR2DS1 | 3806 | 212646 | 30 | |
| 495 | KIR2DS1 | 3806 | 212646 | 100 | |
| 495 | KIR2DS1 | 3806 | 235634 | 10 | |
| 495 | KIR2DS1 | 3806 | 235634 | 30 | |
| 495 | KIR2DS1 | 3806 | 235634 | 100 | |
| 496 | KIR2DS3 | 3808 | 213159 | 10 | |
| 496 | KIR2DS3 | 3808 | 213159 | 30 | |
| 496 | KIR2DS3 | 3808 | 213159 | 100 | |
| 496 | KIR2DS3 | 3808 | 235633 | 10 | |
| 496 | KIR2DS3 | 3808 | 235633 | 30 | |
| 496 | KIR2DS3 | 3808 | 235633 | 100 | |
| 506 | MOXD1 | 26002 | 111923 | 10 | 37.7 |
| 506 | MOXD1 | 26002 | 111923 | 30 | 44.7 |
| 506 | MOXD1 | 26002 | 111923 | 100 | |
| 506 | MOXD1 | 26002 | 235615 | 10 | 36.9 |
| 506 | MOXD1 | 26002 | 235615 | 30 | 46.4 |
| 506 | MOXD1 | 26002 | 235615 | 100 | |
| 514 | PEPD | 5184 | 105302 | 10 | 20.6 |
| 514 | PEPD | 5184 | 105302 | 30 | 6.5 |
| 514 | PEPD | 5184 | 105302 | 100 | 6 |
| 514 | PEPD | 5184 | 212688 | 10 | 55 |
| 514 | PEPD | 5184 | 212688 | 30 | 20.1 |
| 514 | PEPD | 5184 | 212688 | 100 | 32.4 |

TABLE 9

| | | | | | |
|---|---|---|---|---|---|
| Target | Target | | | siRNA sense sequence | SEQ-ID |
| No | Symbol | Gene Id | sirna_id | (21-mer) | No |

| | | | | | |
|---|---|---|---|---|---|
| 2 | HLCS | 3141 | siRNA ID | GCCUGAACCUUCUCUUGAGTT | 1 |
| 2 | HLCS | 3141 | 117852 | GGACGGUAUGGAGCAUGUUTT | 2 |
| 2 | HLCS | 3141 | 117853 | CGAAAGUUAACAAAACUCCTT | 3 |
| 3 | SC4MOL | 6307 | 117416 | CCAUUCGUUUAUUAGAAACTT | 4 |
| 3 | SC4MOL | 6307 | 117417 | CGUAAACCUUCCUGAAAGATT | 5 |
| 3 | SC4MOL | 6307 | 117418 | CCUUUAAUUACCUUCCUAGTT | 6 |
| 4 | CASP1 | 834 | 42626 | GACUCAUUGAACAUAUGCATT | 7 |
| 4 | CASP1 | 834 | 42711 | GAAUAUGCCUGUUCCUGUGTT | 8 |
| 7 | CTSE | 1510 | 105039 | GGCCCAUAUAUUGCAUUUATT | 9 |
| 7 | CTSE | 1510 | 105040 | GGUGUCAUUUGACAUGGUUTT | 10 |
| 7 | CTSE | 1510 | 105041 | GGAGGCAGAUAAUGCUGGUTT | 11 |
| 8 | FRK | 2444 | 1147 | GGCAGACAAGUCAACCGUGTT | 12 |
| 8 | FRK | 2444 | 1243 | GGCAUGGCCACUACUUUGUTT | 13 |
| 8 | FRK | 2444 | 1338 | GGACAGUCAAGGUGAUAUATT | 14 |
| 9 | HMBS | 3145 | 8225 | GGAAUGCAUGUAUGCUGUGTT | 15 |
| 9 | HMBS | 3145 | 117844 | CCAAUCCUACUAAUAAACCTT | 16 |
| 10 | HSD17B4 | 3295 | 106087 | GGAAUAUAUGGCAACUUUGTT | 17 |
| 10 | HSD17B4 | 3295 | 106089 | GGGCACACUACACUAUUAATT | 18 |
| 11 | LCN2 | 3934 | 121011 | CGAGUUACCUCGUCCGAGUTT | 19 |
| 11 | LCN2 | 3934 | 121012 | GCAUGCUAUGGUGUUCUUCTT | 20 |
| 12 | OXTR | 5021 | 1766 | GGUGCACAUCUUCUCUCUGTT | 21 |
| 12 | QXTR | 5021 | 1859 | GGCCUACAUCACAUGGAUCTT | 22 |
| 12 | OXTR | 5021 | 1947 | GGUACCUAUCAGUUUGUAUTT | 23 |
| 13 | PPP1R3C | 5507 | 142834 | CGACGACAUUUUGUGAAUATT | 24 |
| 13 | PPP1R3C | 5507 | 142836 | CCGAUUACUUAAGUUUCCGTT | 25 |
| 16 | TPI1 | 7167 | 43820 | GAGAGAAGGCAUGUCUUUGTT | 26 |
| 16 | TPI1 | 7167 | 43916 | GAGAAGGCAUGUCUuUGGGTT | 27 |
| 18 | SLC30A2 | 7780 | 117693 | GCCAGAAUACAAGUAUGUATT | 28 |
| 18 | SLC30A2 | 7780 | 117695 | CCAUCCUGAGAGAUGUGAUTT | 29 |
| 19 | ULK1 | 8408 | 118260 | GCGAAUUUUGUGUGAUUUCTT | 30 |
| 19 | ULK1 | 8408 | 118261 | CCCAAGCACUUUAUGCAUATT | 31 |
| 22 | SPUVE | 11098 | 19104 | GGCCAAGCAAUAUCUGUCUTT | 32 |
| 22 | SPUVE | 11098 | 19196 | GGGUCUUCAGGAAAGUCUCTT | 33 |
| 22 | SPUVE | 11098 | 212941 | CCUAUGUGAAAGGAACCCATT | 34 |
| 22 | SPUVE | 11098 | 212942 | CGAGACCUAUGACUUGCUCTT | 35 |
| 23 | PASK | 23178 | 978 | GGUCUGAACCAGUGGAUGUTT | 36 |
| 23 | PASK | 23178 | 103354 | GGACCAGCAAAUCACUGCCTT | 37 |
| 26 | MYLIP | 29116 | 118397 | GGAGCAGACUAGGCAUAUCTT | 38 |
| 26 | MYLIP | 29116 | 118398 | GCAGACUAGGCAUAUCUUUTT | 39 |
| 27 | PKD1L2 | 114780 | 104830 | GGAGGCCAUUUGGUCUUCATT | 40 |
| 27 | PKD1L2 | 114780 | 104835 | GGCGAUAUGGAGGUGUACATT | 41 |
| 28 | BPHL | 670 | 119221 | CGGUUUCAUGCCGACUUCATT | 42 |
| 28 | BPHL | 670 | 214767 | GGAAGAGAGCAUGAUAUAUTT | 43 |
| 29 | USP3 | 9960 | 105141 | GGCAGCCAGACUGCAUUUATT | 44 |
| 29 | USP3 | 9960 | 214567 | CCAACCAUAAGAAAUCAGATT | 45 |
| 31 | KCNK17 | 89822 | 43781 | GCUCUAAGGAAGACUUCAATT | 46 |
| 31 | KCNK17 | 89822 | 212814 | GGAUGCUAUCCAGAGGGACTT | 47 |
| 31 | KCNK17 | 89822 | 212815 | GGAAGACUUCAAGUCCCAATT | 48 |
| 32 | WEE1 | 7465 | 405 | GGUAUAUUCAUUCAAUGUCTT | 49 |
| 32 | WEE1 | 7465 | 103582 | GGACAGUGUCGUCGUAGAATT | 50 |
| 32 | WEE1 | 7465 | 103636 | GGCUGGAUGGAUGCAUUUATT | 51 |
| 32 | WEE1 | 7465 | 212739 | CCUGCUAAGAGAAUUACAATT | 52 |
| 34 | RNUT1 | 10073 | 117371 | CCAUUCUAGAUUGCAUUUATT | 53 |
| 34 | RNUT1 | 10073 | 214780 | GGGUUCUUCCCAUAGCCCATT | 54 |
| 35 | M6PR | 4074 | 111157 | GCGUGGCAAAGUCCAAGAUTT | 55 |
| 35 | M6PR | 4074 | 214744 | GGCAUGUGGUUUUGACCUUTT | 56 |
| 36 | MGC39650 | 147011 | 38979 | GGUGAUCAAGAAGGUAAAGTT | 57 |
| 36 | MGC39650 | 147011 | 214871 | CGUGAGCCGAUCAUUAAGCTT | 58 |
| 37 | FLJ22761 | 80201 | 121933 | GGAAUCAUUGCAUGCUUAATT | 59 |
| 37 | FLJ22761 | 80201 | 214839 | GCCAUCAAGAGGAGAAACGTT | 60 |
| 38 | PAPOLG | 64895 | 119829 | GCCUUGAUAUAAGGUGUAUTT | 61 |
| 38 | PAPOLG | 64895 | 214280 | CCAUUUGGAGUGUUUGAAGTT | 62 |
| 39 | DNM1L | 10059 | 19471 | GGAAAUAAUAAGGGAGUAATT | 63 |
| 39 | DNM1L | 10059 | 214799 | GGCUAGCCAGAGAAUUACCTT | 64 |
| 43 | LCN7 | 64129 | 105753 | GUGGCAGUGUGACCAAGAATT | 65 |
| 43 | LCN7 | 64129 | 214577 | GCCAGGACACCUCAAGUCUTT | 66 |
| 45 | RASGRP2 | 10235 | 120445 | GGCUCUGCUAGACCAAGAATT | 67 |
| 45 | RASGRP2 | 10235 | 214874 | GCAGCCUAAUCGACAUAGATT | 68 |
| 51 | PRPSAP2 | 5636 | 117084 | GCUCCUGAUCAUGGUGUAUTT | 69 |
| 51 | PRPSAP2 | 5636 | 214750 | CCAACUUUUUAUGUCGGUUTT | 70 |
| 52 | SLC26A10 | 65012 | 119926 | GGAGAAAAGGAGACUUCAATT | 71 |
| 52 | SLC26A10 | 65012 | 214589 | CCUCAUCAUGUGGAGUGGATT | 72 |
| 56 | TNFRSF13B | 23495 | 111813 | CCCUAAGCAAUGUGCAUACTT | 73 |
| 56 | TNFRSF13B | 23495 | 214802 | GCAAGGCAAGUUCUAUGACTT | 74 |
| 62 | CTSK | 1513 | 105010 | GGAAGAGAGUUGUAUGUACTT | 75 |
| 62 | CTSK | 1513 | 214550 | GGCUGGAGAUUUUCACAUATT | 76 |
| 72 | D4ST-1 | 113189 | 112330 | GGAUGUGCUGCCUAAGUAUTT | 77 |
| 72 | D4ST-1 | 113189 | 214285 | GGCCUUUGAGGUUGUGACUTT | 78 |
| 73 | APCL | 10297 | 121576 | CGUGUAAAUAGUGGUAAAUTT | 79 |
| 73 | APCL | 10297 | 214783 | CGAACAGUGAUCUACGUCCTT | 80 |
| 79 | ARHGEF1 | 9138 | 119422 | CCGAUCACAAAGCCUUCUATT | 81 |
| 79 | ARHGEF1 | 9138 | 214561 | GCCUUCUACGUCCUUUUUATT | 82 |
| 81 | MCCC1 | 56922 | 118817 | GCCAAAAAACUGGGUGUACTT | 83 |
| 81 | MCCC1 | 56922 | 214276 | CCAACAUUGACUUCUUACUTT | 84 |
| 88 | GALR2 | 8811 | 4818 | GGUGACACGCAUGAUCCUCTT | 85 |
| 89 | GALR2 | 8811 | 44971 | GCACUACCAACCUGUUCAUTT | 86 |
| 88 | GALR2 | 8811 | 45063 | GCAUCCUGACGGUUGAUGUTT | 87 |
| 88 | GALR2 | 8811 | 212858 | CCUGUGUUUCAUCCUGUGCTT | 88 |
| 117 | SMPD1 | 6609 | 8630 | GGUUACAUCGCAUAGUGCCTT | 89 |
| 117 | SMPD1 | 6609 | 8725 | GGUCUAUUCACCGCCAUCATT | 90 |
| 117 | SMPD1 | 6609 | 8816 | GGAGACAAAGUGCAUAUAATT | 91 |
| 117 | SMPD1 | 6609 | 212695 | GCCCAAAUGCUGCUGUGGUTT | 92 |
| 143 | SCP2 | 6342 | 117110 | CCAGGCAUGUGUUGGCUAUTT | 93 |
| 143 | SCP2 | 6342 | 119182 | CCUCCUGAUCAAUAAGUAUTT | 94 |
| 143 | SCP2 | 6342 | 214903 | CGAACUCCUUACUUAUGAATT | 95 |
| 163 | CCM1 | 889 | 15563 | GGAACGACAGUGGGUAGAUTT | 96 |
| 163 | CCM1 | 889 | 214883 | GGCUGGUCUCGUGGUAAAATT | 97 |
| 171 | PAFAH2 | 5051 | 119066 | GCGAGUGUUUACGGGUGUUTT | 98 |
| 171 | PAFAH2 | 5051 | 214710 | CCCAUGAGCUCCUAUCAAGTT | 99 |
| 180 | GAD2 | 2572 | 9108 | GGCACAGGUGUAAAUAUAGTT | 100 |
| 180 | GAD2 | 2572 | 214716 | CGUGCUCUUCUGUUCUCAATT | 101 |
| 205 | HTR2C | 3358 | 1758 | GGCCAUCAUGAAGAUUGCUTT | 102 |
| 205 | HTR2C | 3358 | 1852 | GGGACGAAGAAAAGGUGUUTT | 103 |
| 205 | HTR2C | 3358 | 1940 | GGUGAUGAAUUUACGAUCATT | 104 |
| 205 | HTR2C | 3358 | 144642 | GCACUUUCAAUCGUCAUCATT | 105 |
| 205 | HTR2C | 3358 | 212705 | GCCCAGUAGCAGCUAUAGUTT | 106 |
| 215 | LOC345667 | 11174 | 104231 | GGAGGUGGUCUGUAAAAGGTT | 107 |
| 215 | LOC345667 | 11174 | 104232 | GGCAUCUAGUGACUGUCUATT | 108 |
| 215 | LOC345667 | 11174 | 104267 | GGGAGACUUGCUUACUGUGTT | 109 |
| 215 | LOC345667 | 11174 | 212853 | GGUUCAGAAUUCUUAAGUCTT | 110 |
| 237 | GPR3 | 2827 | 1785 | GGAUGUGCAGAAAGUGCUGTT | 111 |
| 237 | GPR3 | 2827 | 212766 | CCUCUCUACACCUAUCUUATT | 112 |
| 240 | FLJ32389 | 126393 | 122036 | CCAAACUGUACAGACUCUCTT | 113 |
| 240 | FLJ32389 | 128393 | 214864 | CCAGAUAUCCUCGGCAACCTT | 114 |
| 241 | SERPINA7 | 6906 | 118553 | GCCAAGCAGGAGAUUAACATT | 115 |
| 241 | SERPINA7 | 6906 | 214548 | GGCCAUUGGCUAAUUGCACTT | 116 |
| 242 | DCTD | 1635 | 119612 | CCGAUGUGAAAGGCUGUAGTT | 117 |
| 242 | DCTD | 1635 | 214555 | GCAAGAUUGUCAUUGACUUTT | 118 |
| 261 | ACLY | 47 | 116939 | GGAGUUCUAUGUCUGCAUCTT | 119 |
| 261 | ACLY | 47 | 116940 | GCACGAAGUCACAAUCUUUTT | 120 |
| 261 | ACLY | 47 | 214886 | GCAAUUCGAGAUUACCAGGTT | 121 |
| 273 | AGXT2L1 | 64850 | 112236 | CGACAACAUUGUUGAGUAUTT | 122 |
| 273 | AGXT2L1 | 64850 | 112237 | GCCAACGAGUUAGGCUUACTT | 123 |
| 273 | AGXT2L1 | 64850 | 214281 | CCGAAAGUGUGACCUCUGATT | 124 |
| 291 | ARSE | 415 | 119012 | GGCUAUGCCACUGGACUCATT | 125 |
| 291 | ARSE | 415 | 214706 | CGAGUUCCUGAUGCAUUAUTT | 126 |
| 292 | ITGB8 | 3696 | 11202 | GGAUUUCAUUUCAGGUGGATT | 127 |
| 292 | ITGB8 | 3696 | 214742 | CCCUCACAAUUUGUCUCAGTT | 128 |
| 306 | NR2F2 | 7026 | 5922 | GGCCAUAGUCCUGUUCACCTT | 129 |
| 306 | NR2F2 | 7026 | 45374 | GAGCUUCUUCAAGCGCAGCTT | 130 |
| 306 | NR2F2 | 7026 | 212809 | GCUGUUUGUGUUGAAUGCGTT | 131 |
| 309 | ADAM18 | 8749 | 21148 | GGGAUUUUCGGUUAUUAUATT | 132 |
| 309 | ADAM18 | 8749 | 104119 | GGGCUCAGUAAAAUGUGGUTT | 133 |
| 309 | ADAM18 | 8749 | 104120 | GGGAAAGGGAUAUGUAAUATT | 134 |
| 309 | ADAM18 | 8749 | 212787 | GCAAGAUUUGAGCAUAUAATT | 135 |
| 334 | ARHGEF2 | 9181 | 119230 | GGUAAUCUACAGUGAGCUGTT | 136 |
| 334 | ARHGEF2 | 9181 | 214562 | CCAACAUUGCUGGACAUUUTT | 137 |
| 352 | PRP2 | 134285 | 43202 | GCAUUGGGAUAUUAAGUAGTT | 138 |
| 352 | LOC134285 | 134285 | 46549 | GUCCUGCUCUCCAUGUUUGTT | 139 |
| 352 | LOC134285 | 134285 | 128215 | CCGCUAAACGAGACAGACATT | 140 |
| 352 | LOC134285 | 134285 | 212841 | GGGACAGAUCCAGAUUAUGTT | 141 |
| 363 | PLA2R1 | 22925 | 108254 | GGUACGCUGUUAAGUAUUATT | 142 |
| 363 | PLA2R1 | 22925 | 214723 | GCACAUGAGCAGUAAAACATT | 143 |
| 390 | CREB5 | 9586 | 116759 | GCAAGCGGAAUAUCUCGAUTT | 144 |
| 390 | CREB5 | 9586 | 116760 | CCUUUUCUGUAUAUAGCCATT | 145 |
| 390 | CREB5 | 9586 | 214882 | GCAUAAUACCAUCACUACUTT | 146 |
| 413 | FEN1 | 2237 | 121476 | GCUACUUUGGCCGUAAGGUTT | 147 |
| 413 | FEN1 | 2237 | 214763 | GCUCUUCUUGGAACCUGAGTT | 148 |
| 435 | PRSS15 | 9361 | 105664 | GAGAUGACAGCAAUGAGUCTT | 149 |
| 435 | PRSS15 | 9361 | 212757 | GCAGCUAAAGAUCAUGAAGTT | 150 |
| 435 | PRSS15 | 9361 | 212758 | GCUAAAGAUCAUCAAGAAGTT | 151 |
| 441 | SYNJ1 | 8867 | 104702 | GCCAAUCAAGGGUACAUACTT | 152 |
| 441 | SYNJ1 | 8867 | 212746 | GGCAUUUUAGAACACUUAATT | 153 |
| 441 | SYNJ1 | 8867 | 212747 | GGCCAUUGAUGUUUUGCUATT | 154 |
| 459 | PCYT1B | 9468 | 111523 | CGAAGUUAUCAGAGAUGCUTT | 155 |
| 459 | PCYT1B | 9468 | 214260 | CCUCCAGAGAGGGUAUACATT | 156 |
| 486 | FLJ21736 | 79984 | 119838 | GCAGAUCUUCUCCGUUUCATT | 157 |
| 486 | FLJ21736 | 79984 | 235619 | CCACUUGACUCUCUGUAAATT | 158 |
| 489 | GPR10 | 2834 | 103837 | CCGUCUAUGUGUCGGUGUUTT | 159 |
| 489 | GPR10 | 2834 | 235614 | CCUAUCACGUGGAGCUCAATT | 160 |
| 495 | KIR2DS1 | 3806 | 212646 | GCAUGGCGUGUGUUGGGUUTT | 161 |
| 495 | KIR2DS1 | 3806 | 235634 | CCAUCCUCCUCUUCUUUCUTT | 162 |
| 496 | KIR2DS3 | 3808 | 213159 | GCAUGGCAUGUGUUGGGUUTT | 163 |
| 496 | KIR2DS3 | 3808 | 235633 | CAGGAAACCCUUCAAAUAGTT | 164 |
| 498 | LOC135896 | 135896 | 213242 | CCAUUAUGAGCCCAAGAAUTT | 165 |
| 498 | LOC135896 | 135896 | 235629 | GGCUGCAUCGCUCAAAUCUTT | 166 |
| 506 | MOXD1 | 26002 | 111923 | GCUGCAUACAUGUGACAUATT | 167 |
| 506 | MOXD1 | 26002 | 235615 | GCCUUACCAUACUUUGAUCTT | 168 |
| 507 | MPN2 | 339501 | 113991 | CCAGGGAAUCUCCCUAACUTT | 169 |
| 507 | MPN2 | 339501 | 235626 | CCCAGUGGUAUGAGGUGAATT | 170 |
| 514 | PEPD | 5184 | 105302 | GUACGUAGAUGAGAUUGCCTT | 171 |
| 514 | PEPD | 5184 | 212688 | CCAAACAGUGCUGUUUCCCTT | 172 |

TABLE 10

| Target | Target | Gene | | |
|---|---|---|---|---|
| No | Symbol | Id | RefSeq. Acc. | Target description |
| 2 | HLCS | 3141 | NM_000411 | Homo sapiens holocarboxylase synthetase (biotin- |
| | | | | [proprionyl-Coenzyme A-carboxylase |
| 3 | SC4MOL | 6307 | NM_006745 | Homo sapiens sterol-C4-methyl oxidase-like (SC4MOL), |
| | | | | mRNA. |
| 4 | CASP1 | 834 | NM_033295 | Homo sapiens caspase 1, apoptosis-related cysteine |
| | | | | protease (interleukin 1, beta, convertase) |
| 7 | CTSE | 1510 | NM_001910 | Homo sapiens cathepsin E (CTSE), transcript variant 1, |
| | | | | mRNA. |
| 8 | FRK | 2444 | NM_002031 | Homo sapiens fyn-related kinase (FRK), mRNA. |
| 9 | HMBS | 3145 | NM_000190 | Homo sapiens hydroxymethylbilane synthase(HMBS), |
| | | | | mRNA. |
| 10 | HSD17B4 | 3295 | NM_000414 | Homo sapiens hydroxysteroid (17-beta) dehydrogenase 4 |
| | | | | (HSD17B4), mRNA. |
| 11 | LCN2 | 3934 | NM_005564 | Homo sapiens lipocalin 2 (oncogene 24p3) (LCN2), mRNA. |
| 12 | OXTR | 5021 | NM_000916 | Homo sapiens oxytocin receptor (OXTR), mRNA. |
| 13 | PPP1R3C | 5507 | NM_005398 | Homo sapiens protein phosphatase 1, regulatory (inhibitor) |
| | | | | subunit 3C (PPP1R3C), mRNA. |
| 16 | TPI1 | 7167 | NM_000365 | Homo sapiens triosephosphate isomerase 1 (TPI1), mRNA. |
| 18 | SLC30A2 | 7780 | NM_032513 | Homo sapiens solute carrier family 30 (zinc transporter), |
| | | | | member 2 (SLC30A2), mRNA. |
| 19 | ULK1 | 8408 | NM_003565 | Homo sapiens unc-51-like kinase 1 (C. elegans) (ULK1), |
| | | | | mRNA. |
| 22 | SPUVE | 11098 | NM_007173 | Homo sapiens protease, serine, 23 (PRSS23), mRNA. |
| 23 | PASK | 23178 | NM_015148 | Homo sapiens PAS domain containing serine/threonine |
| | | | | kinase (PASK), mRNA. |
| 26 | MYLIP | 29116 | NM_013262 | Homo sapiens myosin regulatory light chain interacting |
| | | | | protein (MYLIP), mRNA. |
| 27 | PKD1L2 | 114780 | NM_182740 | Homo sapiens polycystic kidney disease 1-like 2 (PKD1L2), |
| | | | | transcript variant 2, mRNA. |
| 28 | BPHL | 670 | NM_004332 | Homo sapiens biphenyl hydrolase-like (serine hydrolase; |
| | | | | breast epithelial mucin-associated antigen) |
| 29 | USP3 | 9960 | NM_006537 | Homo sapiens ubiquitin specific protease 3 (USP3), mRNA. |
| 31 | KCNK17 | 89822 | NM_031460 | Homo sapiens potassium channel, subfamily K, member 17 |
| | | | | (KCNK17), mRNA. |
| 32 | WEE1 | 7465 | NM_003390 | Homo sapiens WEE1 homolog (S. pombe) (WEE1), mRNA. |
| 34 | RNUT1 | 10073 | NM_005701 | Homo sapiens RNA, U transporter 1 (RNUT1), mRNA. |
| 35 | M6PR | 4074 | NM_002355 | Homo sapiens mannose-6-phosphate receptor (cation |
| | | | | dependent) (M6PR), mRNA. |
| 36 | MGC39650 | 147011 | NM_152465 | Homo sapiens hypothetical protein MGC39650 (MGC39650), |
| | | | | mRNA. |
| 37 | FLJ22761 | 80201 | NM_025130 | Homo sapiens hypothetical protein FLJ22761 (FLJ22761), |
| | | | | mRNA. |
| 38 | PAPOLG | 64895 | NM_022894 | Homo sapiens poly(A) polymerase gamma (PAPOLG), |
| | | | | mRNA. |
| 39 | DNM1L | 10059 | NM_012062 | Homo sapiens dynamin 1-like (DNM1L), transcript variant 1, |
| | | | | mRNA. |
| 43 | LCN7 | 64129 | NM_022164 | Homo sapiens lipocalin 7 (LCN7), mRNA. |
| 45 | RASGRP2 | 10235 | NM_153819 | Homo sapiens RAS guanyl releasing protein 2 (calcium and |
| | | | | DAG-regulated) (RASGRP2), |
| 51 | PRPSAP2 | 5636 | NM_002767 | Homo sapiens phosphoribosyl pyrophosphate synthetase- |
| | | | | associated protein 2 (PRPSAP2), mRNA. |
| 52 | SLC26A10 | 65012 | NM_133489 | Homo sapiens solute carrier family 26, member 10 |
| | | | | (SLC26A10), mRNA. |
| 56 | TNFRSF13B | 23495 | NM_012452 | Homo sapiens tumor necrosis factor receptor superfamily, |
| | | | | member 13B (TNFRSF13B), mRNA. |
| 62 | CTSK | 1513 | NM_000396 | Homo sapiens cathepsin K (pycnodysostosis) (CTSK), |
| | | | | mRNA. |
| 72 | D4ST1 | 113189 | NM_130468 | Homo sapiens dermatan 4 sulfotransferase 1 (D4ST1), |
| | | | | mRNA. |
| 73 | APCL/APC2 | 10297 | NM_005883 | Homo sapiens adenomatosis polyposis coil 2 (APC2), mRNA. |
| 79 | ARHGEF1 | 9138 | NM_004706 | Homo sapiens Rho guanine nucleotide exchange factor |
| | | | | (GEF) 1 (ARHGEF1), transcript variant 2, |
| 81 | MCCC1 | 56922 | NM_020166 | Homo sapiens methylcrotonoyl-Coenzyme A carboxylase 1 |
| | | | | (alpha) (MCCC1), mRNA. |
| 88 | GALR2 | 8811 | NM_003857 | Homo sapiens galanin receptor 2 (GALR2), mRNA. |
| 117 | SMPD1 | 6609 | NM_000543 | Homo sapiens sphingomyelin phosphodiesterase 1, acid |
| | | | | lysosomal (acid sphingomyelinase) (SMPD1) |
| 143 | SCP2 | 6342 | NM_002979 | Homo sapiens sterol carrier protein 2 (SCP2), mRNA. |
| 163 | CCM1 | 889 | NM_194456 | Homo sapiens cerebral cavernous malformations 1 (CCM1), |
| | | | | transcript variant 1, mRNA. |
| 171 | PAFAH2 | 5051 | NM_000437 | Homo sapiens platelet-activating factor acetylhydrolase 2, |
| | | | | 40 kDa (PAFAH2), mRNA. |
| 180 | GAD2 | 2572 | NM_000818 | Homo sapiens glutamate decarboxylase 2 (pancreatic islets |
| | | | | and brain, 65 kDa (GAD2), mRNA. |
| 205 | HTR2C | 3358 | NM_000868 | Homo sapiens 5-hydroxytryptamine (serotonin) receptor 2C |
| | | | | (HTR2C), mRNA. |
| 215 | LOC345667 | 11174 | NM_197941 | Homo sapiens a-disintegrin-and-metalloprotease with |
| | | | | thrombospondin type 1 motif 6 |
| 215 | LOC345667 | 345667 | NM_197941 | Homo sapiens similar to ADAMTS-10 precursor |
| 237 | GPR3 | 2827 | NM_005281 | Homo sapiens G protein-coupled receptor 3 (GPR3), mRNA. |
| 240 | FLJ32389 | 126393 | NM_144617 | Homo sapiens heat shock protein, alpha-crystallin-related, B6 |
| | | | | (HSPB6), mRNA. |
| 241 | SERPINA7 | 6906 | NM_000354 | Homo sapiens serine (or cysteine) proteinase inhibitor, clade |
| | | | | A (alpha-1 antiproteinase, antitrypsin) |
| 242 | DCTD | 1635 | NM_001921 | Homo sapiens dCMP deaminase (DCTD), mRNA. |
| 261 | ACLY | 47 | NM_198830 | Homo sapiens ATP citrate lyase (ACLY), transcript variant 2, |
| | | | | mRNA. |
| 273 | AGXT2L1 | 64850 | NM_031279 | Homo sapiens alanine-glyoxylate aminotransferase 2-like 1 |
| | | | | (AGXT2L1), mRNA. |
| 291 | ARSE | 415 | NM_000047 | Homo sapiens arylsulfatase E (chondrodysplasia punctata 1) |
| | | | | (ARSE), mRNA. |
| 292 | ITGB8 | 3696 | NM_002214 | Homo sapiens integrin, beta 8 (ITGB8), mRNA. |
| 306 | NR2F2 | 7026 | NM_021005 | Homo sapiens nuclear receptor subfamily 2, group F, |
| | | | | member 2 (NR2F2), mRNA. |
| 309 | ADAM18 | 8749 | NM_014237 | Homo sapiens a disintegrin and metalloproteinase domain 18 |
| | | | | (ADAM18), mRNA. |
| 334 | ARHGEF2 | 9181 | NM_004723 | Homo sapiens rho/rac guanine nucleotide exchange factor |
| | | | | (GEF) 2 (ARHGEF2), mRNA. |
| 352 | LOC134285 | 134285 | NM_173490 | Homo sapiens hypothetical protein LOC134285 |
| | | | | (LOC134285), mRNA. |
| 363 | PLA2R1 | 22925 | NM_007366 | Homo sapiens phospholipase A2 receptor 1, 180 kDa |
| | | | | (PLA2R1), mRNA. |
| 390 | CREB5 | 9586 | NM_182899 | Homo sapiens cAMP responsive element binding protein 5 |
| | | | | (CREB5), mRNA. |
| 413 | FEN1 | 2237 | NM_004111 | Homo sapiens flap structure-specific endonuclease 1 (FEN1), |
| | | | | mRNA. |
| 435 | PRSS15 | 9361 | NM_004793 | Homo sapiens protease, serine, 15 (PRSS15), nuclear gene |
| | | | | encoding mitochondrial protein, |
| 441 | SYNJ1 | 8867 | NM_003895 | Homo sapiens synaptojanin 1 (SYNJ1), transcript variant 1, |
| | | | | mRNA. |
| 459 | PCYT1B | 9468 | NM_004845 | Homo sapiens phosphate cytidylyltransferase 1, choline, beta |
| | | | | isoform (PCYT1B), mRNA. |
| 486 | FLJ21736 | 79984 | NM_024922 | Homo sapiens esterase 31 |
| 489 | GPR10 | 2834 | NM_004248 | Homo sapiens prolactin releasing hormone receptor |
| 495 | KIR2DS1 | 3806 | NM_014512 | Homo sapiens killer cell immunoglobulin-like receptor 1 |
| 496 | KIR2DS3 | 3806 | NM_012313 | Homo sapiens killer cell immunoglobulin-like receptor 3 |
| 498 | LOC135896 | 135866 | NM_001005221 | Homo sapiens olfactory receptor, family 4, subfamily F, |
| | | | | member 29 |
| 506 | MOXD1 | 26002 | NM_015529 | Homo sapiens monooxygenase, DBH-like 1 |
| 507 | MPN2 | 339501 | NM_183062 | Homo sapiens marapsin 2 |
| 514 | PEPD | 5184 | NM_000285 | Homo sapiens peptidase D |

TABLE 11

| | | | | LDL-Dil run1 | LDL-Dil | LDL-Dil run2 | LDL-Dil |
|---|---|---|---|---|---|---|---|
| Target No | Target Symbol | Gene Id | siRNA ID | Mean % | run1 SD % | Mean % | run2 SD % |
| 2 | HLCS | 3141 | 117851 | 426.6 | 88.5 | | |
| 2 | HLCS | 3141 | 117852 | 432.1 | 111.4 | | |
| 2 | HLCS | 3141 | 117853 | 734.4 | 43.3 | | |
| 3 | SC4MOL | 6307 | 117416 | 366.7 | 53.0 | | |
| 3 | SC4MOL | 6307 | 117417 | 345.9 | 27.2 | | |
| 3 | SC4MOL | 6307 | 117418 | 563.4 | 120.6 | | |
| 4 | CASP1 | 834 | 42626 | 285.6 | 75.4 | 269.4 | 118.7 |
| 4 | CASP1 | 834 | 42711 | 456.2 | 162.2 | 250.9 | 153.6 |
| 7 | CTSE | 1510 | 105039 | 546.3 | 49.5 | 637.2 | 42.8 |
| 7 | CTSE | 1510 | 105040 | 114.4 | 16.2 | 103.1 | 17.0 |
| 7 | CTSE | 1510 | 105041 | 171.8 | 46.2 | 152.8 | 34.6 |
| 8 | FRK | 2444 | 1147 | 214.8 | 6.3 | 196.7 | 44.1 |
| 8 | FRK | 2444 | 1243 | 289.4 | 12.2 | 305.7 | 9.8 |
| 8 | FRK | 2444 | 1338 | 57.4 | 16.0 | 57.9 | 16.7 |
| 9 | HMBS | 3145 | 8225 | 461.5 | 41.1 | | |
| 9 | HMBS | 3145 | 117844 | 240.0 | 23.8 | | |
| 10 | HSD17B4 | 3295 | 106087 | 504.1 | 31.0 | | |
| 10 | HSD17B4 | 3295 | 106089 | 168.1 | 24.1 | 225.9 | 6.6 |
| 11 | LCN2 | 3934 | 121011 | 408.0 | 96.1 | | |
| 11 | LCN2 | 3934 | 121012 | 281.3 | 47.5 | | |
| 12 | OXTR | 5021 | 1766 | 223.2 | 29.7 | 197.2 | 22.6 |
| 12 | OXTR | 5021 | 1859 | 112.2 | 11.7 | 98.2 | 7.1 |
| 12 | OXTR | 5021 | 1947 | 341.8 | 52.4 | 313.6 | 8.2 |
| 13 | PPP1R3C | 5507 | 142834 | 259.3 | 69.1 | | |
| 13 | PPP1R3C | 5507 | 142836 | 291.4 | 81.0 | | |
| 16 | TPI1 | 7167 | 43820 | 268.6 | 76.3 | | |
| 16 | TPI1 | 7167 | 43916 | 613.4 | 61.9 | | |
| 18 | SLC30A2 | 7780 | 117693 | 361.0 | 48.1 | | |
| 18 | SLC30A2 | 7780 | 117695 | 417.5 | 25.0 | | |
| 19 | ULK1 | 8408 | 118260 | 359.7 | 84.5 | | |
| 19 | ULK1 | 8408 | 118261 | 900.1 | 81.2 | | |
| 22 | SPUVE | 11098 | 19104 | 425.1 | 14.8 | 644.6 | 57.9 |
| 22 | SPUVE | 11098 | 19196 | 378.1 | 11.9 | 262.9 | 5.9 |
| 22 | SPUVE | 11098 | 212941 | 33.8 | 5.1 | 26.1 | 0.4 |
| 22 | SPUVE | 11098 | 212942 | 151.4 | 37.2 | | |
| 23 | PASK | 23178 | 978 | 220.5 | 11.7 | | |
| 23 | PASK | 23178 | 103354 | 426.8 | 55.7 | | |
| 26 | MYLIP | 29116 | 118397 | 600.0 | 39.7 | | |
| 26 | MYLIP | 29116 | 118398 | 427.7 | 10.9 | | |
| 27 | PKD1L2 | 114780 | 104830 | 196.5 | 22.2 | 258.0 | 30.5 |
| 27 | PKD1L2 | 114780 | 104835 | 643.6 | 75.3 | | |
| 28 | BPHL | 670 | 119221 | 1438.1 | 168.4 | | |
| 28 | BPHL | 670 | 214767 | 212.0 | 16.0 | | |
| 29 | USP3 | 9960 | 105141 | 1754.7 | 235.5 | | |
| 29 | USP3 | 9960 | 214567 | 81.7 | 14.0 | | |
| 31 | KCNK17 | 89822 | 43781 | 668.4 | 52.8 | 1237.7 | 1.7 |
| 31 | KCNK17 | 89822 | 212814 | 68.8 | 3.9 | | |
| 31 | KCNK17 | 89822 | 212815 | 80.5 | 12.4 | | |
| 32 | WEE1 | 7465 | 405 | 180.2 | 4.2 | 188.5 | 19.0 |
| 32 | WEE1 | 7465 | 103582 | 206.2 | 30.3 | 188.8 | 29.3 |
| 32 | WEE1 | 7465 | 103636 | 1082.7 | 66.5 | 1574.7 | 286.1 |
| 32 | WEE1 | 7465 | 212739 | 94.0 | 23.4 | 149.1 | 7.0 |
| 34 | RNUT1 | 10073 | 117371 | 1078.8 | 131.4 | | |
| 34 | RNUT1 | 10073 | 214780 | 143.6 | 20.4 | | |
| 35 | M6PR | 4074 | 111157 | 994.9 | 56.9 | | |
| 35 | M6PR | 4074 | 214744 | 92.7 | 10.9 | | |
| 36 | MGC39650 | 147011 | 38979 | 1220.6 | 147.4 | | |
| 36 | MGC39650 | 147011 | 214871 | 105.6 | 20.5 | | |
| 37 | FLJ22761 | 80201 | 121933 | 780.4 | 25.7 | | |
| 37 | FLJ22761 | 80201 | 214839 | 90.2 | 27.1 | | |
| 38 | PAPOLG | 64895 | 119829 | 1316.8 | 157.7 | | |
| 38 | PAPOLG | 64895 | 214280 | 219.6 | 18.8 | 181.0 | 4.3 |
| 39 | DNM1L | 10059 | 19471 | 549.3 | 58.1 | | |
| 39 | DNM1L | 10059 | 214799 | 409.8 | 36.5 | | |
| 43 | LCN7 | 64129 | 105753 | 1224.3 | 79.9 | | |
| 43 | LCN7 | 64129 | 214577 | 59.6 | 14.7 | | |
| 45 | RASGRP2 | 10235 | 120445 | 945.5 | 215.1 | | |
| 45 | RASGRP2 | 10235 | 214874 | 50.8 | 9.3 | | |
| 51 | PRPSAP2 | 5636 | 117084 | 1329.6 | 118.7 | | |
| 51 | PRPSAP2 | 5636 | 214750 | 127.5 | 23.4 | | |
| 52 | SLC26A10 | 65012 | 119926 | 1670.2 | 49.7 | | |
| 52 | SLC26A10 | 65012 | 214589 | 99.5 | 21.4 | | |
| 56 | TNFRSF13B | 23495 | 111813 | 882.2 | 73.0 | | |
| 56 | TNFRSF13B | 23495 | 214802 | 252.6 | 90.2 | | |
| 62 | CTSK | 1513 | 105010 | 773.5 | 220.9 | | |
| 62 | CTSK | 1513 | 214550 | 265.0 | 33.7 | | |
| 72 | D4ST-1 | 113189 | 112330 | 606.4 | 27.7 | 801.4 | 72.1 |
| 72 | D4ST-1 | 113189 | 214285 | 148.0 | 18.9 | 149.3 | 11.5 |
| 73 | APCL | 10297 | 121576 | 746.2 | 76.7 | | |
| 73 | APCL | 10297 | 214783 | 493.9 | 15.5 | | |
| 79 | ARHGEF1 | 9138 | 119422 | 843.4 | 82.4 | | |
| 79 | ARHGEF1 | 9138 | 214561 | 253.0 | 11.6 | | |
| 81 | MCCC1 | 56922 | 118817 | 528.8 | 15.9 | | |
| 81 | MCCC1 | 56922 | 214276 | 39.3 | 9.6 | 35.0 | 8.1 |
| 88 | GALR2 | 8811 | 4818 | 97.2 | 16.0 | 106.1 | 14.9 |
| 88 | GALR2 | 8811 | 44971 | 585.7 | 52.5 | 638.9 | 88.2 |
| 88 | GALR2 | 8811 | 45063 | 77.0 | 13.7 | 81.7 | 29.9 |
| 88 | GALR2 | 8811 | 212858 | 173.5 | 15.7 | 229.8 | 12.4 |
| 117 | SMPD1 | 6609 | 8630 | 293.5 | 17.6 | 262.1 | 20.3 |
| 117 | SMPD1 | 6609 | 8725 | 222.3 | 51.9 | 167.8 | 22.8 |
| 117 | SMPD1 | 6609 | 8816 | 666.0 | 192.1 | | |
| 117 | SMPD1 | 6609 | 212695 | 243.0 | 35.6 | | |
| 143 | SCP2 | 6342 | 119182 | 676.7 | 75.9 | | |
| 143 | SCP2 | 6342 | 214903 | 47.6 | 8.7 | | |
| 163 | CCM1 | 889 | 15563 | 229.6 | 30.4 | | |
| 163 | CCM1 | 889 | 214883 | 795.9 | 148.6 | | |
| 171 | PAFAH2 | 5051 | 119066 | 385.6 | 71.3 | | |
| 171 | PAFAH2 | 5051 | 214710 | 430.2 | 77.1 | | |
| 180 | GAD2 | 2572 | 9108 | 491.9 | 145.2 | | |
| 180 | GAD2 | 2572 | 214716 | 300.0 | 43.1 | | |
| 205 | HTR2C | 3358 | 1758 | 166.5 | 27.1 | 171.1 | 9.0 |
| 205 | HTR2C | 3358 | 1852 | 404.6 | 48.1 | 421.5 | 52.4 |
| 205 | HTR2C | 3358 | 1940 | 58.9 | 10.9 | 60.2 | 0.9 |
| 205 | HTR2C | 3358 | 144642 | 75.0 | 13.5 | | |
| 205 | HTR2C | 3358 | 212705 | 161.2 | 25.0 | | |
| 215 | LOC345667 | 11174 | 104231 | 274.2 | 19.6 | 247.1 | 14.1 |
| 215 | LOC345667 | 11174 | 104232 | 238.9 | 30.7 | 254.0 | 19.1 |
| 215 | LOC345667 | 11174 | 104267 | 34.7 | 7.9 | 36.3 | 3.4 |
| 215 | LOC345667 | 11174 | 212853 | 691.6 | 168.0 | | |
| 237 | GPR3 | 2827 | 1785 | 452.1 | 51.8 | 644.0 | 93.4 |
| 237 | GPR3 | 2827 | 212766 | 132.2 | 15.7 | | |
| 240 | FLJ32389 | 126393 | 122036 | 410.7 | 91.6 | | |
| 240 | FLJ32389 | 126393 | 214864 | 160.4 | 21.5 | | |
| 241 | SERPINA7 | 6906 | 118553 | 472.2 | 42.0 | | |
| 241 | SERPINA7 | 6906 | 214548 | 879.2 | 107.2 | | |
| 242 | DCTD | 1635 | 119612 | 685.4 | 60.3 | | |
| 242 | DCTD | 1635 | 214555 | 304.2 | 17.8 | | |
| 261 | ACLY | 47 | 116939 | 786.4 | 89.4 | | |
| 261 | ACLY | 47 | 214886 | 242.0 | 30.2 | | |
| 273 | AGXT2L1 | 64850 | 112237 | 369.4 | 92.9 | | |
| 273 | AGXT2L1 | 64850 | 214281 | 73.4 | 7.2 | 91.2 | 11.6 |
| 291 | ARSE | 415 | 119012 | 448.3 | 100.5 | | |
| 291 | ARSE | 415 | 214706 | 404.4 | 35.9 | | |
| 292 | ITGB8 | 3696 | 11202 | 286.5 | 35.1 | | |
| 292 | ITGB8 | 3696 | 214742 | 299.2 | 11.2 | | |
| 306 | NR2F2 | 7026 | 5922 | 370.5 | 65.9 | 483.1 | 109.7 |
| 306 | NR2F2 | 7026 | 45374 | 512.2 | 56.6 | 575.2 | 46.1 |
| 306 | NR2F2 | 7026 | 212809 | 96.9 | 10.6 | | |
| 309 | ADAM18 | 8749 | 21148 | 103.4 | 9.0 | 130.6 | 29.4 |
| 309 | ADAM18 | 8749 | 104119 | 86.5 | 27.3 | 95.0 | 17.2 |
| 309 | ADAM18 | 8749 | 104120 | 200.0 | 10.5 | 198.5 | 34.9 |
| 309 | ADAM18 | 8749 | 212787 | 515.1 | 8.9 | 547.4 | 30.2 |
| 334 | ARHGEF2 | 9181 | 119230 | 296.2 | 71.6 | | |
| 334 | ARHGEF2 | 9181 | 214562 | 334.6 | 18.9 | | |
| 352 | PRP2 | 134285 | 43202 | 356.9 | 43.3 | | |
| 352 | LOC134285 | 134285 | 46549 | 116.9 | 5.5 | 110.9 | 17.8 |
| 352 | LOC134285 | 134285 | 128215 | 84.3 | 4.5 | | |
| 352 | LOC134285 | 134285 | 212841 | 59.2 | 4.2 | | |
| 363 | PLA2R1 | 22925 | 108254 | 364.3 | 36.6 | | |
| 363 | PLA2R1 | 22925 | 214723 | 67.2 | 6.2 | | |
| 390 | CREB5 | 9586 | 116760 | 292.1 | 66.1 | | |
| 390 | CREB5 | 9586 | 214882 | 139.2 | 20.1 | | |
| 413 | FEN1 | 2237 | 121476 | 268.3 | 27.9 | | |
| 413 | FEN1 | 2237 | 214763 | 195.5 | 15.1 | | |
| 435 | PRSS15 | 9361 | 105664 | 334.5 | 21.5 | 357.9 | 13.1 |
| 435 | PRSS15 | 9361 | 212757 | 71.9 | 6.9 | | |
| 435 | PRSS15 | 9361 | 212758 | 308.8 | 16.0 | | |
| 441 | SYNJ1 | 8867 | 104702 | 375.3 | 12.5 | 419.8 | 11.9 |
| 441 | SYNJ1 | 8867 | 212746 | 41.3 | 5.5 | | |
| 441 | SYNJ1 | 8867 | 212747 | 211.3 | 45.9 | | |
| 459 | PCYT1B | 9468 | 111523 | 313.2 | 24.3 | | |
| 459 | PCYT1B | 9468 | 214260 | 309.2 | 13.2 | 340.4 | 30.6 |
| 486 | FLJ21736 | 79984 | 119838 | 486.4 | 27.2 | | |
| 486 | FLJ21736 | 79984 | 235619 | 308.9 | 34.1 | | |
| 489 | GPR10 | 2834 | 103837 | 573.3 | 59.1 | | |
| 489 | GPR10 | 2834 | 235614 | 222.4 | 10.1 | | |
| 495 | KIR2DS1 | 3806 | 212646 | 830.9 | 16.5 | | |
| 495 | KIR2DS1 | 3806 | 235634 | 312.5 | 41.6 | | |
| 496 | KIR2DS3 | 3808 | 213159 | 884.3 | 43.1 | | |
| 496 | KIR2DS3 | 3808 | 235633 | 337.0 | 2.7 | | |
| 498 | LOC135896 | 135896 | 213242 | 665.1 | 14.1 | | |
| 498 | LOC135896 | 135896 | 235629 | 131.6 | 9.4 | | |
| 506 | MOXD1 | 26002 | 111923 | 378.7 | 13.1 | | |
| 506 | MOXD1 | 26002 | 235615 | 343.3 | 73.0 | | |
| 507 | MPN2 | 339501 | 113991 | 276.2 | 35.2 | | |
| 507 | MPN2 | 339501 | 235626 | 307.6 | 54.9 | | |
| 514 | PEPD | 5184 | 105302 | 218.9 | 29.3 | 273.5 | 58.1 |
| 514 | PEPD | 5184 | 212688 | 301.3 | 62.5 | | |

TABLE 12

| | | | | Proliferation | Proliferation | Proliferation | Proliferation |
|---|---|---|---|---|---|---|---|
| Target No | Target Symbol | Gene Id | siRNA ID | run1 Mean % | run1 SD % | run2 Mean % | run2 SD % |
| 2 | HLCS | 3141 | 117851 | 87.0 | 2.8 | | |
| 2 | HLCS | 3141 | 117852 | 63.8 | 15.4 | | |
| 2 | HLCS | 3141 | 117853 | 103.0 | 6.6 | | |
| 3 | SC4MOL | 6307 | 117416 | 106.8 | 9.2 | | |
| 3 | SC4MOL | 6307 | 117417 | 78.9 | 6.3 | | |
| 3 | SC4MOL | 6307 | 117418 | 91.4 | 13.5 | | |
| 4 | CASP1 | 834 | 42626 | 104.4 | 8.3 | | |
| 4 | CASP1 | 834 | 42711 | 102.1 | 9.8 | | |
| 7 | CTSE | 1510 | 105039 | 125.9 | 4.7 | | |
| 7 | CTSE | 1510 | 105040 | 125.0 | 5.0 | | |
| 7 | CTSE | 1510 | 105041 | 117.3 | 16.3 | | |
| 8 | FRK | 2444 | 1147 | 110.3 | 4.7 | | |
| 8 | FRK | 2444 | 1243 | 119.8 | 14.1 | | |
| 8 | FRK | 2444 | 1338 | 127.9 | 8.4 | | |
| 9 | HMBS | 3145 | 8225 | 99.7 | 3.5 | | |
| 9 | HMBS | 3145 | 117844 | 109.2 | 13.3 | | |
| 10 | HSD17B4 | 3295 | 106087 | 98.7 | 6.8 | | |
| 10 | HSD17B4 | 3295 | 106089 | 81.6 | 4.2 | 82.1 | 15.4 |
| 11 | LCN2 | 3934 | 121011 | 93.7 | 16.8 | | |
| 11 | LCN2 | 3934 | 121012 | 113.9 | 20.4 | | |
| 12 | OXTR | 5021 | 1766 | 103.1 | 9.1 | | |
| 12 | OXTR | 5021 | 1859 | 103.7 | 8.2 | | |
| 12 | OXTR | 5021 | 1947 | 114.2 | 8.3 | | |
| 13 | PPP1R3C | 5507 | 142834 | 108.9 | 14.8 | | |
| 13 | PPP1R3C | 5507 | 142836 | 104.3 | 10.4 | | |
| 16 | TPI1 | 7167 | 43820 | 102.9 | 5.1 | | |
| 16 | TPI1 | 7167 | 43916 | 97.3 | 3.2 | | |
| 18 | SLC30A2 | 7780 | 117693 | 101.4 | 2.0 | | |
| 18 | SLC30A2 | 7780 | 117695 | 120.1 | 3.1 | | |
| 19 | ULK1 | 8408 | 118260 | 100.8 | 4.6 | | |
| 19 | ULK1 | 8408 | 118261 | 96.1 | 8.5 | | |
| 20 | GLP2R | 9340 | 5053 | 123.0 | 5.0 | | |
| 20 | GLP2R | 9340 | 5146 | 121.2 | 4.8 | | |
| 20 | GLP2R | 9340 | 5237 | 107.7 | 11.1 | | |
| 22 | SPUVE | 11098 | 19104 | 83.5 | 12.5 | 97.4 | 5.1 |
| 22 | SPUVE | 11098 | 19196 | 90.3 | 14.6 | 115.2 | 1.2 |
| 22 | SPUVE | 11098 | 212941 | 93.4 | 4.0 | 101.1 | 0.2 |
| 22 | SPUVE | 11098 | 212942 | 99.4 | 10.7 | | |
| 23 | PASK | 23178 | 978 | 104.26 | 4.35 | | |
| 23 | PASK | 23178 | 103354 | 90.19 | 2.06 | | |
| 24 | OR52A1 | 23538 | 2061 | 113.7 | 3.1 | | |
| 24 | OR52A1 | 23538 | 2153 | 114.2 | 2.6 | | |
| 24 | OR52A1 | 23538 | 2240 | 81.6 | 7.2 | | |
| 25 | RGS17 | 26575 | 20024 | 95.6 | 7.0 | | |
| 25 | RGS17 | 26575 | 20115 | 91.2 | 6.9 | | |
| 26 | MYLIP | 29116 | 118397 | 91.6 | 15.2 | | |
| 26 | MYLIP | 29116 | 118398 | 110.6 | 5.3 | | |
| 27 | PKD1L2 | 114780 | 104830 | 91.8 | 11.1 | 93.2 | 7.0 |
| 27 | PKD1L2 | 114780 | 104835 | 82.5 | 6.3 | | |
| 28 | BPHL | 670 | 119221 | 92.9 | 14.3 | | |
| 28 | BPHL | 670 | 214767 | 106.0 | 6.0 | | |
| 29 | USP3 | 9960 | 105141 | 84.0 | 9.3 | | |
| 29 | USP3 | 9960 | 214567 | 106.4 | 6.1 | | |
| 31 | KCNK17 | 89822 | 43781 | 73.0 | 2.6 | 94.8 | 5.1 |
| 31 | KCNK17 | 89822 | 212814 | 118.0 | 17.5 | | |
| 31 | KCNK17 | 89822 | 212815 | 103.0 | 2.6 | | |
| 32 | WEE1 | 7465 | 405 | 98.4 | 12.9 | | |
| 32 | WEE1 | 7465 | 103582 | 112.7 | 3.1 | | |
| 32 | WEE1 | 7465 | 103636 | 90.5 | 3.8 | | |
| 32 | WEE1 | 7465 | 212739 | 35.7 | 11.2 | 52.9 | 8.8 |
| 34 | RNUT1 | 10073 | 117371 | 66.7 | 9.3 | | |
| 34 | RNUT1 | 10073 | 214780 | 104.1 | 4.3 | | |
| 35 | M6PR | 4074 | 111157 | 72.8 | 2.7 | | |
| 35 | M6PR | 4074 | 214744 | 112.1 | 26.0 | | |
| 36 | MGC39650 | 147011 | 38979 | 77.3 | 20.6 | | |
| 36 | MGC39650 | 147011 | 214871 | 82.4 | 6.0 | | |
| 37 | FLJ22761 | 80201 | 121933 | 92.4 | 9.2 | | |
| 37 | FLJ22761 | 80201 | 214839 | 119.8 | 3.3 | | |
| 38 | PAPOLG | 64895 | 119829 | 92.2 | 13.0 | | |
| 38 | PAPOLG | 64895 | 214280 | 102.5 | 29.7 | 86.8 | 14.9 |
| 39 | DNM1L | 10059 | 19471 | 88.8 | 7.1 | | |
| 39 | DNM1L | 10059 | 214799 | 107.3 | 3.9 | | |
| 42 | FKSG79 | 84636 | 6196 | 82.6 | 14.7 | | |
| 42 | FKSG79 | 84636 | 214845 | 105.4 | 16.3 | | |
| 43 | LCN7 | 64129 | 105753 | 83.4 | 11.2 | | |
| 43 | LCN7 | 64129 | 214577 | 98.6 | 11.2 | | |
| 45 | RASGRP2 | 10235 | 120445 | 92.1 | 3.2 | | |
| 45 | RASGRP2 | 10235 | 214874 | 71.2 | 19.6 | | |
| 51 | PRPSAP2 | 5636 | 117084 | 87.8 | 15.2 | | |
| 51 | PRPSAP2 | 5636 | 214750 | 103.8 | 22.9 | | |
| 52 | SLC26A10 | 65012 | 119926 | 87.2 | 5.9 | | |
| 52 | SLC26A10 | 65012 | 214589 | 114.7 | 5.2 | | |
| 54 | OBP2A | 29991 | 121179 | 80.3 | 9.5 | | |
| 54 | OBP2A | 29991 | 214904 | 74.8 | 5.7 | | |
| 56 | TNFRSF13B | 23495 | 111813 | 63.3 | 6.1 | | |
| 56 | TNFRSF13B | 23495 | 214802 | 92.1 | 9.8 | | |
| 62 | CTSK | 1513 | 105010 | 87.7 | 11.9 | | |
| 62 | CTSK | 1513 | 214550 | 89.6 | 5.2 | | |
| 72 | D4ST-1 | 113189 | 112330 | 85.2 | 5.6 | 88.2 | 12.3 |
| 72 | D4ST-1 | 113189 | 214285 | 117.2 | 19.8 | 103.6 | 8.7 |
| 73 | APCL | 10297 | 121576 | 95.3 | 23.9 | | |
| 73 | APCL | 10297 | 214783 | 64.7 | 8.2 | | |
| 79 | ARHGEF1 | 9138 | 119422 | 103.8 | 20.4 | | |
| 79 | ARHGEF1 | 9138 | 214561 | 98.2 | 10.1 | | |
| 81 | MCCC1 | 56922 | 118817 | 85.7 | 5.4 | | |
| 81 | MCCC1 | 56922 | 214276 | 99.8 | 16.5 | 84.0 | 3.1 |
| 88 | GALR2 | 8811 | 4818 | 136.1 | 7.5 | | |
| 88 | GALR2 | 8811 | 44971 | 96.8 | 5.4 | | |
| 88 | GALR2 | 8811 | 45063 | 126.0 | 6.9 | | |
| 88 | GALR2 | 8811 | 212858 | 94.0 | 6.0 | 84.3 | 13.0 |
| 117 | SMPD1 | 6609 | 8630 | 92.9 | 8.0 | | |
| 117 | SMPD1 | 6609 | 8725 | 92.4 | 5.6 | | |
| 117 | SMPD1 | 6609 | 8816 | 94.5 | 1.7 | | |
| 117 | SMPD1 | 6609 | 212695 | 102.2 | 10.0 | | |
| 143 | SCP2 | 6342 | 119182 | 95.7 | 3.2 | | |
| 143 | SCP2 | 6342 | 214903 | 71.3 | 9.7 | | |
| 163 | CCM1 | 889 | 15563 | 81.7 | 14.4 | | |
| 163 | CCM1 | 889 | 214883 | 69.2 | 31.6 | | |
| 171 | PAFAH2 | 5051 | 119066 | 95.3 | 10.1 | | |
| 171 | PAFAH2 | 5051 | 214710 | 105.3 | 11.1 | | |
| 173 | NLGN3 | 54413 | 121059 | 117.4 | 17.8 | | |
| 173 | NLGN3 | 54413 | 214826 | 109.0 | 6.4 | | |
| 179 | VN1R2 | 317701 | 43139 | 91.6 | 4.2 | | |
| 179 | VN1R2 | 317701 | 43208 | 89.8 | 4.5 | | |
| 179 | VN1R2 | 317701 | 43274 | 121.2 | 16.0 | | |
| 179 | VN1R2 | 317701 | 212843 | 91.7 | 7.3 | | |
| 180 | GAD2 | 2572 | 9108 | 88.0 | 8.3 | | |
| 180 | GAD2 | 2572 | 214716 | 84.1 | 7.3 | | |
| 205 | HTR2C | 3358 | 1758 | 124.4 | 3.3 | | |
| 205 | HTR2C | 3358 | 1852 | 119.6 | 12.3 | | |
| 205 | HTR2C | 3358 | 1940 | 117.3 | 5.6 | | |
| 205 | HTR2C | 3358 | 144642 | 89.8 | 10.4 | | |
| 205 | HTR2C | 3358 | 212705 | 96.2 | 3.6 | | |
| 215 | LOC345667 | 11174 | 104231 | 117.6 | 3.2 | | |
| 215 | LOC345667 | 11174 | 104232 | 128.2 | 7.2 | | |
| 215 | LOC345667 | 11174 | 104267 | 118.2 | 9.9 | | |
| 215 | LOC345667 | 11174 | 212853 | 80.6 | 13.6 | | |
| 237 | GPR3 | 2827 | 1785 | 71.7 | 2.4 | 96.5 | 17.2 |
| 237 | GPR3 | 2827 | 212766 | 115.2 | 5.0 | | |
| 240 | FLJ32389 | 126393 | 122036 | 116.0 | 5.9 | | |
| 240 | FLJ32389 | 126393 | 214864 | 116.5 | 7.5 | | |
| 241 | SERPINA7 | 6906 | 118553 | 99.6 | 19.0 | | |
| 241 | SERPINA7 | 6906 | 214548 | 99.4 | 7.3 | | |
| 242 | DCTD | 1635 | 119612 | 94.2 | 4.9 | | |
| 242 | DCTD | 1635 | 214555 | 101.1 | 10.8 | | |
| 261 | ACLY | 47 | 116939 | 82.9 | 31.2 | | |
| 261 | ACLY | 47 | 214886 | 91.1 | 20.5 | | |
| 273 | AGXT2L1 | 64850 | 112237 | 86.0 | 0.8 | | |
| 273 | AGXT2L1 | 64850 | 214281 | 105.9 | 1.1 | 109.2 | 20.2 |
| 291 | ARSE | 415 | 119012 | 103.0 | 13.4 | | |
| 291 | ARSE | 415 | 214706 | 85.4 | 10.0 | | |
| 292 | ITGB8 | 3696 | 11202 | 116.4 | 20.3 | | |
| 292 | ITGB8 | 3696 | 214742 | 67.6 | 1.7 | | |
| 306 | NR2F2 | 7026 | 5922 | 101.6 | 8.2 | 89.4 | 21.3 |
| 306 | NR2F2 | 7026 | 45374 | 128.5 | 7.8 | | |
| 306 | NR2F2 | 7026 | 212809 | 104.4 | 13.4 | | |
| 309 | ADAM18 | 8749 | 21148 | 133.6 | 5.4 | | |
| 309 | ADAM18 | 8749 | 104119 | 114.7 | 5.7 | | |
| 309 | ADAM18 | 8749 | 104120 | 103.3 | 6.7 | | |
| 309 | ADAM18 | 8749 | 212787 | 76.6 | 3.4 | 82.3 | 13.2 |
| 334 | ARHGEF2 | 9181 | 119230 | 87.1 | 10.5 | | |
| 334 | ARHGEF2 | 9181 | 214562 | 107.3 | 11.4 | | |
| 352 | PRP2 | 134285 | 43202 | 94.9 | 2.7 | | |
| 352 | LOC134285 | 134285 | 46549 | 96.8 | 4.8 | | |
| 352 | LOC134285 | 134285 | 128215 | 97.3 | 23.2 | | |
| 352 | LOC134285 | 134285 | 212841 | 85.1 | 12.9 | | |
| 363 | PLA2R1 | 22925 | 108254 | 100.0 | 15.7 | | |
| 363 | PLA2R1 | 22925 | 214723 | 108.5 | 3.2 | | |
| 390 | CREB5 | 9586 | 116760 | 110.1 | 13.2 | | |
| 390 | CREB5 | 9586 | 214882 | 99.4 | 8.7 | | |
| 413 | FEN1 | 2237 | 121476 | 99.5 | 7.2 | | |
| 413 | FEN1 | 2237 | 214763 | 100.8 | 8.4 | | |
| 435 | PRSS15 | 9361 | 105664 | 83.2 | 4.1 | 107.9 | 9.5 |
| 435 | PRSS15 | 9361 | 212757 | 110.9 | 12.5 | | |
| 435 | PRSS15 | 9361 | 212758 | 94.4 | 17.8 | | |
| 441 | SYNJ1 | 8867 | 104702 | 105.8 | 1.9 | 112.4 | 11.6 |
| 441 | SYNJ1 | 8867 | 212746 | 91.0 | 3.2 | | |
| 441 | SYNJ1 | 8867 | 212747 | 110.6 | 15.9 | | |
| 452 | SULT4A1 | 25830 | 111874 | 91.9 | 8.9 | | |
| 452 | SULT4A1 | 25830 | 214271 | 111.7 | 18.6 | 93.1 | 12.2 |
| 459 | PCYT1B | 9468 | 111523 | 75.4 | 4.6 | | |
| 459 | PCYT1B | 9468 | 214260 | 117.0 | 5.0 | 95.7 | 9.1 |
| 486 | FLJ21736 | 79984 | 119838 | 99.7 | 10.8 | | |
| 486 | FLJ21736 | 79984 | 235619 | 113.0 | 11.4 | | |
| 489 | GPR10 | 2834 | 103837 | 108.6 | 9.9 | | |
| 489 | GPR10 | 2834 | 235614 | 98.6 | 9.6 | | |
| 495 | KIR2DS1 | 3806 | 212646 | 62.4 | 13.1 | | |
| 495 | KIR2DS1 | 3806 | 235634 | 104.4 | 5.1 | | |
| 496 | KIR2DS3 | 3808 | 213159 | 93.7 | 13.2 | | |
| 496 | KIR2DS3 | 3808 | 235633 | 99.8 | 8.6 | | |
| 498 | LOC135896 | 135896 | 213242 | 88.5 | 11.2 | | |
| 498 | LOC135896 | 135896 | 235629 | 104.8 | 14.1 | | |
| 506 | MOXD1 | 26002 | 111923 | 95.6 | 19.1 | | |
| 506 | MOXD1 | 26002 | 235615 | 116.1 | 20.7 | | |
| 507 | MPN2 | 339501 | 113991 | 93.3 | 12.0 | | |
| 507 | MPN2 | 339501 | 235626 | 105.9 | 16.5 | | |
| 514 | PEPD | 5184 | 105302 | 76.9 | 8.4 | 99.7 | 17.6 |
| 514 | PEPD | 5184 | 212688 | 108.9 | 20.3 | | |

1. Method for identifying a compound as being useful in the treatment or prophylaxis of a disease, comprising the steps of
(a) providing a first cell expressing a target polypeptide selected from the group listed in Table 10, or a fragment, or a derivative thereof;
(b) exposing said first cell to a candidate compound;
(c) determining a first level of an activity or property, said activity or property being affected by an activity or property of said target polypeptide; and
(d) selecting or discarding said candidate compound, based on a comparison of said first level of said activity or property with a reference level of said activity or property;
characterised in that
said disease is A disease selected from the group comprising cardiovascular diseases, disorders of lipid metabolism or atherosclerosis.

2. Use of a method of claim 1 for the screening for substances useful in the treatment or prophylaxis of A disease selected from the group comprising cardiovascular diseases, disorders of lipid metabolism or atherosclerosis.

3. Method of claim 1 or use of claim 2, wherein said host cell expresses said target polypeptide above wild-type level.

4. Method or use of any of claims 1 to 3, wherein said target polypeptide expression is recombinant polypeptide expression.

5. Method or use of any of claims 1 to 4, wherein said compound is selected if said first level of said activity or property is lower than said reference level of said activity or property.

6. Method or use of any of claims 1 to 4, wherein said compound is selected if said first level of said activity or property is higher than said reference level of said activity or property.

7. Method or use of any of claims 1 to 6, wherein said reference level is a level obtained from a second cell expressing the target polypeptide at a lower level as compared to said first cell.

8. Method or use of any of claims 1 to 6, wherein said reference level is the level obtained with said first cell in the absence of the candidate compound.

9. Method or use of any of claims 1 to 8, wherein said method further comprises contacting the host cell with a known agonist or antagonist of the target polypeptide before determining the first level.

10. Method or use of any of claims 1 to 9, wherein said activity or property being affected by said activity or property of said target polypeptide is binding affinity of said compound to said target polypeptide.

11. Use of a method, said method comprising the steps of
(a) culturing a population of cells expressing a target polypeptide listed in Table 10, or a functional fragment or derivative thereof;
(b) determining a first level of expression and/or activity of said target protein in said population of cells;
(c) exposing said population of cells to a compound, or a mixture of compounds;
(d) determining a second level of expression and/or activity of said target polypeptide in said population of cells during or after said exposure of said population of cells to the compound, or the mixture of compounds; and
(e) comparing said first and said second level;
for the screening for substances useful in the treatment or prophylaxis of A disease selected from the group comprising cardiovascular diseases, disorders of lipid metabolism or atherosclerosis.

12. Method or use of any of claims 1 to 11, wherein said first level of an activity or property is determined with a reporter, said reporter being controlled by a promoter responsive to at least one second messenger.

13. Method or use of claim 12, wherein said at least one second messenger is cyclic AMP, or Ca2+, or both.

14. Method or use of claim 12 or 13, wherein said promoter is a cyclic AMP-responsive promoter, an NF-KB responsive promoter, a NF-AT responsive promoter, or a promoter responsive to transcription factors or to nuclear hormone receptors.

15. Method or use of any of claims 12 to 14, wherein the reporter is luciferase or beta-galactosidase.

16. Method or use of any of claims 1 to 15, wherein the compound is a low molecular weight compound.

17. Method or use of any of claims 1 to 15, wherein the compound is a polypeptide.

18. Method or use of any of claims 1 to 15, wherein the compound is a lipid.

19. Method or use of any of claims 1 to 15, wherein the compound is a natural compound.

20. Method or use of any of claims 1 to 15, wherein the compound is an antibody or a nanobody.

21. Method for identifying a compound as being useful in the treatment or prophylaxis of a disease, comprising the steps of
(a) contacting said compound with a target polypeptide selected from the group listed in Table 10, or a fragment, or a derivative thereof;
(b) detect binding of said compound to said target polypeptide or detect a change in activity of said target polypeptide;
(c) selecting said compound If binding is detected in step (b) or if a change in activity is detected in step (b);
characterised in that
said disease is A disease selected from the group comprising cardiovascular diseases, disorders of lipid metabolism or atherosclerosis.

22. Use of a method of claim 21 for screening for compounds, useful in the treatment or prophylaxis of A disease selected from the group comprising cardiovascular diseases, disorders of lipid metabolism or atherosclerosis.

23. Method or use of any of claims 21 to 22, wherein binding is detected in vitro.

24. Method or use of any of claims 21 to 23, wherein said target polypeptide is a recombinant polypeptide.

25. Method or use of any of claims 21 to 24, wherein said compound is selected if the binding affinity is equal to or lower than 10 micromolar.

26. Method or use of any of claims 21 to 25, wherein said compound is a low molecular weight compound.

27. Method or use of any of claims 21 to 25, wherein said compound is a polypeptide, or a lipid, or a natural compound, or an antibody or a nanobody.

28. Use of a compound that inhibits an activity and/or the expression of any of the polypeptides listed in Table 10 in the manufacture of a medicament for the treatment or prophylxis of A disease selected from the group comprising cardiovascular diseases, disorders of lipid metabolism or atherosclerosis.

29. Use of claim 28, wherein said compound is identified according to any one of the methods or uses of claims 1 to 27.

30. Use of an agent inhibiting the expression of a polypeptide selected from the group listed in Table 10 for the preparation of a medicament for the treatment or prophylaxis of A disease selected from the group comprising cardiovascular diseases, disorders of lipid metabolism or atherosclerosis.

31. Use of claim 30, wherein said agent is selected from the group consisting of an antisense RNA encoding said polypeptide;
a ribozyme that cleaves the polyribonucleotide encoding said polypeptide;
an antisense oligodeoxynucleotide (ODN) enconding said polypeptide;
a small interfering RNA (siRNA) that is sufficiently homologous to a portion of the polyribonucleotide such that said siRNA is capable of inhibiting the polyribonucleotide that would otherwise cause the production of said polypeptide;
a small interfering RNA (siRNA) having the sequence of any of SEQ ID NO:1 to 172;
a microRNA (miRNA) suitable for inhibition of a polypeptide selected from the group listed in Table 10; or
a short hairpin RNA (shRNA) suitable for silencing expression of a polypeptide selected from the group listed in Table 10.

32. Use of claim 31, wherein the nucleotide sequence of said agent is present in a vector.

33. Use of claim 32, wherein the vector is an adenovirus, a retrovirus, an alphavirus, an adeno-associated virus (AAV), a lentivirus, a herpes simplex virus (HSV) or a sendai virus.

34. Use of any of claims 31 to 33, wherein said agent is siRNA, and said siRNA comprises a sense strand of 17 to 31 nucleotides which is identical to a region of the coding sequence, or its complementary sequence, of any of the polypeptides of Table 10.

35. Use of claim 34, wherein the siRNA further comprises a cleavable loop region connecting the sense and the antisense strand.

36. Vector comprising any of SEQ ID NO:1 to 172

37. Use of a vector of claim 36 as a medicament.

38. Use of a vector of claim 37 for the manufacture of a medicament useful in the treatment or prophylaxis of A disease selected from the group comprising cardiovascular diseases, disorders of lipid metabolism or atherosclerosis.

39. Use according to claim 37 or 38, wherein the vector is an adenoviral, retroviral, adeno-associated viral, lentiviral or a sendaiviral vector.

40. Method for diagnosing a pathological condition involving A disease selected from the group comprising cardiovascular diseases, disorders of lipid metabolism or atherosclerosis, or a susceptibility to said condition in a subject, comprising
(a) obtaining a sample of the subject's mRNA corresponding to a polypeptide selected from the group listed in Table 10, or a sample of the subject's genomic DNA corresponding to a polypeptide of Table 10;
(b) determining the nucleic acid sequence of said mRNA or said genomic DNA;
(c) obtaining the nucleic acid sequence encoding said polypeptide of Table 10 from a public database; and
(d) identifying any difference(s) between the nucleic acid sequences determined in step (b) and (c);
wherein a pathological condition involving a disease selected from the group comprising cardiovascular diseases, disorders of lipid metabolism or a disease selected from the group comprising cardiovascular diseases, disorders of lipid metabolism or atherosclerosis, or a susceptibility to such a condition in a subject is diagnosed, if such difference(s) are identified in step (d).

41. Method for diagnosing a pathological condition involving A disease selected from the group comprising cardiovascular diseases, disorders of lipid metabolism or atherosclerosis or a susceptibility to such a condition in a subject, comprising
(a) determining the amount of a polypeptide of Table 10 in a biological sample of said subject; and
(b) comparing the amount determined in (a) with a the amount of the polypeptide in a healthy subject;
wherein an increase or a decrease of the amount of said polypeptide compared to the amount present in a healthy subject is indicative of the presence of the pathological condition.
